(19) United States
(12) Patent Application Publication
(10) Pub. No.: US 2023/0227492 A1
(43) Pub. Date: Jul. 20, 2023

(54) MONOMERIC AND OLIGOMERIC COMPOUND EMBODIMENTS AS CONTRACEPTIVES AND THERAPIES AND METHODS OF MAKING AND USING THE SAME

(71) Applicants: The USA as represented by the Secretary, Department of Health and Human Services, Bethesda, MD (US); Ash Stevens, LLC, Detroit, MI (US)

(72) Inventors: Min Lee, San Diego, CA (US); Diana Blithe, Silver Spring, MD (US); Ming-Teh Lin, Farmington Hills, MI (US); Pranab Gupta, West Bloomfield, MI (US); Bashir Kaskar, Troy, MI (US)

(73) Assignees: The USA as represented by the Secretary, Department of Health and Human Services, Bethesda, MD (US); Ash Stevens, LLC, Detroit, MI (US)

(21) Appl. No.: 18/009,571

(22) PCT Filed: Jun. 10, 2021
(86) PCT No.: PCT/US2021/036809

§ 371 Date: Dec. 9, 2022

Related U.S. Application Data
(60) Provisional application No. 63/037,952, filed on Jun. 11, 2020.

(51) Int. Cl. C07J 43/00; C07J 1/00; A61P 15/16

(52) U.S. Cl. CPC C07J 43/003; C07J 1/0074; C07J 1/0096; A61P 15/16; C07B 2200/05

(57) ABSTRACT

Disclosed herein are monomeric and oligomeric compound embodiments for use as contraceptive agents. Monomeric compound embodiments disclosed herein comprise substituents that facilitate the ability of the compounds to exhibit progestogenic, androgenic, and estrogenic activity, which can prevent or inhibit bone density loss in subjects. Oligomeric compound embodiments disclosed herein provide the ability to control receptor activation and/or treatment by incorporating a tunable linker group which couples steroidal-based compounds to one another or with therapeutic agents and facilitates selective cleavage of the monomeric components of the oligomeric compound.

### CROSS REFERENCE TO RELATED APPLICATION

This application claims the benefit of the earlier filing date of U.S. Provisional Application No. 63/037,952, filed on Jun. 11, 2020, the entirety of which is incorporated herein by reference.

### ACKNOWLEDGMENT OF GOVERNMENT SUPPORT

This invention was made with government support under project number ZHD008981-01, awarded by the National Institutes of Health, Eunice Kennedy Shriver National Institute of Child Health and Human Development. The government has certain rights in the invention.

### FIELD

Disclosed herein monomeric and oligomeric compound embodiments for use as contraceptive agents and/or therapeutics, and methods of making and using the same.

### BACKGROUND

While species of contraceptive agents based on steroidal compounds exist in the art for female contraception, the development of male contraceptives is less advanced and there are no approved male contraceptives currently available for use. The male contraceptive agents currently in clinical trial, lack the ability to exhibit estrogenic properties and thus can lead to decreased circulating estrogen in subjects. Decreased estrogen circulation has a negative impact on bone density and can lead to osteoporosis. As such, there exists a need in the art for contraceptive agents that can provide androgenic and progestogenic properties and that can further exhibit estrogenic properties thereby reducing or inhibiting bone density loss. There also exists a need in the art for contraceptive agents that can serve dual purpose roles, such as steroidal receptor activation/deactivation in combination with therapeutic activity against diseases or other maladies.

### SUMMARY

Disclosed herein are embodiments of a compound of Formula I, or a pharmaceutically acceptable salt, a prodrug, a solvate, or a tautomer thereof

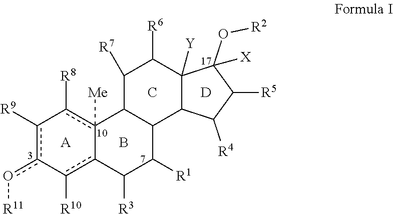

wherein

R¹ is selected from H, D, halogen, aliphatic, aromatic, heteroaliphatic, haloaliphatic, haloheteroaliphatic, heteroaliphatic, or an organic functional group;

R² is selected from —C(O)R^{a}, —C(O)OR^{a}, —C(O)NR^{b}R^{c}, wherein each R^{a} independently is selected from aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; —P(O)(OR^{a})₂, wherein each R^{a} independently is H, aliphatic, aromatic, heteroaliphatic, haloaliphatic, haloheteroaliphatic, heteroaliphatic, or an organic functional group; or —S(O)₂R^{a} wherein R^{a} is H, aliphatic, aromatic, heteroaliphatic, haloaliphatic, haloheteroaliphatic, heteroaliphatic, or an organic functional group;

each of R³, R⁴, R⁵, R⁶, R⁷, R⁸, R⁹, and R¹⁰ independently is selected from H, D, aliphatic, aromatic, heteroaliphatic, haloaliphatic, haloheteroaliphatic, heteroaliphatic, or an organic functional group;

R¹¹, if present, is selected from hydrogen, aliphatic, heteroaliphatic, aromatic, heteroaliphatic, haloaliphatic, haloheteroaliphatic, or an organic functional group; X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; and

Y is aliphatic.

Also disclosed are embodiments of a pharmaceutically acceptable composition, comprising a compound according to any or all of the above compound embodiments, a pharmaceutically acceptable salt, a prodrug, a solvate, or a tautomer thereof and a pharmaceutically acceptable excipient.

Also disclosed are embodiments of a dosage form, comprising a compound according to any or all of the above compound embodiments, or a pharmaceutically acceptable salt, a prodrug, a solvate, or a tautomer thereof; or the pharmaceutically acceptable composition according to any or all of the above composition embodiments.

Also disclosed are embodiments of an oligomer compound, comprising a first steroidal-based compound covalently coupled to a first linker group via an oxygen atom attached to a functional group positioned at C17 of the steroidal-based compound, and wherein the first linker group is further covalently coupled to a second steroidal-based compound or a therapeutic agent.

Also disclosed are embodiments of a composition comprising the oligomer according to any or all of the above oligomer embodiments, or a pharmaceutically acceptable salt, a prodrug, a solvate, or a tautomer thereof; and a pharmaceutically acceptable excipient.

Also disclosed are embodiments of a dosage form, comprising an oligomer according to any or all of the above oligomer embodiments, or a pharmaceutically acceptable salt, a prodrug, a solvate, or a tautomer thereof.

Also disclosed are embodiments of a method, comprising administering to a subject a compound according to any or all of the above compound embodiments, or an oligomer according to any or all of the above oligomer embodiments.

Also disclosed herein are embodiments of a method, comprising administering to a subject a therapeutically active amount of the dosage form according to any or all of the above dosage form embodiments.

Also disclosed are uses of the compound according to any or all of the above compound embodiments, or an oligomer according to any or all of the above oligomer embodiments.

Also disclosed are embodiments of a method for making the compound according to any or all of the above compound embodiments, comprising: performing a conjugate addition and deprotection reaction on a protecting-group containing precursor compound using a lithium compound, a catalyst, a Grignard reagent, and a silyl reagent to provide a substituted, deprotected product; and functionalizing the substituted, deprotected product to provide the compound; wherein the protecting-group containing precursor compound has a formula

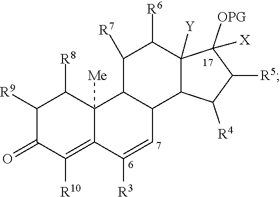

and the substituted, deprotected product has a formula

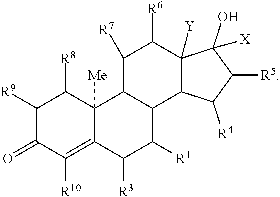

Also disclosed are embodiments of a method for making the oligomer according to any or all of the above oligomer embodiments, comprising: covalently coupling a linker group precursor and (i) one of the first steroidal-based compound or the second steroidal-based compound, or (ii) the therapeutic agent using an esterifying reagent to form either a linker-functionalized steroidal-based compound or a linker-functionalized therapeutic agent; and covalently coupling the linker-functionalized steroidal-based compound to the other of the first or second steroidal-based compound; or covalently coupling the linker-functionalized therapeutic agent to the first steroidal-based compound.

The foregoing and other objects and features of the present disclosure will become more apparent from the following detailed description, which proceeds with reference to the accompanying figures.

### BRIEF DESCRIPTION OF THE DRAWINGS

FIG. 1 is a graph of ventral prostate gland average weight from castrated weanling rats with a single dose administration of 1.25 μmol CDB 4866 (a DMA homodimer), CDB 4867 (a DMA homodimer), CDB 4868 (a DMA homodimer), and CDB 4877 (a testosterone homodimer) in a 10% ethyl alcohol/90% sesame oil formulation.

FIG. 2 is a graph of Levator ani muscle average weight from castrated weanling rats with a single dose administration of 1.25 CDB 4866, CDB 4867, CDB 4868, and CDB 4877 in a 10% ethyl alcohol/90% sesame oil formulation.

FIG. 3 is a graph summarizing ventral prostate average weights for oligomeric compound embodiments CDB 4877, CDB 4866, CDB 4867, and CDB 4868.

FIG. 4 is a graph summarizing Levator ani average weights for oligomeric compound embodiments CDB 4877, CDB 4866, CDB 4867, and CDB 4868.

FIG. 5 is a graph summarizing ventral prostate weights for rats administered different doses of a vehicle, testosterone, and 7α-methyl testosterone, using a Hershberger assay and which shows the androgenic potency of 7α-methyl testosterone.

FIG. 6 is a graph summarizing average McPhail index values for rabbits administered different doses of a vehicle, testosterone, and 7α-methyl testosterone, using a Clauberg assay, and which shows the dose response of endometrial transformation and progestogenic potency of 7α-methyl testosterone.

### DETAILED DESCRIPTION

### I. Overview of Terms

The following explanations of terms and/or symbols are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

Certain functional group terms used herein include a symbol “-”, which is used to show how the defined functional group attaches to, or within, the compound to which it is bound. Also, a dashed bond (i.e., “---”) as used in certain formulas described herein indicates an “optional” bond to a substituent or atom of the formula other than hydrogen in the sense that the bond (and in some embodiments, the substituent) may or may not be present. In any formulas comprising a dashed bond, if the optional bond and/or any corresponding substituent is not present, then the valency requirements of any atom(s) bound thereto is completed by a bond to a hydrogen atom. Solely by way of example, in the following formula, the dashed bond between C10 and the illustrated methyl group may be present, or this bond and methyl substituent may be absent and instead a C10-H bond is present. Also, the dashed bonds in ring A indicate that double bonds may be present, or not present, in which case a single bond is present and the corresponding carbon atoms are bound to hydrogen atoms, in addition to any other substituents already bound thereto.

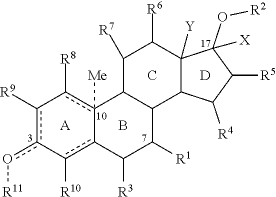

The symbol “ [symbol] ” is used to indicate a bond disconnection in abbreviated structures/formulas provided herein. A person of ordinary skill in the art recognizes that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

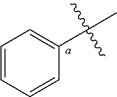 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g. around the amide bond or between two directly attached rings such as pyridinyl rings, biphenyl groups, and the like, atropisomers are also possible and are also specifically included in the compounds disclosed herein.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

As used herein, the term “substituted” refers to all subsequent modifiers in a term, for example in the term “substituted aliphatic-aromatic,” substitution may occur on the “aliphatic” portion, the “aromatic” portion or both portions of the aliphatic-aromatic group.

“Substituted,” when used to modify a specified group or moiety, means that at least one, and perhaps two or more, hydrogen atoms of the specified group or moiety is independently replaced with the same or different substituent groups. In a particular embodiment, a group, moiety, or substituent may be substituted or unsubstituted, unless expressly defined as either “unsubstituted” or “substituted.” Accordingly, any of the functional groups specified herein may be unsubstituted or substituted unless the context indicates otherwise or a particular structural formula precludes substitution. In particular embodiments, a substituent may or may not be expressly defined as substituted but is still contemplated to be optionally substituted. For example, an “aliphatic” or a “cyclic” moiety may be unsubstituted or substituted, but an “unsubstituted aliphatic” or an “unsubstituted cyclic” is not substituted. In one embodiment, a group that is substituted has at least one substituent up to the number of substituents possible for a particular moiety, such as 1 substituent, 2 substituents, 3 substituents, or 4 substituents.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

Acyl Halide: —C(O)X, wherein X is a halogen, such as Br, F, I, or Cl.

Administer (or Administering or Administration): To expose a subject to one or more monomeric compound embodiments, one or more oligomeric compound embodiments, or any compositions and/or formulations thereof. Modes of administration are discussed herein and can include, but are not limited to, topical, ocular, oral, buccal, systemic, nasal, injection (such as intravenous, intraperitoneal, subcutaneous, intramuscular, or intrathecal), transdermal (e.g., by mixing with a penetrating agent, such as DMSO), rectal, vaginal, a form suitable for administration by inhalation or insufflation, a form suitable for implantation, or any combination thereof. Administration as used herein also contemplates self-administration wherein the exposed subject carries out the administration.

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Aliphatic groups may be substituted with one or more groups other than hydrogen, such as aliphatic, heteroaliphatic, haloaliphatic, haloheteroaliphatic, aromatic, or an organic functional group.

Aliphatic-aromatic: An aromatic group that is or can be coupled to a compound disclosed herein, wherein the aromatic group is or becomes coupled through an aliphatic group. Aliphatic-aromatic groups may be substituted with one or more groups other than hydrogen, such as aliphatic, heteroaliphatic, haloaliphatic, haloheteroaliphatic, aromatic, or an organic functional group.

Aliphatic-aryl: An aryl group that is or can be coupled to a compound disclosed herein, wherein the aryl group is or becomes coupled through an aliphatic group. Aliphatic-aryl groups may be substituted with one or more groups other than hydrogen, such as aliphatic, heteroaliphatic, haloaliphatic, haloheteroaliphatic, aromatic, or an organic functional group.

Aliphatic-heteroaryl: A heteroaryl group that is or can be coupled to a compound disclosed herein, wherein the heteroaryl group is or becomes coupled through an aliphatic group. Aliphatic-heteroaryl groups may be substituted with one or more groups other than hydrogen, such as aliphatic, heteroaliphatic, haloaliphatic, haloheteroaliphatic, aromatic, or an organic functional group.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). Alkenyl groups may be substituted with one or more groups other than hydrogen, such as aliphatic, heteroaliphatic, haloaliphatic, haloheteroaliphatic, aromatic, or an organic functional group.

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds). Alkoxy groups may be substituted with one or more groups other than hydrogen, such as aliphatic, heteroaliphatic, haloaliphatic, haloheteroaliphatic, aromatic, or an organic functional group.

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl). Alkyl groups may be substituted with one or more groups other than hydrogen, such as aliphatic, heteroaliphatic, haloaliphatic, haloheteroaliphatic, aromatic, or an organic functional group.

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). Alkenyl groups may be substituted with one or more groups other than hydrogen, such as aliphatic, heteroaliphatic, haloaliphatic, haloheteroaliphatic, aromatic, or an organic functional group.

Amide: —C(O)NR^{b}R^{c} or —NR^{b}C(O)R^{c} wherein each of R^{b} and R^{c} independently is selected from hydrogen, aliphatic, heteroaliphatic, haloaliphatic, haloheteroaliphatic, aromatic, or an organic functional group and can be substituted with one or more groups other than hydrogen, such as aliphatic, heteroaliphatic, haloaliphatic, haloheteroaliphatic, aromatic, or an organic functional group.

Amino: —NR^{b}R^{c}, wherein each of R^{b} and R^{c} independently is selected from hydrogen, aliphatic, heteroaliphatic, haloaliphatic, haloheteroaliphatic, aromatic, or an organic functional group, and can be substituted with one or more groups other than hydrogen, such as aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example

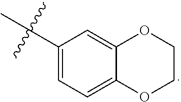 [SMILES: *c1ccc2c(c1)OCCO2]

However, in certain examples, context or express disclosure may indicate that the point of attachment is through a non-aromatic portion of the condensed ring system. For example,

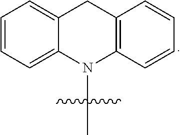 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms, and in some embodiments having at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic. Aroxy groups may be substituted with one or more groups other than hydrogen, such as aliphatic, heteroaliphatic, haloaliphatic, haloheteroaliphatic, aromatic, or an organic functional group.

Azo: —N═NR^{a} wherein R^{a} is hydrogen, aliphatic, heteroaliphatic, haloaliphatic, haloheteroaliphatic, aromatic, or an organic functional group. Azo groups may be substituted with one or more groups other than hydrogen, such as aliphatic, heteroaliphatic, haloaliphatic, haloheteroaliphatic, aromatic, or an organic functional group.

Cancer: A malignant neoplasm that has undergone characteristic anaplasia with loss of differentiation, increased rate of growth, invasion of surrounding tissue, and is capable of metastasis. Features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

Examples of hematological cancers include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of cancer with solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma, breast cancer or colon cancer.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Residual cancer is cancer that remains in a subject after any form of treatment given to the subject to reduce or eradicate the cancer. In several embodiments, administration of a therapeutically effective amount of a disclosed oligomeric compound embodiment to a subject with or at risk of a cancer (such as a leukemia) delays progression of the cancer, and/or reduces a sign or symptom of the cancer.

Carbamate: —OC(O)NR^{b}R^{c}, wherein each of R^{b} and R^{c} independently is selected from hydrogen, aliphatic, heteroaliphatic, haloaliphatic, haloheteroaliphatic, aromatic, or an organic functional group. Carbamate groups can be substituted with one or more groups other than hydrogen, such as aliphatic, heteroaliphatic, haloaliphatic, haloheteroaliphatic, aromatic, or an organic functional group.

Carbonate: —OC(O)OR^{a}, wherein R^{a} is selected from aliphatic, heteroaliphatic, haloaliphatic, haloheteroaliphatic, aromatic, or an organic functional group. Carbonate groups can be substituted with one or more groups other than hydrogen, such as aliphatic, heteroaliphatic, haloaliphatic, haloheteroaliphatic, aromatic, or an organic functional group. In independent embodiments, R^{a} can be hydrogen.

Carboxyl: —C(O)OH.

Carboxylate: —C(O)O— or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Carrier: An excipient that serves as a component capable of delivering a monomeric and/or oligomeric compound embodiment described herein. In some embodiments, a carrier can be a suspension aid, solubilizing aid, or aerosolization aid. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical formulations to be administered can contain minor amounts of non-toxic auxiliary substances, such as wetting or emulsifying agents, preservatives, and pH buffering agents and the like, for example sodium acetate or sorbitan monolaurate.

Cyano: —CN.

Disulfide: —SSR^{a}, wherein R^{a} is selected from hydrogen, aliphatic, heteroaliphatic, haloaliphatic, haloheteroaliphatic, aromatic, or an organic functional group. Disulfide groups can be substituted with one or more groups other than hydrogen, such as aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group. Dithiocarboxylic groups can be substituted with one or more groups other than hydrogen, such as aliphatic, heteroaliphatic, haloaliphatic, haloheteroaliphatic, aromatic, or an organic functional group.

Ester: —C(O)OR^{a} or —OC(O)R^{a}, wherein R^{a} is selected from aliphatic, heteroaliphatic, haloaliphatic, haloheteroaliphatic, aromatic, or an organic functional group. Ester groups can be substituted with one or more groups other than hydrogen, such as aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Ether groups can be substituted with one or more groups other than hydrogen, such as aliphatic, heteroaliphatic, haloaliphatic, haloheteroaliphatic, aromatic, or an organic functional group.

Halo (or halide or halogen): Fluoro, chloro, bromo, or iodo. In some embodiments, halo can also include astatine.

Haloaliphatic: An aliphatic group wherein one or more hydrogen atoms, such as one to 10 hydrogen atoms, independently is replaced with a halogen atom, such as fluoro, bromo, chloro, or iodo.

Haloaliphatic groups can be substituted with one or more groups other than hydrogen, such as aliphatic, heteroaliphatic, haloaliphatic, haloheteroaliphatic, aromatic, or an organic functional group.

Haloaliphatic-aryl: An aryl group that is or can be coupled to a compound disclosed herein, wherein the aryl group is or becomes coupled through a haloaliphatic group. Haloaliphatic-aryl groups can be substituted with one or more groups other than hydrogen, such as aliphatic, heteroaliphatic, haloaliphatic, haloheteroaliphatic, aromatic, or an organic functional group.

Haloaliphatic-heteroaryl: A heteroaryl group that is or can be coupled to a compound disclosed herein, wherein the heteroaryl group is or becomes coupled through a haloaliphatic group. Haloaliphatic-heteroaryl groups can be substituted with one or more groups other than hydrogen, such as aliphatic, heteroaliphatic, haloaliphatic, haloheteroaliphatic, aromatic, or an organic functional group.

Haloalkyl: An alkyl group wherein one or more hydrogen atoms, such as one to 10 hydrogen atoms, independently is replaced with a halogen atom, such as fluoro, bromo, chloro, or iodo. Haloalkyl groups can be substituted with one or more groups other than hydrogen, such as aliphatic, heteroaliphatic, haloaliphatic, haloheteroaliphatic, aromatic, or an organic functional group. In an independent embodiment, haloalkyl can be a CX₃ group, wherein each X independently can be selected from fluoro, bromo, chloro, or iodo.

Haloheteroaliphatic: A heteroaliphatic group wherein one or more hydrogen atoms, such as one to 10 hydrogen atoms, independently is replaced with a halogen atom, such as fluoro, bromo, chloro, or iodo. Haloheteroaliphatic groups can be substituted with one or more groups other than hydrogen, such as aliphatic, heteroaliphatic, haloaliphatic, haloheteroaliphatic, aromatic, or an organic functional group.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. Heteroaliphatic groups can be substituted with one or more groups other than hydrogen, such as aliphatic, heteroaliphatic, haloaliphatic, haloheteroaliphatic, aromatic, or an organic functional group.

Heteroaliphatic-aryl: An aryl group that is or can be coupled to a compound disclosed herein, wherein the aryl group is or becomes coupled through a heteroaliphatic group. Heteroaliphatic-aryl groups can be substituted with one or more groups other than hydrogen, such as aliphatic, heteroaliphatic, haloaliphatic, haloheteroaliphatic, aromatic, or an organic functional group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Hormone: A compound belonging to a class of signaling molecules (e.g., eicosanoids, steroids, and amino acid/protein derivatives), which are produced by glands of multicellular organisms and that are transported through the organism to by the circulatory system to different organs to regulate physiological and behavioral characteristics.

Ketone: —C(O)R^{a}, wherein R^{a} is selected from aliphatic, heteroaliphatic, haloaliphatic, haloheteroaliphatic, aromatic, or an organic functional group. Ketone groups can be substituted with one or more groups other than hydrogen, such as aliphatic, heteroaliphatic, haloaliphatic, haloheteroaliphatic, aromatic, or an organic functional group.

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic; phosphonate; phosphate; silyl ether; sulfinyl; sulfonamide; thial; or combinations thereof. Organic functional groups can be substituted with one or more groups other than hydrogen, such as aliphatic, heteroaliphatic, haloaliphatic, haloheteroaliphatic, aromatic, or an organic functional group.

Oxime: —CR^{a}═NOH, wherein R^{a} is hydrogen, aliphatic, heteroaliphatic, haloaliphatic, haloheteroaliphatic, aromatic, or an organic functional group. Oxime groups can be substituted with one or more groups other than hydrogen, such as aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group. Peroxy groups can be substituted with one or more groups other than hydrogen, such as aliphatic, heteroaliphatic, haloaliphatic, haloheteroaliphatic, aromatic, or an organic functional group.

Pharmaceutically Acceptable Excipient: A substance, other than a compound that is included in a formulation of the compound. As used herein, an excipient may be incorporated within particles of a pharmaceutical composition, or it may be physically mixed with particles of a pharmaceutical composition. An excipient also can be in the form of a solution, suspension, emulsion, or the like. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethylene glycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin. In independent embodiments, water is not intended as a pharmaceutically acceptable excipient.

Pharmaceutically Acceptable Salt: Pharmaceutically acceptable salts of a monomeric and/or oligomeric compound embodiment described herein that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed monomeric and/or oligomeric compound embodiments form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphate can be substituted with one or more groups other than hydrogen, such as aliphatic, heteroaliphatic, haloaliphatic, haloheteroaliphatic, aromatic, or an organic functional group.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphonate group can be substituted with one or more groups other than hydrogen, such as aliphatic, heteroaliphatic, haloaliphatic, haloheteroaliphatic, aromatic, or an organic functional group.

Prodrug: Compound embodiments disclosed herein that are transformed, most typically in vivo, to yield a biologically active compound, particularly the parent compound, for example, by hydrolysis in the gut or enzymatic conversion. Common examples of prodrug moieties include, but are not limited to, pharmaceutically acceptable ester, carbonate, amide, and/or carbamate forms of a compound having an active form bearing a carboxylic acid moiety and/or a hydroxyl group. Examples of pharmaceutically acceptable esters of the compound embodiments of the present disclosure include, but are not limited to, esters of phosphate groups and carboxylic acids, such as aliphatic esters, particularly alkyl esters (for example C₁₋₆alkyl esters). Other prodrug moieties include phosphate esters, such as —CH₂—O—P(O)(OR^{a})₂ or a salt thereof, wherein R^{a} is hydrogen or aliphatic (e.g., C₁₋₆alkyl). Acceptable esters also include cycloalkyl esters and arylalkyl esters such as, but not limited to, benzyl. Examples of pharmaceutically acceptable amides of the compound embodiments of this disclosure include, but are not limited to, primary amides, and secondary and tertiary alkyl amides (for example with between one and six carbons). Amides and esters of disclosed exemplary embodiments of compound embodiments according to the present disclosure can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Silyl Ether: —OSiR^{b}R^{c}, wherein each of R^{b} and R^{c} independently is selected from hydrogen, aliphatic, heteroaliphatic, haloaliphatic, haloheteroaliphatic, aromatic, or an organic functional group. Silyl ether groups can be substituted with one or more groups other than hydrogen, such as aliphatic, heteroaliphatic, haloaliphatic, haloheteroaliphatic, aromatic, or an organic functional group.

Steroidal-Based Compound: An organic compound comprising ring system typically comprised of a skeleton having at least four fused carbocyclic rings; however, in some embodiments, steroids can also include compounds wherein one or more of the typical four fused rings are open (not fused), such as in secosteroids. In oligomeric compound embodiments disclosed herein, a steroid can be any naturally or non-naturally occurring steroid, including, but not limited to, fungal steroids, animal steroids, plant steroids, corticosteroids (including glucocorticoids and mineralcorticoids), sex steroids (including progestogens, androgens, and estrogens), neurosteroids, aminosteroid neuromuscular blocking agents, secosteroids, and any analog and/or derivative thereof.

Subject: Mammals and other animals, such as humans, companion animals (e.g., dogs, cats, rabbits, etc.), utility animals, and feed animals; thus, disclosed methods are applicable to both human therapy and veterinary applications.

Sulfinyl: —S(O)R^{a}, wherein R^{a} is selected from hydrogen, aliphatic, heteroaliphatic, haloaliphatic, haloheteroaliphatic, aromatic, or an organic functional group. Sulfinyl groups can be substituted with one or more groups other than hydrogen, such as aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group. Sulfonyl groups can be substituted with one or more groups other than hydrogen, such as aliphatic, heteroaliphatic, haloaliphatic, haloheteroaliphatic, aromatic, or an organic functional group.

Sulfonamide: —SO₂NR^{b}R^{c} or —N(R^{b})SO₂R^{c}, wherein each of R^{b} and R^{c} independently is selected from hydrogen, aliphatic, heteroaliphatic, haloaliphatic, haloheteroaliphatic, aromatic, or an organic functional group. Sulfonamide groups can be substituted with one or more groups other than hydrogen, such as aliphatic, heteroaliphatic, haloaliphatic, haloheteroaliphatic, aromatic, or an organic functional group.

Sulfonate: —SO₃, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Symptom (or Sign): Any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to, any measurable parameters such as tests for detecting a neurodegenerative disorder or disease.

Therapeutic Agent: A component of certain oligomeric compound embodiments disclosed herein. In some embodiments, the therapeutic agent is a biologically active compound. Exemplary, but non-limiting, therapeutic agents can include gonadotropin-releasing hormone (“GnRH”) antagonists and/or agonists, E3 ubiquitin ligase recruiting ligands, anticancer agents (e.g., chemotherapy agents), kinase antagonists and/or agonists, GPCR antagonists and/or agonists, antimalarial agents, antifungal agents, antiviral agents, antibacterial agents, immunosuppressants, anti-inflammatory agents, pulmonary agents, and the like.

Therapeutically Effective Amount: An amount of a monomeric and/or oligomeric compound embodiment sufficient to bind to an androgen, progesterone, and/or estrogen receptor (either as an agonist and/or an antagonist); to promote or cause fertility suppression in a subject, and in particular embodiments, a male subject; and/or treat a specified disorder or disease, or to ameliorate or eradicate one or more of its symptoms and/or to prevent the occurrence of the disease or disorder. The amount of a compound which constitutes a “therapeutically effective amount” will vary depending on the compound, the disease state and its severity, the age of the patient to be treated, and the like. The therapeutically effective amount can be determined by a person of ordinary skill in the art.

Thial: —C(S)H.

Thiocarboxylic acid: —C(O)SH, or —C(S)OH.

Thiocyanate: —S—CN or —N═C═S.

Thioester: —C(O)SR^{a} or —C(S)OR^{a} wherein R^{a} is selected from hydrogen, aliphatic, heteroaliphatic, haloaliphatic, haloheteroaliphatic, aromatic, or an organic functional group. Thioester groups can be substituted with one or more groups other than hydrogen, such as aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic. Thioether groups can be substituted with one or more groups other than hydrogen, such as aliphatic, heteroaliphatic, haloaliphatic, haloheteroaliphatic, aromatic, or an organic functional group.

Thioketone: —C(S)R^{a} wherein R^{a} is selected from hydrogen, aliphatic, heteroaliphatic, haloaliphatic, haloheteroaliphatic, aromatic, or an organic functional group. Thioketone groups can be substituted with one or more groups other than hydrogen, such as aliphatic, heteroaliphatic, haloaliphatic, haloheteroaliphatic, aromatic, or an organic functional group.

Treating/Treatment: Treatment of a disease or condition of interest in a subject, particularly a human, canine, or feline having the disease or condition of interest, and includes by way of example, and without limitation:

(i) prophylactic administration to prevent the disease or condition from occurring in a subject, or to ameliorate symptoms associated with the condition if required in particular, when such subject is predisposed to the condition but has not yet been diagnosed as having it;

(ii) inhibiting the disease or condition, for example, arresting or slowing its development;

(iii) relieving the disease or condition, for example, causing regression of the disease or condition or a symptom thereof; or

(iv) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### I. INTRODUCTION

In contrast to current male contraceptive agents, compound embodiments disclosed herein not only exhibit androgenic and/or progestogenic activity, but they also are able to undergo aromatization to further provide estrogenic activity, thereby providing the ability to mitigate and/or prevent the bone density loss observed with conventional steroid male contraceptives. Monomeric compound embodiments of the present disclosure comprise a unique substitution pattern in the A ring of the compounds that facilitates aromatization of the compounds to thus provide monomeric compound embodiments having an estradiol backbone. In particular embodiments, the monomeric compound embodiments comprise an aliphatic substituent (e.g., a methyl group) at the C10 position, which facilitates aromatization. The monomeric compound embodiments of the present disclosure also can comprise functional groups (e.g., progroups) at certain positions that facilitate formulating and administrating the monomeric compound embodiments to subjects in vivo. The monomeric compound embodiments of the present disclosure can further comprise substituents at the C7 carbon that promote androgen receptor recognition. In view of one or more of these unique structural features, the monomeric compound embodiments of the present disclosure can possess androgenic, progestogenic, and estrogenic activity and thus can not only exhibit male contraceptive capabilities, but also can avoid deleterious effects on bone density by promoting estrogen analogue circulation. Also contemplated by the present disclosure are compound embodiments wherein the ring bearing the C10 carbon comprising the aliphatic substituent has been aromatized, the compound embodiments further comprising the functional groups that facilitate formulating and administrating the monomeric compound embodiments to subjects in vivo.

Also disclosed herein are oligomeric compound embodiments that exhibit androgenic, progestogenic, estrogenic, glucocorticoid, and/or mineralocorticoid activities and that comprise monomeric components that are coupled together through different types of linker groups that facilitate selective cleavage of the two monomeric components. The oligomeric compound embodiments can comprise monomeric components that can be selected from any of the monomeric compound embodiments disclosed herein, other steroidal compounds (e.g., steroidal compounds that lack C10 substitution, or steroidal compounds known in the art), and/or therapeutic agents (e.g., anticancer agents, gonadotropin-releasing hormone antagonist/agonists, E3 ubiquitin ligase recruiting ligands, kinase antagonist and agonist, GPCR antagonist and agonist, antimalarial agents, and other types of therapeutic agents).

### II. MONOMER COMPOUND EMBODIMENTS

Disclosed herein are embodiments of a monomeric compound capable of being used as a contraceptive agent for animals, particularly humans (including males and/or females). In some embodiments, the monomeric compound has one or more structural features that facilitate its ability to provide androgenic and/or progestogenic activity. In particular embodiments, the monomeric compound comprises one or more structural features that facilitate their use as contraceptive agents. Certain monomeric compound embodiments can comprise a substituent at C10, which promotes the ability to aromatize in situ (e.g., in vivo, after administration to a subject) to thereby provide estrogenic activity in addition to any androgenic and/or progestogenic activity. In yet some additional embodiments, the monomeric compound can comprise a substituent at the C7 position, which can facilitate binding to receptors. Such structural features, as well as others, are discussed herein. Also disclosed herein are oligomeric compound embodiments that comprise one or more of the disclosed monomeric compound embodiments, other steroidal compounds, and/or therapeutic agents. These oligomeric compounds can provide glucocorticoid and/or mineralocorticoid activity, in addition to androgenic, progestogenic, and/or estrogenic activity.

In particular embodiments, the monomeric compound embodiments can have structures represented by Formulas I, II, and/or III, illustrated below. In some embodiments, the monomeric compound can be in the form of a pharmaceutically acceptable salt, a prodrug, a solvate, or a tautomer thereof.

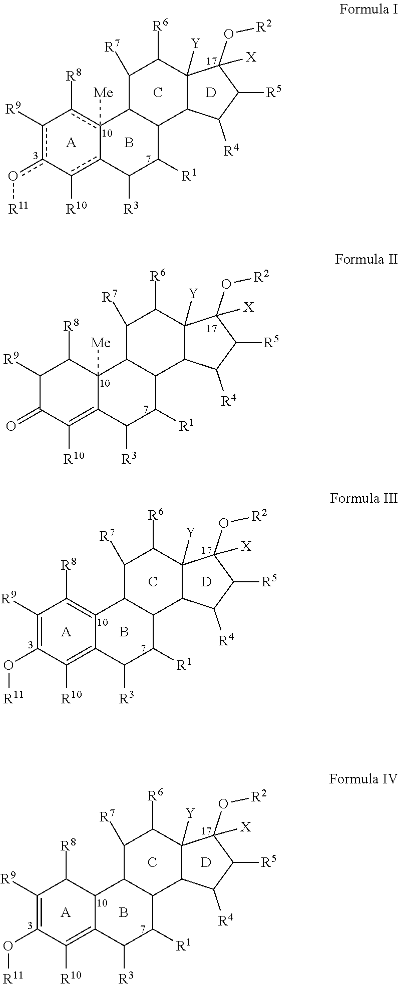

With reference to Formulas I, II, III, and/or VI the following variable recitations can apply (the C3, C7, C10, and C17 positions are indicated with the numbers “3,” “7, “10,” and “17” in Formulas I, II, III, and/or IV):
R¹ is selected from H, deuterium (or “D”), halogen, aliphatic, aromatic, heteroaliphatic, haloaliphatic, haloheteroaliphatic, or an organic functional group;
R² is selected from —C(O)R^{a}, —C(O)OR^{a}, —C(O)NR^{b}R^{c}, wherein each R^{a} independently is selected from aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; —P(O)(OR^{a})₂, wherein each R^{a} independently is H, aliphatic, aromatic, heteroaliphatic, haloaliphatic, haloheteroaliphatic, heteroaliphatic, or an organic functional group; or —S(O)₂R^{a} wherein R^{a} is H, aliphatic, aromatic, heteroaliphatic, haloaliphatic, haloheteroaliphatic, heteroaliphatic, or an organic functional group; or R² can be H or D when either (i) both ring A is not an aromatic ring and R¹ is not, or is other than, methyl or ethyl; or (ii) when ring A is an aromatic ring and when R¹ is other than H;
each of R³, R⁴, R⁵, R⁶, R⁷, R⁸, R⁹, and R¹⁰ independently is selected from H, D, aliphatic, aromatic, heteroaliphatic, haloaliphatic, haloheteroaliphatic, heteroaliphatic, or an organic functional group;
R¹¹ (if present, such as in Formulas III, IV and/or when ring A of Formula I is aromatized or contains a conjugated diene) can be selected from hydrogen, aliphatic, heteroaliphatic, aromatic, heteroaliphatic, haloaliphatic, haloheteroaliphatic, or an organic functional group;
X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; and
Y is aliphatic.

In particular embodiments, the compound can have a structure represented by Formulas I, II, III, and/or IV wherein:
R¹ is selected from H, D, Cl, F, I, Br, alkyl, alkenyl, alkynyl, aryl, heteroaryl, heteroalkyl, heteroalkenyl, heteroalkynyl, or any combination of these groups;
R² is selected from —C(O)R^{a}, —C(O)OR^{a}, —C(O)NR^{b}R^{c}, wherein each R^{a} independently is selected from alkyl, alkenyl, alkynyl, aryl, heteroaryl, heteroalkyl, heteroalkenyl, heteroalkynyl, halogen, or another organic functional group, and each of R^{b} and R^{c} independently is H, alkyl, alkenyl, alkynyl, aryl, heteroaryl, heteroalkyl, heteroalkenyl, heteroalkynyl, halogen, or another organic functional group; —P(O)(OR^{a})₂, wherein each R^{a} independently is H, alkyl, alkenyl, alkynyl, aryl, heteroaryl, heteroalkyl, heteroalkenyl, heteroalkynyl, or an organic functional group; or —S(O)₂R^{a} wherein R^{a} is H, alkyl, alkenyl, alkynyl, aryl, heteroaryl, heteroalkyl, heteroalkenyl, heteroalkynyl, or an organic functional group;
each of R³, R⁴, R⁵, R⁶, R⁷, R⁸, R⁹, and R¹⁰ independently is selected from H, D, —OH, halogen, alkyl, alkenyl, alkynyl, aryl, heteroaryl, heteroalkyl, heteroalkenyl, heteroalkynyl, or any combination of these groups;
R¹¹ is selected from H, D, alkyl, or is selected from a group recited for R² (e.g., —C(O)R^{a}, —C(O)OR^{a}, —C(O)NR^{b}R^{c}, wherein each R^{a} independently is selected from alkyl, alkenyl, alkynyl, aryl, heteroaryl, heteroalkyl, heteroalkenyl, heteroalkynyl, halogen, or another organic functional group, and each of R^{b} and R^{c} independently is H, alkyl, alkenyl, alkynyl, aryl, heteroaryl, heteroalkyl, heteroalkenyl, heteroalkynyl, halogen, or another organic functional group; —P(O)(OR^{a})₂, wherein each R^{a} independently is H, alkyl, alkenyl, alkynyl, aryl, heteroaryl, heteroalkyl, heteroalkenyl, heteroalkynyl, or an organic functional group; or —S(O)₂R^{a} wherein R^{a} is H, alkyl, alkenyl, alkynyl, aryl, heteroaryl, heteroalkyl, heteroalkenyl, heteroalkynyl, or an organic functional group);
X is selected from H, D, alkyl, alkenyl, alkynyl, heteroalkyl, heteroalkenyl, heteroalkynyl, —OH, —C(O)R^{a}, —C(O)OR^{a}, or —C(O)NR^{b}R^{c}, wherein each R^{a} independently is alkyl, alkenyl, alkynyl, aryl, heteroaryl, heteroalkyl, heteroalkenyl, heteroalkynyl, or an organic functional group, and each of R^{b} and R^{c} independently is H, alkyl, alkenyl, alkynyl, aryl, heteroaryl, heteroalkyl, heteroalkenyl, heteroalkynyl, or an organic functional group; and
Y is alkyl, alkenyl, or alkynyl.
   With reference to such embodiments, the organic functional group can be selected from any organic functional group disclosed herein. And, any of the particular aliphatic and/or heteroaliphatic groups described above can be cyclic or acyclic versions of such groups and can include any deuterated versions thereof.

In yet additional embodiments, the compound can have a structure represented by Formulas I, II, III, and/or IV wherein:
R¹ is selected from H, D, Cl, F, I, Br, lower alkyl (e.g., methyl, ethyl, propyl, butyl, pentyl, hexyl, heptyl, octyl, nonyl, or decyl, including cyclic and acyclic versions thereof); or phenyl; R² is selected from —C(O)R^{a}, —C(O)OR^{a}, or —C(O)NR^{b}R^{c}, wherein each R^{a} independently is selected from Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl, and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; —P(O)(OR^{a})₂, wherein each R^{a} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; or —S(O)₂R^{a} wherein R^{a} is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group;
each of R³, R⁴, R⁵, R⁶, R⁷, R⁸, R⁹, and R¹⁰ independently is selected from H, D, —OH, Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, or any combination of these groups;
R¹¹ is H, D, or —C(O)OR^{a}, wherein each R^{a} independently is selected from alkyl (such as lower alkyl);
X is selected from H, D, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, —OH, —C(O)R^{a}, —C(O)OR^{a}, or —C(O)NR^{b}R^{c}, wherein each R^{a} independently is Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl, and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; and
Y is lower alkyl (e.g., methyl, ethyl, propyl, butyl, pentyl, hexyl, heptyl, octyl, nonyl, or decyl, including cyclic and acyclic versions thereof).
   With reference to such embodiments, the organic functional group can be selected from any organic functional group disclosed herein. And, any of the particular aliphatic and/or heteroaliphatic groups described above can be cyclic or acyclic versions of such groups and can include any deuterated versions thereof.

In representative embodiments of Formula II, R¹ is selected from H, Cl, F, I, Br, methyl, ethyl, t-butyl, or phenyl; R² is selected from —C(O)Cl, —C(O)Me, —C(O)Et, —C(O)nPr, —C(O)(CH₂)₅CH₃, —C(O)(CH₂)₉CH₃, —C(O)(CH₂)₁₀CH₃, —C(O)(CH₂)₇C(H)═C(H)(CH₂)₇CH₃, —C(O)(CH₂)₇C(H)═C(H)CH₂C(H)═C(H)(CH₂)₄CH₃, —C(O)O(CH₂)₉CH₃, —C(O)OMe, —C(O)OC(CH₃)₃, —C(O)O(CH₂)₃CH₃, —C(O)O(CH₂)₄CH₃, —C(O)NH(CH₂)₉CH₃, —C(O)O(CH₂)₁₁CH₃, —C(O)NH(CH₂)₁₁CH₃, —C(O)N(CH₃)CH₃, —C(O)N(H)CH₃, —C(O)N(CH₂)₄CH₃, —C(O)N(H)(CH₂)₄CH₃, —C(O)N(CH₃)C(O)N(H)CH₃, or —S(O)₂Ph-p-Me; R³ is H or D; R⁴, R⁵, and R⁶ are H; R⁷ is H or Me; R⁸ is H; R⁹ is H or D; R¹⁰ is H or D; X is selected from H, —CCH; and Y is methyl or ethyl. In representative embodiments of Formula III, R¹ is selected from H, Cl, F, I, Br, methyl, ethyl, t-butyl, or phenyl; R² is selected from —C(O)Cl, —C(O)Me, —C(O)Et, —C(O)nPr, —C(O)(CH₂)₅CH₃, —C(O)(CH₂)₉CH₃, —C(O)(CH₂)₁₀CH₃, —C(O)(CH₂)₇C(H)═C(H)(CH₂)₇CH₃, —C(O)(CH₂)₇C(H)═C(H)CH₂C(H)═C(H)(CH₂)₄CH₃, —C(O)O(CH₂)₉CH₃, —C(O)OMe, —C(O)OC(CH₃)₃, —C(O)O(CH₂)₃CH₃, —C(O)O(CH₂)₄CH₃, —C(O)NH(CH₂)₉CH₃, —C(O)O(CH₂)₁₁CH₃, —C(O)NH(CH₂)₁₁CH₃, —C(O)N(CH₃)CH₃, —C(O)N(H)CH₃, —C(O)N(CH₂)₄CH₃, —C(O)N(H)(CH₂)₄CH₃, —C(O)N(CH₃)C(O)N(H)CH₃, or —S(O)₂Ph-p-Me; R³ is H or D; R⁴, R⁵, and R⁶ are H; R⁷ is H or Me; R⁸ is H; R⁹ is H or D; R¹⁰ is H or D; R¹¹ is H or D; X is selected from H, —CCH; and Y is methyl or ethyl. In yet additional embodiments of Formula II, R¹ is phenyl; R² is hydrogen; R³ is H or D; R⁴, R⁵, and R⁶ are H; R⁷ is H or Me; R⁸ is H; R⁹ is H or D; R¹⁰ is H or D; X is selected from H, —CCH; Y is methyl or ethyl. In yet additional embodiments of Formula III, R¹ is aliphatic or aromatic; R² is hydrogen; R³ is H or D; R⁴, R⁵, and R⁶ are H; R⁷ is H or Me; R⁸ is H; R⁹ is H or D; and R¹⁰ is H or D; R¹¹ is H or D; X is selected from H, —CCH; and Y is methyl or ethyl. In representative embodiments of Formula IV, R¹-R¹⁰ are H, Y is ethyl, X is —CCH, and R¹¹ is —C(O)O(CH₂)·CH₃, wherein n is an integer selected from 0 to 15, such as 0 to 12, or 0 to 10, or 0 to 8, or 0 to 6, or 0 to 4, or 0 to 3, or 0 to 2. In some such embodiments, n is 0, 1, 2, 3, 4, 5, 6, 7, 8, 9, 10, 11, 12, 13, 14, or 15.

In some embodiments, the monomeric compound has a structure satisfying Formulas IA, IB, IIA, IIB, IIIA, IIIB, IVA, or IVB below, which illustrate particular stereochemistries of particular enantiomeric carbons atoms of the compounds. In some embodiments of Formulas IA, IB, IIA, IIB, IIIA, IIIB, IVA, or IVB the monomeric compound can be in the form of a pharmaceutically acceptable salt, a prodrug, a solvate, or a tautomer thereof.

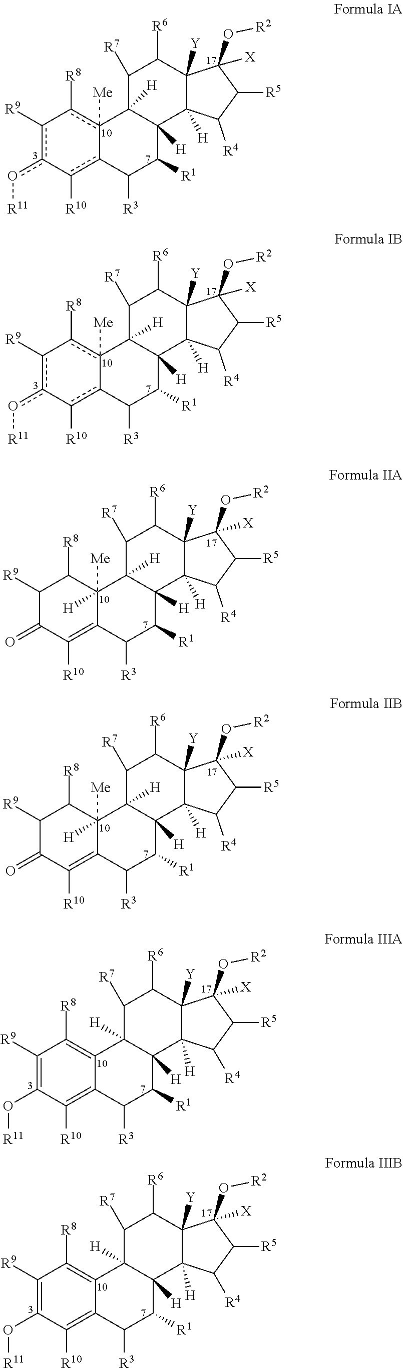

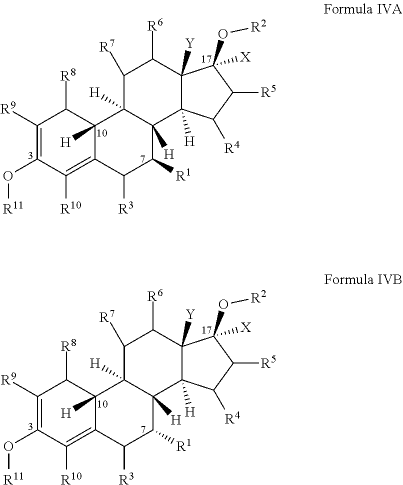

With reference to each of Formulas IA, IB, IIA, IIB, IIIA, IIIB, IVA, or IVB each of R¹, R², R³, R⁴, R⁵, R⁶, R⁷, R⁸, R⁹, R¹⁰, Y, and X can be as recited above for any substituent recitations provided for Formulas I, II, III and/or IV.

In some embodiments, the monomeric compound has a structure satisfying Formulas V, VI, VII, and/or VIII below. In some embodiments of Formulas V, VI, VII, or VIII, the monomeric compound can be in the form of a pharmaceutically acceptable salt, a prodrug, a solvate, or a tautomer thereof.

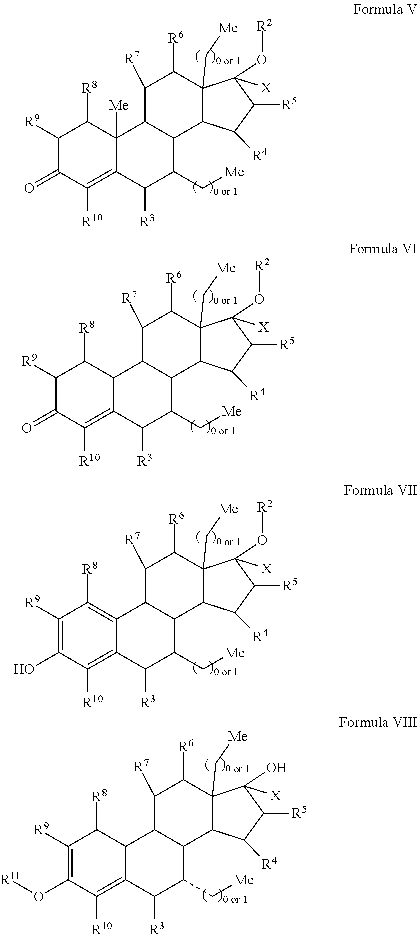

With reference to Formulas V, VI, VII, and/or VIII, each of R², R³, R⁴, R⁵, R⁶, R⁷, R⁸, R⁹, R¹⁰, and X can be as recited above for any substituent recitations provided for Formulas I, II, III and/or IV. In particular embodiments of Formulas V, VI, and/or VII (and/or any of the Formulas provided above), R² or R¹¹ (with respect to Formula VIII) is —C(O)(CH₂)ₙCH₃, wherein n is an integer selected from 0 to 15, such as 0 to 12, or 0 to 10, or 0 to 8, or 0 to 6, or 0 to 4, or 0 to 2 (e.g., 2, 3, 4, 6, 7, 8, 9, 10, or 11); and in an independent embodiment n is not 0, 1, or 5. In yet additional embodiments of Formulas V, VI, and/or VII (and/or any of the Formulas provided above), R² or R¹¹ (with respect to Formula VIII) is —C(O)O(CH₂)ₙCH₃, wherein n is an integer selected from 0 to 15, such as 0 to 12, or 0 to 10, or 0 to 8, or 0 to 6, or 0 to 4, or 0 to 2 (e.g., 0, 1, 2, 3, 4, 5, 6, 7, 8, 9, 10, 11, 12, 13, 14, or 15). In yet additional embodiments of Formulas V, VI, and/or VII (and/or any of the Formulas provided above), R² or R¹¹ (with respect to Formula VIII) is —C(O)NR^{b}R^{c}, wherein R^{b} is H or lower alkyl (e.g., methyl, ethyl, propyl, butyl, pentyl, hexyl, heptyl, octyl, nonyl, or decyl, including cyclic and acyclic versions thereof) and R^{c} is lower alkyl (e.g., methyl, ethyl, propyl, butyl, pentyl, hexyl, heptyl, octyl, nonyl, or decyl, including cyclic and acyclic versions thereof) or heteroaliphatic (e.g., amide having a formula —C(O)NR^{b}R^{c}). In yet additional embodiments of Formulas V, VI, and/or VII (and/or any of the Formulas provided above), R² or R¹¹ (with respect to Formula VIII) is —S(O)₂R^{a} wherein R^{a} is aromatic (e.g., Ph, which can be substituted with one or more substituents). In some embodiments of Formulas V, VI, and/or VII (and/or any of the Formulas provided above), each of R³, R⁴, R⁵, R⁶, R⁷, R⁸, R⁹, and R¹⁰ is hydrogen or deuterium. In particular embodiments of Formulas V, VI, and/or VII (and/or any of the Formulas provided above), X is hydrogen or —CCH. In particular embodiments of Formula VIII, R¹¹ is —C(O)O(CH₂)ₙCH₃, wherein n is an integer selected from 0 to 15, such as 0 to 12, or 0 to 10, or 0 to 8, or 0 to 6, or 0 to 4, or 0 to 3, or 0 to 2. In some such embodiments, n is 0, 1, 2, 3, 4, 5, 6, 7, 8, 9, 10, 11, 12, 13, 14, or 15.

In some embodiments, the monomeric compound has a structure satisfying any one or more of Formulas VA-VD, VA′-VD′, and VA″-VD″; Formulas VIA-VID, VIA′-VID′, and VIA″-VID″; and/or Formulas VIIA-VIID, VIIA′-VIID′, and VIIA″-VIID″, which show representative stereoisomers of the formulas disclosed herein.

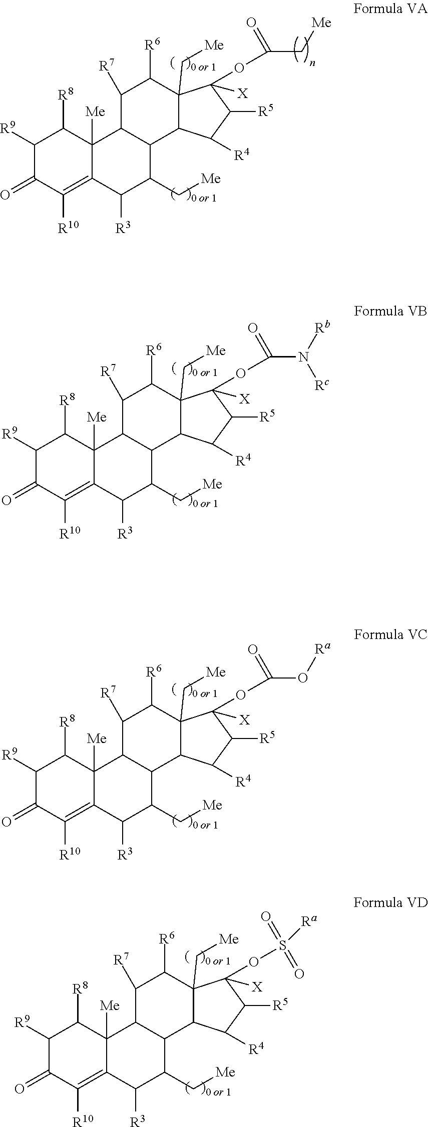

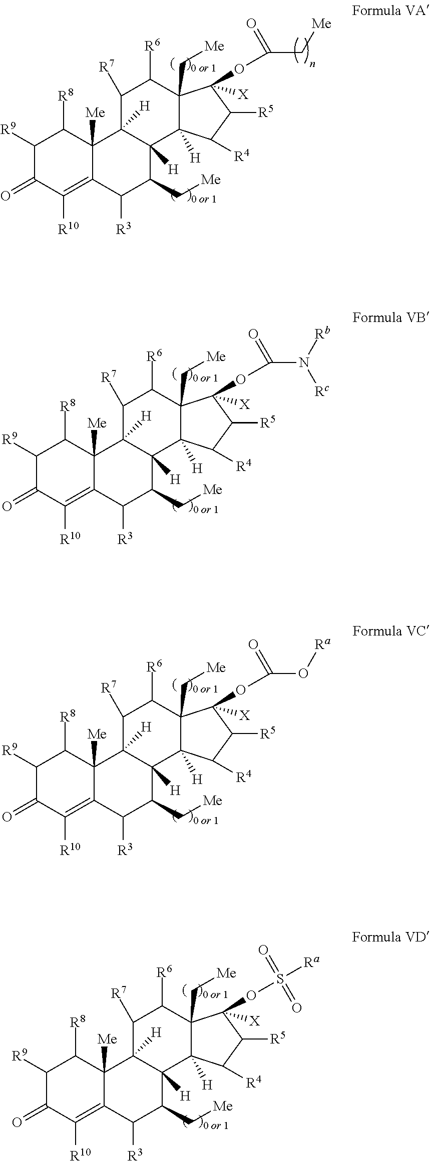

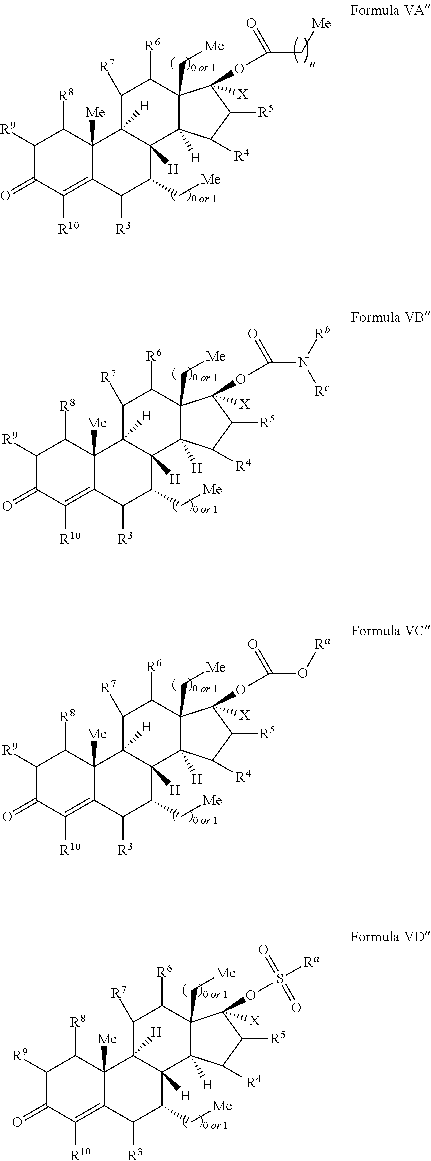

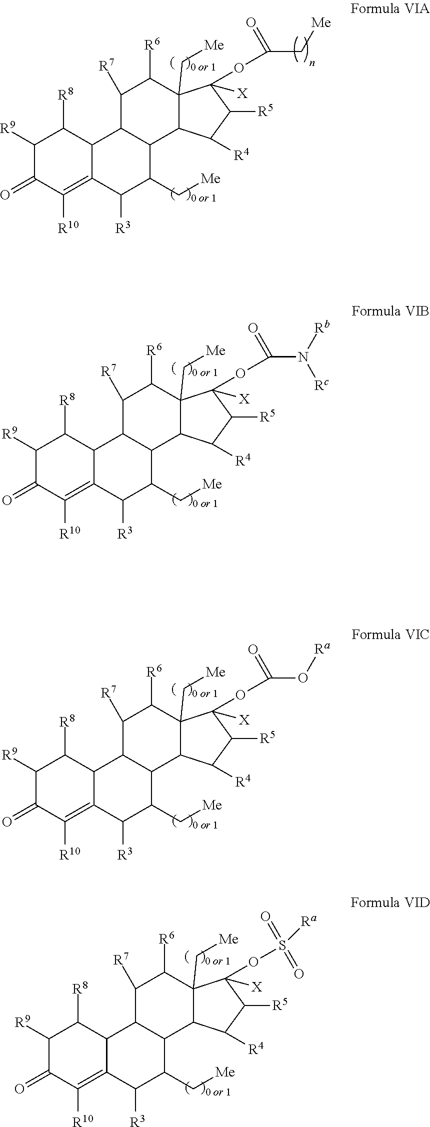

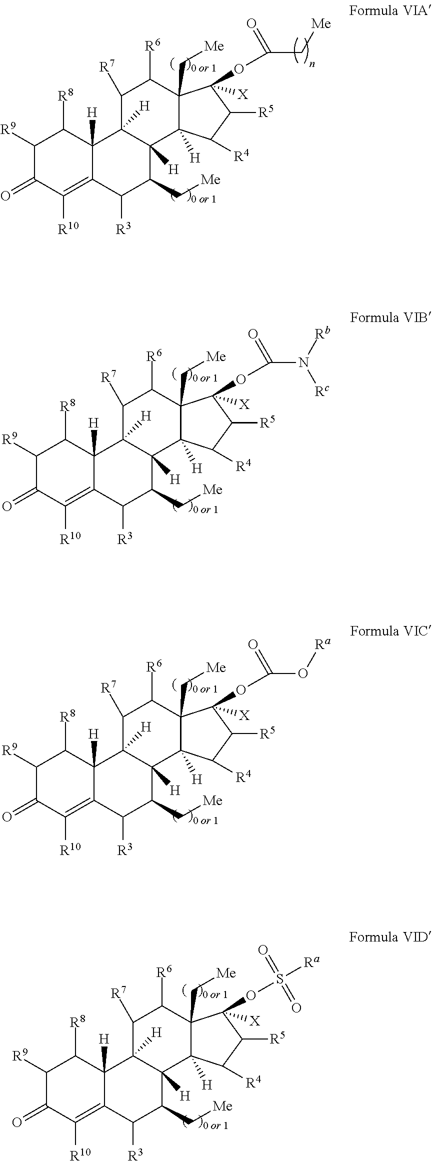

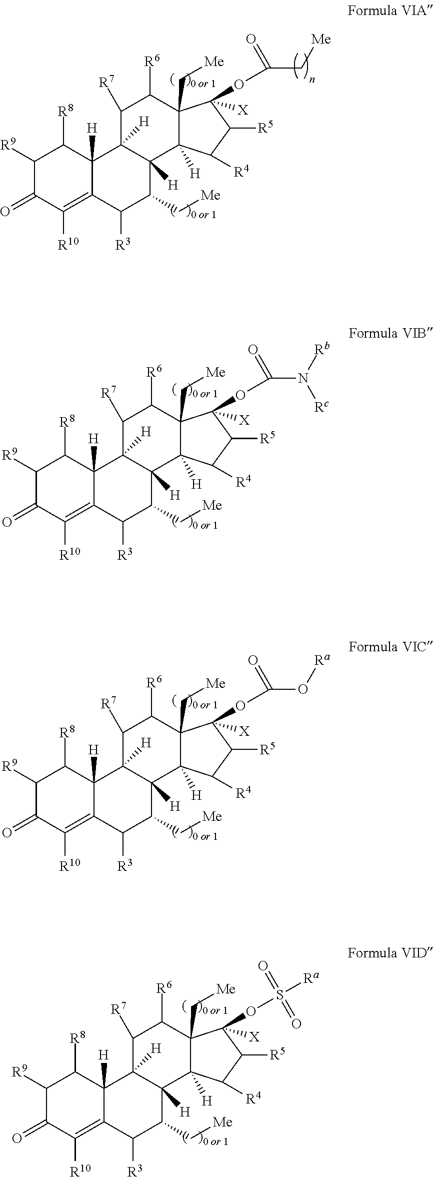

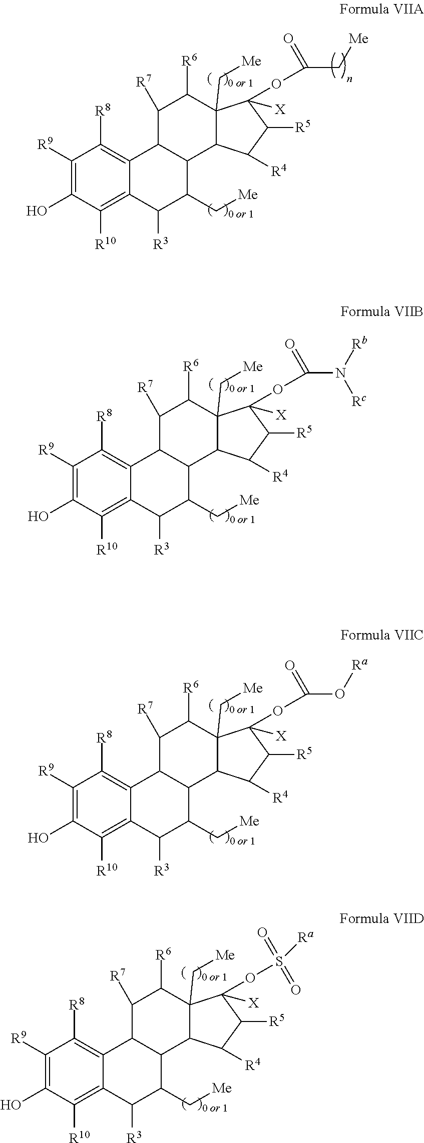

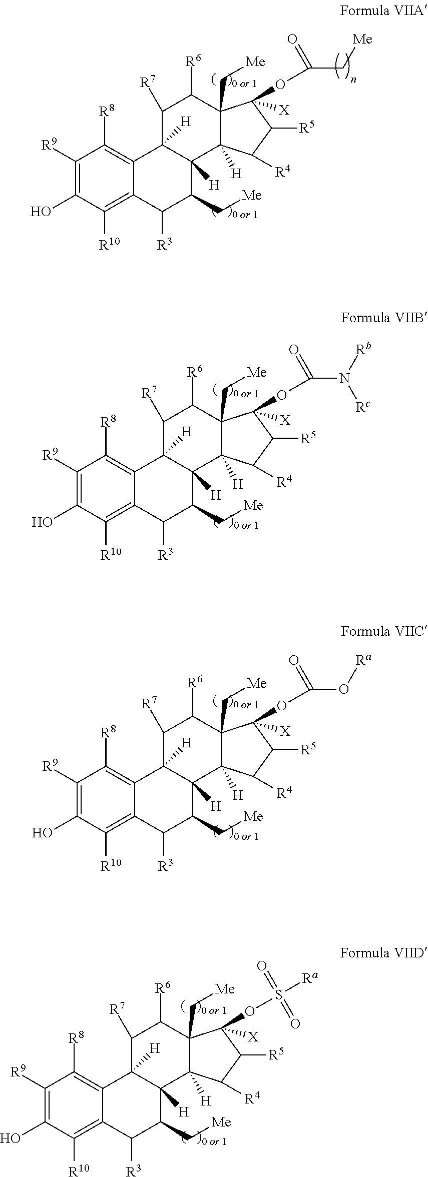

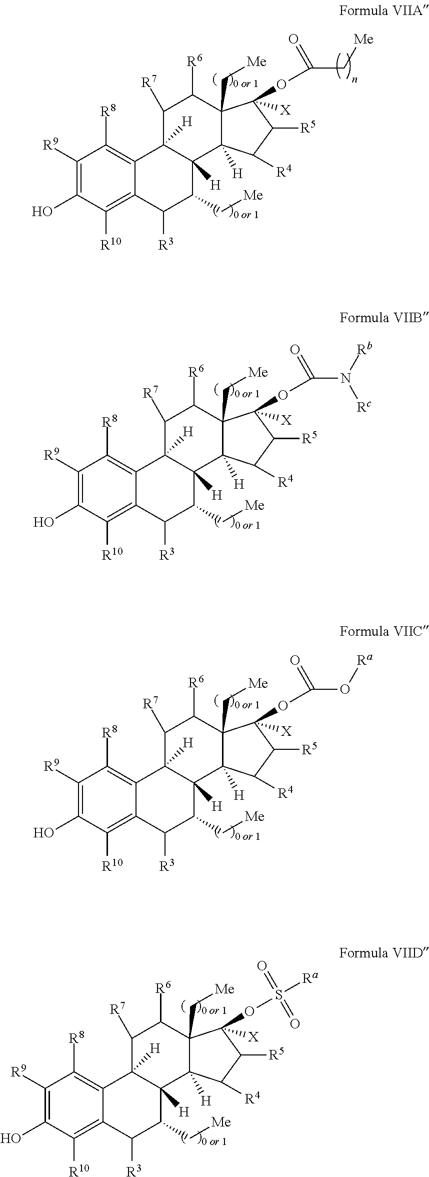

With reference to Formulas VA-VD, VA′-VD′, and VA″-VD″; Formulas VIA-VID, VIA′-VID′, and VIA″-VID″; and/or Formulas VIIA-VIID, VIIA′-VIID′, and VIIA″-VIID″, each of R², R³, R⁴, R⁵, R⁶, R⁷, R⁸, R⁹, R¹⁰, X, n, R^{a}, R^{c}, and R^{c} can be as recited above for any of the Formulas described above. In independent embodiments of any or all of the above formulas, if R² is —C(O)OCH₃, —C(O)O(CH₂)₅CH₃, —C(O)O(CH₂)₉CH₃, or —C(O)O(CH₂)₁₁CH₃, and each of Y and R¹ is methyl, and R⁷ is methyl or halogen, then the compound comprises a methyl group attached to the C10 atom. In some independent embodiments, the monomeric compound is not 11β-ethyl-19-nortestosterone-17-methylcarbonate, 11β-ethyl-19-nortestosterone-17-decylcarbonate, 11β-ethyl-19-nortestosterone-17-dodecylcarbonate, 11β-methyl-19-nortestosterone-17-methylcarbonate, 11β-methyl-19-nortestosterone-17-decylcarbonate, or 11β-methyl-19-nortestosterone-17-(trans-4-n-butylcyclohexyl) carbonate. In independent embodiments of any or all of the above formulas, R² is not, or is other than any of the following: —C(O)(CH₂)₅CH₃; —C(O)CH₂SO₂OR, wherein R is lower alkyl, cyclopentyl, cyclohexyl, cycloheptyl, phenyl, phenoxy-lower-alkyl, and lower-alkoxy-phenyl; —C(O)CH₂SO₂OEt; —C(O)Ph; —C(O)Me; —C(O)Et; —C(O)OCH₂adamantyl; —C(O)Oadamantyl;

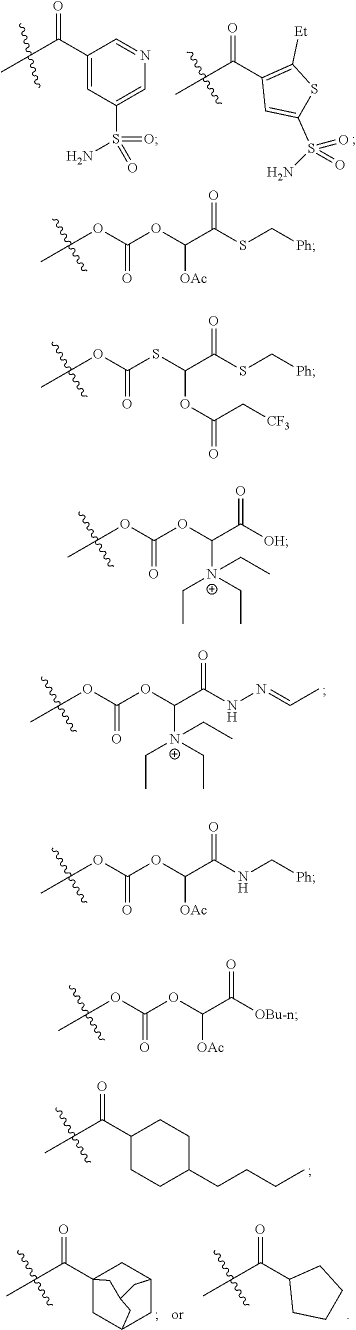

In exemplary embodiments, the monomeric compound can be selected from any of the following (with reference to any stereocenters bearing tertiary carbon atoms with hydrogens bound thereto, but not illustrated, the stereochemistry can be as illustrated above for any of Formulas IA, IB, IIA, IIB, IIIA, IIIB, IVA, and IVB):

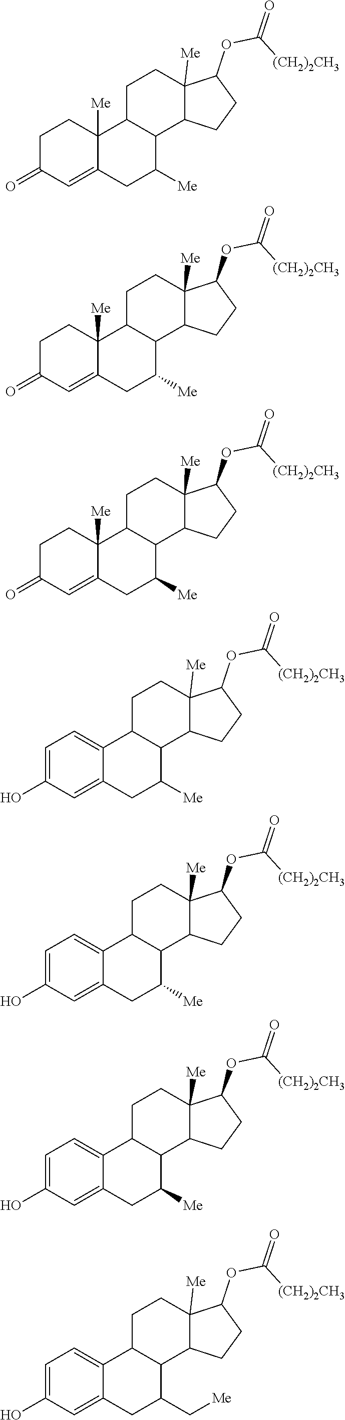

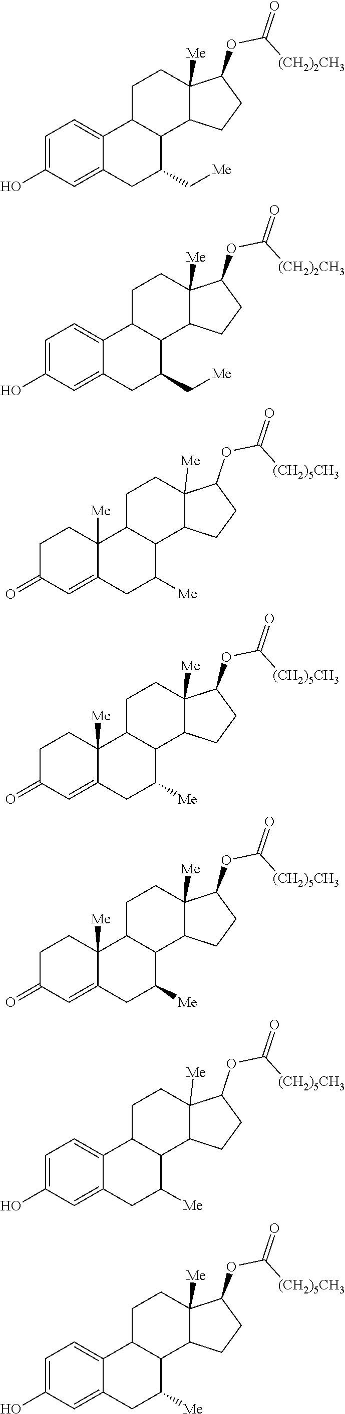

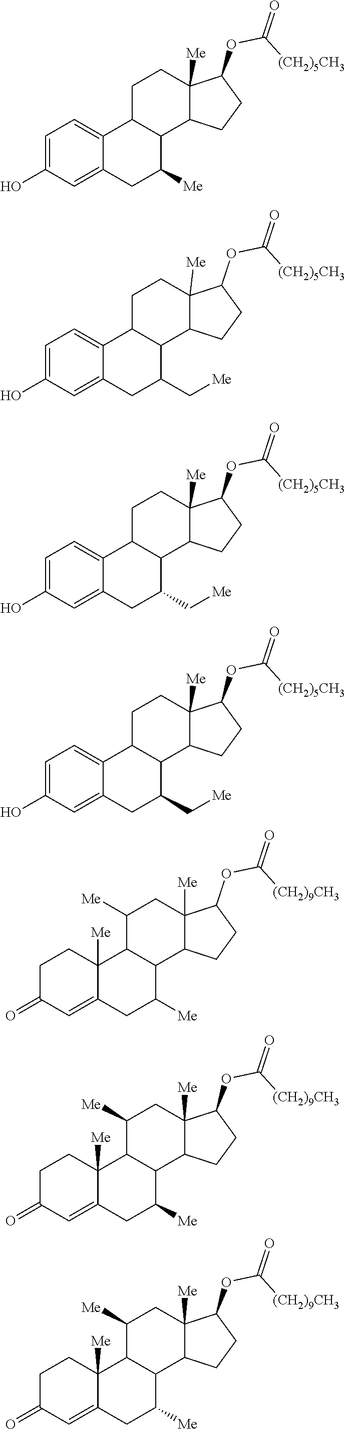

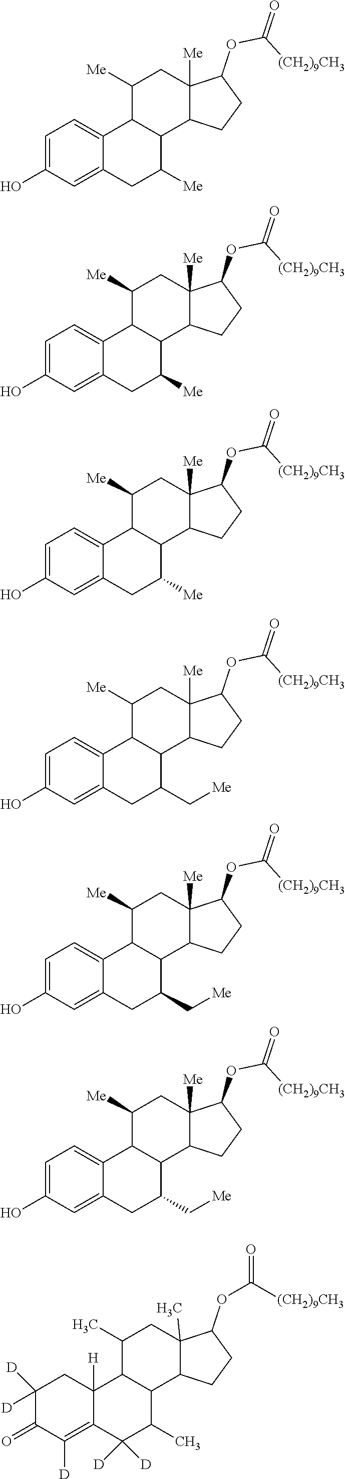

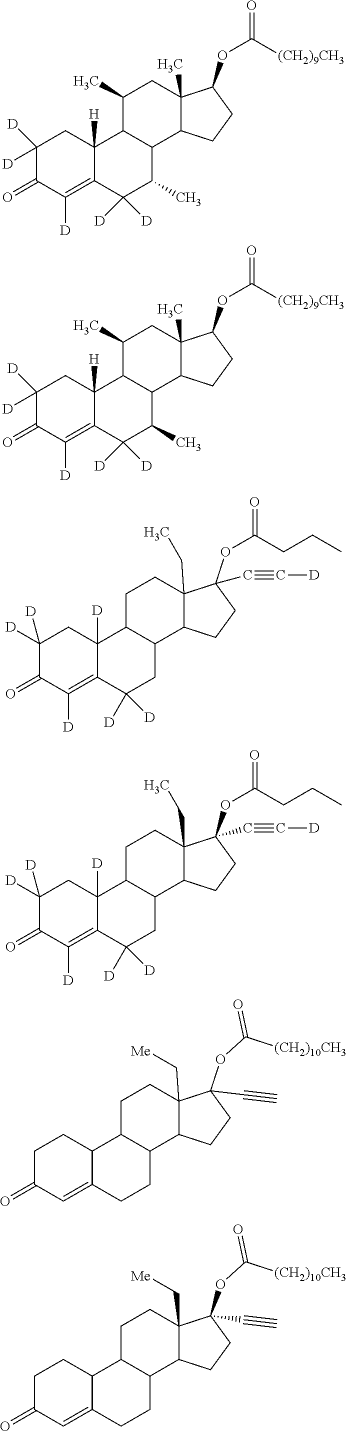

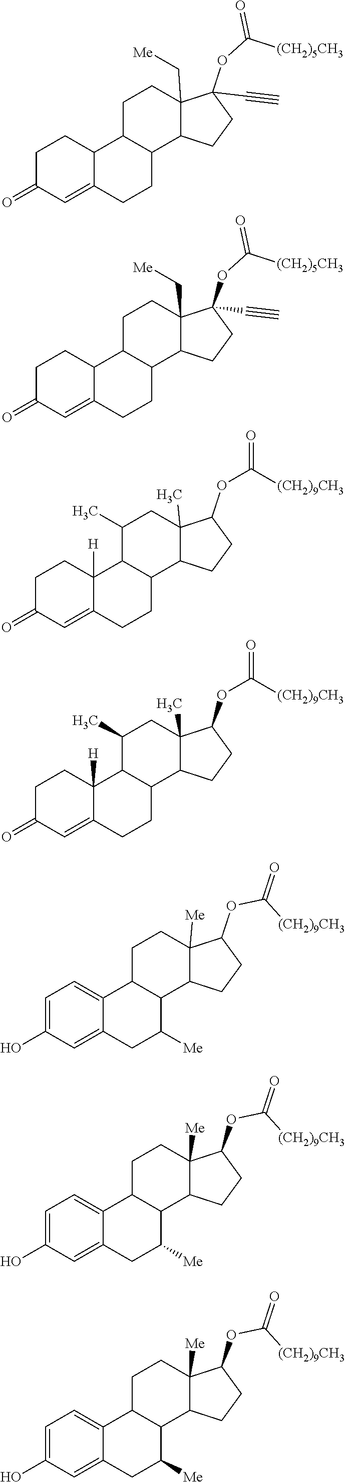

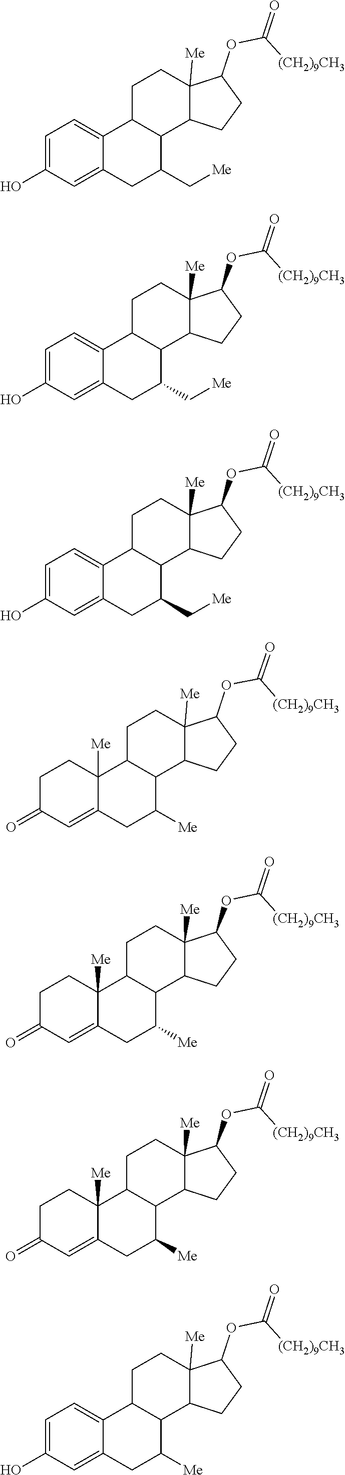

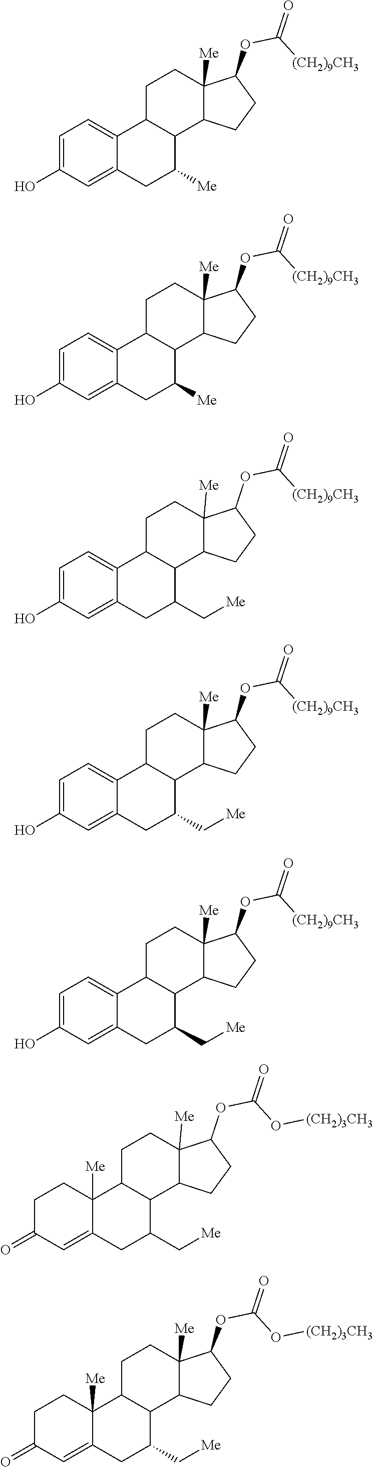

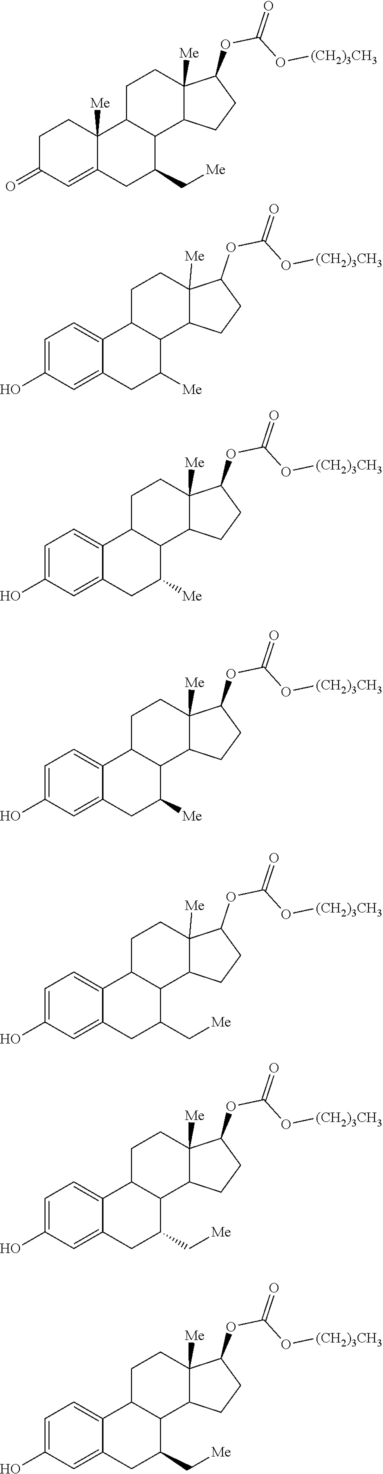

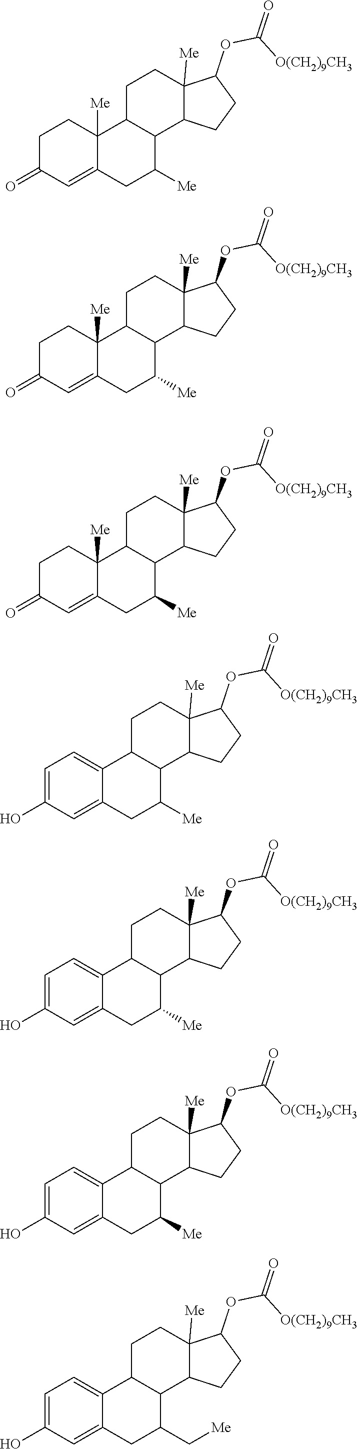

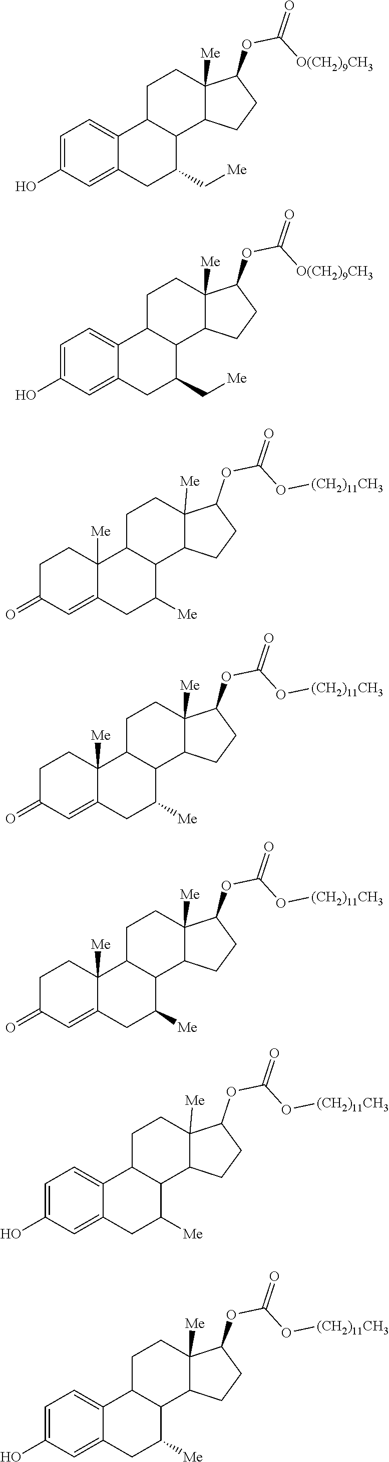

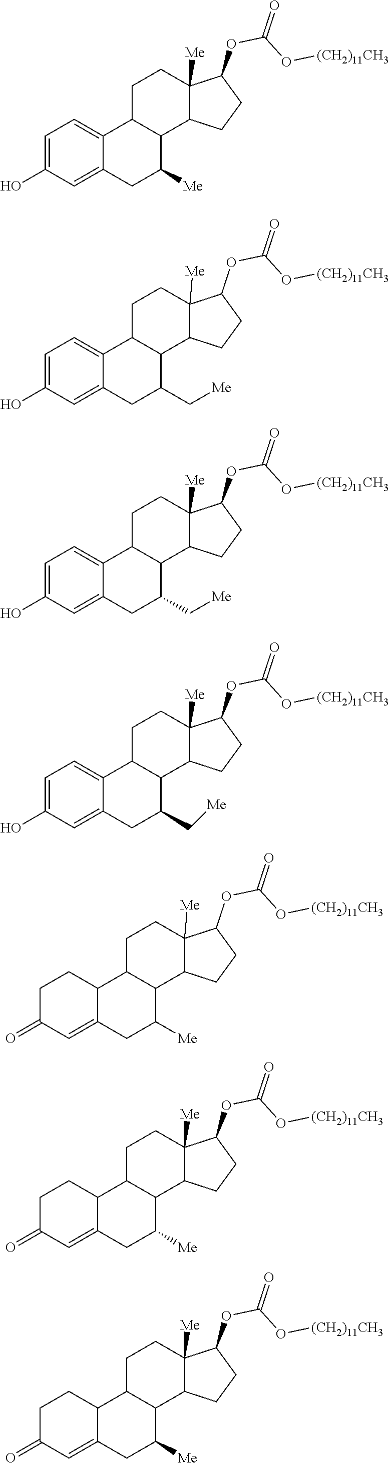

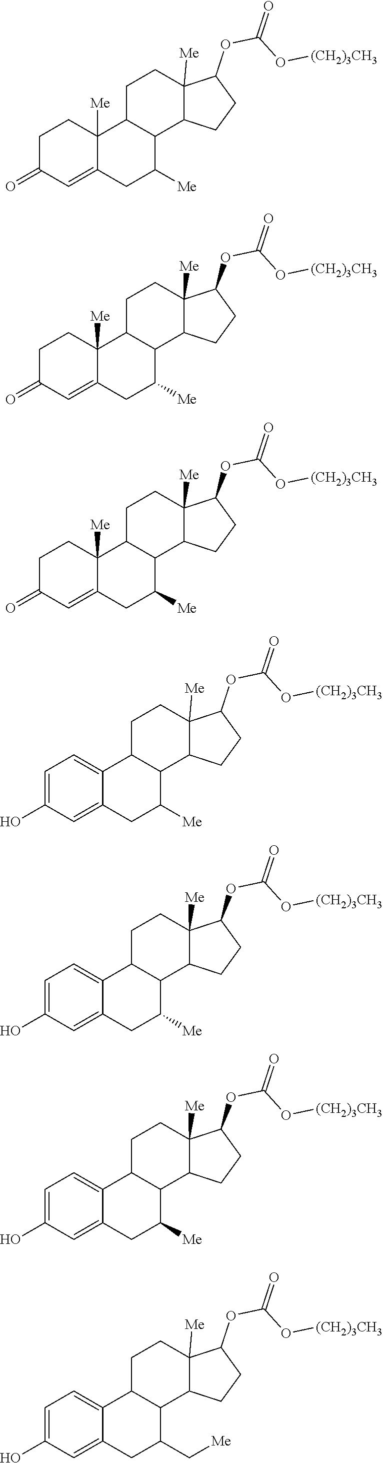

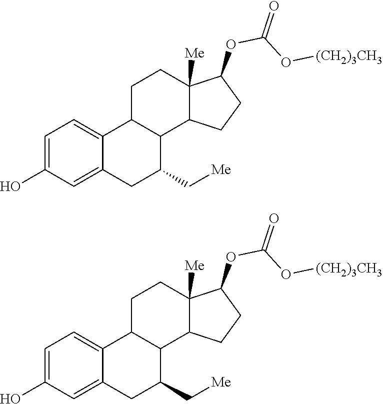

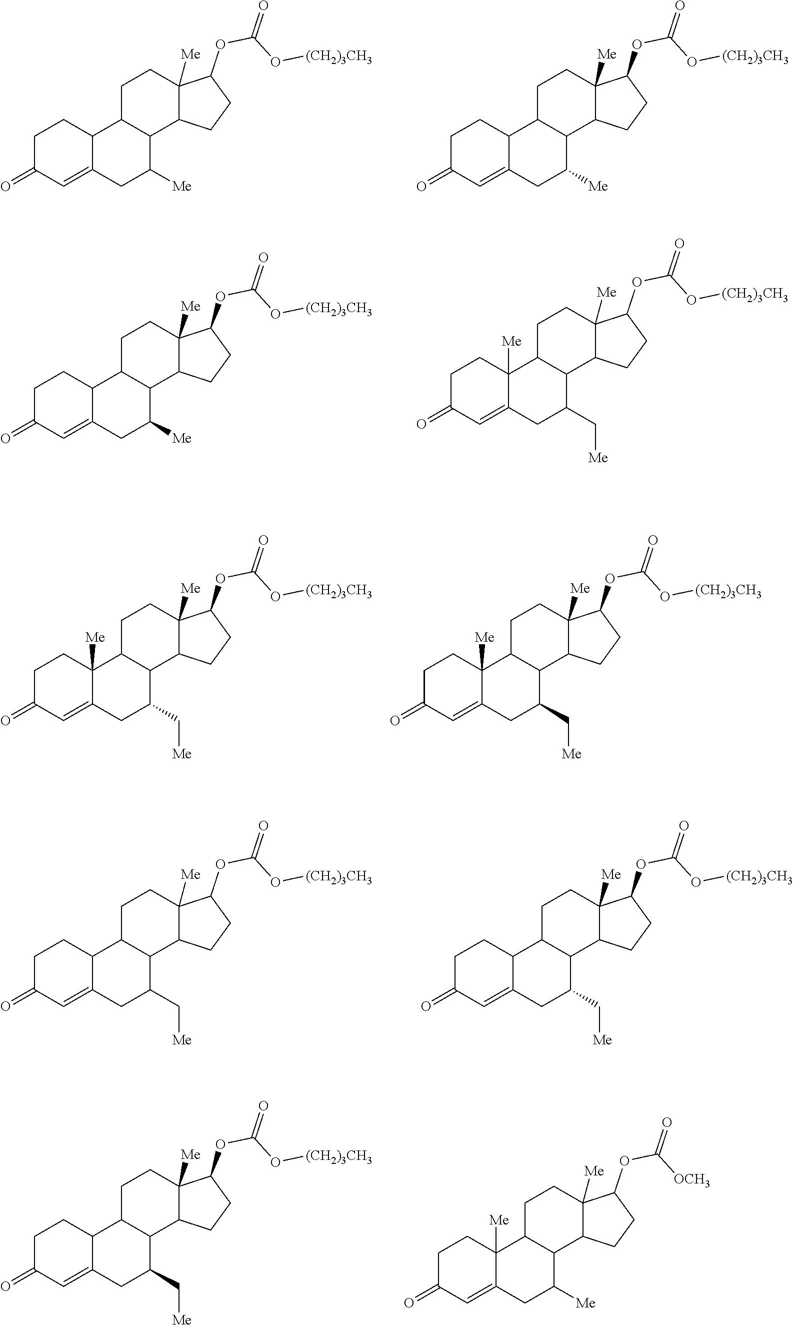

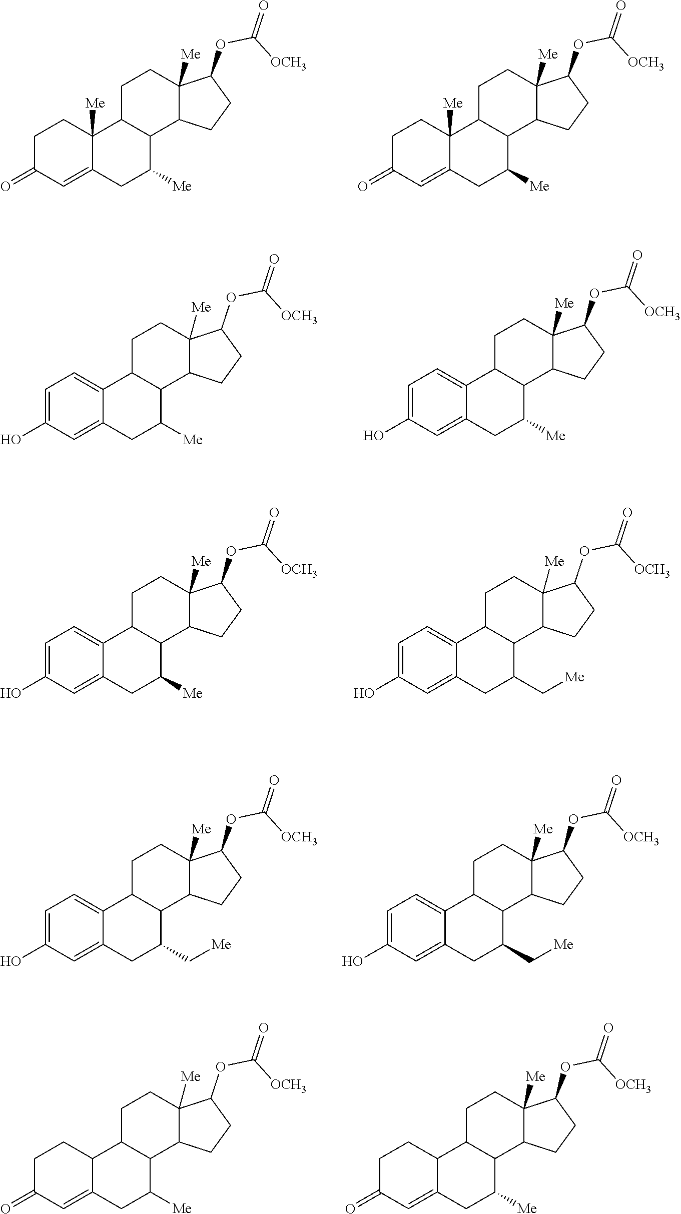

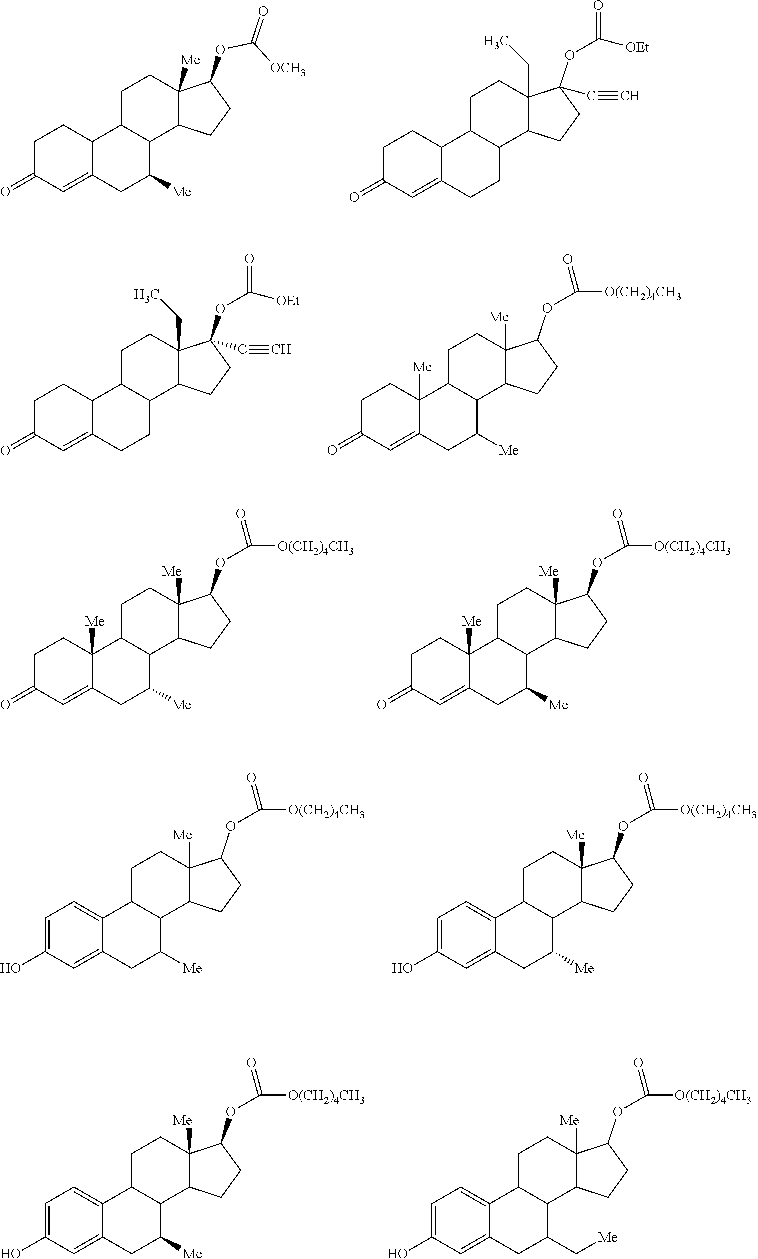

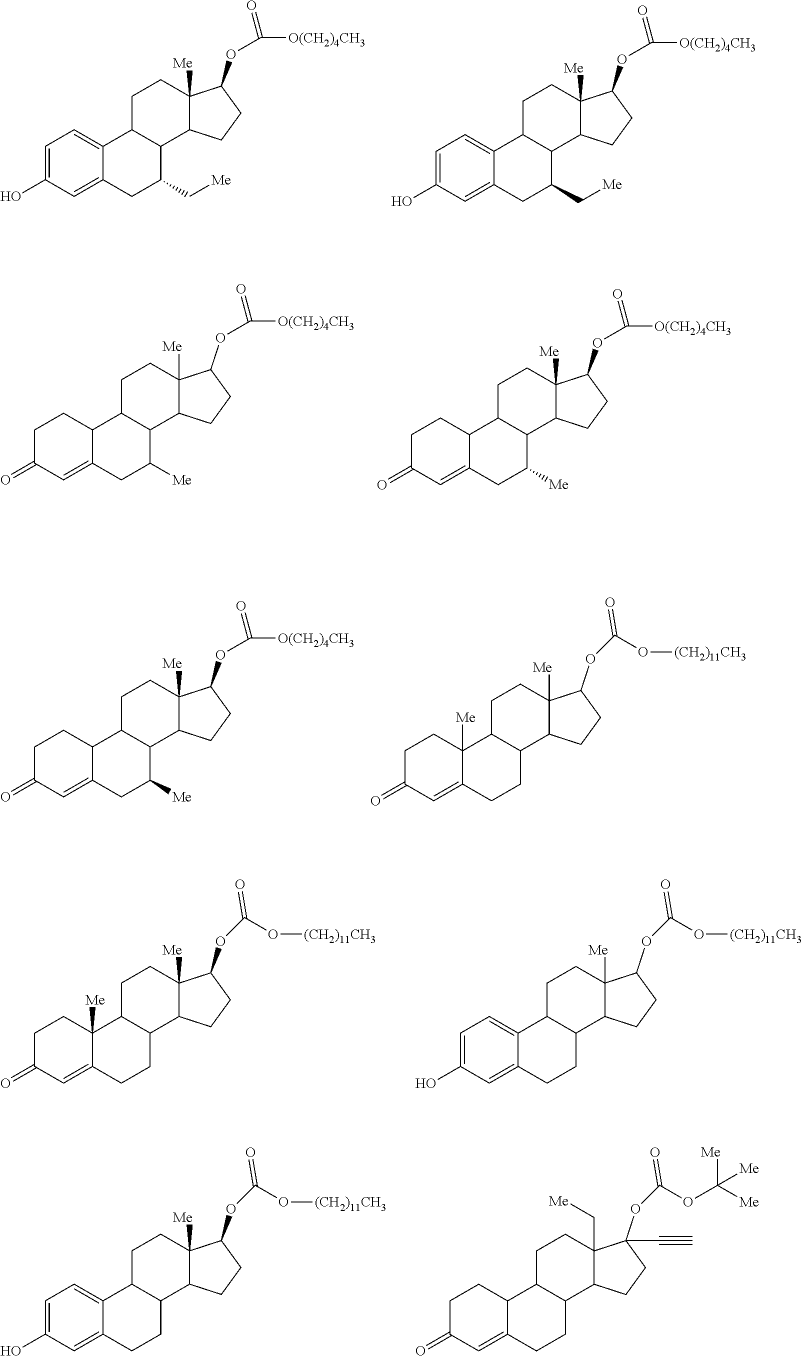

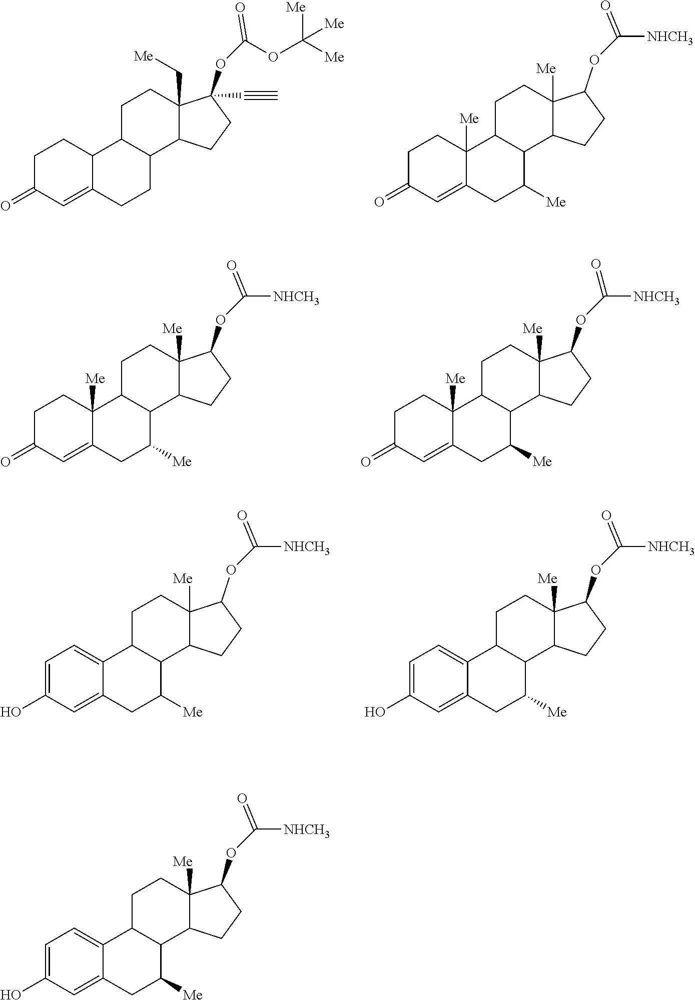

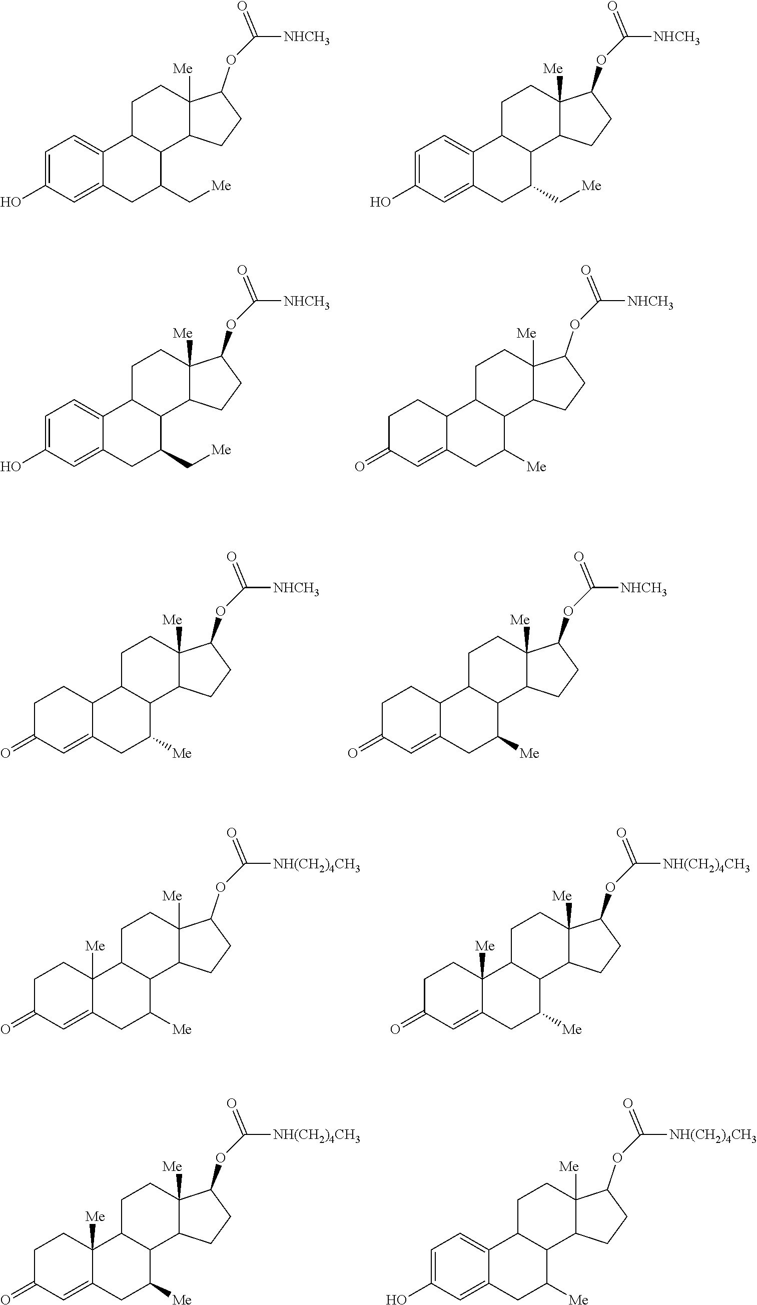

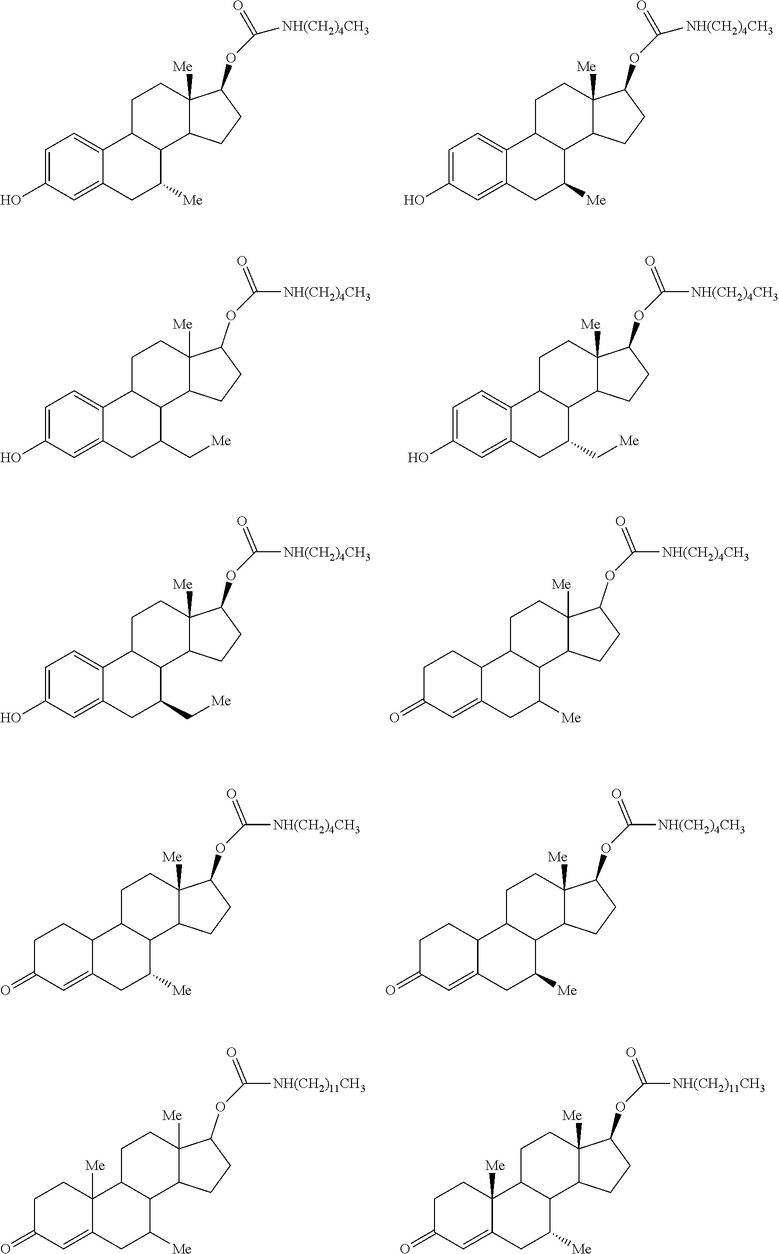

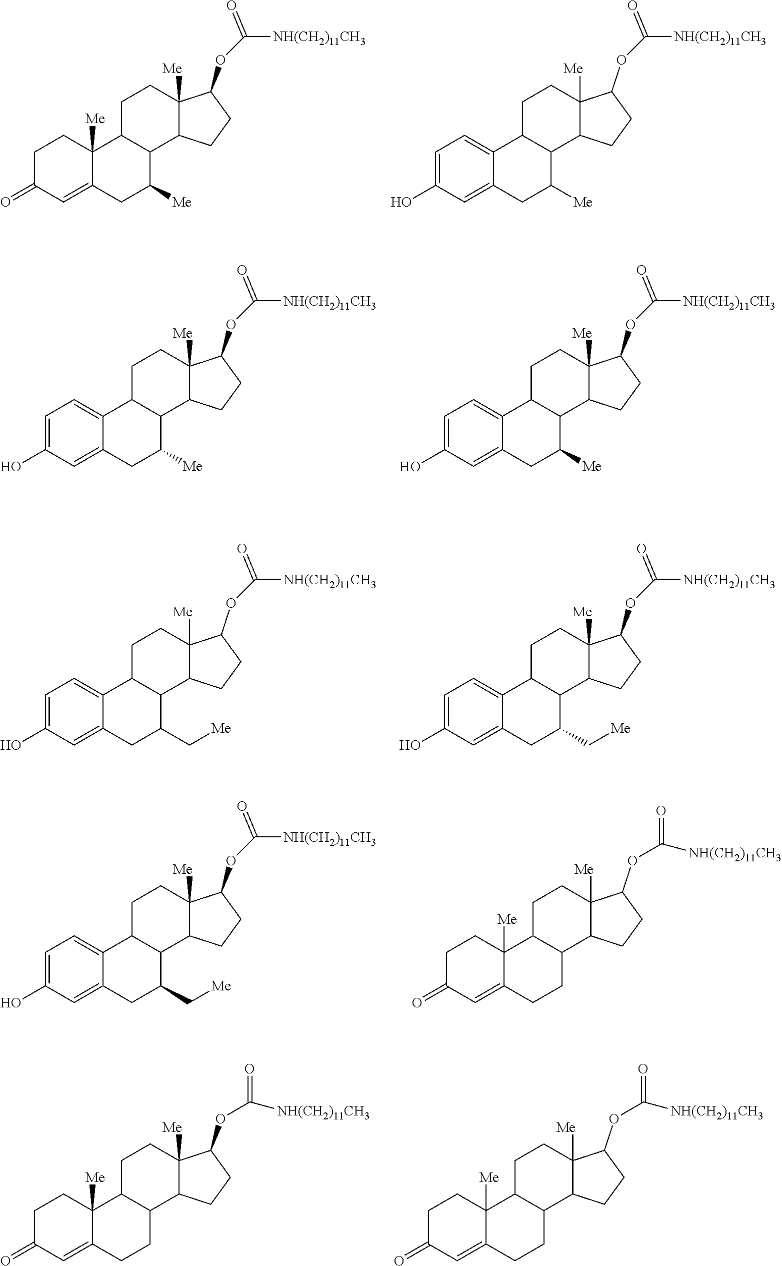

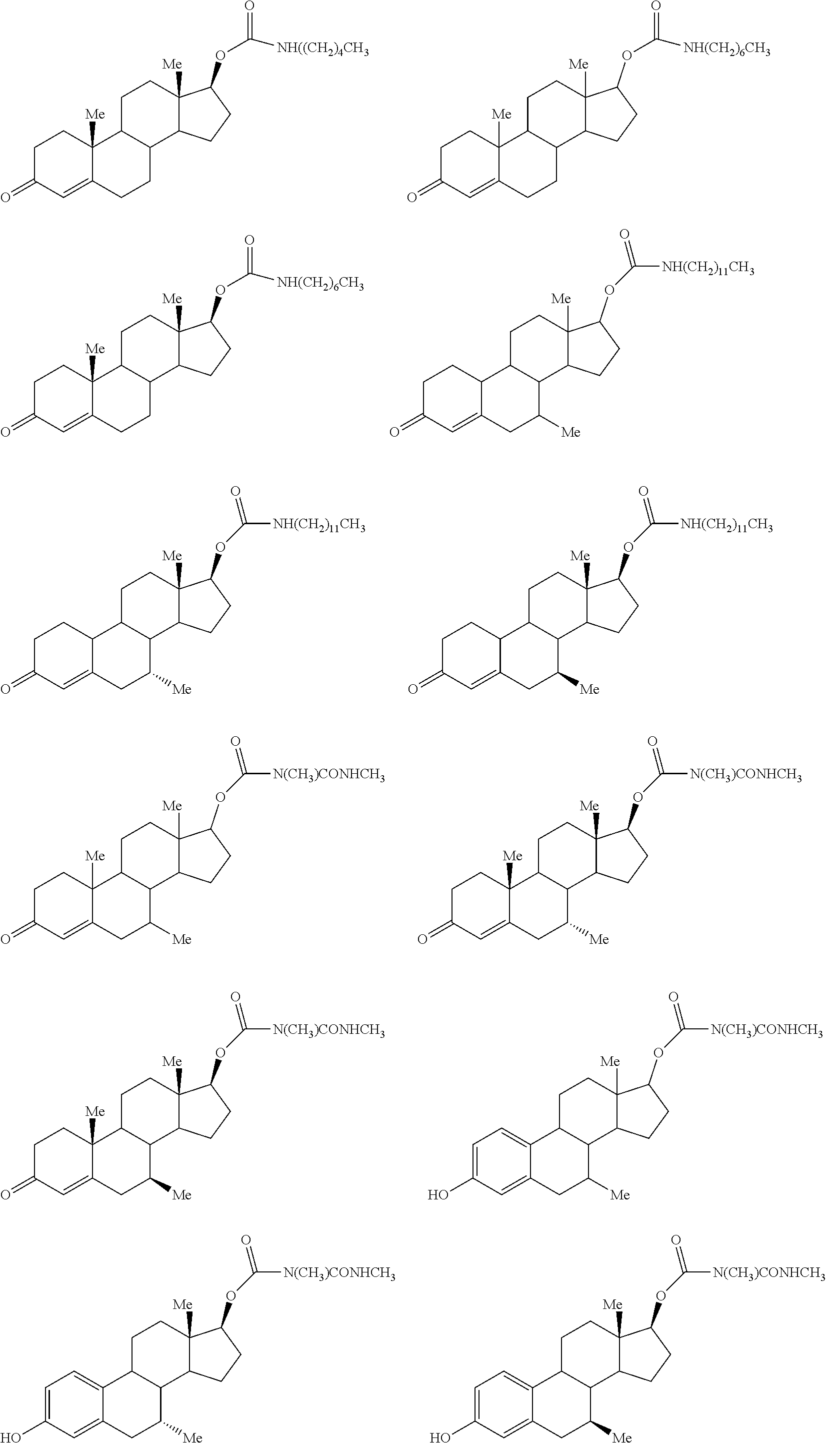

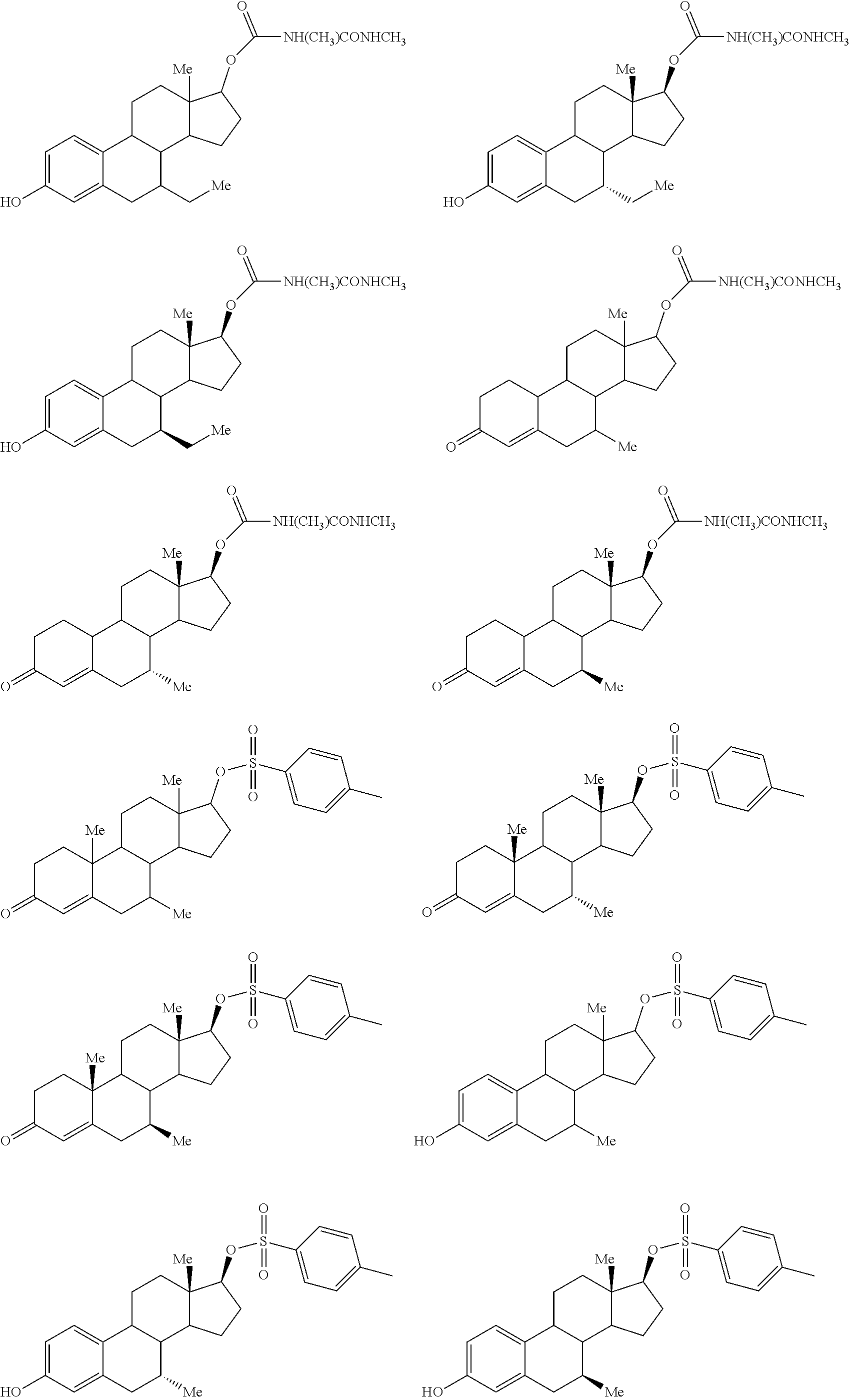

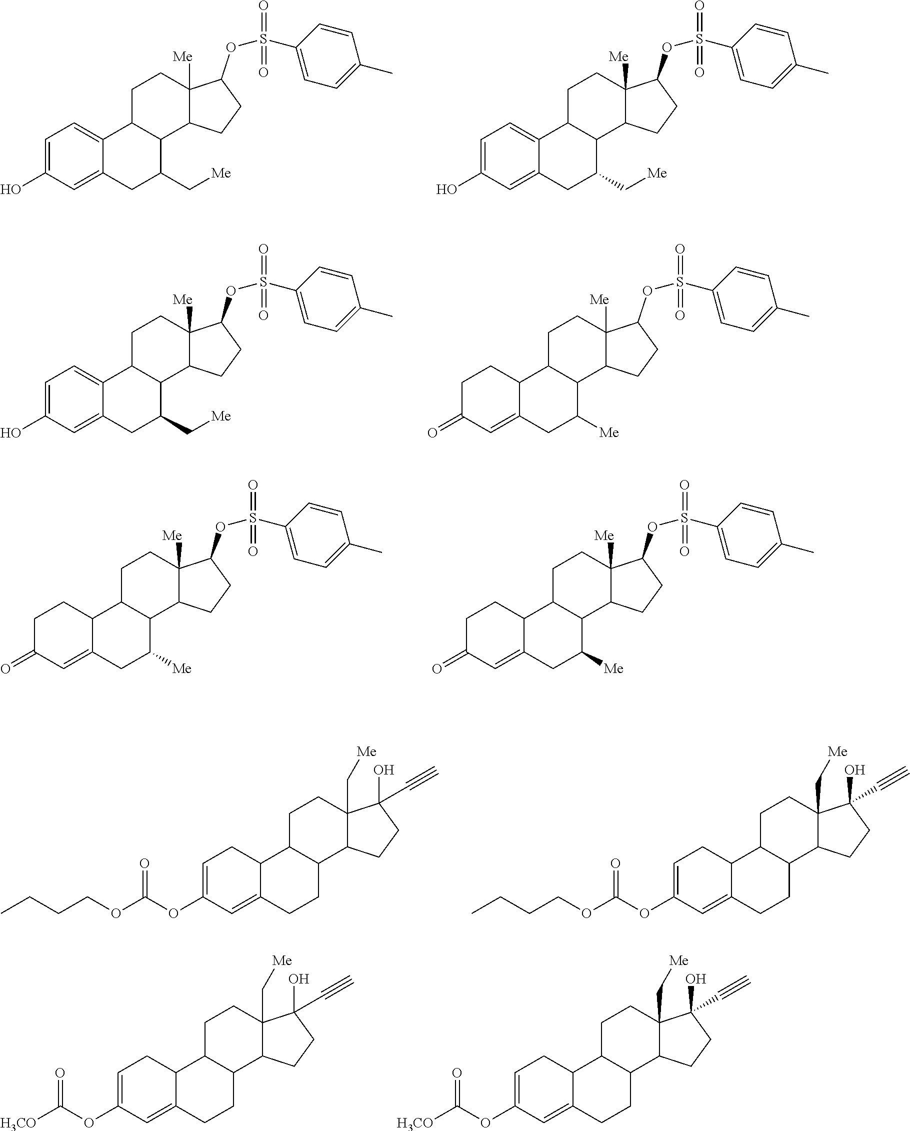

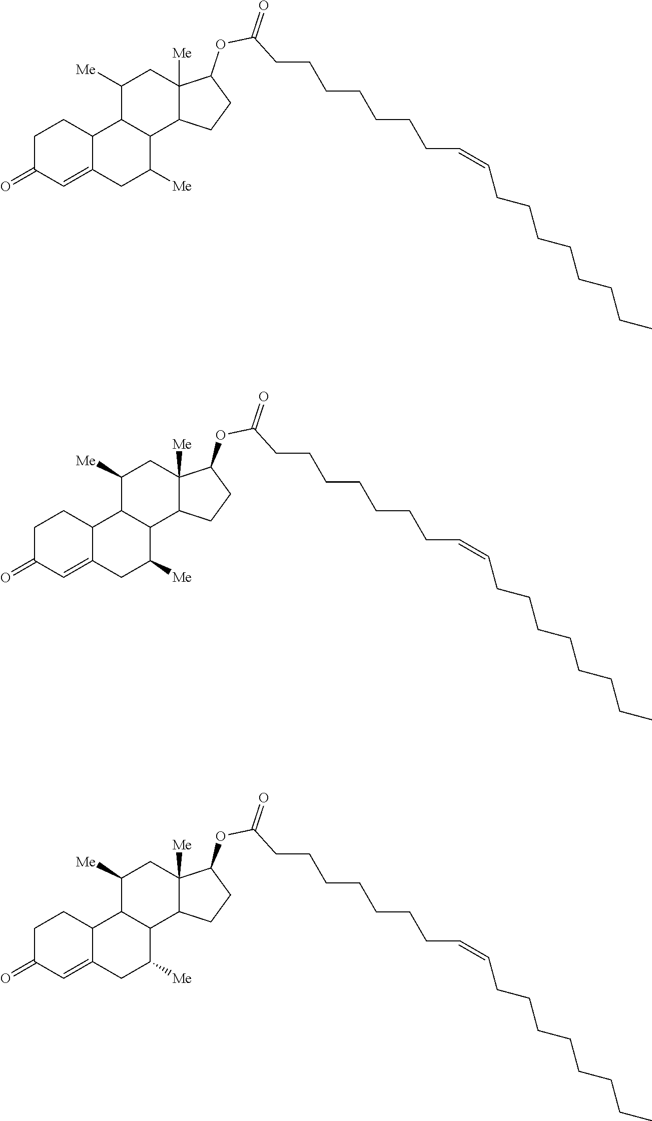 [SMILES: CCCCCCCC/C=C\CCCCCCCC(=O)OC1CCC2C3C(C)CC4=CC(=O)CCC4C3C(C)CC12C ; CCCCCCCC/C=C\CCCCCCCC(=O)O[C@H]1CCC2C3C(C4CCC(=O)C=C4C[C@@H]3C)[C@@H](C)C[C@@]21C ; CCCCCCCC/C=C\CCCCCCCC(=O)O[C@H]1CCC2C3C(C4CCC(=O)C=C4C[C@H]3C)[C@@H](C)C[C@@]21C]

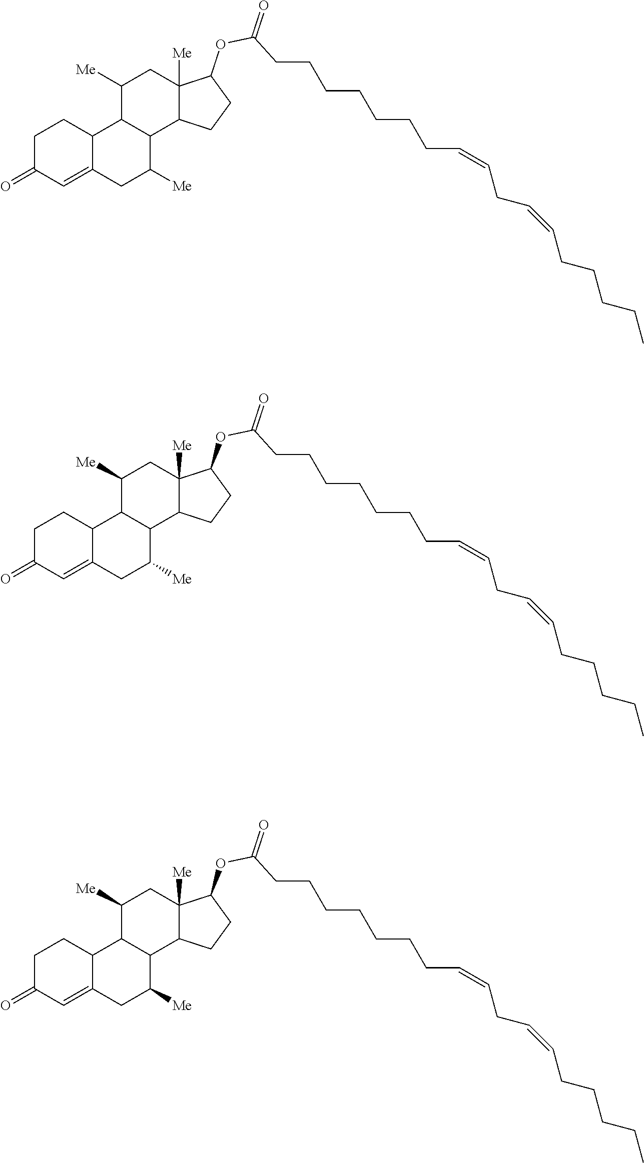 [SMILES: CCCCC/C=C\C/C=C\CCCCCCCC(=O)OC1CCC2C3C(C)CC4=CC(=O)CCC4C3C(C)CC12C ; CCCCC/C=C\C/C=C\CCCCCCCC(=O)O[C@H]1CCC2C3C(C4CCC(=O)C=C4C[C@H]3C)[C@@H](C)C[C@@]21C ; CCCCC/C=C\C/C=C\CCCCCCCC(=O)O[C@H]1CCC2C3C(C4CCC(=O)C=C4C[C@@H]3C)[C@@H](C)C[C@@]21C]

Also disclosed are embodiments of oligomeric compounds comprising combinations of steroidal-based compounds and/or therapeutic agents that are covalently bound with a linker group. In particular embodiments, if the oligomeric compound comprises one or more steroidal-based compound, at least one such steroidal-based compound is attached to the linker group by a functional group located at the C17 position of the steroidal-based compound. In some embodiments, the steroidal-based compound of an oligomeric compound embodiment has a structure of Formula IX, below, including a pharmaceutically acceptable salt, a prodrug, a solvate, or a tautomer thereof.

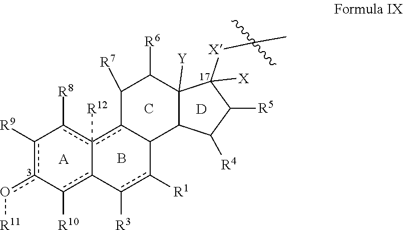

With reference to Formula IX, the wavy line indicates the position to which at least one linker group according to Formula X (described below) is attached to the steroidal-based compound. For Formula IX, each of X, Y, R³, R⁴, R⁵, R⁶, R¹, R⁹, and R¹⁰ can be as recited above for any of the Formulas described above (e.g., as recited for any of Formulas I, II, III, IV, V, VI, VII, IA, IB, IIA, IIB, IIIA, IIIB, IVA, IVB, VA-VD, VA′-VD′, and VA″-VD″; Formulas VIA-VID, VIA′-VID′, and VIA″-VID″; and/or Formulas VIIA-VIID, VIIA′-VIID′, and VIIA″-VIID″); X′ can be oxygen or —C(O)(CH₂)ₚ—, wherein p is an integer selected from 1 to 10, such as 1 to 8, or 1 to 6, or 1 to 4, or 1 or 2; R⁷ can be as recited for any of the formulas described above, or can be a ═O group; R¹ can be H, D, halogen, aliphatic, heteroaliphatic, aromatic, heteroaliphatic, haloaliphatic, haloheteroaliphatic, or an organic functional group; R¹¹ (if present) can be selected from hydrogen, aliphatic, heteroaliphatic, aromatic, heteroaliphatic, haloaliphatic, haloheteroaliphatic, an organic functional group, or another linker group according to Formula X; and R¹² (if present) can be hydrogen or aliphatic.

In some particular embodiments, each of R³, R⁴, R⁵, R⁶, R⁸, R⁹, and R¹⁰ are hydrogen or deuterium; each of R⁷, R¹², and Y independently is lower alkyl; R¹ is Cl, F, I, Br, alkyl, alkenyl, alkynyl, aryl, heteroaryl, heteroalkyl, heteroalkenyl, heteroalkynyl, or any combination of these groups; X is hydrogen, —OH, or —CCH; R¹¹ (if present) is hydrogen, lower alkyl, a linker group according to Formula X, —C(O)Ph, or —C(Z)(CH₂)_{q}CH₃, wherein Z is S, O, or NH and q is an integer selected from 0 to 10, such as 0 to 8, or 0 to 6, or 0 to 4, or 0 to 2. In some such embodiments, q is 0, 1, 2, 3, 4, 5, 6, 7, 8, 9, or 10.

In some embodiments, the steroidal-based compound can be an estradiol-based compound in which case the A ring is an aromatic ring and R¹¹ is present. In yet other embodiments, the steroidal-based compound can be a prednisone-based compound in which case the A ring comprises two double bonds and R¹¹ is not present (and thus ring A comprises a carbonyl group). In yet other embodiments, the steroidal-based compound can be a testosterone-based compound in which case ring A comprises a single double bond and R¹¹ is not present. In yet additional embodiments, the steroidal-based compound can be a dihydrotestosterone-based compound, in which case ring A does not comprise any double bonds and R¹¹ is not present.

In some embodiments, the oligomeric compound is a dimer compound that is a homodimer having two of the same steroidal-based compounds bound together. In some other embodiments, the oligomeric compound can be a dimer compound that is a heterodimer having two different steroidal-based compounds bound together or a combination of a steroidal-based compound and a therapeutic agent. In yet additional embodiments, the oligomeric compound embodiments can comprise three or more steroidal-based compounds (which can be the same or different from one another); a combination of two or more steroidal-based compounds and one or more therapeutic agents; or a combination of a steroidal-based compound and two or more therapeutic agents. In some embodiments comprising three or more steroidal-based compounds, two of the steroidal-based compounds can each be coupled to a third steroidal-based compound such that the third steroidal-based compound is coupled to the oxygen atom at the C17 positions of each other steroidal-based compound through a linker group and wherein each of these other steroidal-based compounds are coupled, via the linker group, to the oxygen atoms present at C3 and C17 of the third steroidal-based compound.

The oligomeric compound embodiments comprise at least one linker group that facilitates coupling the various components of the oligomeric compound together (wherein each of the various components, that is, any steroidal-based compound and/or any therapeutic agent, are generically referred to herein as “monomeric components”). The linker group also can be selected to facilitate selective cleavage of the monomeric components from one another, which provides the ability to control delivery rates, timing, and/or activity of the various monomeric components. In some embodiments, the linker group can be selected to provide a selective cleavage event such that one monomeric component is cleaved and “activated” while the other retains the linker group (or a portion thereof) and remains in an “un-activated” form. In some additional embodiments, the linker group can be selected to control the rate of release of the monomeric components. The linker group also can be selected to facilitate selective activation and/or deactivation of a receptor to which a monomeric component may bind. In particular embodiments, the linker group is attached to a functional group of steroidal-based compound located at the C17 position. In an independent embodiment, the linker group is not attached at the C7 position of a steroidal-based compound. Embodiments of the oligomeric compound embodiments can be used for providing fertility suppression, hormone replacements therapy, hypogonadism treatment, bone protection, contraception, endometriosis, metabolic diseases, and the like.

Linker groups of the present disclosure can have a structure represented by Formula X, below.

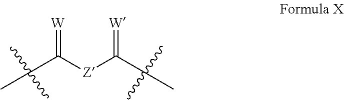

With reference to Formula X, each of W and W′ independently can be selected from oxygen, sulfur, or NR^{d}, wherein R^{d} can be H, aliphatic, heteroaliphatic, aromatic, heteroaliphatic, haloaliphatic, haloheteroaliphatic, or a combination thereof; and Z′ can be selected from aliphatic, heteroaliphatic, aromatic, heteroaliphatic, haloaliphatic, haloheteroaliphatic, or a combination thereof. The wavy lines of Formula X indicate the site of disconnection from the monomeric component. In particular embodiments, each of W and W′ independently are oxygen or sulfur and Z′ is selected from —(CH₂)ₘ—; —O(CH₂)ₘO—; —O[(CH₂)ₘO]_{m′}; —NR^{e}(CH₂)ₘNR^{e}—; or —(CH₂)ₘNR^{c}C(O)(CH₂)ₘ—; —(CH₂)ₘNR^{e}C(O)NR^{e}(CH₂)_{m′}; —[(CH₂)ₘO]_{m′}—; —NR^{e}[(CH₂)ₘNR^{e}]_{m′}; —(CH₂)ₘNR^{e}—; —[(CH₂)ₘNR^{e}]_{m′}—; —O(CH₂)ₘNR^{e}—, —O[(CH₂)ₘNR^{e}]_{m′}; or —NR^{e}[(CH₂)ₘO]_{m′} wherein R^{e} can be H, aliphatic, heteroaliphatic, aromatic, heteroaliphatic, haloaliphatic, haloheteroaliphatic, or a combination thereof and each of m and m′ independently is an integer ranging from 1 to 20, such as 1 to 15, or 1 to 10, or 1 to 5 (e.g., 1, 2, 3, 4, 5, 6, 7, 8, 9, 10, 11, 12, 13, 14, 15, 16, 17, 18, 19, 20, or higher). Representative linker groups are illustrated below.

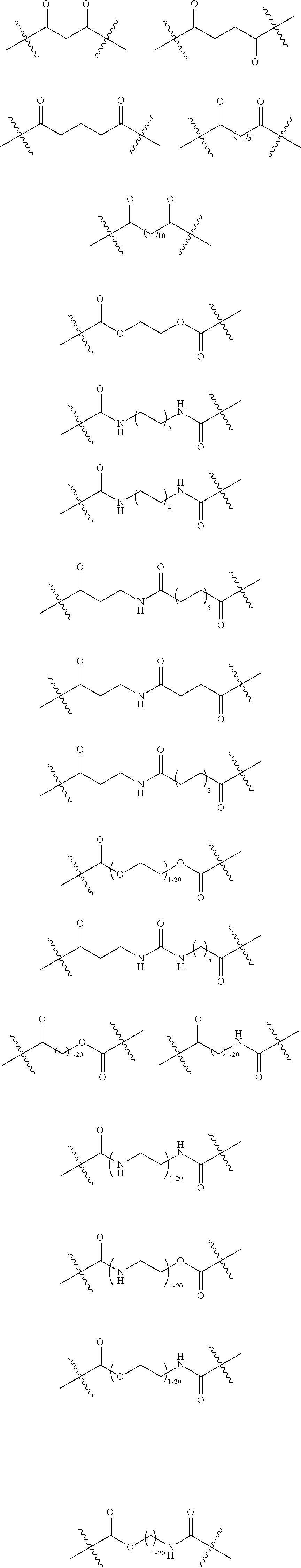

As described herein, some oligomeric compound embodiments can comprise a therapeutic agent that is attached to a steroidal-based compound through a linker group. Such embodiments can provide additional therapeutic activity in addition to any fertility suppression, hormone replacements, hypogonadism treatments, contraception, and/or bone protection treatments. In such embodiments, the therapeutic agent can be an agent that provides anticancer properties, a GnRH antagonist and agonist, E3 ubiquitin ligase recruiting ligands, kinase antagonist and agonist, GPCR antagonist and agonist, anticancer agent, an antimalarial agent, and other types of therapeutic agents. In particular embodiments, the therapeutic agent can be hydroxychloroquine, cisplatin, carboplatin, camptothecin, cyclophosphamide, doxorubicin, fluorouracil, methotrexate, paclitaxel, taxotere, prednisone, glasdegib, amifampridine, apalutamide, avatrombopag, baloxavir marboxil, bictegravir, binimetinib, dacomitinib, doravirine, duvelisib, elagolix, encorafenib, eravacycline, fosravuconazole, fruquintinib, gilteritinib, ivosidenib, larotrectinib, lorlatinib, omadacycline, plazomicin, plitidepsin, pyrotinib, revefenacin, rifamycin, roxadustat, sarecycline, tafenoquine, talazoparib, tecovirimat, tezacaftor, relugolix, degarelix, ganirelix, cetrorelix, abarelix, enzalutamide, and other therapeutics.

In some embodiments, the steroidal-based compound of the oligomeric compound can be selected from a steroid compound comprising a substituent at positions C7 and/or C10. In such embodiments, the steroidal-based compound can have a structure according to any one of the Formulas described herein, wherein R² is replaced with a linker group according to Formula X.

In yet additional embodiments, the steroidal-based compound can be any natural and/or non-natural steroid, including, but not limited to, levonorgestrel, 11β-methyl-19-nortestosterone, 7α-methylnortestosterone, 7α-ethyl estradiol, 7β-ethyl estradiol, 7α-methyl estradiol, 7β-methyl estradiol, 7α-methyl testosterone, 7β-methyl testosterone, 7α-ethyl testosterone, 7β-ethyl testosterone, 7α-t-butyl testosterone, 7β-t-butyl testosterone, 7α-phenyl testosterone, 7β-phenyl testosterone, dimethandrolone, prednisone, estradiol, dexamethasone, testosterone, 7α-methyl dihydrotestosterone, 7-methyl dihydrotestosterone, (7R,11S,13S,17S)-7,11,13-trimethyl-7,8,9,11,12,13,14,15,16,17-decahydro-6H-cyclopenta[a]phenanthrene-3,17-diol, (11S,13S,17S)-11,13-dimethyl-7,8,9,11,12,13,14,15,16,17-decahydro-6H-cyclopenta[a]phenanthrene-3,17-diol, (13S,17R)-13-ethyl-17-ethynyl-2,3,8,9,10,11,12,13,14,15,16,17-dodecahydro-1H-cyclopenta[a]phenanthrene-3,17-diyl dibutyrate, (11S,13S,17S)-17-hydroxy-11,13-dimethyl-1,2,6,7,8,9,10,11,12,13,14,15,16,17-tetradecahydro-3H-cyclopenta[a]phenanthren-3-one-2,2,4,6,6,10-d6, (11S,13S,17S)-17-hydroxy-11,13-dimethyl-1,2,6,7,8,11,12,13,14,15,16,17-dodecahydro-3H-cyclopenta[a]phenanthren-3-one, (10R,13S,17R)-13-ethyl-17-ethynyl-17-hydroxy-1,2,6,7,8,9,10,11,12,13,14,15,16,17-tetradecahydro-3H-cyclopenta[a]phenanthren-3-one-2,2,4,6,6,10-d6, (13S,17R)-13-ethyl-17-(ethynyl-d)-17-hydroxy-1,2,6,7,8,9,10,11,12,13,14,15,16,17-tetradecahydro-3H-cyclopenta[a]phenanthren-3-one-2,2,4,6,6,10-d6, (10R,11S,13S,17S)-17-hydroxy-11,13-dimethyl-1,2,6,7,8,9,10,11,12,13,14,15,16,17-tetradecahydro-3H-cyclopenta[a]phenanthren-3-one-2,2,4,6,6,7,7-d7, or (7R,10R,11S,13S,17S)-17-hydroxy-7,11,13-trimethyl-1,2,6,7,8,9,10,11,12,13,14,15,16,17-tetradecahydro-3H-cyclopenta[a]phenanthren-3-one-2,2,4,6,6-d5.

Representative structures of such steroidal-based compounds are provided below (and also can include a pharmaceutically acceptable salt, a prodrug, a solvate, or a tautomer thereof):

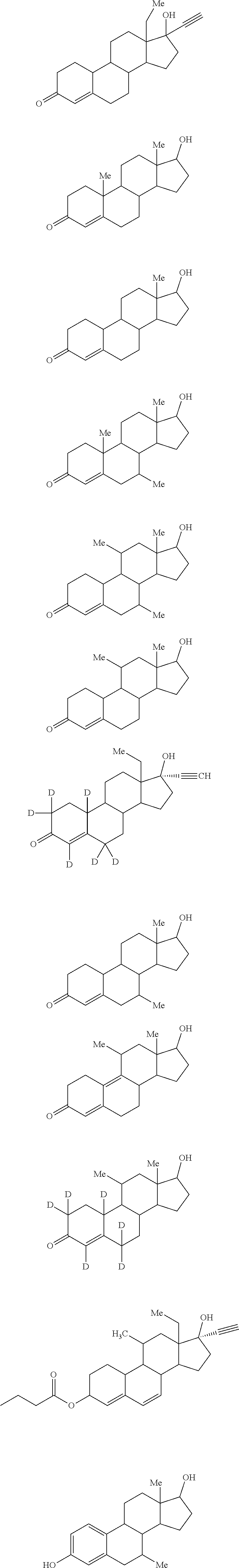

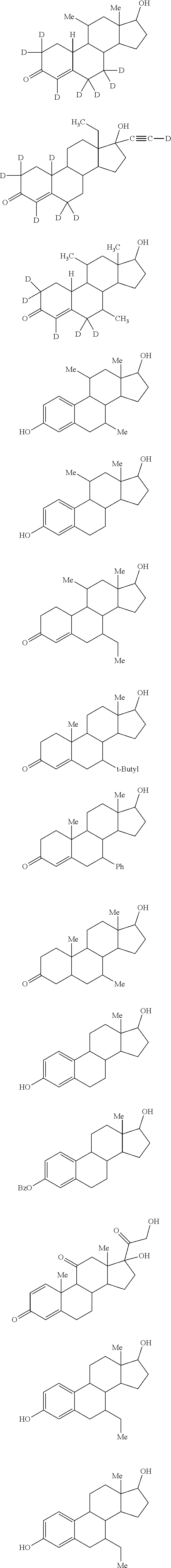

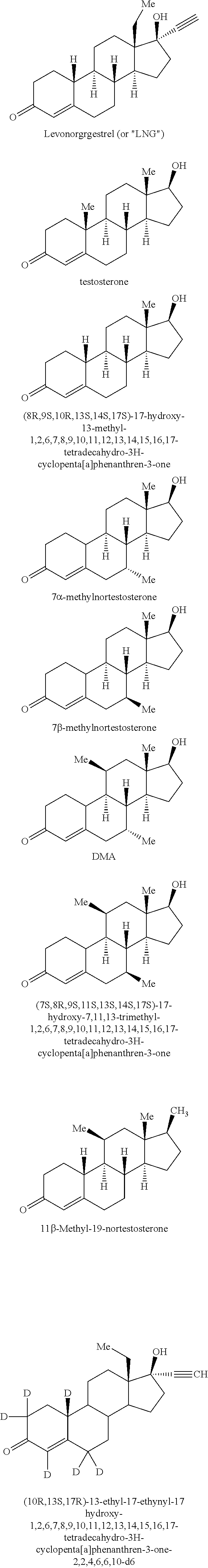

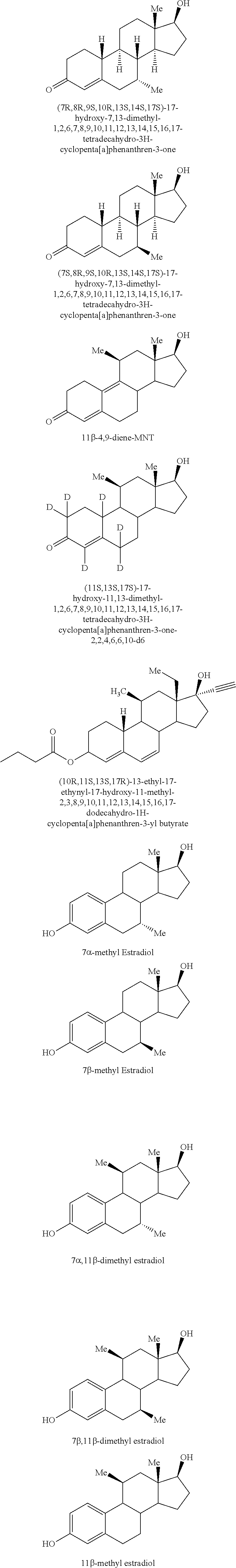

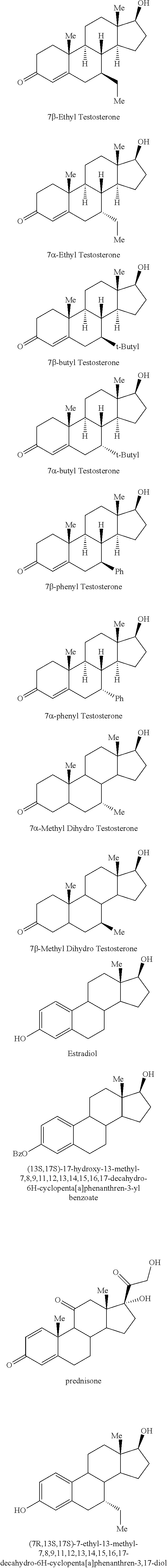

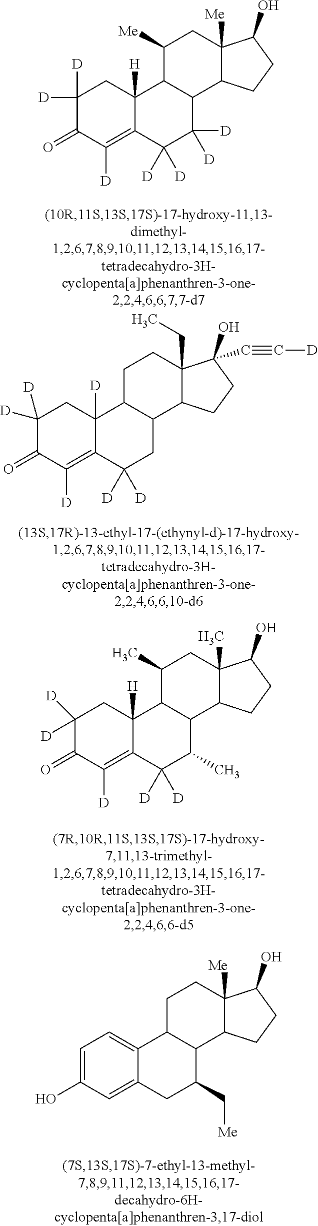 [SMILES: [2H]C1=C2[C@H](CC([2H])([2H])C1=O)C1C(C3CC[C@H](O)[C@@]3(C)C[C@@H]1C)C([2H])([2H])C2([2H])[2H] ; [2H]C#C[C@]1(O)CCC2C3CC([2H])([2H])C4=C([2H])C(=O)C([2H])([2H])CC4([2H])C3CC[C@@]21CC ; [2H]C1=C2[C@H](CC([2H])([2H])C1=O)C1C(C3CC[C@H](O)[C@@]3(C)C[C@@H]1C)[C@H](C)C2([2H])[2H] ; CC[C@H]1Cc2cc(O)ccc2C2CC[C@@]3(C)C(CC[C@@H]3O)C21]

In such embodiments, a first linker group is attached at the C17 hydroxyl group (or the hydroxyl group of the C17 carbonyl group in the case of prednisone) and in some embodiments, a second linker group can be attached at the C3 hydroxyl group. In such embodiments, the hydrogen atom of each such hydroxyl group is not present.

Representative oligomeric compound embodiments are provided below.

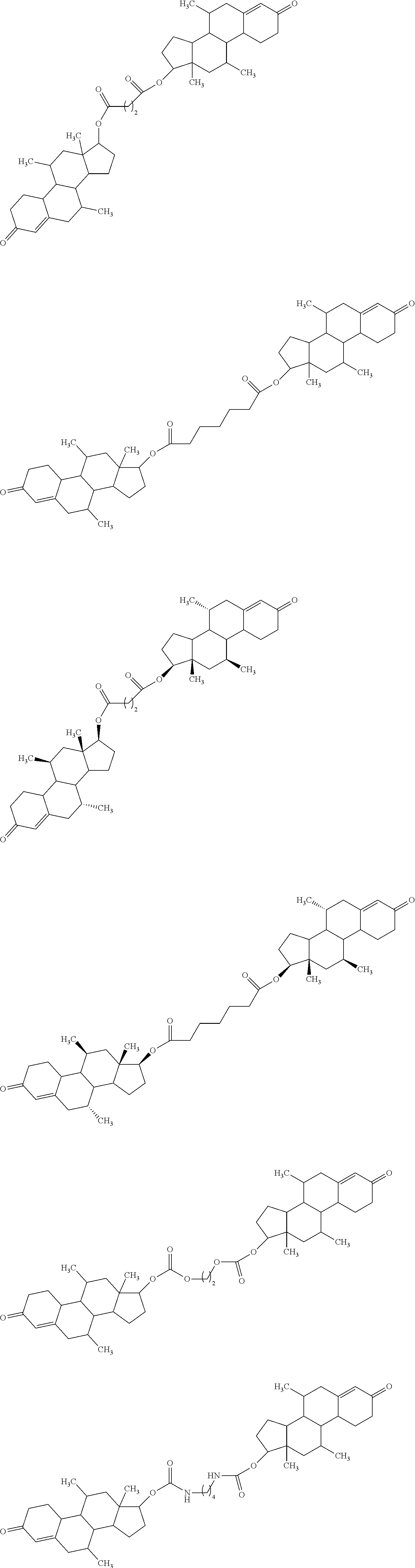

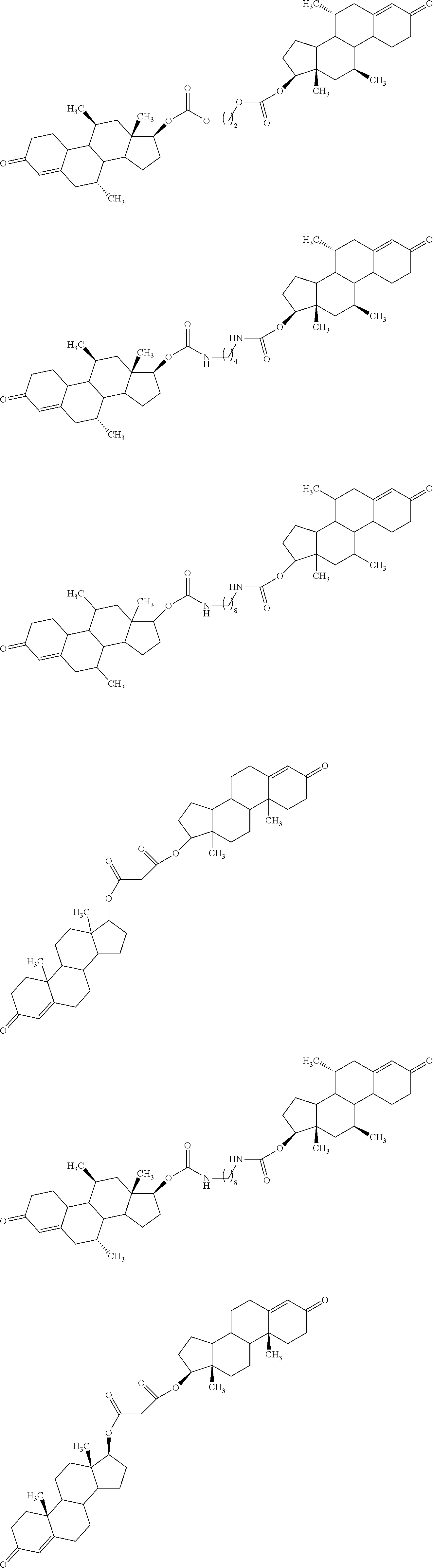

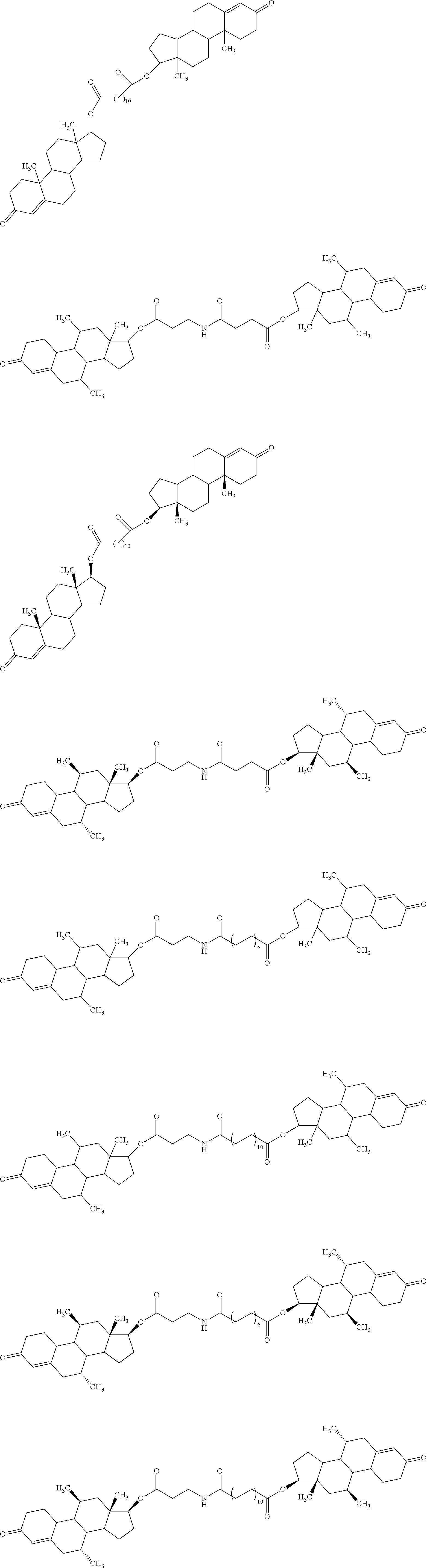

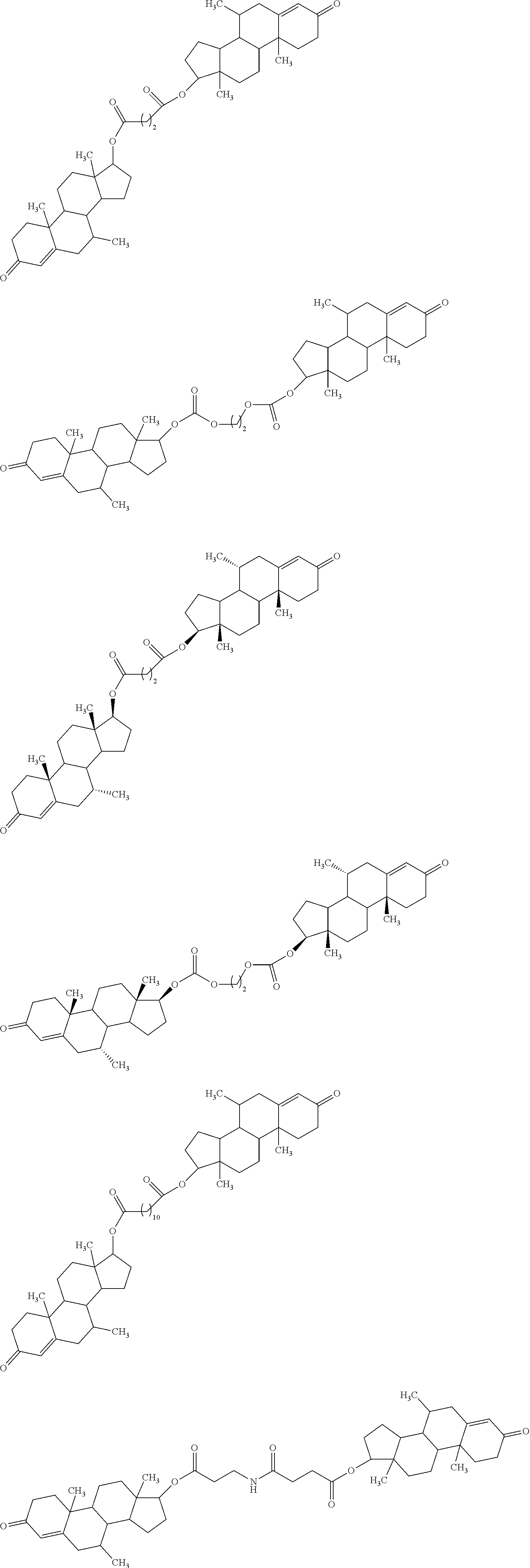

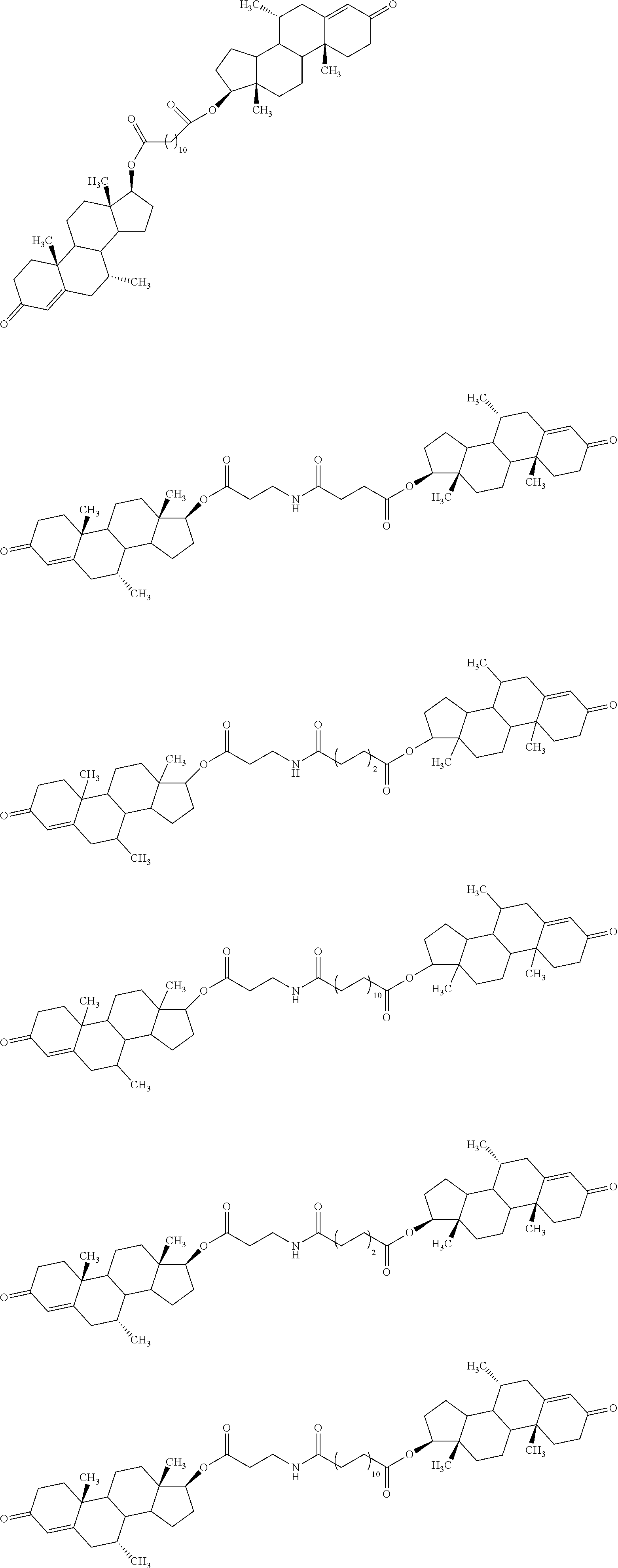

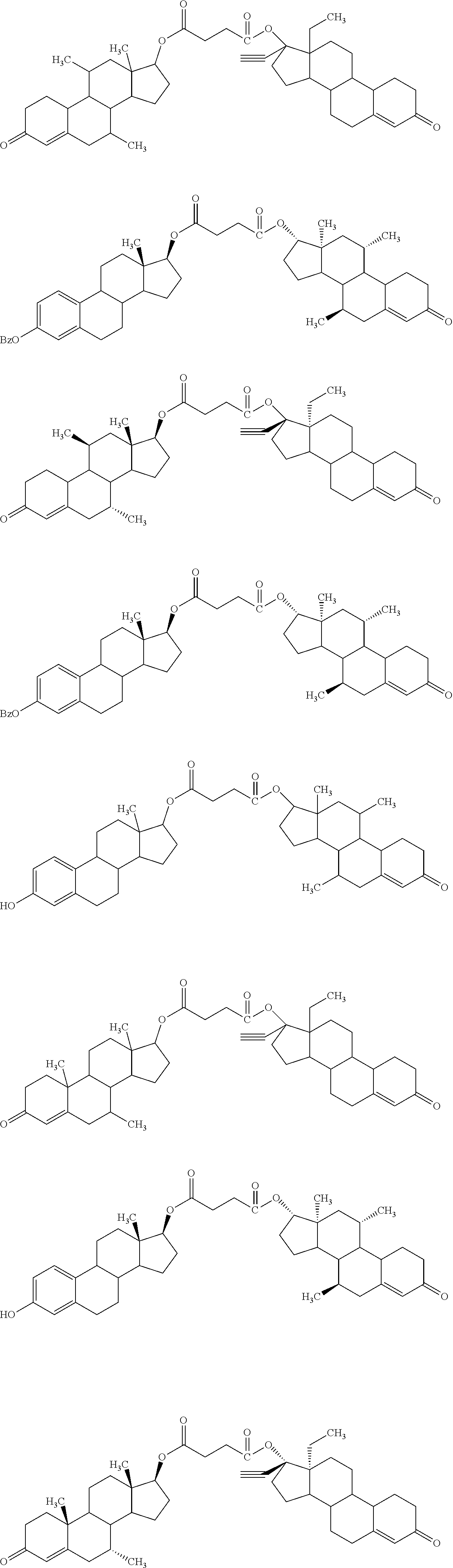

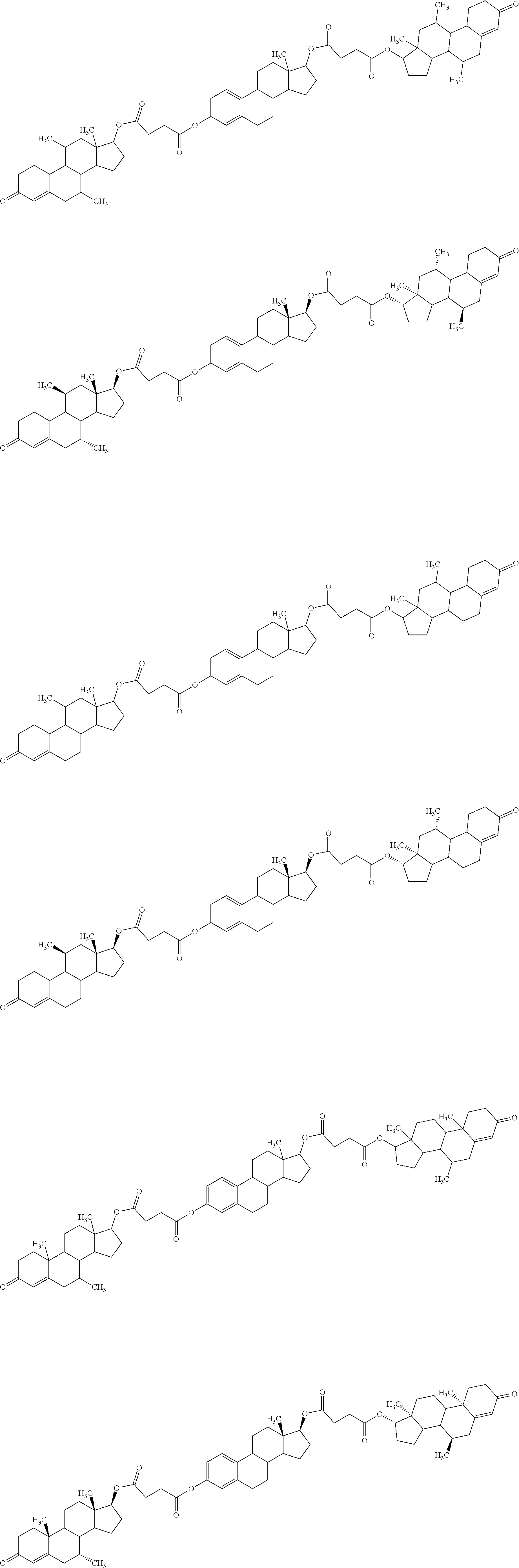

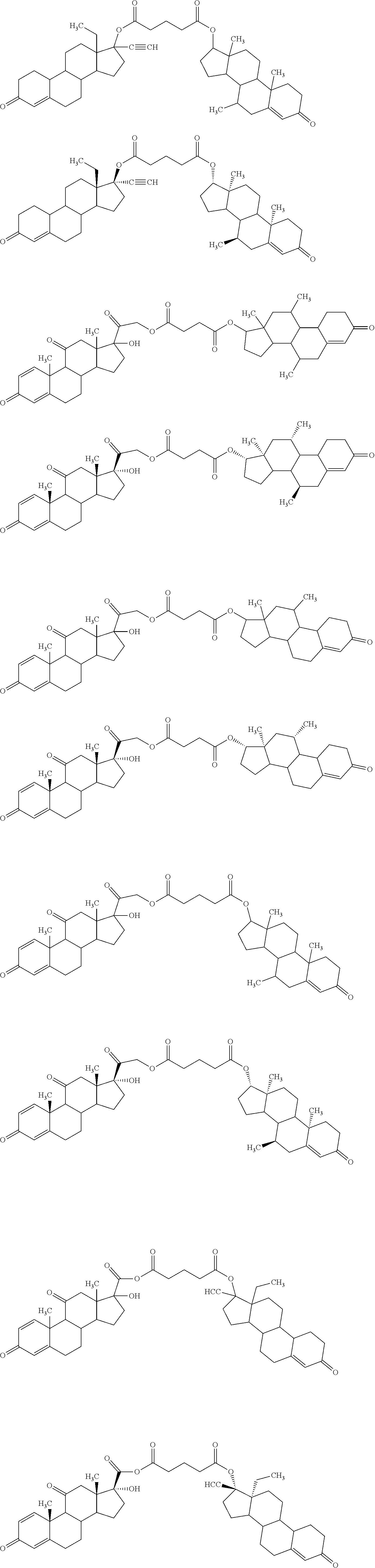

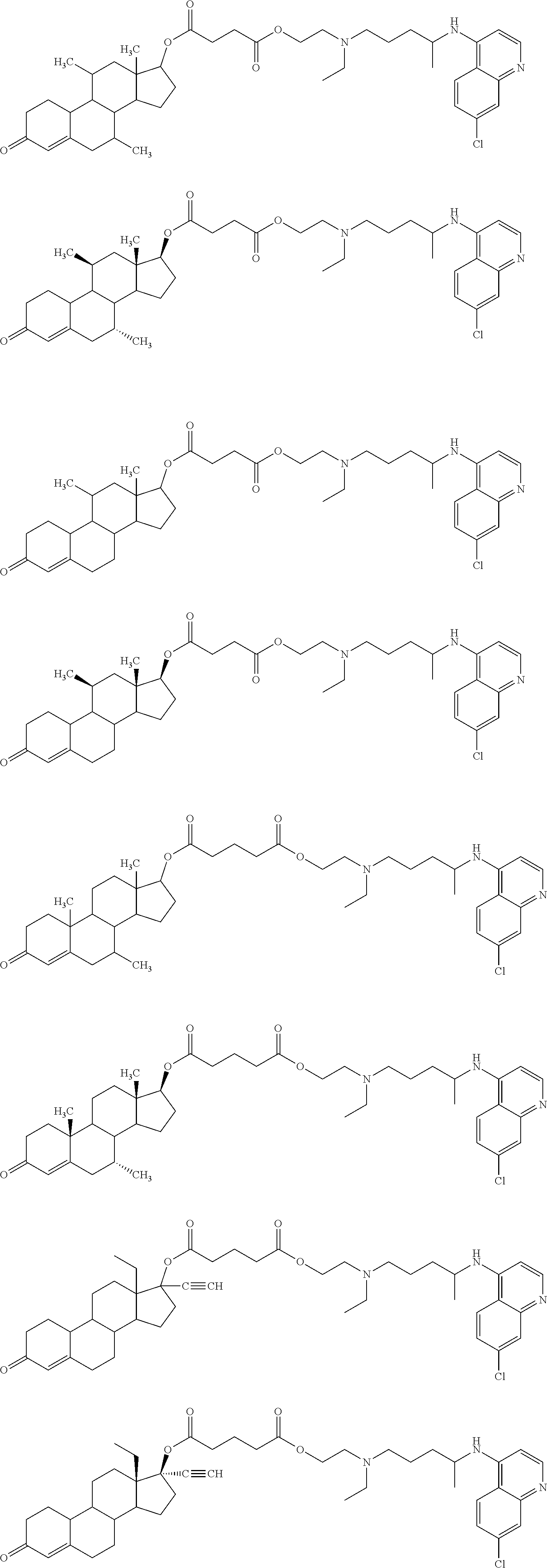

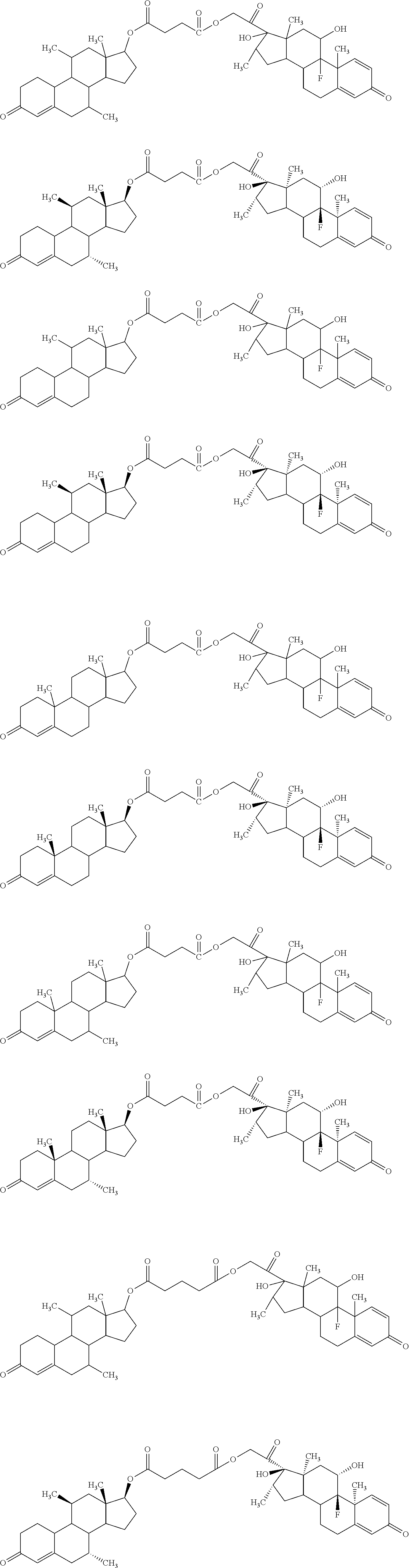

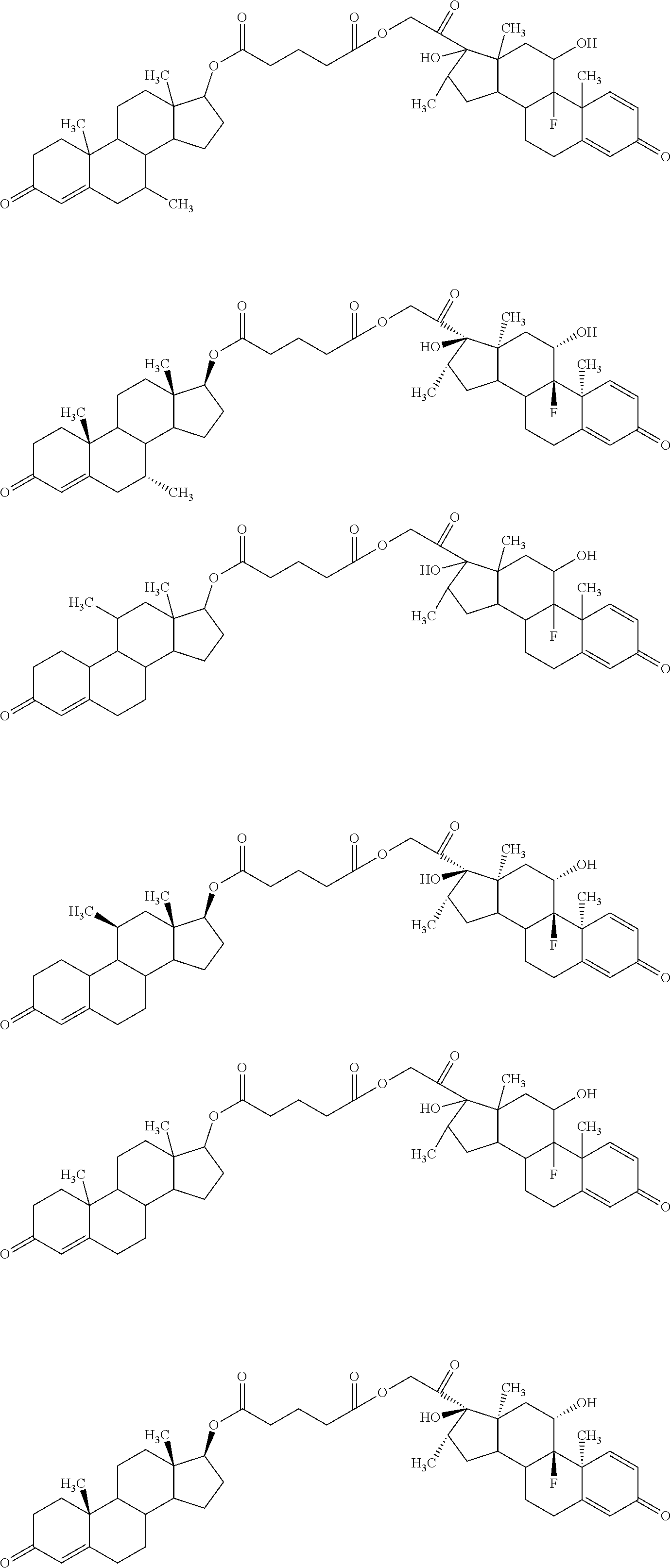

Also disclosed herein are embodiments of a pharmaceutically acceptable composition and/or a formulation comprising any one or more of the monomeric compound embodiments and/or oligomeric compound embodiments of the present disclosure. In some embodiments, the pharmaceutically acceptable composition can comprise a single monomeric compound and/or a single oligomeric compound as described above, or a plurality of such compounds that can be the same or different. In some embodiments, the pharmaceutically acceptable composition can comprise a pharmaceutically acceptable salt, stereoisomer, tautomer, solvate, or prodrug of the one or more compounds. In some embodiments, a formulation can comprise the compound or a composition thereof, and one or more additional components, as discussed herein.

Pharmaceutically acceptable composition and/or formulation embodiments comprising one or more of the monomeric compound embodiments and/or oligomeric compound embodiments of the present disclosure typically comprise the monomeric compound and/or the oligomeric compound (or a plurality of such compounds) in a therapeutically effective amount. In some additional embodiments, the amount of the monomeric compound embodiments and/or oligomeric compound embodiments ranges from greater than 0% up to 99% total weight percent. In some embodiments, pharmaceutically acceptable composition and/or formulation embodiments comprising one or more of the monomeric compound and/or oligomeric compound embodiments disclosed herein comprise from greater than 0 wt % to 95 wt %, such as 0.001 wt % to 95% wt %, or 0.01 wt % to 95 wt %, or 0.1 wt % to 95 wt %, or 1 wt % to 95 wt %, or 5 wt % to 95 wt %, or 10 wt % to 95 wt %, or 25 wt % to 95 wt % of the monomeric compound and/or oligomeric compound (or a plurality of such compounds) based on the total weight percent of the pharmaceutically acceptable composition or formulation. In some embodiments, pharmaceutically acceptable composition and/or formulation embodiments comprising one or more of the monomeric and/or oligomeric compound embodiments disclosed herein comprise from greater than 0 wt % to 95 wt %, such as greater than 0 wt % to 90% wt %, or greater than 0 wt % to 85 wt %, or greater than 0 wt % to 80 wt %, or greater than 0 wt % to 75 wt %, or greater than 0 wt % to 70 wt %, or greater than 0 wt % to 65 wt %, or greater than 0 wt % to 60 wt %, or greater than 0 wt % to 55 wt %, or greater than 0 wt % to 50 wt % or lower of the monomeric compound and/or oligomeric compound (or a plurality of such compounds) based on the total weight percent of the pharmaceutically acceptable composition or formulation. The remaining weight percent of the composition and/or formulation can be made up of any one or more of the other compositional components described below.

Pharmaceutically acceptable composition and/or formulation embodiments can further comprise a pharmaceutically-acceptable excipient, such as, but not limited to, an adjuvant, a carrier, a stabilizer, or combinations thereof. The pharmaceutically acceptable composition also can include additional components, such as diluents, fillers, binding agents, moisturizing agents, preservatives, acids, and the like, and any and all combinations thereof. In particular embodiments, the pharmaceutically-acceptable excipient is selected from an aqueous suspending vehicle (or “ASV”), which can comprise carboxymethylcellulose, sodium salt, sodium chloride, benzyl alcohol, TWEEN® 80, and distilled water; sesame oil; ethyl alcohol; benzyl benzoate; castor oil; or combinations thereof. In exemplary embodiments, the pharmaceutically-acceptable excipient can comprise ethyl alcohol in sesame oil (10:90 v/v) or benzyl benzoate (or “BBZ”) in castor oil (30:70 w/w).

The monomeric compound embodiments and/or oligomeric compound embodiments of the present disclosure (or compositions and/or formulations thereof) can be administered in the form of solids, liquids, and/or lotions. Suitable solid forms of administration include, but are not limited to, tablets, capsules, powders, solid dispersions, and the like containing the monomeric and/or oligomeric compounds (or compositions and/or formulations thereof). Suitable liquid or lotion forms include, but are not limited to, oil-in-water or water-in-oil emulsions, aqueous gel compositions, or liquids or lotions comprising the monomeric and/or oligomeric compounds (or compositions and/or formulations thereof) formulated for use as foams, films, sprays, ointments, pessary forms, suppository forms, creams, liposomes or in other forms embedded in a matrix for the slow or controlled release of the monomeric compound embodiment and/or oligomeric compound embodiment (or compositions and/or formulations thereof) to the skin or surface onto which it has been applied or is in contact. In particular disclosed embodiments, a dermal patch can be used to facilitate dosing and delivering the monomeric compound embodiment and/or oligomeric compound embodiment (or compositions and/or formulations thereof). In some additional embodiments, a microneedle array can be used to facilitate dosing and delivering the monomeric compound embodiment and/or oligomeric compound embodiment of the present disclosure (or compositions and/or formulations thereof). In yet additional embodiments, the monomeric compound embodiment and/or oligomeric compound embodiment (or compositions and/or formulations thereof) can be formulated as a gel for topical administration.

The monomeric compound embodiments and/or oligomeric compound embodiments of the present disclosure (or compositions and/or formulations thereof) may be formulated so as to be suitable for a variety of modes of administration, including, but not limited to, topical, ocular, oral, buccal, systemic, nasal, injection (such as intravenous, intraperitoneal, subcutaneous, intramuscular, or intrathecal), transdermal (e.g., by mixing with a penetrating agent, such as DMSO), rectal, vaginal, a form suitable for administration by inhalation or insufflation, a form suitable for implantation, or any combination thereof.

For oral or buccal administration, the monomeric compound embodiment and/or oligomeric compound embodiment (or compositions and/or formulations thereof) may take the form of lozenges, tablets, or capsules prepared by using conventional means with pharmaceutically acceptable excipients that would be recognized by people of ordinary skill in the art with the benefit of the present disclosure. The tablets or capsules may be coated by methods well known in the art with, for example, sugars, films, or delayed-release, sustained-release, and/or enteric coatings.

Liquid preparations of the monomeric compound embodiment and/or oligomeric compound embodiment (or compositions and/or formulations thereof) for oral administration may take the form of, for example, elixirs, solutions, syrups or suspensions, or they may be presented as a dry product for constitution with water or other suitable vehicle before use. Preparations for oral administration also may be suitably formulated to give controlled release of the compound or the composition.

For topical administration, the monomeric compound embodiment and/or oligomeric compound embodiment (or compositions and/or formulations thereof) can be formulated as solutions, lotions, gels, ointments, creams, suspensions, and the like. For transmucosal administration, penetrants appropriate to the barrier to be permeated can be used.

Systemic formulations include those designed for administration by injection, e.g., subcutaneous, intravenous, intramuscular, intrathecal or intraperitoneal injection, as well as those designed for transdermal, transmucosal oral or pulmonary administration. Useful injectable preparations include sterile suspensions, solutions or emulsions of the monomeric compound embodiment and/or oligomeric compound embodiment (or compositions and/or formulations thereof) in aqueous or oily vehicles. The composition may also contain formulating agents, such as suspending, stabilizing and/or dispersing agents.

For rectal routes of administration, the monomeric compound embodiment and/or oligomeric compound embodiment (or compositions and/or formulations thereof) may be formulated as solutions (for retention enemas) suppositories or ointments containing conventional suppository bases, such as cocoa butter or other glycerides.

For nasal administration or administration by inhalation or insufflation, the monomeric compound embodiment and/or oligomeric compound embodiment (or compositions and/or formulations thereof) can be conveniently delivered in the form of an aerosol spray from pressurized packs or a nebulizer with the use of a suitable propellant. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the monomeric compound embodiment and/or oligomeric compound embodiment (or compositions and/or formulations thereof) and a suitable powder base such as lactose or starch.

### IV. METHOD EMBODIMENTS

A. Synthesis

Monomeric compound embodiments disclosed herein can be made using synthetic methods described herein. In some embodiments, the monomeric compound embodiments can be made using a precursor 100 comprising a double bond between C6 and C7 as illustrated in Scheme 1. The R¹ group can be added at the C7 position using suitable conjugate addition conditions, such as by exposing the precursor 100 to a lithium compound (e.g., LiCl), a catalyst (e.g., CuI), a Grignard reagent comprising the desired R¹ group (e.g., R¹MgCl), and a silyl reagent (e.g., TMSCl). The protecting group of the C17-hydroxyl group can be removed during this step. The resulting free-hydroxyl compound 102 can then be converted to the desired R²-functionalized compound 104 using suitable conditions, depending on the type of R² groups desired in the product. In some embodiments, the method can comprise one or more purification steps, such as using column chromatography to purify free-hydroxyl compound 102 and/or R²-functionalized compound 104. In yet additional embodiments, one or more crystallization steps can be used. Aromatized versions of the compounds can be generated upon reaction with an aromatase enzyme.

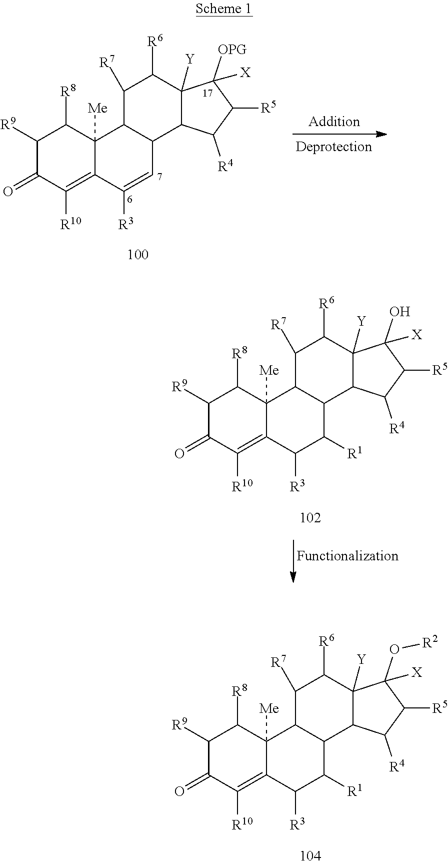

Representative methods for making particular compound embodiments according to the present disclosure are provided in the Examples section. A person of ordinary skill in the art will recognize that method embodiments described herein can be adapted to make other compound embodiments contemplated by the present disclosure that may not be expressly illustrated herein, particularly with the benefit of the present disclosure.

Also disclosed herein are embodiments of a method for making oligomeric compound embodiments disclosed herein. In some embodiments, the method can comprise preparing a steroidal-based compound 200, wherein X″ can be a free hydroxyl group and/or a carbonyl-containing group (e.g., —C(O)(CH₂)ₚOH, wherein p is an integer selected from 1 to 10, such as 1 to 8, or 1 to 6, or 1 to 4, or 1 or 2). The free hydroxyl or carbonyl-containing group can be used as a handle to attach the linker group as illustrated in Scheme 2. In some embodiments, the linker group can be coupled to compound 200 using a linker group precursor (e.g., an anhydride-based version of Formula X, wherein the carbon atoms directly attached to W and W′ are joined together through an oxygen atom; or an acid-based version of Formula X, wherein the carbon atoms directly attached to W and W′ each further is bound to a hydroxyl group) and a suitable esterifying reagent (e.g., 4-dimethylaminopyridine [or “DMAP” ] or the like). In some embodiments, the linker group can first be coupled to the steroidal-based compound, followed by a further coupling step wherein an additional steroidal-based compound (labeled as “SC” in product 202 of Scheme 2) or a therapeutic agent (labeled as “TA” in product 202 of Scheme 2) can be coupled to provide the oligomeric compound embodiment. This additional coupling step whereby the additional steroidal-based compound or the therapeutic agent is bound to the linker group can be accomplished using suitable coupling reagents (e.g., dicyclohexyl carbodiimide [or “DCC” ] with DMAP, and the like). In some embodiments, the linker group precursor can first be coupled to the additional steroidal-based compound or therapeutic agent to form a conjugate and then the conjugate can be coupled to the steroidal-based compound.

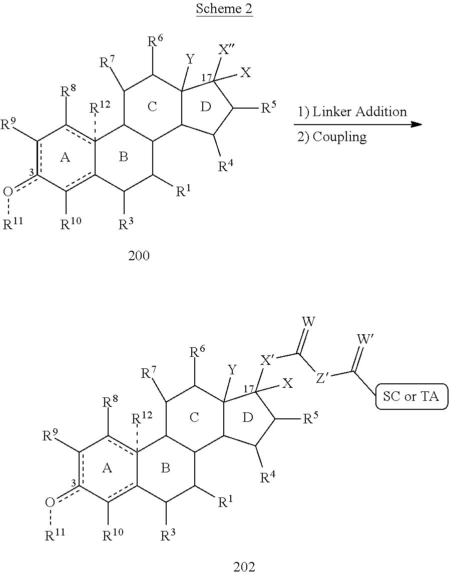

Representative methods for making particular oligomeric compound embodiments according to the present disclosure are provided in the Examples section. A person of ordinary skill in the art will recognize that method embodiments described herein can be adapted to make other oligomeric compound embodiments contemplated by the present disclosure that may not be expressly illustrated herein, particularly with the benefit of the present disclosure.

In yet additional embodiments, product 202 from Scheme 2 can be further coupled with another steroidal-based compound and/or therapeutic agent by either directly coupling this third component to the oxygen atom at the C3 position or by coupling this oxygen atom with a second linker group and then coupling the second linker group to the third component using techniques described herein.

B. Method of Use

Disclosed herein are method embodiments for using the monomeric compound embodiments and/or oligomeric compound embodiments (or compositions and/or formulations thereof) according to the present disclosure. In some embodiments, monomeric compound embodiments of the present disclosure can be used as contraceptive agents, and particularly as male contraceptive agents. Embodiments of the monomeric compounds are capable of inhibiting fertility of a subject, particularly male subjects, and thus can function as effective male contraceptives. The monomeric compound embodiments have low to no toxicity. In some embodiments, the monomeric compound (or compounds derived in vivo from such monomeric compound embodiments) exhibits androgenic activity, progestogenic activity, or both. Upon administration to a subject, the monomeric compound (or compound derived in vivo from the monomeric compound embodiment) also is able to undergo aromatization by interaction with an aromatase to provide a corresponding estradiol-based compound. A schematic showing the aromatase-driven aromatization of testosterone to estradiol is illustrated below solely by way of example.

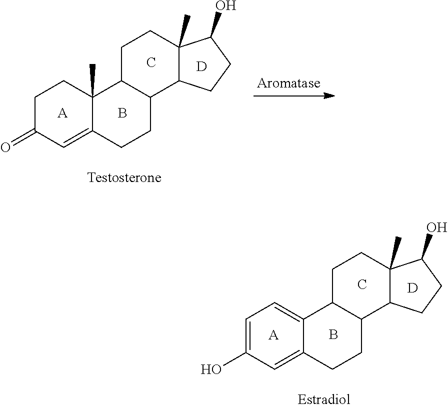

Monomeric compound embodiments of the present disclosure are able to utilize a similar mechanism to thereby confer estrogenic activity to the compound, in addition to its androgenic and/or progestogenic activities. As such, monomeric compound embodiments of the present disclosure are able to prevent issues associated with estrogenic deficiency, such as (but not limited to) bone density loss and/or ineffective carbohydrate/lipid metabolism. Estrogenic deficiency is a side-effect associated with many male contraceptives known in the art, but which can be avoided and/or limited by using the present monomeric compound embodiments as they are able to be converted to estradiol-containing compound embodiments and thus can serve as estrogen surrogates and/or can be used to treat osteoporosis. In yet some additional embodiments, the monomeric compound can be used as an endocrine therapy (or other hormone therapy), a hormone replacement, an endometriosis therapy, a metabolic disease therapy, a hypogonadism therapy (e.g., hypogonadotropic eunuchoidism, fertile eunuch syndrome, prepubertal panhypopituitarism, postpubertal pituitary failure, Klinefelter's syndrome, Reifenstein's syndrome, functional prepubertal castration syndrome, male “Turner's syndrome,” Sertoli cell-only syndrome, adult seminiferous tubule failure, adult Leydig cell failure, and the like), and the like.

A method of using the monomeric compound embodiments of the present disclosure is described. In some embodiments, the method comprises administering a monomeric compound of any one of the Formulas described herein, or a composition or formulation thereof, to a subject. In yet additional embodiments, the method comprises administering a monomeric compound of any one of the Formulas described herein, or a composition or formulation thereof, to a male subject. In some embodiments, the monomeric compound is administered using any mode of administration discussed herein. In particular embodiments, the monomeric compound embodiment can be administered via oral administration, injection, transdermal administration, inhalation, implantation, or the like. In some embodiments, the method comprises administering a monomeric compound of any one of the Formulas described herein, or a composition or formulation thereof, to a male subject to alter the activity of an androgen and/or progesterone receptor.

Oligomeric compound embodiments of the present disclosure also can be used as contraceptive agents. In some embodiments, the oligomeric compound embodiments are used as male and/or female contraceptive agents. In yet additional embodiments, the oligomeric compound can be used as a contraceptive agent as well as an endocrine therapy. The monomeric components of the oligomeric compound can include steroidal-based compounds discussed herein, and thus can provide the androgenic, progestogenic, estrogenic, glucocorticoid, and/or mineralocorticoid activity of such steroidal-based compound embodiments. In yet additional embodiments, the oligomeric compound can comprise a therapeutic agent coupled to one or more steroidal-based compound embodiments. In such embodiments, the oligomeric compound can be used as a dual therapy whereby it can provide contraceptive properties as well as an additional therapeutic benefit, such as cancer therapy (e.g., chemotherapy, leukemia therapy, protein degradation therapy, and the like), sickle cell therapy, immunosuppression, autoimmune therapy, cardiovascular therapy, an antifungal therapy, an antibacterial therapy, an antiviral therapy, an endometriosis therapy, a metabolic disease therapy, pulmonary therapy, gastrointestinal therapy, GnRH therapy, hypogonadism therapy (e.g., hypogonadotropic eunuchoidism, fertile eunuch syndrome, prepubertal panhypopituitarism, postpubertal pituitary failure, Klinefelter's syndrome, Reifenstein's syndrome, functional prepubertal castration syndrome, male “Turner's syndrome”, Sertoli cell-only syndrome, adult seminiferous tubule failure, adult Leydig cell failure, and the like), a sarcopenia therapy, a muscle atrophy therapy, and the like.

A method of using the oligomeric compound embodiments of the present disclosure is described. In some embodiments, the method comprises administering an oligomer compound embodiment comprising a steroidal-based compound of Formula IX and/or a therapeutic agent (or a composition or formulation thereof), to a subject. In yet additional embodiments, the method comprises administering an oligomer compound embodiment comprising a steroidal-based compound of Formula IX and/or a therapeutic agent (or a composition or formulation thereof) to a male subject. In some embodiments, the subject can have (or be predisposed) to another malady, such as cancer. In some embodiments, the oligomeric compound is administered using any mode of administration discussed herein. In particular embodiments, the oligomeric compound embodiment can be administered via oral administration, injection, transdermal administration, inhalation, implantation, or the like. In some embodiments, the method comprises administering an oligomer compound embodiment comprising a steroidal-based compound of Formula IX and/or a therapeutic agent (or a composition or formulation thereof), to a male subject to alter the activity of an androgen, progesterone, estrogen, glucocorticoid, and/or mineralocorticoid receptor. In some embodiments, the method comprises administering an oligomer compound embodiment comprising a steroidal-based compound of Formula IX and/or a therapeutic agent (or a composition or formulation thereof), to a male subject to alter the activity of an androgen, progesterone, estrogen, glucocorticoid, and/or mineralocorticoid receptor.

The dosage used in method embodiments of the present disclosure will depend on certain factors, such as the age, weight, general health, and severity of the condition of the subject being treated, as will be understood by a person of ordinary skill in the art with the benefit of the present disclosure. Dosage also may be tailored to the sex and/or species of the subject. Dosage and frequency of administration may also depend on whether the compound (or a composition thereof, or any pharmaceutically acceptable salt, prodrug, stereoisomer, tautomer, or solvate of the compound) is formulated for treating acute episodes of a disease or for prophylactically treating a disease. Effective dosages can be estimated initially from in vitro assays. For example, an initial dosage for use in subjects can be formulated to achieve a circulating blood or serum concentration, of active compound that is at or above an IC₅₀ or EC₅₀ of the particular compound as measured in an in vitro assay, such as any of the assays described in the Examples section below. Dosages can be calculated to achieve such circulating blood or serum concentrations considering the bioavailability of the particular compound.

Dosage amounts, such as therapeutically effective amounts, of the compound (or a composition thereof, or any pharmaceutically acceptable salt, a prodrug, a solvate, or a tautomer thereof) for a subject will typically be in the range of from greater than 0 mg/kg/day (such as 0.0001 mg/kg/day, 0.001 mg/kg/day, or 0.01 mg/kg/day) to 100 mg/kg/day. In some embodiments, the dosage (or therapeutically effective amount) may range from 0.1 mg/kg/day to 30 mg/kg/day, such as 1 mg/kg/day to 10 mg/kg/day. In particular embodiments, the compound (or a composition thereof) can be administered once per day with or without food, as opposed to twice per day with food as required by current oral treatments for hormonal diseases and/or disorders, particularly hypogonadism.

### V. OVERVIEW OF SEVERAL EMBODIMENTS

Disclosed herein are embodiments of a compound of Formula I, or a pharmaceutically acceptable salt, a prodrug, a solvate, or a tautomer thereof

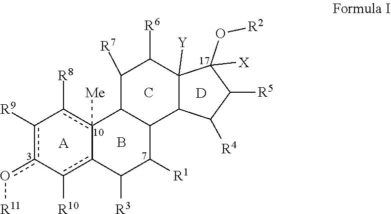

wherein

R¹ is selected from aliphatic, H, D, halogen, aromatic, heteroaliphatic, haloaliphatic, haloheteroaliphatic, heteroaliphatic, or an organic functional group;

R² is selected from —C(O)R^{a}, —C(O)OR^{a}, —C(O)NR^{b}R^{c}, wherein each R^{a} independently is selected from aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; —P(O)(OR^{a})₂, wherein each R^{a} independently is H, aliphatic, aromatic, heteroaliphatic, haloaliphatic, haloheteroaliphatic, heteroaliphatic, or an organic functional group; or —S(O)₂R^{a} wherein R^{a} is H, aliphatic, aromatic, heteroaliphatic, haloaliphatic, haloheteroaliphatic, heteroaliphatic, or an organic functional group;

each of R³, R⁴, R⁵, R⁶, R⁷, R⁸, R⁹, and R¹⁰ independently is selected from H, aliphatic, D, aromatic, heteroaliphatic, haloaliphatic, haloheteroaliphatic, heteroaliphatic, or an organic functional group;

R¹¹, if present, is selected from hydrogen, aliphatic, heteroaliphatic, aromatic, heteroaliphatic, haloaliphatic, haloheteroaliphatic, or an organic functional group;

X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; and

Y is aliphatic; and

provided that R² is not, or is other than, any of the following:

—C(O)(CH₂)₅CH₃; —C(O)CH₂SO₂OR, wherein R is methyl, ethyl, cyclopentyl, cyclohexyl, cycloheptyl, phenyl, phenoxy-lower-alkyl, and lower-alkoxy-phenyl; —C(O)CH₂SO₂OEt; —C(O)Ph; —C(O)Me; —C(O)Et; —C(O)OCH₂adamantyl; —C(O)Oadamantyl;

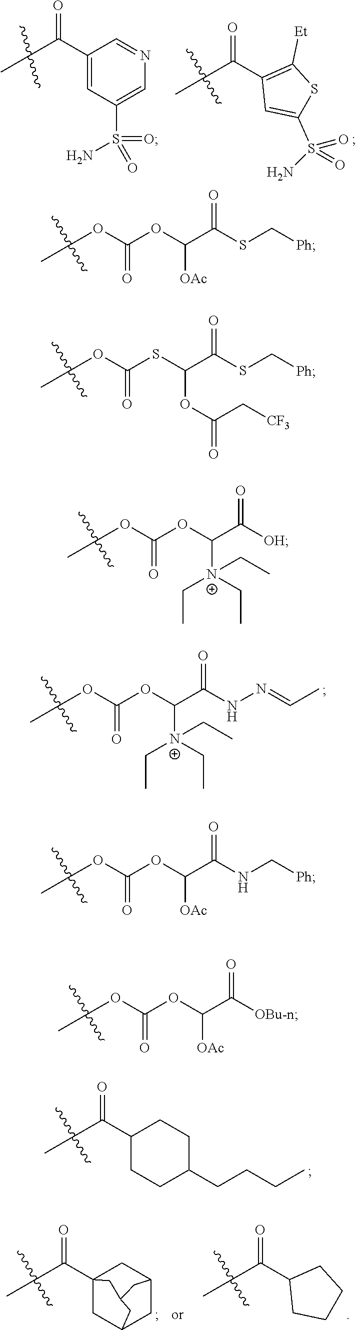

In any or all of the above embodiments, the following can apply:

R¹ is selected from alkyl, H, D, Cl, F, I, Br, alkenyl, alkynyl, aryl, heteroaryl, heteroalkyl, heteroalkenyl, heteroalkynyl, or any combination of these groups; R² is selected from —C(O)R^{a}, —C(O)OR^{a}, —C(O)NR^{b}R^{c}, wherein each R^{a} independently is selected from alkyl, alkenyl, alkynyl, aryl, heteroaryl, heteroalkyl, heteroalkenyl, heteroalkynyl, halogen, or another organic functional group, and each of R^{b} and R^{c} independently is H, alkyl, alkenyl, alkynyl, aryl, heteroaryl, heteroalkyl, heteroalkenyl, heteroalkynyl, halogen, or another organic functional group; —P(O)(OR^{a})₂, wherein each R^{a} independently is H, alkyl, alkenyl, alkynyl, aryl, heteroaryl, heteroalkyl, heteroalkenyl, heteroalkynyl, or an organic functional group; or —S(O)₂R^{a} wherein R^{a} is H, alkyl, alkenyl, alkynyl, aryl, heteroaryl, heteroalkyl, heteroalkenyl, heteroalkynyl, or an organic functional group;

each of R³, R⁴, R⁵, R⁶, R⁷, R⁸, R⁹, and R¹⁰ independently is selected from H, D, —OH, halogen, alkyl, alkenyl, alkynyl, aryl, heteroaryl, heteroalkyl, heteroalkenyl, heteroalkynyl, or any combination of these groups;

R¹¹, if present, is H, D, or a group selected from R²;

X is selected from H, D, aliphatic, heteroaliphatic, —OH, —C(O)R^{a}, —C(O)OR^{a}, or —C(O)NR^{b}R^{c}, wherein each R^{a} independently is alkyl, alkenyl, alkynyl, aryl, heteroaryl, heteroalkyl, heteroalkenyl, heteroalkynyl, or an organic functional group; and each of R^{b} and R^{c} independently is H, alkyl, alkenyl, alkynyl, aryl, heteroaryl, heteroalkyl, heteroalkenyl, heteroalkynyl, or an organic functional group; and

Y is alkyl, alkenyl, or alkynyl.

In any or all of the above embodiments, the following can apply:

R¹ is selected from lower alkyl, Cl, F, I, Br, or phenyl;

R² is selected from —C(O)R^{a}, —C(O)OR^{a}, or —C(O)NR^{b}R^{c}, wherein each R^{a} independently is selected from Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl, and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, C₁, Br, F, I, or another organic functional group; —P(O)(OR^{a})₂, wherein each R^{a} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; or —S(O)₂R^{a} wherein R^{a} is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group;

each of R³, R⁴, R⁵, R⁶, R⁷, R⁸, R⁹, and R¹⁰ independently is selected from H, D, —OH, Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, or any combination of these groups;

X is selected from H, D, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, —OH, —C(O)R^{a}, —C(O)OR^{a}, or —C(O)NR^{b}R^{c}, wherein each R^{a} independently is Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl; and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; and

Y is lower alkyl.

In any or all of the above embodiments, the following can apply:

R¹ is selected from methyl, ethyl, Cl, F, I, Br, t-butyl, or phenyl;

R² is selected from —C(O)Me, —C(O)Et, —C(O)nPr, —C(O)(CH₂)₅CH₃, —C(O)(CH₂)₉CH₃, —C(O)(CH₂)₁₀CH₃, —C(O)(CH₂)₇C(H)═C(H)(CH₂)₇CH₃, —C(O)(CH₂)₇C(H)═C(H)CH₂C(H)═C(H)(CH₂)₄CH₃, —C(O)Cl, —C(O)O(CH₂)₉CH₃, —C(O)OMe, —C(O)OC(CH₃)₃, —C(O)O(CH₂)₃CH₃, —C(O)O(CH₂)₄CH₃, —C(O)NH(CH₂)₉CH₃, —C(O)O(CH₂)₁₁CH₃, —C(O)NH(CH₂)₁₁CH₃, —C(O)N(CH₃)CH₃, —C(O)N(H)CH₃, —C(O)N(CH₂)₄CH₃, —C(O)N(H)(CH₂)₄CH₃, —C(O)N(CH₃)C(O)N(H)CH₃, or —S(O)₂Ph-p-Me;

each of R³, R⁹, and R¹⁰ independently is H or D;

R⁴, R⁵, and R⁶ are H;

R⁷ is H or Me;

R⁸ is H;

X is H or —CCH; and

Y is methyl or ethyl.

In any or all of the above embodiments, the compound can be represented by Formulas II, III, or IV as described herein, or a pharmaceutically acceptable salt, a prodrug, a solvate, or a tautomer thereof

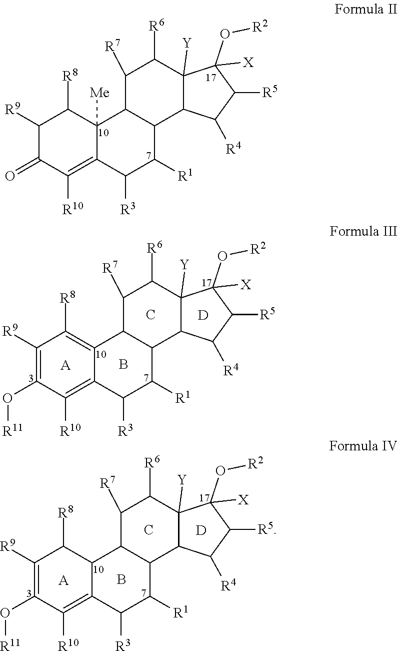

In any or all of the above embodiments, the compound has a Formula II, and R¹ is phenyl; R² is hydrogen; R³ is H or D; R⁴, R⁵, and R⁶ are H; R⁷ is H or Me; R⁸ is H; R⁹ is H or D; R¹⁰ is H or D; X is selected from H, —CCH; Y is methyl or ethyl.

In any or all of the above embodiments, compound has a Formula III and R¹ is aliphatic or aromatic; R² is hydrogen; R³ is H or D; R⁴, R⁵, and R⁶ are H; R⁷ is H or Me; R⁸ is H; R⁹ is H or D; and R¹⁰ is H or D; R¹¹ is H or D; X is selected from H, —CCH; and Y is methyl or ethyl.

In any or all of the above embodiments, the compound can be represented by Formulas IA, IB, IIA, IIB, IIIA, IIIB, IVA, or IVB as described herein, or a pharmaceutically acceptable salt, a prodrug, a solvate, or a tautomer thereof.

In any or all of the above embodiments, the compound can be represented by Formulas V, VI, VII, or VIII, as described herein, or a pharmaceutically acceptable salt, a prodrug, a solvate, or a tautomer thereof.

In any or all of the above embodiments, R² is —C(O)(CH₂)·CH₃ or —C(O)O(CH₂)ₙCH₃, wherein n is an integer selected from 0 to 15; —C(O)NR^{b}R^{c}, wherein R^{b} is H or lower alkyl and R^{c} is lower alkyl, or heteroaliphatic; or —S(O)₂R^{a} wherein R^{a} is aromatic; each of R³, R⁴, R⁵, R⁶, R⁷, R⁸, R⁹, and R¹⁰ is H or D; and X is H or —CCH.

In any or all of the above embodiments, the compound is represented by Formulas VA-VD, VA′-VD′, and VA″-VD″; Formulas VIA-VID, VIA′-VID′, and VIA″-VID″; and/or Formulas VIIA-VIID, VIIA′-VIID′, and VIIA″-VIID″, as described herein, or a pharmaceutically acceptable salt, a prodrug, a solvate, or a tautomer thereof.

In any or all of the above embodiments, the compound can be any species as disclosed herein.

Also disclosed are embodiments of a pharmaceutically acceptable composition, comprising a compound according to any or all of the above compound embodiments, a pharmaceutically acceptable salt, a prodrug, a solvate, or a tautomer thereof and a pharmaceutically acceptable excipient.

In any or all of the above embodiments, the pharmaceutically acceptable composition is formulated for injection.

Also disclosed are embodiments of a dosage form, comprising a compound according to any or all of the above compound embodiments, or a pharmaceutically acceptable salt, a prodrug, a solvate, or a tautomer thereof; or the pharmaceutically acceptable composition according to any or all of the above composition embodiments.

In any or all of the above embodiments, the dosage form is a tablet, a capsule, an implant, a patch, a microneedle array, an aerosol, or gel.

Also disclosed are embodiments of an oligomer compound, comprising a first steroidal-based compound covalently coupled to a first linker group via an oxygen atom attached to a functional group positioned at C17 of the steroidal-based compound, and wherein the first linker group is further covalently coupled to a second steroidal-based compound or a therapeutic agent.

In any or all of the above embodiments, the first steroidal-based compound has a Formula IX, or a pharmaceutically acceptable salt, a prodrug, a solvate, or a tautomer thereof

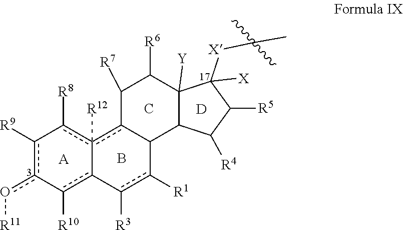

wherein X′ is bound to the first linker group; and wherein

R¹ is selected from H, D, halogen, aliphatic, heteroaliphatic, aromatic, heteroaliphatic, haloaliphatic, haloheteroaliphatic, or an organic functional group;

each of R³, R⁴, R⁵, R⁶, R⁸, R⁹, and R¹⁰ independently is selected from H, D, aliphatic, aromatic, heteroaliphatic, haloaliphatic, haloheteroaliphatic, heteroaliphatic, or an organic functional group;

R⁷ is selected from ═O, H, D, aliphatic, aromatic, heteroaliphatic, haloaliphatic, haloheteroaliphatic, heteroaliphatic, or an organic functional group;

R¹¹, when present, is selected from H, aliphatic, heteroaliphatic, aromatic, heteroaliphatic, haloaliphatic, haloheteroaliphatic, an organic functional group, or a second linker group;

R¹², when present, is H, D, or aliphatic;

X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group;

X′ can be oxygen or —C(O)(CH₂)ₚ—, wherein p is an integer selected from 1 to 10; and

Y is aliphatic.

In any or all of the above embodiments, each of R³, R⁴, R⁵, R⁶, R⁸, R⁹, and R¹⁰ are H or D;

each of R⁷, R¹², and Y independently is lower alkyl;

R¹ is Cl, F, I, Br, alkyl, alkenyl, alkynyl, aryl, heteroaryl, heteroalkyl, heteroalkenyl, heteroalkynyl, or any combination thereof;

X is hydrogen, —OH, or —CCH;

R¹¹, when present, is H, lower alkyl, or a second linker group, —C(O)Ph, or —C(Z)(CH₂)_{q}CH₃, wherein Z is S, O, or NH and q is an integer selected from 0 to 10.

In any or all of the above embodiments, the first steroidal-based compound is selected from any of the steroidal-based compounds disclosed herein and/or any or all of the above compound embodiments, or a pharmaceutically acceptable salt, a prodrug, a solvate, or a tautomer thereof; and wherein the first linker is attached to the oxygen atom of the C17 hydroxyl group, or a hydroxyl group of a —C(O)CH₂OH group attached at C17; and the second linker group, if present, is attached to the oxygen atom of the C3 hydroxyl group.

In any or all of the above embodiments, the first linker group and/or the second linker group has a Formula X

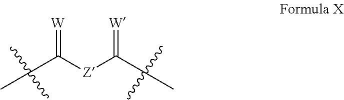

wherein each of W and W′ independently is selected from oxygen, sulfur, or NR^{d}, wherein R^{d} is H, aliphatic, heteroaliphatic, aromatic, heteroaliphatic, haloaliphatic, haloheteroaliphatic, or a combination thereof; and Z′ is selected from aliphatic, heteroaliphatic, aromatic, heteroaliphatic, haloaliphatic, haloheteroaliphatic, or a combination thereof.

In any or all of the above embodiments, each of W and W′ independently are oxygen or sulfur and Z′ is selected from —(CH₂)ₘ—; —O(CH₂)ₘO—; —NR^{e}(CH₂)ₘNR^{e}—; or —(CH₂)ₘNR^{c}C(O)(CH₂)_{m′}, wherein R^{d} is H, aliphatic, heteroaliphatic, aromatic, heteroaliphatic, haloaliphatic, haloheteroaliphatic, or a combination thereof and each of m and m′ independently is an integer ranging from 1 to 20.

In any or all of the above embodiments, the first and/or second linker is selected from

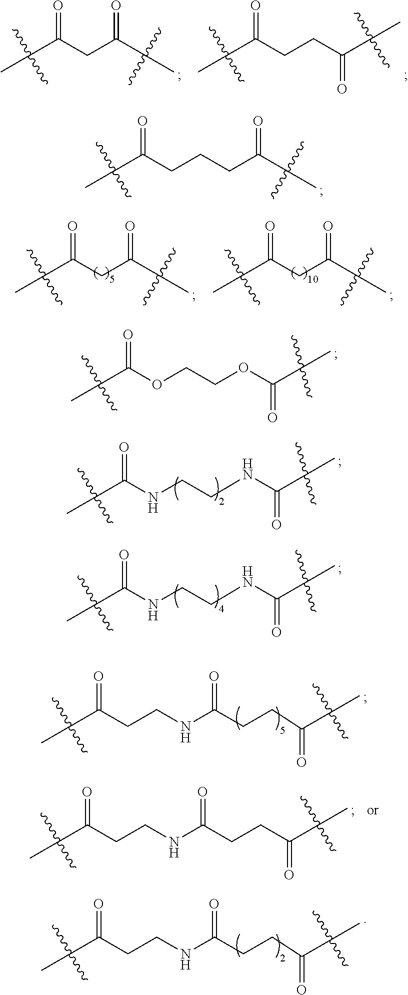

In any or all of the above embodiments, the oligomer compound comprises the therapeutic agent, which is a gonadotropin-releasing hormone (“GnRH”) antagonist and/or agonist, a E3 ubiquitin ligase recruiting ligand, an anticancer agent, a kinase antagonist and/or agonist, a GPCR antagonist and/or agonist, an antimalarial agent, an antifungal agent, an antiviral agent, an antibacterial agent, an immunosuppressant, an anti-inflammatory agent, or a pulmonary agent.

In any or all of the above embodiments, the oligomer compound comprises the second steroidal-based compound, which is the same or different from the first steroidal-based compound, and wherein the first linker group is covalently coupled to the first steroidal-based compound via the functional group at the C17 position and via a functional group at the C17 position of the second steroidal-based compound.

In any or all of the above embodiments, the oligomer compound is a homodimer wherein the first steroidal-based compound and the second steroidal-based compound are the same.

In any or all of the above embodiments, the oligomer compound is a heterodimer wherein the first steroidal-based compound and the second steroidal-based compound are the different.

In any or all of the above embodiments, the oligomer compound comprises a third steroidal-based compound.

In any or all of the above embodiments, the oligomer compound comprises a therapeutic agent and wherein the first linker group is covalently coupled to the first steroidal-based compound via the functional group at the C17 position and via a functional group of the therapeutic agent.

In any or all of the above embodiments, the oligomer compound further comprises an additional steroidal-based compound.

In any or all of the above embodiments, the oligomer compound is selected from an oligomer compound species as described herein, or a pharmaceutically acceptable salt, a prodrug, a solvate, or a tautomer thereof.

Also disclosed are embodiments of a composition comprising the oligomer compound according to any or all of the above oligomer compound embodiments, or a pharmaceutically acceptable salt, a prodrug, a solvate, or a tautomer thereof; and a pharmaceutically acceptable excipient.

In any or all of the above embodiments, the composition is formulated for injection.

Also disclosed are embodiments of a dosage form, comprising an oligomer compound according to any or all of the above oligomer compound embodiments, or a pharmaceutically acceptable salt, a prodrug, a solvate, or a tautomer thereof.

In any or all of the above embodiments, the dosage form is a tablet, a capsule, an implant, a patch, a microneedle array, an aerosol, or gel.

Also disclosed are embodiments of a method, comprising administering to a subject a compound according to any or all of the above compound embodiments, or an oligomer compound according to any or all of the above oligomer compound embodiments.

Also disclosed herein are embodiments of a method, comprising administering to a subject a therapeutically active amount of the dosage form according to any or all of the above dosage form embodiments.

In any or all of the above embodiments, the subject is a male subject.

In any or all of the above embodiments, the method is a hormonal therapy method.

In any or all of the above embodiments, the hormonal therapy is male contraception.

In any or all of the above embodiments, the compound, the oligomer compound, or the dosage form is administered orally, transdermally, or by injection.

Also disclosed are uses of the compound according to any or all of the above compound embodiments, or an oligomer compound according to any or all of the above oligomer compound embodiments.

In any or all of the above embodiments, the compound or the oligomer compound binds to an androgen receptor, a progesterone receptor, an estrogen receptor, a glucocorticoid receptor, and/or a mineralocorticoid receptor.

In any or all of the above embodiments, the use further comprises treating a disease or disorder selected from cancer, sickle cell anemia, leukemia, an autoimmune disorder, a cardiovascular disease, a fungal disease, a bacterial disease, a viral disease, endometriosis, a metabolic disease, a pulmonary disease, a gastrointestinal disease, a hypogonadism disorder, or any combination thereof.

Also disclosed are embodiments of a method for making the compound according to any or all of the above compound embodiments, comprising: performing a conjugate addition and deprotection reaction on a protecting-group containing precursor compound using a lithium compound, a catalyst, a Grignard reagent, and a silyl reagent to provide a substituted, deprotected product; and functionalizing the substituted, deprotected product to provide the compound; wherein the protecting-group containing precursor compound has a formula

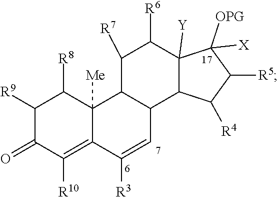

and the substituted, deprotected product has a formula

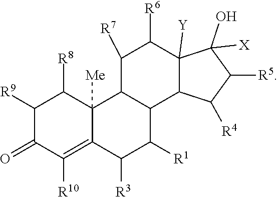

In any or all of the above embodiments, the lithium compound is LiCl; the catalyst is CuI; the Grignard reagent has a formula R¹MgCl, wherein R¹ is aliphatic, aromatic, heteroaliphatic, haloaliphatic, haloheteroaliphatic, heteroaliphatic, or a combination thereof; and the silyl reagent is TMSCL.

Also disclosed are embodiments of a method for making the oligomer compound according to any or all of the above oligomer compound embodiments, comprising: covalently coupling a linker group precursor and (i) one of the first steroidal-based compound or the second steroidal-based compound, or (ii) the therapeutic agent using an esterifying reagent to form either a linker-functionalized steroidal-based compound or a linker-functionalized therapeutic agent; and covalently coupling the linker-functionalized steroidal-based compound to the other of the first or second steroidal-based compound; or covalently coupling the linker-functionalized therapeutic agent to the first steroidal-based compound.

In any or all of the above embodiments, the linker group precursor is an anhydride or an acid and wherein the esterifying reagent is DMAP.

### VI. EXAMPLES

### A. Monomeric Compound Embodiments

### Example 1

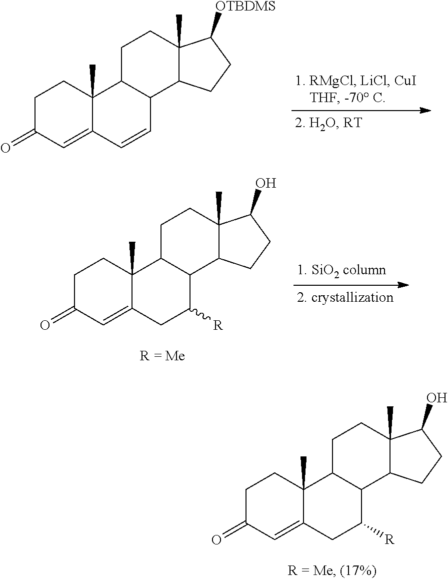

Under nitrogen, a mixture of LiCl (0.3 g, 6.9 mmol) and CuI (0.6 g, 3.1 mmol) in dry THF (14 mL) was stirred at room temperature for 1 hour. The resulting clear solution was added to a stirred, cold (−10° C.) solution of the silyl ether starting material (2.5 g, 6.2 mmol) in dry THF (14 mL), followed by the addition of TMSCl (4.3 mL, 34 mmol). The resulting mixture was then cooled to −75° C. (dry ice/acetone bath). The commercial 3.0 M MeMgCl in THF (5 mL, 15 mmol) was diluted with same amount of THF (5 mL) then added dropwise, over a period of 1 hour, into the reaction mixture and temp was kept at ˜−75° C. during addition. After addition, stirring was continued for 3 hours at −75° C., then water (10 mL) was added slowly and carefully. The resulting acidic reaction mixture was stirred at room temperature overnight. The mixture was diluted with EtOAc (50 mL) and the organic layer was separated then washed successively with 50% conc. NH₄OH (1×20 mL), 50% conc. NH₄OH/conc. NH₄Cl (1:1, 1×20 mL), water (1×20 mL) and brine (1×10 mL). All the aqueous washings were re-extracted with EtOAc (1×30 mL). The organic extracts were combined, dried over MgSO₄ and evaporated to dryness. The residual gum (˜2.0 g, HPLC: 7α:7β≈5:1) was dissolved in acetone (˜4 mL) and stored in a freezer overnight. The crystallized solid was collected and rinsed with cold acetone, air-dried to obtain a 1st crop product (0.4 g). The filtrate was stripped and the residue (1.6 g) was chromatographed over a flash silica gel column (50 g), using 0˜5% acetone/CH₂Cl₂. Based on TLC, good fractions were pooled and concentrated to afford a 2nd crop solid (0.2 g). These two crops of solid were combined (˜0.6 g) and dissolved in boiling IPA (˜6 mL) and cooled at room temperature overnight. The crystallized solid was collected and rinsed with cold IPA, air-dried to constant wt. to give pure 7α-methyl testosterone (CDB 4910) as an off-white solid, 320 mg (17% yield), mp. 211-214° C. The later fractions from column containing 7β-methyl testosterone were combined and evaporated to obtain an off-white solid, 0.1 g (CDB 4911).

### Example 2

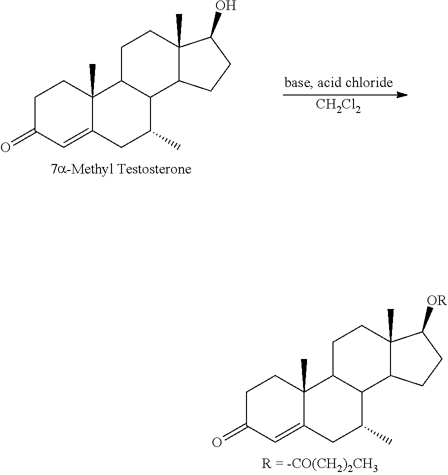

Under nitrogen, a mixture of trifluoroacetic anhydride (0.3 g, 1.4 mmol) and butyric acid (0.12 g, 1.4 mmol) in dry CH₂Cl₂ (3 mL) was stirred at room temperature for 30 minutes. 7α-Methyl testosterone (0.27 g, 0.89 mmol) was dissolved in dry CH₂Cl₂ (2 mL) and added. The resulting reaction mixture was stirred at room temperature for 2 hours. TLC of a mini workup showed no starting material. The reaction mixture was quenched with water (3 mL). The organic layer was separated and washed successively with 5% NaHCO₃(aq) (1×5 mL), water (1×5 mL) and brine (1×5 mL). All the aqueous phases were combined and re-extracted with CH₂Cl₂ (20 mL). The combined CH₂Cl₂ phase was dried (MgSO₄), filtered and evaporated. The crude residue (0.4 g) was loaded onto a flash silica gel column (35 g), eluted with 0˜5% EtOAc/CH₂Cl₂. Based on TLC, good fractions were pooled, concentrated and pumped under high vacuum to afford pure CB 4912 as a white solid, 0.25 g (75%), mp. 96-100° C.

### Example 3

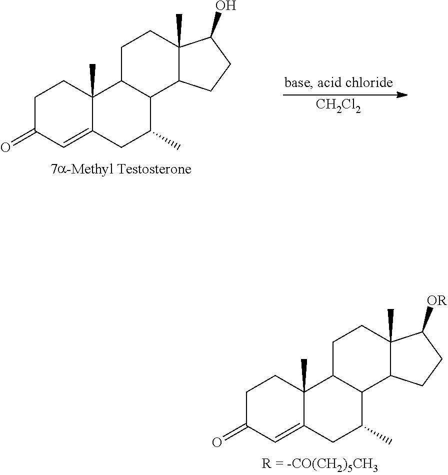

Under nitrogen, to a stirred solution of 7α-methyl testosterone (0.25 g, 0.83 mmol) in CH₂Cl₂ (5 mL), cooled in an ice/water bath, was added pyridine (0.1 mL, 1.2 mmol). After stirring for 30 minutes, heptanoyl chloride (0.16 g, 1.1 mmol) in CH₂Cl₂ (2 mL) was added. The resulting reaction mixture was stirred at 0° C. for 30 minutes. TLC indicated completion. The reaction mixture was stripped and the residue was dissolved in 5% MTBE/hexanes (10 mL), washed successively with water (1×5 mL), 10% H₃PO₄(aq) (1×5 mL), saturated NaHCO₃(aq) (1×5 mL) and brine (1×5 mL), then dried (MgSO₄), filtered and evaporated to dryness. The residual oil (0.37 g) was passed through a flash silica gel column (35 g) and eluted with 0˜3% EtOAc/CH₂Cl₂. The product containing fractions were pooled and stripped, vacuum pumped to dryness to afford pure CDB 4913 as colorless thick gum, 0.33 g (96%).

### Example 4

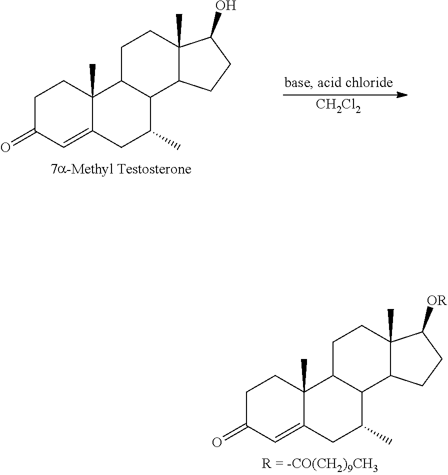

Under nitrogen, to a stirred solution of 7α-methyl testosterone (0.25 g, 0.83 mmol) in CH₂Cl₂ (5 mL), cooled in an ice/water bath, was added pyridine (0.1 mL, 1.2 mmol). After stirring for 30 minutes, undecanoyl chloride (0.25 g, 1.2 mmol) in CH₂Cl₂ (2 mL) was added. The resulting reaction mixture was stirred at 0° C. for 30 minutes. TLC indicated completion. The reaction mixture was stripped and the residue was dissolved in 5% MTBE/hexanes (10 mL), washed successively with water (1×5 mL), 10% H₃PO₄(aq) (1×5 mL), saturated NaHCO₃(aq) (1×5 mL) and brine (1×5 mL), then dried (MgSO₄), filtered and evaporated to dryness. The residual oil (0.50 g) was passed through a flash silica gel column (35 g) and eluted with 0˜3% EtOAc/CH₂Cl₂. The product containing fractions were pooled and stripped, vacuum pumped to dryness to obtain pure CDB 4914 as an off-white solid, 0.32 g (82%), mp. 52-54° C.

### Example 5

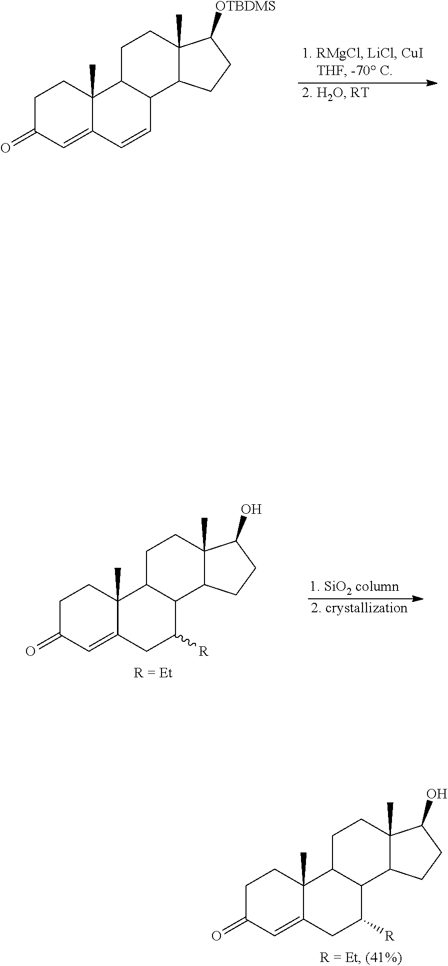

Under nitrogen, a mixture of LiCl (0.3 g, 6.9 mmol) and CuI (0.6 g, 3.1 mmol) in dry THF (14 mL) was stirred at room temperature for 1 hour. The resulting clear solution was added to a stirred, cold (−10° C.) solution of the silyl ether starting material (2.5 g, 6.2 mmol) in dry THF (14 mL), followed by the addition of TMSCl (4.3 mL, 34 mmol). The resulting mixture was then cooled to −75° C. (dry ice/acetone bath). The commercial 2.0 M EtMgCl in THF (8 mL, 16 mmol) was diluted with same amount of THF (8 mL) then added dropwise, over a period of 1 hour, into the reaction mixture and temp was kept at ˜−75° C. during addition. After addition, stirring was continued for 3 hours at −75° C., then water (10 mL) was added slowly and carefully. The resulting acidic reaction mixture was stirred at room temperature overnight. The mixture was diluted with EtOAc (50 mL) and the organic layer was separated then washed successively with 50% conc. NH₄OH (1×20 mL), 50% conc. NH₄OH/conc. NH4Cl (1:1, 1×20 mL), water (1×20 mL) and brine (1×10 mL). All the aqueous washings were re-extracted with EtOAc (1×30 mL). The organic extracts were combined, dried over MgSO₄ and evaporated to dryness. The residual solid (2.0 g, HPLC: 7α:7β≈3:2) was dissolved in minimum boiling IPA (˜16 mL) and stored at room temperature overnight. The crystallized solid was collected and rinsed with cold IPA, air-dried to obtain a 1st crop product [0.91 g, HPLC: 95.4% (7α)+4.6% (7β)]. The filtrate was stripped and the residue (1.0 g) was chromatographed over a flash silica gel column (40 g), using 5˜15% EtOAc/CH₂Cl₂. Based on TLC, good fractions were pooled and concentrated to afford a 2nd crop solid [0.29 g, HPLC: 98.2% (7α)+1.0% (7β)]. These two crops of solid were combined (1.2 g) and dissolved in minimum boiling IPA (˜13 mL) and cooled at room temperature overnight. The crystallized solid was collected and rinsed with cold IPA, air-dried to constant wt. to afford pure 7α-ethyl testosterone (CDB 4918) as a white crystalline solid, 0.80 g (41% yield), mp. 218-220° C., HPLC: 99.5% (7α)+0.4% (7β). The later fractions from column containing 7β-ethyl testosterone were combined and evaporated to obtain an off-white solid, 0.17 g (CDB 4924).

### Example 6

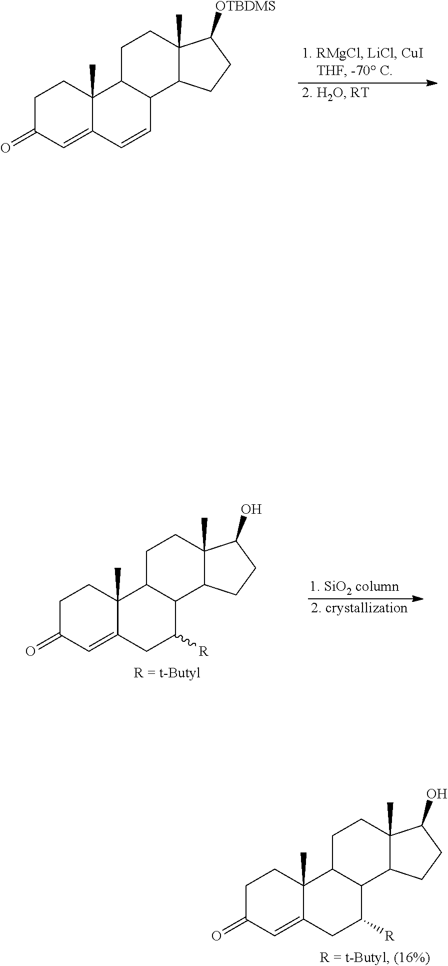

Under nitrogen, a mixture of LiCl (0.12 g, 2.8 mmol) and CuI (0.24 g, 1.2 mmol) in dry THF (10 mL) was stirred at room temperature for 1 hour The resulting clear solution was added to a stirred, cold (−10° C.) solution of the silyl ether starting material (1.0 g, 2.5 mmol) in dry THF (10 mL), followed by the addition of TMSCl (1.7 mL, 14 mmol). The resulting mixture was then cooled to −75° C. (dry ice/acetone bath). The commercial 1.0 M t-Butyl MgCl in THF (7 mL, 7 mmol) was then added dropwise, over a period of 1 hour, into the reaction mixture and temp was kept at ˜−75° C. during addition. After addition, stirring was continued for 3 hours at −75° C., then water (5 mL) was added slowly and carefully. The resulting acidic reaction mixture was stirred at room temperature overnight. The mixture was diluted with EtOAc (30 mL) and the organic layer was separated then washed successively with 50% conc. NH₄OH (1×10 mL), 50% conc. NH₄OH/conc. NH₄Cl (1:1, 1×10 mL), water (1×10 mL) and brine (1×10 mL). All the aqueous washings were re-extracted with EtOAc (1×30 mL). The organic extracts were combined, dried over MgSO₄ and evaporated to dryness. The crude residue (1.0 g, HPLC: 7α:7β≈3:2) was chromatographed over a flash silica gel column (40 g), using 5˜15% EtOAc/CH₂Cl₂. Based on TLC, good fractions were pooled, concentrated and pumped under high vacuum to obtain pure 7α-t-Butyl Testosterone (CDB 4919) as a white foam, 0.14 g (16% yield), with HPLC: 98.9% (7α)+0.5% (7β).

### Example 7

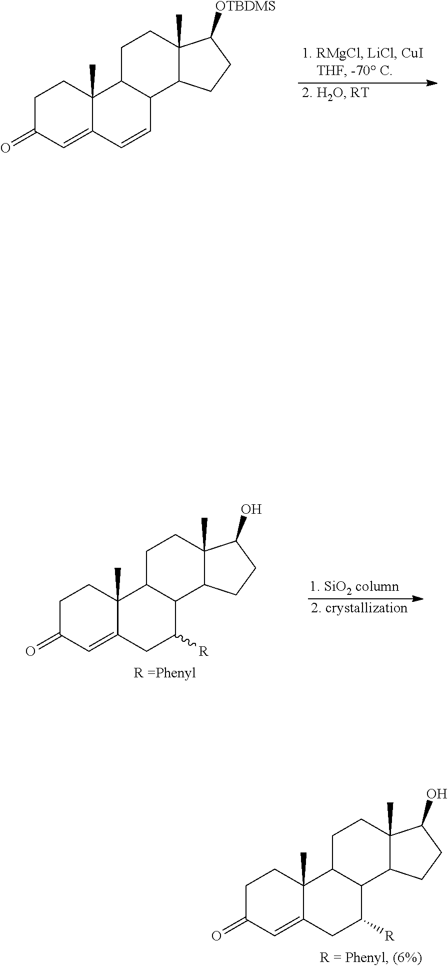

Under nitrogen, a mixture of LiCl (0.18 g, 4.2 mmol) and CuI (0.36 g, 1.8 mmol) in dry THF (10 mL) was stirred at room temperature for 1 hour. The resulting clear solution was added to a stirred, cold (−10° C.) solution of the silyl ether starting material (1.5 g, 3.7 mmol) in dry THF (10 mL), followed by the addition of TMSCl (2.6 mL, 21 mmol). The resulting mixture was then cooled to −75° C. (dry ice/acetone bath). The commercial 2.0 M PhMgCl in THF (5.5 mL, 11 mmol) was diluted with same amount of THF (5.5 mL) then added dropwise, over a period of 1 hour, into the reaction mixture and temp was kept at ˜−75° C. during addition. After addition, stirring was continued for additional 3 hours at −75° C., then water (8 mL) was added slowly and carefully. The resulting acidic reaction mixture was stirred at room temperature overnight. The mixture was diluted with EtOAc (30 mL) and the organic layer was separated then washed successively with 50% conc. NH₄OH (1×10 mL), 50% conc. NH₄OH/conc. NH₄Cl (1:1, 1×10 mL), water (1×10 mL) and brine (1×10 mL). All the aqueous washings were re-extracted with EtOAc (1×30 mL). The organic extracts were combined, dried over MgSO₄ and evaporated to dryness. The crude residue (1.5 g, HPLC: 7α:7β≈1:4) was chromatographed over a flash silica gel column (50 g), using 5˜15% EtOAc/CH₂Cl₂. Based on TLC, good fractions were pooled, concentrated and pumped under high vacuum to obtain 7α-Phenyl Testosterone (CDB 4920) as a white foam, 85 mg (6% yield), CDB 4920 with HPLC: 93.1% (7α)+3.5% (7β). The later fractions from column containing 7β-Phenyl Testosterone were combined and evaporated to obtain an off-white solid, 0.42 g (31%), as CDB 4921.

### Example 8

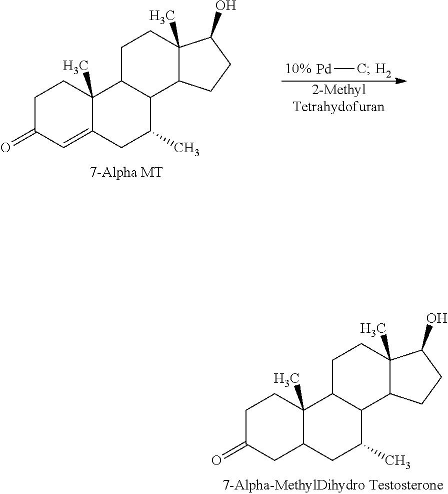

7-α-Methyl Testosterone (HPLC: 98%, 0.50 g) was dissolved in 2-methylTHF (50 mL). The solution was purged with nitrogen and 10% Pd—C(0.1 g) was added to the solution. The resulting mixture was hydrogenated at 20 psi for 2 hours (TLC: UV absorbing starting material is not detected). The mixture was filtered over a Celite pad. The filtrate was concentrated and the residue (mixture of 3 spots by TLC, one major) was purified over silica column to isolate the major spot (compound does not absorb any UV, column fractions were isolated 40 mL fractions and charred with methanol-sulfuric acid). Pure fractions were stripped to obtain a white solid (ca. 370 mg). This material was suspended in MTBE (20 mL), stirred for 15 minutes and separated solid was collected. This material was dried to obtain 342 mg of a white solid CDB 4922).

### Example 9

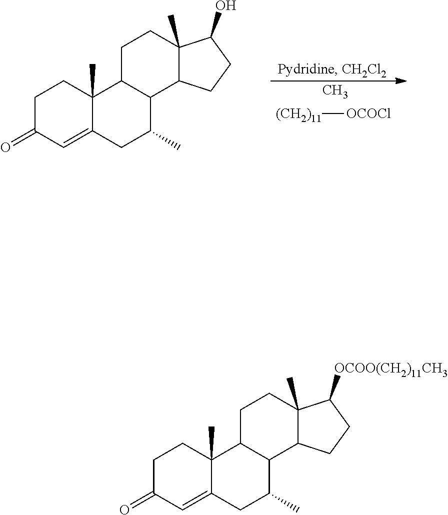

7α-Methyl Testosterone (0.10 g, 0.33 mmol) was dissolved in CH₂Cl₂ (10 mL). Under nitrogen, to the stirred solution pyridine (0.05 g, 0.63 mmol) was added and the solution was cooled in an ice-bath (5-100° C.). To the resulting solution, dodecyl chloroformate (0.15 g, 0.6 mmol) was added and the solution was stirred at room temperature for 4 hours [TLC: ca. 25% starting material]. To the mixture additional pyridine (25 mg) and dodecyl chloroformate (0.10 g) were added and the resulting mixture was stirred for additional 2 hours. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the carbonate ester (88 mg) as thick gum. Additional 100 mg compound was reacted in the same manner as described above to get additional carbonate ester (90 mg). Both batches were combined and the combined material (160 mg) was re purified over silica column to obtain Dodecyl carbonate ester as thick gum (135 mg, CDB 4923).

### Example 10

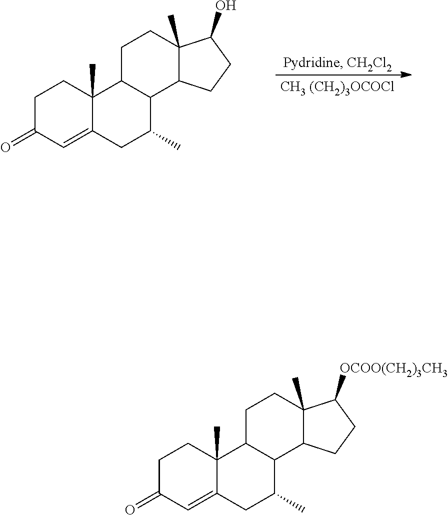

7α-Methyl Testosterone (0.10 g, 0.33 mmol) was dissolved in CH₂Cl₂ (10 mL). Under nitrogen, to the stirred solution pyridine (0.05 g, 0.63 mmol) was added and the solution was cooled in an ice-bath (5-10° C.). To the resulting solution, butyl chloroformate (0.10 g, 0.7 mmol) was added and the solution was stirred at room temperature for 4 hours [TLC: ca. 25% starting material]. To the mixture additional pyridine (25 mg) and butyl chloroformate (0.05 g) was added and the resulting mixture was stirred for additional 2 hours. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the carbonate ester (92 mg) as a thick oil (CDB 4925).

### Example 11

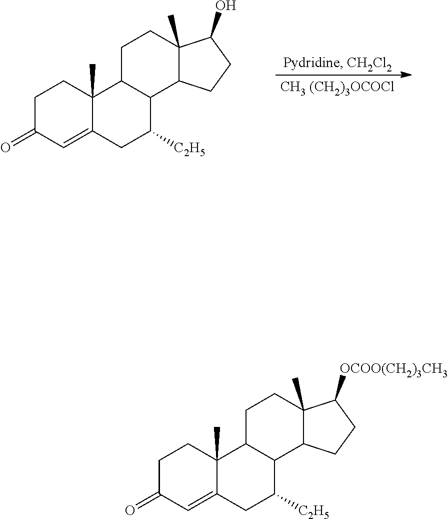

7α-Ethyl Testosterone (0.10 g, 0.3 mmol) was dissolved in CH₂Cl₂ (10 mL). Under nitrogen, to the stirred solution pyridine (0.05 g, 0.63 mmol) was added and the solution was cooled in an ice-bath (5-100 C). To the resulting solution, butyl chloroformate (0.10 g, 0.7 mmol) was added and the solution was stirred at room temperature for 4 hours [TLC: ca. 25% starting material]. To the mixture additional pyridine (25 mg) and butyl chloroformate (0.05 g) were added and the resulting mixture was stirred for additional 2 hours. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the carbonate ester (81 mg) as thick glass foam (CDB 4926).

### Example 12

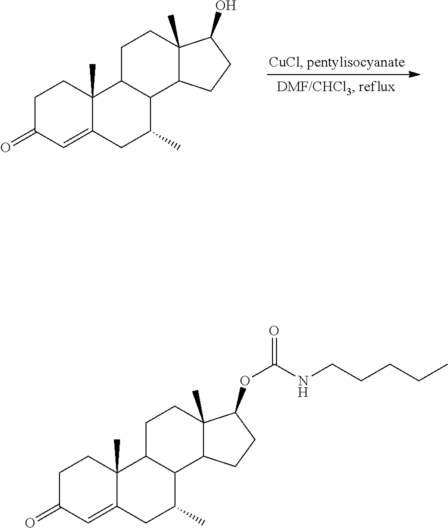 [SMILES: C[C@@H]1CC2=CC(=O)CC[C@]2(C)C2CC[C@@]3(C)C(CC[C@@H]3O)C21 ; "CuCl, pentylisocyanate" ; CCCCCNC(=O)O[C@H]1CCC2C3C(CC[C@@]21C)[C@@]1(C)CCC(=O)C=C1C[C@H]3C]

Under nitrogen, to a stirred mixture of 7α-MT (0.20 g, 0.66 mmol) and CuCl (68 mg, 0.68 mmol) in dry DMF (3 mL) was added a solution of pentylisocyanate (0.30 g, 2.64 mmol) in CHCl₃ (3 mL). The resulting reaction mixture was heated at reflux overnight, then cooled to room temperature and stripped. The residue was partitioned between EtOAc (20 mL) and water (5 mL). The water layer was separated and re-extracted with EtOAc (2×5 mL). The combined EtOAc solution was washed with brine (1×5 mL), dried over MgSO₄, filtered and evaporated. The resulting crude product (0.51 g) was purified via a flash silica gel column (40 g) and eluted with 0˜8% EtOAc/CH₂Cl₂. Fractions containing pure product were pooled, stripped and pumped under high vacuum to constant wt. to afford pure CDB 4949, 0.13 g (47%), as yellowish foam. ¹H NMR partial (400 MHz, CDCl₃): δ 5.73 (s, 1H, Olefinic-H), 4.60 (br.s, 1H), 4.55 (t, 1H, J=8.4 Hz), 3.15 (m, 2H), 0.90 (t, 3H, J=6.8 Hz, CH₃), 0.81 (s, 3H, CH₃), 0.77 (d, 3H, J=7.2 Hz, CH₃). HPLC: 91.3%. LC/MS: 416.3 (M+1).

### Example 13

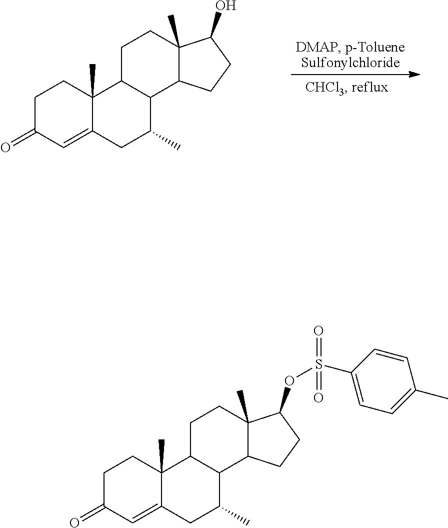 [SMILES: C[C@@H]1CC2=CC(=O)CC[C@]2(C)C2CC[C@@]3(C)C(CC[C@@H]3O)C21 ; "DMAP, p-TolueneSulfonylchloride" ; Cc1ccc(S(=O)(=O)O[C@H]2CCC3C4C(CC[C@@]32C)[C@@]2(C)CCC(=O)C=C2C[C@H]4C)cc1]

Under nitrogen, to a stirred solution of 7α-MT (0.20 g, 0.66 mmol) and DMAP (0.12 g, 0.98 mmol) in CHCl₃ (5 mL) at room temperature was added a solution of p-toluene sulfonylchloride (0.19 g, 1.0 mmol) in CHCl₃ (5 mL). The resulting reaction mixture was heated at reflux overnight, then cooled to room temperature and stripped. The residue was partitioned between EtOAc (20 mL) and sat. aqueous NaHCO₃ (5 mL). The water layer was separated and re-extracted with EtOAc (2×5 mL). The combined EtOAc solution was washed with brine (1×5 mL), dried over MgSO₄, filtered and evaporated. The resulting crude product (0.42 g) was purified via a flash silica gel column (40 g) and eluted with 0˜8% EtOAc/CH₂Cl₂. Fractions containing pure product were pooled, stripped and pumped under high vacuum to constant wt. to yield pure CDB 4950, 0.26 g (86%), as white foam. ¹H NMR partial (400 MHz, CDCl₃): δ 7.78 (d, 2H, J=8.0 Hz, Ar—H), 7.33 (d, 2H, J=8.0 Hz, Ar—H), 5.71 (s, 1H, Olefinic-H), 4.26 (t, 1H, J=8.8 Hz), 2.45 (s, 3H, CH₃), 1.17 (s, 3H, CH₃), 0.85 (s, 3H, CH₃), 0.71 (d, 3H, J=6.8 Hz, CH₃). HPLC: 99.0%. LC/MS: 457.3 (M+1).

### Example 14

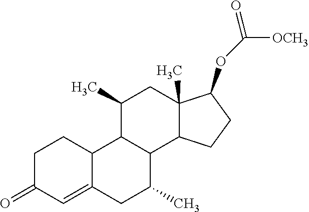 [SMILES: COC(=O)O[C@H]1CCC2C3C(C4CCC(=O)C=C4C[C@H]3C)[C@@H](C)C[C@@]21C]

Under nitrogen, to a solution of DMA (1.0 g, 3.3 mmol) in dichloromethane (20 mL), pyridine (0.50 g, 6.3 mmol) was added. The resulting clear solution was cooled with stirring at 0° C. (ice/water bath) and treated with methyl chloroformate (0.38 g, 4.0 mmol). The cooling bath was removed and the reaction mixture was stirred at room temperature. After 1 hour, TLC indicated ˜50% completion. Additional methyl chloroformate (0.28 g, 2.8 mmol) was added, and stirring was continued at room temperature for additional 1 hour. TLC showed only trace DMA left. The reaction mixture was diluted with dichloromethane (20 mL) and washed successively with 10% H₃PO₄(aq) (2×10 mL) and brine (1×10 mL). The organic phase was dried (MgSO₄) and evaporated. The crude residue (1.1 g) was purified via a fine silica gel column (30 g) using 0˜4% EtOAc/CH₂Cl₂ gradient to afford pure product CDB 4718, 0.90 g (76%), white solid, Mp. 151-153° C. ¹HNMR (partial) (400 MHz, CDCl₃): δ 0.79 (d, 3H, C-7α CH₃), 0.96 (s, 3H, C-18 CH3), 1.08 (d, 3H, C-11 CH₃), 3.78 (s, 3H, C-2′ CH3), 4.49 (m, 1H, C-17 CH), 5.86 (s, 1H, C-4 CH) ppm. LC-MS: 361.2, (Calcd.: 360.5). Thin Layer Chromatography: Analtech Silica Gel GF, EtOAC/CH₂Cl₂ (1:9)=0.60.

### Example 15

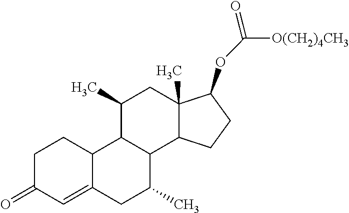

Under nitrogen, to a solution of DMA (1.0 g, 3.3 mmol) in dichloromethane (20 mL), pyridine (0.50 g, 6.3 mmol) was added. The resulting clear solution was cooled with stirring at 0° C. (ice/water bath) and treated with amyl chloroformate (0.60 g, 4.0 mmol). The cooling bath was removed and the reaction mixture was stirred at room temperature. After 3 hours, TLC indicated-50% completion. Additional amyl chloroformate (0.42 g, 2.8 mmol) was added, and stirring was continued at room temperature for additional 1 hour. TLC showed only trace DMA left. The reaction mixture was diluted with dichloromethane (20 mL) and washed successively with 10% H₃PO₄(aq) (2×10 mL) and brine (1×10 mL). The organic phase was dried (MgSO₄) and evaporated. The crude residue (1.8 g) was purified via a fine silica gel column (35 g) using 0˜4% EtOAc/CH₂Cl₂ gradient to afford pure product CDB 4872, 1.2 g (87%), as colorless oil. ¹HNMR (partial) (400 MHz, CDCl₃): δ 0.79 (d, 3H, C-7α CH3), 0.92 (t, 3H, C-6′ CH3), 0.96 (s, 3H, C-18 CH3), 1.08 (d, 3H, C-11 CH3), 4.12 (t, 2H, C-2′ CH2), 4.50 (m, 1H, C-17 CH), 5.86 (s, 1H, C-4 CH) ppm. LC-MS: 417.2, (Calcd.: 416.6), TLC, EtOAC/CH₂Cl₂ (1:9)=0.65.

### Example 16

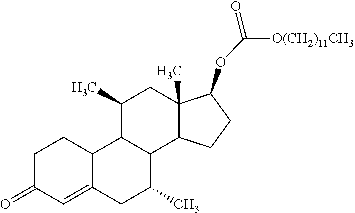

Under nitrogen, to a solution of DMA (1.0 g, 3.3 mmol) in dichloromethane (20 mL), pyridine (0.50 g, 6.3 mmol) was added. The resulting clear solution was cooled with stirring at 0° C. (ice/water bath) and treated with dodecyl chloroformate (1.0 g, 4.0 mmol). The cooling bath was removed and the reaction mixture was stirred at room temperature. After 1 hour, TLC indicated-50% completion. Additional dodecyl chloroformate (0.70 g, 2.8 mmol) was added, and stirring was continued at room temperature for additional 1 hour. TLC showed only trace DMA left. The reaction mixture was diluted with dichloromethane (20 mL) and washed successively with 10% H₃PO₄(aq) (2×10 mL) and brine (1×10 mL). The organic phase was dried (MgSO₄) and evaporated. The crude residue (2.3 g) was purified via a fine silica gel column (40 g) using 0˜4% EtOAc/CH₂Cl₂ gradient to afford pure product CDB 4830, 1.4 g (82%), as low melting white solid. ¹HNMR (partial) (400 MHz, CDCl₃): δ 0.80 (d, 3H, C-7α CH3), 0.90 (t, 3H, C-6′ CH3), 0.96 (s, 3H, C-18 CH3), 1.10 (d, 3H, C-11p CH3), 4.12 (t, 2H, C-2′ CH2), 4.48 (m, 1H, C-17 CH), 5.86 (s, 1H, C-4 CH) ppm. LC-MS: 515.3, (Calcd.: 514.8) TLC, EtOAC/CH₂Cl₂ (1:9)=0.65.

### Example 17

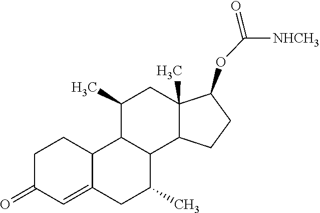 [SMILES: CNC(=O)O[C@H]1CCC2C3C(C4CCC(=O)C=C4C[C@H]3C)[C@@H](C)C[C@@]21C]

Under nitrogen, to a stirred slurry of DMA (1.0 g, 3.3 mmol) and CuCl (0.34 g, 3.4 mmol) in dry DMF (10 mL), N-succinimidyl N-methylcarbamate (0.76 g, 4.4 mmol) was added. The reaction mixture was heated (oil bath at 80˜85° C.) overnight. TLC indicated ˜50% completion. Additional N-succinimidyl N-methylcarbamate (0.40 g, 2.3 mmol) was added, and heating was continued for additional 24 hours. TLC showed ˜70% completion. The reaction mixture was cooled to room temperature and diluted with EtOAc (30 mL) and washed with water (1×30 mL). The aqueous layer was separated and extracted with EtOAc (1×30 mL). The combined EtOAc solution was washed with brine (1×10 mL), dried (MgSO₄) and evaporated. The crude residue (1.46 g) was purified via a fine silica gel column (40 g) using 0˜6% EtOAc/CH₂Cl₂ gradient to give a pale yellow gum (0.86 g) which still contaminated with some starting DMA. A portion (0.14 g) of this gum was further purified via chromatotron to afford 80 mg of a rather clean product for characterization (CDB 4873). ¹HNMR (partial) (400 MHz, CDCl₃): δ 0.79 (d, 3H, C-7α CH₃), 0.90 (s, 3H, C-18 CH₃), 1.08 (d, 3H, C-1 lB CH_{3}}, 2.80 (d, 3H, C-2′ CH₃₎, 4.50 (m, 1H, C-17 CH), 4.68 (br.s, 1H, NH), 5.85 (s, 1H, C-4 CH) ppm. LC-MS: 360.0, (Calcd.: 359.5), TLC, EtOAC/CH₂Cl₂ (1:9)=0.32.

### Example 18

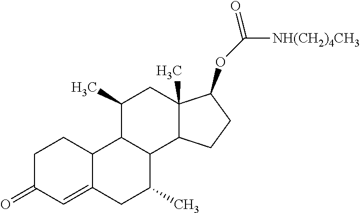

Under nitrogen, to a stirred slurry of DMA (1.0 g, 3.3 mmol) and CuCl (0.34 g, 3.4 mmol) in dry DMF (10 mL), pentyl isocyanate (0.50 g, 4.4 mmol) was added. The reaction mixture was stirred at room temperature. After 1 hour, TLC indicated complete reaction. The reaction mixture was diluted with EtOAc (30 mL) and washed with water (1×30 mL). The aqueous layer was separated and extracted with EtOAc (1×30 mL). The combined EtOAc solution was washed with brine (1×10 mL), dried (MgSO₄) and evaporated. The crude residue (1.8 g) was purified via a fine silica gel column (35 g) using 0˜5% EtOAc/CH₂Cl₂ gradient to afford pure product CDB 4874, 1.10 g (80%), as white foam. ¹HNMR (partial) (400 MHz, CDCl₃): δ 0.79 (d, 3H, C-7α CH₃₎, 0.90 (t, 3H, C-6′ CH₃₎, 0.92 (s, 3H, C-18 CH₃₎, 1.08 (d, 3H, C-11 CH₃₎, 3.15 (m, 2H, C-2′ CH2), 4.50 (m, 1H, C-17 CH), 4.65 (br.s, 1H, NH), 5.85 (s, 1H, C-4 CH) ppm. LC-MS: 416.2, (Calcd.: 415.6), TLC, EtOAC/CH₂Cl₂ (1:9)=0.45.

### Example 19

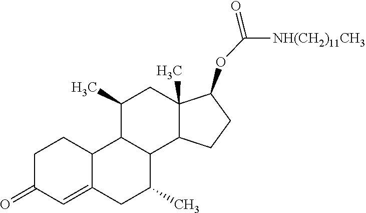

Under nitrogen, to a stirred slurry of DMA (1.0 g, 3.3 mmol) and CuCl (0.34 g, 3.4 mmol) in dry DMF (10 mL), dodecyl isocyanate (0.93 g, 4.4 mmol) was added. The reaction mixture was stirred at room temperature. After 1 hour, TLC indicated complete reaction. The reaction mixture was diluted with EtOAc (30 mL) and washed with water (1×30 mL). The aqueous layer was separated and extracted with EtOAc (1×30 mL). The combined EtOAc solution was washed with brine (1×10 mL), dried (MgSO₄) and evaporated. The crude residue (2.1 g) was purified via a fine silica gel column (40 g) using 0˜5% EtOAc/CH₂Cl₂ gradient to afford pure product CDB 4875, 1.21 g (71%), as off-white solid, Mp. 54-56° C. ¹HNMR (partial) (400 MHz, CDCl₃): δ 0.80 (d, 3H, C-7α CH₃), 0.90 (s, 3H, C-18 CH₃₎, 0.92 (t, 3H, C-6′ CH₃₎, 1.08 (d, 3H, C-11 CH₃₎, 3.18 (m, 2H, C-2′ CH2), 4.50 (m, 1H, C-17 CH), 4.63 (br.s, 1H, NH), 5.85 (s, 1H, C-4 CH) ppm. LC-MS: 514.3, (Calcd.: 513.8), TLC, EtOAC/CH₂Cl₂ (1:9)=0.45.

### Example 20

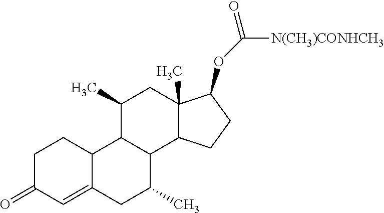

Under nitrogen, to a stirred slurry of DMA (1.0 g, 3.3 mmol) and CuCl (0.34 g, 3.4 mmol) in dry DMF (10 mL), methyl isocyanate (0.25 g, 4.4 mmol) was added. The reaction mixture was stirred at room temperature. After 1 hour, TLC indicated ˜30% reaction. After another 2 hours at room temperature, TLC was unchanged. Additional methyl isocyanate (0.25 g, 4.4 mmol) was added, and stirring was continued at room temperature overnight. TLC showed only trace DMA left. The reaction mixture was diluted with EtOAc (30 mL) and washed with water (1×30 mL). The aqueous layer was separated and extracted with EtOAc (1×30 mL). The combined EtOAc solution was washed with brine (1×10 mL), dried (MgSO₄) and evaporated. The crude residue (1.9 g) was purified via a fine silica gel column (35 g) using 0˜10% EtOAc/CH₂Cl₂ gradient. The product (white foam, 1.25 g) was triturated with hot MTBE (10 mL) for 5 minutes, then cooled to room temperature. The product was collected and air-dried to constant weight to give pure product CDB 4876, 1.07 g (73%), as white crystalline solid, Mp. 185-187° C. ¹HNMR (partial) (400 MHz, CDCl₃): δ 0.80 (d, 3H, C-7α CH₃₎, 0.98 (s, 3H, C-18 CH₃₎, 1.08 (d, 3H, C-11 CH₃₎, 2.88 (d, 3H, NHCH₃₎, 3.25 (s, 3H, NCH₃₎, 4.55 (m, 1H, C-17 CH), 5.86 (s, 1H, C-4 CH), 8.50 (br.s, 1H, NH) ppm. LC-MS: 417.2, (Calcd.: 416.6), TLC, EtOAC/CH₂Cl₂ (1:9)=0.38.

### Example 21

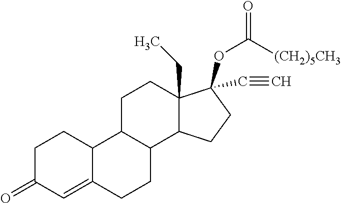

Under nitrogen, a mixture of heptanoic acid (0.83 g, 6.4 mmol) and trifluoroacetic anhydride (2.3 g, 11.0 mmol) in dry toluene (15 mL) was heated (oil bath at 80˜85° C.) for 2 hours. The solution was cooled to room temperature and treated with Levonorgestrel (1.0 g, 3.2 mmol) and solid Na₂CO₃ (0.5 g, 4.7 mmol). The mixture was heated (oil bath at 80-85° C.) for 4 hours. TLC indicated complete reaction. The reaction mixture was cooled to room temperature and diluted with a mixture of EtOAc (30 mL) and water (10 mL). The organic layer was separated and washed successively with sat. NaHCO₃ (1×15 mL) and brine (1×10 mL). The organic phase was dried (MgSO₄) and evaporated. The crude residue (1.50 g) was purified via a fine silica gel column (40 g) using 0˜4% EtOAc/CH₂Cl₂ gradient to afford pure product CDB 4879, 0.79 g (58%), as pale yellow gum. ¹HNMR (partial) (400 MHz, CDCl₃): δ 0.90 (t, 3H, C-7′ CH₃), 1.02 (t, 3H, C-18-CH₃), 2.60 (s, 1H, C══C—H), 5.85 (s, 1H, C-4 CH) ppm. LC-MS: 425.0, (Calcd.: 424.6), TLC, EtOAC/CH₂Cl₂ (1:9)=0.62.

### Example 22

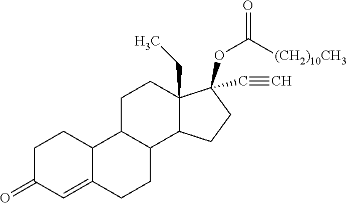

A mixture of Levonorgestrel (1.0 g, 3.2 mmol), dodecanoic anhydride (1.6 g, 4.2 mmol) and DMAP (0.6 g, 5.1 mmol) in dry CH₂Cl₂ (15 mL) was stirred at room temperature overnight. TLC showed ˜50% reaction. Additional dodecanoic anhydride (0.8 g, 2.1 mmol) and DMAP (0.3 g, 2.6 mmol) were added. The reaction mixture was then heated at reflux for additional 24 hours. TLC showed ˜80% reaction. The reaction mixture was cooled to room temperature and diluted with EtOAc (30 mL). The mixture was then washed successively with 10% H₃PO₄ (1×15 mL), sat. NaHCO₃ (1×15 mL) and brine (1×10 mL). The organic phase was dried (MgSO4) and evaporated. The crude residue (2.0 g) was purified via a fine silica gel column (40 g) using with 0˜3% EtOAc/CH₂Cl₂ gradient to afford pure product CDB 4880, 1.25 g (78%), as pale yellow gum. ¹HNMR (partial) (400 MHz, CDCl₃): δ 0.90 (t, 3H, C-12′ CH₃), 1.03 (t, 3H, C-18-CH₃), 2.60 (s, 1H, C═C—H), 5.85 (s, 1H, C-4 CH) ppm. LC-MS: 495.0, (Calcd.: 494.8), TLC, EtOAC/CH₂Cl₂ (1:9)=0.66.

### Example 23

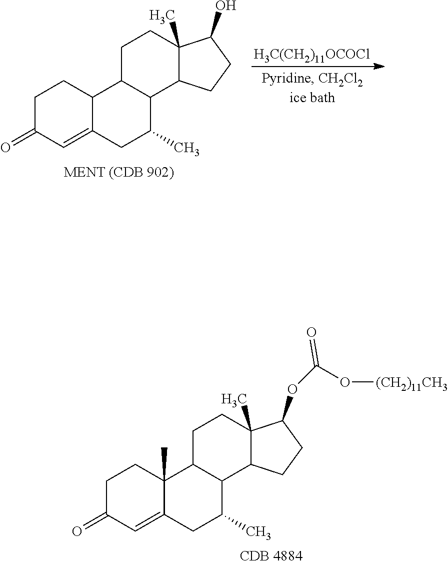

MENT (0.50 g, 1.65 mmol) was dissolved in CH₂Cl₂ (10 mL). To the solution pyridine (0.25 g, 3.15 mmol) was added and the solution was cooled in an ice-bath (5-10° C.). To the resulting solution dodecyl chloroformate (0.75 g, 3.00 mmol) was added and the solution was stirred at room temperature for 18 hours (TLC: some starting material). The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the MENT carbonate ester as low melting solid (0.42 g). HPLC: 98.7%, LC-MS: 501.3 (exact mass: 500.4). ¹H NMR partial (400 MHz, CDCl₃): δ 5.85 (s, 1H, C-4 CH), 4.55 (q, 1H, C-17 CH), 4.12 (t, 2H, OCOO—CH₂), 0.92 (s, 3H, C-18 CH₃), 0.78 (dd, 3H, C-7α CH₃).

### Example 24

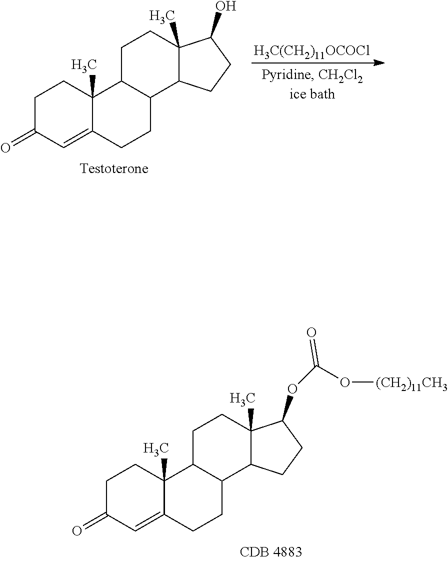

Testosterone (0.50 g, 1.65 mmol) was dissolved in CH₂Cl₂ (10 mL). To the solution pyridine (0.25 g, 3.15 mmol) was added and the solution was cooled in an ice-bath (5-10° C.). To the resulting solution dodecyl chloroformate (0.75 g, 3.00 mmol) was added and the solution was stirred at room temperature for 4 hours (TLC: trace starting material). The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the carbonate ester as low melting solid (0.72 g). HPLC: 99.24%, LC-MS: 501.3 (exact mass: 500.4). ¹HNMR partial (400 MHz, CDCl₃): δ 5.74 (s, 1H, C-4 CH), 4.53 (q, 1H, C-17 CH), 4.13 (t, 2H, OCOO—CH₂), 1.33 (s, 3H, C-11 CH₃), 0.88 (s, 3H, C-18 CH₃).

### Example 25

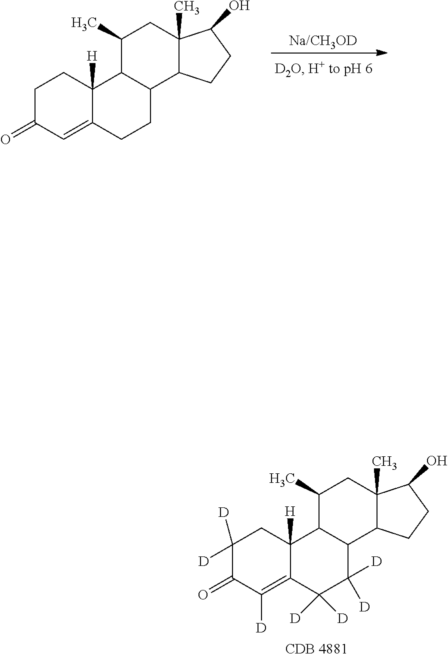 [SMILES: C[C@H]1C[C@@]2(C)C(CC[C@@H]2O)C2CCC3=CC(=O)CC[C@@H]3C21 ; "Na/CH3OD" ; [2H]C1=C2[C@H](CC([2H])([2H])C1=O)C1C(C3CC[C@H](O)[C@@]3(C)C[C@@H]1C)C([2H])([2H])C2([2H])[2H]]

Under nitrogen, freshly cut Na (1.0 g, 43 mmol) was added to CH₃OD (50 mL, 1.2 mmol). The mixture was heated to reflux to obtain a clear solution. D₂O (7.00 mL, 387 mmol) was added to the solution followed by a solution of the compound (0.5 g, 1.7 mmol) in CH3OD (10 mL). The resultant light yellow solution was heated at reflux for 2 hours. The reaction mixture was concentrated to a residue. To the mixture fresh D₂O (7 mL) and CH₃OD (60 mL) were added and the resulting mixture was further heated at reflux for 2 hours. The mixture was cooled and carefully neutralized with 9M solution of H₂SO₄ (concentrated sulfuric acid diluted with equal parts of D₂O). The resulting mixture was extracted with ethyl acetate (2×50 mL). The organics was washed with saturated NaHCO₃ solution (2×30 mL), brine (1×50 mL), dried over MgSO₄, filtered and evaporated to a residue. This material was purified over silica gel column using gradients of CH₂Cl₂/acetone to afford the deuterated compound as white foam (0.28 g). HPLC: 98.6%, LC-MS: 296.1 (exact mass: 295.2). ¹H NMR partial (400 MHz, CDCl₃): δ 3.64 (t, 1H, C-17 CH), 1.08 (d, 3H, C-11 CH₃), 0.93 (s, 3H, C-18 CH₃).

### Example 26

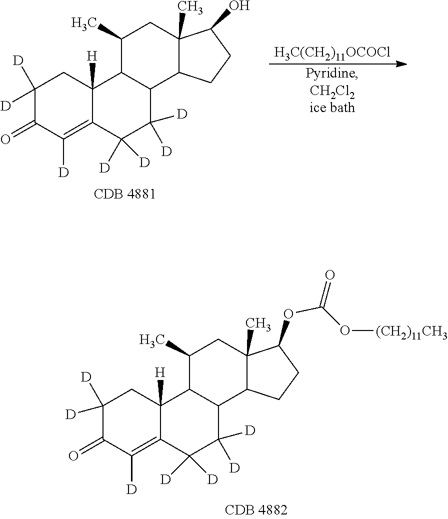

D-7 MNT (CDB 4881, 0.25 g, 0.86 mmol) was dissolved in CH₂Cl₂ (7 mL). To the solution pyridine (0.13 g, 1.58 mmol) was added and the solution was cooled in an ice-bath (5-10° C.). To the resulting solution dodecyl chloroformate (0.37 g, 1.50 mmol) was added and the solution was stirred at room temperature for 4 hours (TLC: trace starting material). The mixture was diluted with CH₂Cl₂ (40 mL) and the organic phase was washed successively with water (1×40 mL), 5% HCl solution (1×40 mL), brine (1×40 mL), dried over MgSO₄, filtered and evaporated. The crude residue was purified over silica gel column using gradients of CH₂Cl₂/acetone to afford the carbonate ester as oil (0.30 g, CDB 4882). HPLC: 98.7%, LC-MS: 507.4 (exact mass: 507.3). ¹H NMR partial (400 MHz, CDCl₃): δ 4.5 (dod, 1H, C-17 CH), 1.08 (d, 3H, C-11 CH₃), 0.98 (s, 3H, C-18 CH₃), 0.95 (t, 3H, undecanoyl CH₃).

### Example 27

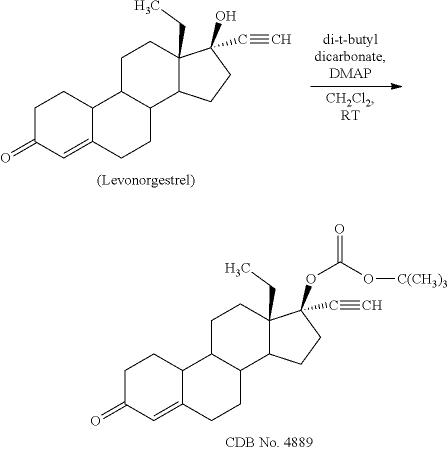 [SMILES: C#C[C@]1(O)CCC2C3CCC4=CC(=O)CCC4C3CC[C@@]21CC ; C#C[C@]1(OC(=O)OC(C)(C)C)CCC2C3CCC4=CC(=O)CCC4C3CC[C@@]21CC]

A mixture of Levonorgestrel (0.31 g, 1.0 mmol), di-t-butyl dicarbonate (0.29 g, 1.3 mmol) and DMAP (0.2 g, 1.7 mmol) in dry CH₂Cl₂ (6 mL) was stirred at room temperature overnight. TLC of a mini work-up showed a less polar new spot along with starting Levonorgestrel in ca. 1:1 ratio. The reaction mixture was worked up anyway. The reaction mixture was stripped and the residue was dissolved in EtOAc (15 mL), washed with 5% H₃PO4(aq) (1×10 mL) and brine (1×10 mL), dried (MgSO₄), filtered then stripped. The residue (0.40 g) was triturated in MTBE (5 mL) at room temperature and filtered, air-dried to recover Levonorgestrel (0.11 g). The mother liquor was stripped and the residue (0.28 g) was purified via a silica gel column (20 g), eluted with 3% EtOAc/CH₂Cl₂, to afford a white solid (0.19 g). HPLC: 97.9%.
The above procedure was scaled up (1.0 g) and modified by using EDC to replace CH₂Cl₂; and the reaction mixture was heated at reflux with additional reagents added. However, still incomplete reaction was observed. The reaction mixture was worked up in the same manner to obtain additional desired carbonate 0.80 g (CDB 4889). HPLC: 95.4%. ¹HNMR (partial) (400 MHz, CDCl₃): δ 1.01 (t, 3H, ethyl CH₃), 1.50 (s, 9H, t-butyl), 2.66 (d, 1H, acetylene), 5.84 (s, 1H, C-4 CH) ppm. LC-MS: 413.2, (MW: 412.6), TLC, EtOAC/CH₂Cl₂ (1:9)=0.57.

### Example 28

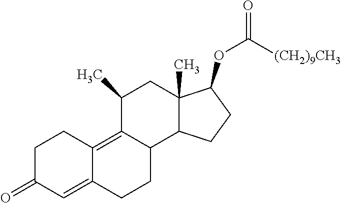

To (11S,13S,17S)-17-hydroxy-11,13-dimethyl-1,2,6,7,8,11,12,13,14,15,16,17-dodecahydro-3H-cyclopenta[a]phenanthren-3-one (2.00 g, 6.99 mmol) was dissolved in CH₂Cl₂ (20 mL). To the solution pyridine (0.66 g, 8.30 mmol) was added and the solution was cooled in an ice-bath (5-10° C.). To the resulting solution undecanoyl chloride (1.72 g, 8.41 mmol) was added and the solution was stirred at room temperature for 1 hour [TLC: complete]. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the ester as colorless oil (2.35 g, CDB 4891). HPLC: 98.7%. LCMS: 454.5 (confirmed).

### Example 29

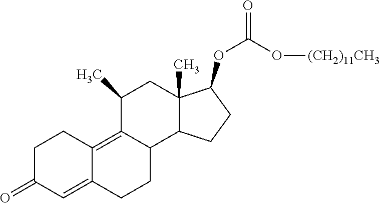

To (11S,13S,17S)-17-hydroxy-11,13-dimethyl-1,2,6,7,8,11,12,13,14,15,16,17-dodecahydro-3H-cyclopenta[a]phenanthren-3-one (2.00 g, 6.99 mmol) was dissolved in CH₂Cl₂ (20 mL). To the solution, pyridine (0.66 g, 8.30 mmol) was added and the solution was cooled in an ice-bath (5-10° C.). To the resulting solution, dodecyl chloroformate (2.08 g, 8.41 mmol) was added and the solution was stirred at room temperature for 5 hours [TLC: trace starting material]. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the ester as colorless oil (1.95 g, CDB 4892). HPLC: 98.2%. LCMS: 498.3 (confirmed).

### Example 30

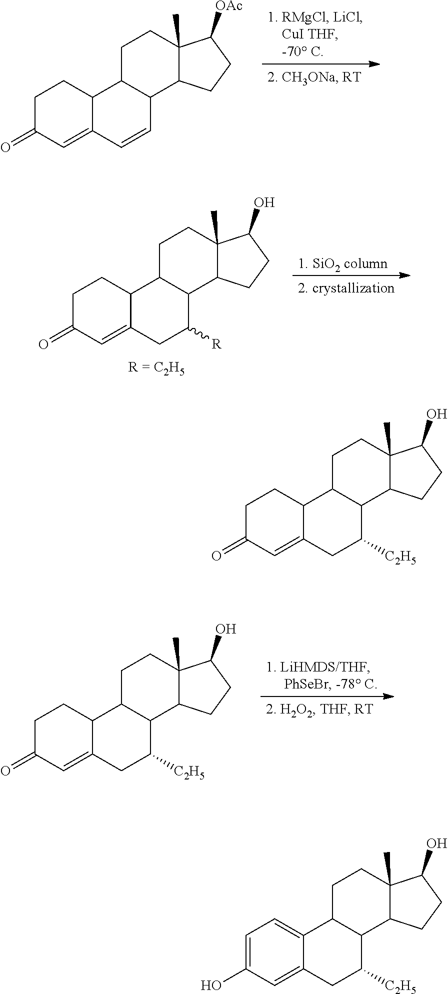

Under nitrogen, a mixture of LiCl (0.6 g, 14 mmol) and CuI (1.2 g, 6.3 mmol) in dry THF (30 mL) was stirred at room temperature for 1 hour. The resulting clear solution was added to a stirred, cold (−10° C.) solution of 17-OAc compound (3.2 g, 10 mmol) in dry THF (30 mL), followed by the addition of TMSCl (8.6 mL, 68 mmol). The resulting mixture was then cooled to −75° C. (dry ice/acetone bath). The commercial 2.0 M EtMgCl in THF (16 mL, 32 mmol) was diluted with same amount of THF (16 mL) then added dropwise, over a period of 1 hour, into the reaction mixture and temp was kept at ˜−75° C. during addition. After addition, stirring was continued for 3 hours at −75° C., then water (10 mL) was added slowly and carefully. The resulting acidic reaction mixture was stirred at room temperature overnight. The mixture was diluted with EtOAc (50 mL) and the organic layer was separated then washed successively with 50% conc. NH₄OH (1×20 mL), 50% conc. NH₄OH/conc. NH₄Cl (1:1, 1×20 mL), water (1×20 mL) and brine (1×10 mL). All the aq. washings were re-extracted with EtOAc (1×30 mL). The organic extracts were combined, dried over MgSO₄ and evaporated to dryness. The residual oil (3.2 g) was dissolved in anhydrous methanol (20 mL). To the stirred solution was added 25% CH₃ONa—CH₃OH (3 mL), and the solution was stirred at room temperature overnight. Next day, the solution was neutralized with Mac-3 resin (4.0 g), stirred for 1 hour, and filtered. The filtrate was evaporated to an oil (2.2 g). The oil was chromatographed over a flash silica gel column (70 g), using 5˜15% EtOAc/CH₂Cl₂. Based on TLC, good upper fractions (7-alpha ethyl) were pooled combined and stripped. The residue was crystallized from boiling MTBE to obtain soft solid (1.1 g).¹H NMR partial (400 MHz, CDCl₃): δ 5.84 (s, 1H, Olefinic-H), 3.68 (t, 1H, C-17 CH), 1.18 (s, 3H, CH₃), 0.85 (t, 3H, CH₃), 0.80 (s, 3H, CH₃). HPLC: 97%. LCMS (M+1): 303.4. Under nitrogen, to a stirred solution of the starting material (0.61 g, 2.0 mmol) in dry THF (10 mL), cooled in a dry ice/acetone bath (˜−78° C.), was added 1.0 M LiHMDS/MTBE (10.0 mL, 10.0 mmol). After stirring for 1.5 hours, phenylselenyl bromide (0.95 g, 4.0 mmol) in dry THF (10 mL) was added. The resulting reaction mixture was stirred for 3 hours with slow warming to 0° C. TLC indicated completion. The reaction mixture was quenched with saturated NH₄C1(aq) (20 mL). The organic layer was separated and washed successively with water (1×20 mL) and brine (1×15 mL), then dried (MgSO₄), filtered and evaporated to dryness. The residual oil (˜1.5 g) was dissolved in THF (20 mL) at room temperature and 30% H₂O₂(aq) (0.4 mL, 3.6 mmol) was added. The resulting reddish solution was stirred at room temperature for 3 hours then quenched with sat. Na₂S₂O₃(aq) (10 mL). The reaction mixture was extracted with EtOAc (2×40 mL). The combined organic layers were washed with water (1×30 mL), brine (1×30 mL), dried over MgSO₄, filtered and stripped. The crude dark red oil (˜1.6 g) was passed through a flash silica gel column (50 g) and eluted with 5˜15% EtOAc/CH₂Cl₂. The product containing fractions were pooled and stripped, vacuum pumped to dryness. This material was again re-purified over silica column to obtain pure fractions, and concentrated. The resulting beige solid (0.40 g) was recrystallized from minimum neat CH₂Cl₂ to obtain pure product as a white solid, 0.25 g (53%) (CDB 4951). ¹HNMR (Partial, 400 MHz, CDCl₃): δ=7.15 (d, J=8.0 Hz, 1H, C₁—H), 6.61 (d, J=8.0 Hz, 1H, C₂—H), 6.54 (s, 1H, C₄—H), 3.75 (t, C₁₇—H), 0.83 (d, J=7.2 Hz, 3H, C₇-Me), 0.91 (t, 3H, CH₃), 0.78 (s, 3H, C₁₃-Me). HPLC: 94.2%. LCMS (M+1): 301.1.

### Example 31

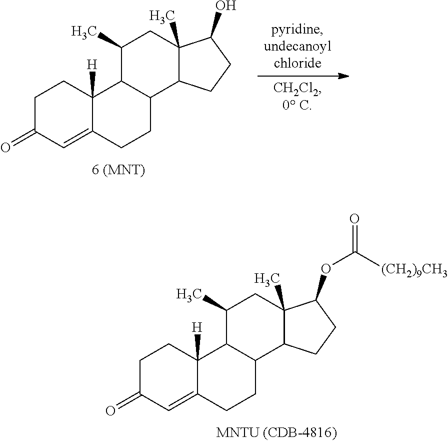

Under nitrogen, to a stirred solution of MNT 6 (1.0 g, 3.5 mmol) in CH₂Cl₂ (10 mL), cooled in an ice/water bath, was added pyridine (0.36 g, 4.6 mmol). After stirring for 30 min., undecanoyl chloride (0.91 g, 4.5 mmol) was added. The resulting reaction mixture was stirred at 0° C. for ½ h. TLC of a mini workup indicated completion. The reaction mixture was stripped and the residue was dissolved/slurried in MTBE (30 mL), washed successively with water (1×10 mL), 1 N HCl (1×10 mL), water (1×10 mL), 5% NaHCO₃(aq) (1×10 mL) and brine (1×10 mL), then dried (MgSO₄), filtered and evaporated to dryness. The crude residue (2.0 g) was purified through a flash silica gel (50 g) column, eluted with 0˜5% acetone/CH₂Cl₂. The fractions containing product were pooled and stripped, pumped under high vacuum. The resulting oil solidified upon cooling in a freezer overnight to afford a white solid, 1.6 g (100%), mp. 36-38° C. ¹H NMR partial (400 MHz, CDCl₃): δ 5.85 (s, 1H, Olefinic-H), 4.56 (dd, 1H, J=7.2 & 8.8 Hz), 1.06 (d, 3H, J=7.6 Hz, CH₃), 0.92 (s, 3H, CH₃), 0.88 (t, 3H, J=6.8 Hz, CH₃). HPLC: 99.3%; LC/MS: 457.5 (M+1).

### Example 32

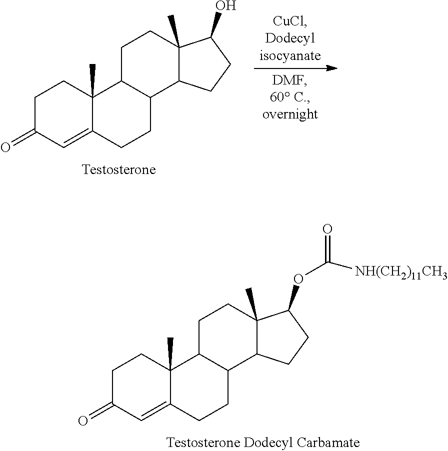

Under N₂, to a stirred mixture of Testosterone (200 mg, 0.69 mmol) and CuCl (72 mg, 0.72 mmol) in dry DMF (5 mL) was added Dodecyl isocyanate (195 mg, 0.92 mmol). The resulting reaction mixture was stirred at RT for 1 h. TLC of a mini work-up indicated only ca.10% completion. Additional Dodecyl isocyanate (150 mg, 0.70 mmol) was added, and the reaction mixture was heated in an oil bath at ca.60° C. overnight. TLC now indicated ca.80% completion. The temperature was raised to ca.80° C. and heated for additional 2 h, then worked up. The reaction mixture was cooled to RT and diluted with EtOAc (20 mL), then washed with water (2×10 mL). The combined aqueous washings were re-extracted with EtOAc (1×15 mL). The EtOAc solutions were combined, washed with brine (1×10 mL), then dried over MgSO₄, filtered and stripped. The residue (0.51 g) was passed through a flash SiO₂ column (50 g) and eluted with 0˜10% EtOAc/CH₂Cl₂. The product containing fractions were pooled and stripped. The resulting gummy residue (0.32 g) was triturated in a mixed solvent of MTBE/pet ether (1:9) at RT and filtered, air dried to constant wt. to afford an off-white solid, 191 mg (55%), mp. 94-96° C. ¹H NMR partial (400 MHz, CDCl₃): δ 5.73 (s, 1H, Olefinic-H), 4.59 (br.s, 1H), 4.54 (t, 1H, J=8.4 Hz), 3.15 (m, 2H), 1.19 (s, 3H, CH₃), 0.88 (t, 3H, J=6.8 Hz, CH₃), 0.80 (s, 3H, CH₃). HPLC: 95.5%; LC/MS: 500.5 (M+1).

### Example 33

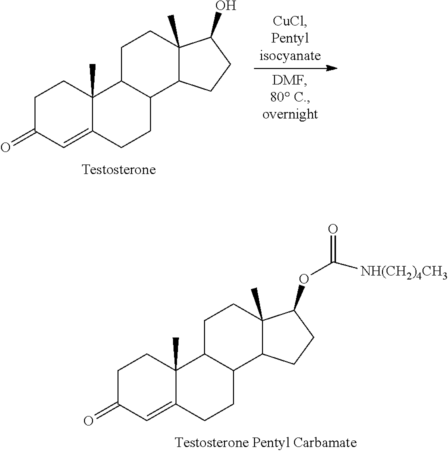

Under N₂, to a stirred mixture of Testosterone (200 mg, 0.69 mmol) and CuCl (72 mg, 0.72 mmol) in dry DMF (5 mL) was added Pentyl isocyanate (120 mg, 1.06 mmol). The resulting reaction mixture was heated in an oil bath at ca.80° C. overnight. The reaction mixture was cooled to RT and diluted with EtOAc (20 mL), then washed with water (2×10 mL). The combined aqueous washings were re-extracted with EtOAc (1×15 mL). The EtOAc solutions were combined, washed with brine (1×10 mL), then dried over MgSO₄, filtered and stripped. The residue (0.56 g) was passed through a flash SiO₂ column (50 g) and eluted with 0˜10% EtOAc/CH₂Cl₂. The product containing fractions were pooled and stripped. The resulting gummy residue (0.28 g) was triturated in a mixed solvent of MTBE/pet ether (1:9) at RT and filtered, air dried to constant wt. to afford an off-white solid, 182 mg (65%), mp. 140-141° C. ¹H NMR partial (400 MHz, CDCl₃): δ 5.73 (s, 1H, Olefinic-H), 4.60 (br.s, 1H), 4.54 (t, 1H, J=8.4 Hz), 3.15 (m, 2H), 1.19 (s, 3H, CH₃), 0.90 (t, 3H, J=6.8 Hz, CH₃), 0.81 (s, 3H, CH₃). HPLC: 99.8%; LC/MS: 402.4 (M+1).

### Example 34

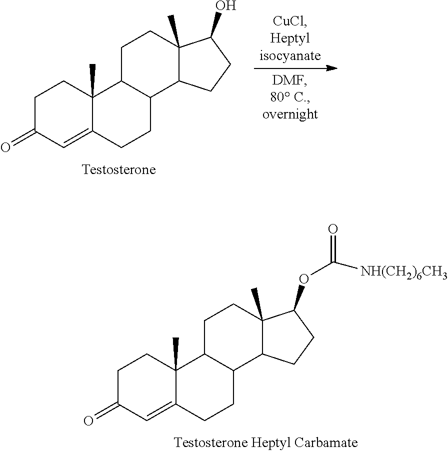

Under N₂, to a stirred mixture of Testosterone (200 mg, 0.69 mmol) and CuCl (72 mg, 0.72 mmol) in dry DMF (5 mL) was added Heptyl isocyanate (150 mg, 1.06 mmol). The resulting reaction mixture was heated in an oil bath at ca.80° C. overnight. The reaction mixture was cooled to RT and diluted with EtOAc (20 mL), then washed with water (2×10 mL). The combined aqueous washings were re-extracted with EtOAc (1×15 mL). The EtOAc solutions were combined, washed with brine (1×10 mL), then dried over MgSO₄, filtered and stripped. The residue (0.58 g) was passed through a flash SiO₂ column (50 g) and eluted with 0˜10% EtOAc/CH₂Cl₂. The product containing fractions were pooled and stripped. The resulting gummy residue (272 mg) was recrystallized from a mixed solvent of MTBE/Hexanes (1:4) at RT then cooled in a freezer overnight. The resulting solid was collected by filtration, air dried to constant wt. to afford a white solid, 232 mg (78%), mp. 81-84° C. ¹H NMR partial (400 MHz, CDCl₃): δ 5.73 (s, 1H, Olefinic-H), 4.60 (br.s, 1H), 4.54 (t, 1H, J=8.4 Hz), 3.15 (m, 2H), 1.19 (s, 3H, CH₃), 0.88 (t, 3H, J=6.8 Hz, CH₃), 0.81 (s, 3H, CH₃). HPLC: 99.3%; LC/MS: 430.5 (M+1).

### Example 35

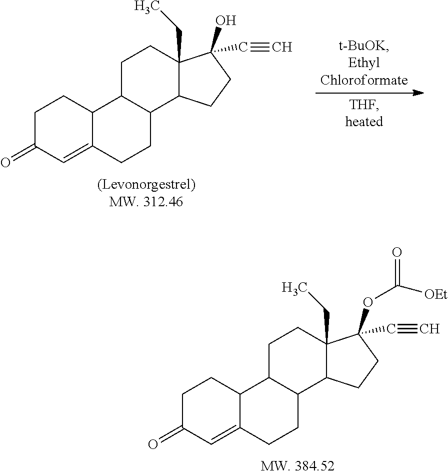 [SMILES: C#C[C@]1(O)CCC2C3CCC4=CC(=O)CCC4C3CC[C@@]21CC ; C#C[C@]1(OC(=O)OCC)CCC2C3CCC4=CC(=O)CCC4C3CC[C@@]21CC]

Under nitrogen, a mixture of Levonorgestrel (0.50 g, 1.6 mmol) and t-BuOK (0.18 g, 1.6 mmol) in dry THF (10 mL) was stirred at RT for 30 minutes, then a solution of Ethyl Chloroformate (0.28 g, 2.6 mmol) in THF (5 mL) was added dropwise through an addition funnel over a period of 30 minutes. After addition, the reaction mixture was heated (oil bath at 50-55° C.) overnight. TLC indicated some un-reacted Levonorgestrel, one less polar and one more polar new spot. Work-up anyway. The reaction mixture was cooled to RT and stripped. The residue was dissolved in EtOAc (30 mL) then washed with water (2×30 mL). The aqueous layers were separated, combined and re-extracted with EtOAc (1×30 mL). The combined EtOAc solution was washed with brine (1×10 mL), dried (MgSO₄), filtered and evaporated. The crude residue (0.50 g) was purified via a flash silica gel column (60 g), eluted with 0˜5% EtOAc/CH₂Cl₂, to isolate the less polar new spot as an off-white solid, 60 mg (9.8%). ¹H NMR partial (400 MHz, CDCl₃): δ 5.83 (s, 1H, Olefinic-H), 4.20 (m, 2H, —OCH₂—), 2.65 (s, 1H, Acetylninic-H), 1.30 (t, 3H, J=6.8 Hz, CH₃), 1.00 (t, 3H, J=7.6 Hz, CH₃). HPLC: 99.1%; LC/MS: 385.3 (M+1).

### Example 36

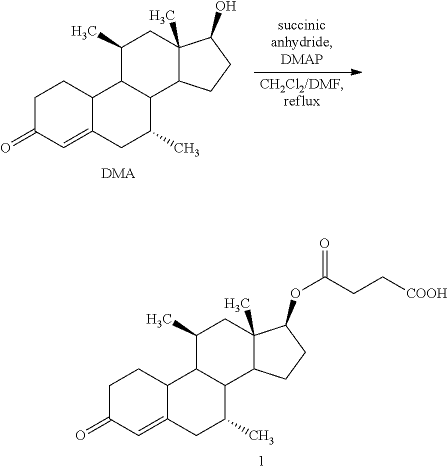

A mixture of DMA (1.5 g, 5.0 mmol), succinic anhydride (1.0 g, 10 mmol) and DMAP (1.2 g, 9.8 mmol) in a mixed solvent of CH₂Cl₂ (25 mL) and DMF (3 mL) was heated at reflux overnight. The reaction mixture was cooled to RT and washed with 10% H₃PO₄(aq) (1×10 mL), brine (1×20 mL), then dried over MgSO₄, filtered and stripped. The resulting oil (2.2 g) was dissolved in MTBE (5 mL) and stood at RT for 1 hr. The crystallized solid was collected by filtration, rinsed with cold MTBE (1×5 mL), air-dried then pumped under high vacuum to constant wt. to afford pure 1 as an off-white solid, 1.0 g (65%).

### Example 37

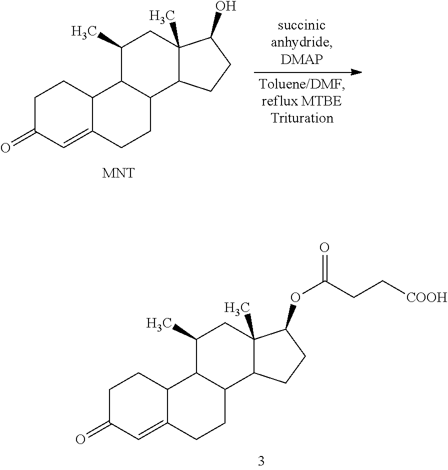

A mixture of MNT (1.0 g, 3.47 mmol), succinic anhydride (0.5 g, 5.0 mmol) and DMAP (0.8 g, 6.54 mmol) in a mixed solvent of Toluene (15 mL) and DMF (2 mL) was heated at reflux overnight. The reaction mixture was cooled to RT and washed with 10% H₃PO₄(aq) (1×10 mL), brine (1×10 mL), then dried over MgSO₄, filtered and stripped. The resulting dark brown oil (1.7 g) was dissolved in CH₂Cl₂ (5 mL) and loaded onto a flash silica gel column (40 g), then eluted with 0˜7% MeOH/CH₂Cl₂. Fractions containing product (by TLC) were pooled and stripped. The resulting residue was triturated in MTBE (5 mL) at RT and filtered, air-dried then pumped under high vacuum to constant wt. to afford pure 3 as an off-white solid, 1.1 g (80%).

### Example 38

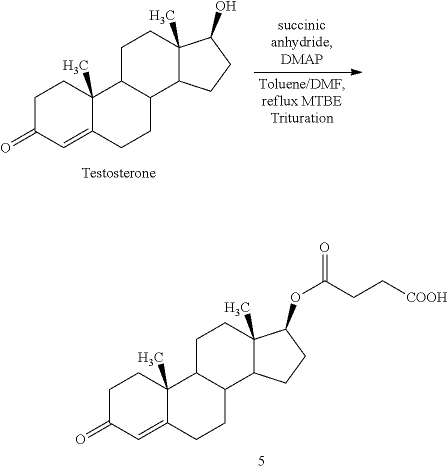

A mixture of Testosterone (1.0 g, 3.4 mmol), succinic anhydride (1.0 g, 10 mmol) and DMAP (1.2 g, 9.8 mmol) in a mixed solvent of CH₂Cl₂ (25 mL) and DMF (3 mL) was heated at reflux overnight. The reaction mixture was cooled to RT, filtered and the solution was washed with 10% H₃PO₄(aq) (1×10 mL), brine (1×20 mL), then dried over MgSO₄, filtered and stripped. The resulting oil (1.6 g) was dissolved in CH₂Cl₂ (5 mL) and loaded onto a flash silica gel column (40 g), then eluted with 0˜7% MeOH/CH₂Cl₂. Fractions containing product (by TLC) were pooled and stripped. The resulting residue was triturated in MTBE (5 mL) at RT and filtered, air-dried then pumped under high vacuum to constant wt. to afford pure 5 as an off-white solid, 1.15 g (82%).

### Example 39

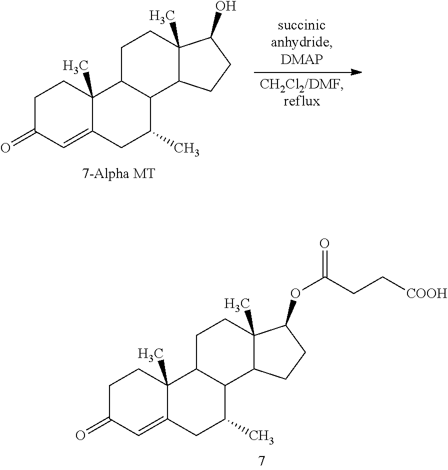

A mixture of 7-AlphaMT (0.5 g, 1.7 mmol), succinic anhydride (0.5 g, 5 mmol) and DMAP (0.6 g, 4.9 mmol) in a mixed solvent of CH₂Cl₂ (12 mL) and DMF (1 mL) was heated at reflux overnight. The reaction mixture was cooled to RT and washed with 10% H₃PO₄(aq) (1×10 mL), brine (1×20 mL), then dried over MgSO₄, filtered and stripped. The resulting oil (0.7 g) was dissolved in MTBE (3 mL) and stood at RT for 1 hr. The crystallized solid was collected by filtration, rinsed with cold MTBE (1×3 mL), air-dried then pumped under high vacuum to constant wt. to afford pure 7 as an off-white solid, 0.51 g (65%). Melting point: 180-181° C.

### Example 40

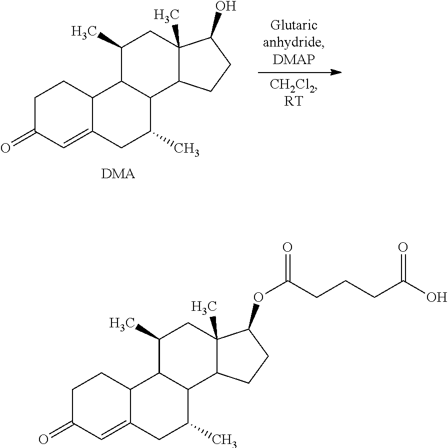

A mixture of DMA (0.5 g, 1.65 mmol), glutaric anhydride (0.4 g, 3.4 mmol) and DMAP (0.4 g, 3.3 mmol) in CH₂Cl₂ (10 mL) was stirred at RT overnight. The reaction mixture was washed with 10% H₃PO₄(aq) (1×10 mL), brine (1×10 mL), then dried over MgSO₄, filtered and stripped. The resulting residue (1.60 g) was dissolved in CH₂Cl₂ (5 mL) and loaded onto a flash silica gel column (40 g), first eluted with 20% EtOAc/CH₂Cl₂ to remove less polar impurity spot, then eluted with 5˜10% MeOH/CH₂Cl₂ to collect product spot. Fractions containing product (by TLC, has some non-polar spot.) were pooled and stripped. The oil was re purified to obtain DMA-glutaric acid adduct as a semi-solid mass (0.31 g).

### Example 41

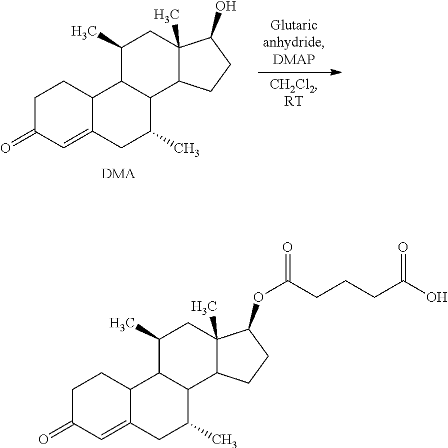

A mixture of DMA (0.5 g, 1.65 mmol), glutaric anhydride (0.4 g, 3.4 mmol) and DMAP (0.4 g, 3.3 mmol) in CH₂Cl₂ (10 mL) was stirred at RT overnight. The reaction mixture was washed with 10% H₃PO₄(aq) (1×10 mL), brine (1×10 mL), then dried over MgSO₄, filtered and stripped. The resulting residue (1.60 g) was dissolved in CH₂Cl₂ (5 mL) and loaded onto a flash silica gel column (40 g), first eluted with 20% EtOAc/CH₂Cl₂ to remove less polar impurity spot, then eluted with 5˜10% MeOH/CH₂Cl₂ to collect product spot. Fractions containing product (by TLC, has some non-polar spot.) were pooled and stripped. The oil was re purified to obtain DMA-glutaric acid adduct as a semi-solid mass (0.31 g).

### Example 42

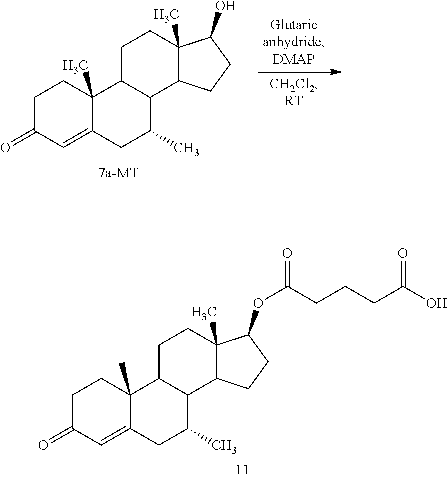

A mixture of 7α-MT (0.5 g, 1.65 mmol), glutaric anhydride (0.4 g, 3.4 mmol) and DMAP (0.4 g, 3.3 mmol) in CH₂Cl₂ (10 mL) was stirred at RT overnight. The reaction mixture was washed with 10% H₃PO₄(aq) (1×10 mL), brine (1×10 mL), then dried over MgSO₄, filtered and stripped. The resulting residue (1.60 g) was dissolved in CH₂Cl₂ (5 mL) and loaded onto a flash silica gel column (40 g), first eluted with 20% EtOAc/CH₂Cl₂ to remove less polar impurity spot, then eluted with 5˜10% MeOH/CH₂Cl₂ to collect product spot. Fractions containing product (by TLC) were pooled and stripped, pumped under high vacuum to constant wt. to afford pure 11 as an off-white solid, 0.5 g (75%).

### Example 43

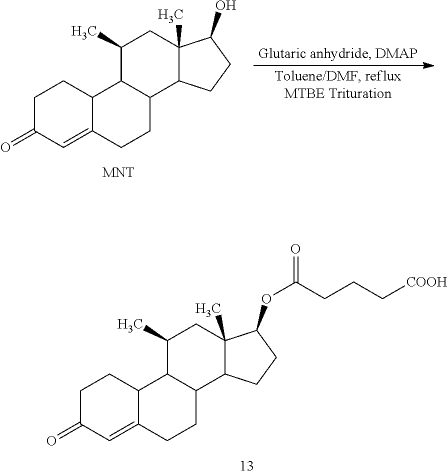

A mixture of MNT (1.0 g, 3.47 mmol), glutaric anhydride (0.53 g, 5.0 mmol) and DMAP (0.8 g, 6.54 mmol) in a mixed solvent of Toluene (15 mL) and DMF (2 mL) was heated at reflux overnight. The reaction mixture was cooled to RT and washed with 10% H₃PO₄(aq) (1×10 mL), brine (1×10 mL), then dried over MgSO₄, filtered and stripped. The resulting dark brown oil (1.7 g) was dissolved in CH₂Cl₂ (5 mL) and loaded onto a flash silica gel column (40 g), then eluted with 0˜7% MeOH/CH₂Cl₂. Fractions containing product (by TLC) were pooled and stripped. The resulting residue was triturated in MTBE (5 mL) at RT and filtered, air-dried then pumped under high vacuum to constant wt. to afford pure 4 as an off-white solid, 0.7 g (70%).

### Example 44

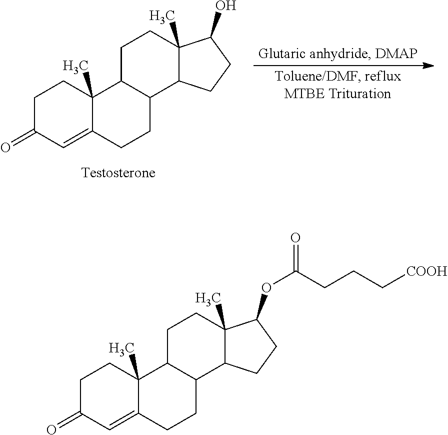

A mixture of Testosterone (1.0 g, 3.47 mmol), glutaric anhydride (0.53 g, 5.0 mmol) and DMAP (0.8 g, 6.54 mmol) in a mixed solvent of Toluene (15 mL) and DMF (2 mL) was heated at reflux overnight. The reaction mixture was cooled to RT and washed with 10% H₃PO₄(aq) (1×10 mL), brine (1×10 mL), then dried over MgSO₄, filtered and stripped. The resulting dark brown oil (1.7 g) was dissolved in CH₂Cl₂ (5 mL) and loaded onto a flash silica gel column (40 g), then eluted with 0˜7% MeOH/CH₂Cl₂. Fractions containing product (by TLC) were pooled and stripped. The resulting residue was repurified and triturated in MTBE (7 mL) at RT and filtered, air-dried then pumped under high vacuum to constant wt. to afford pure as an off-white solid, 0.61 g (62%).

### Example 45

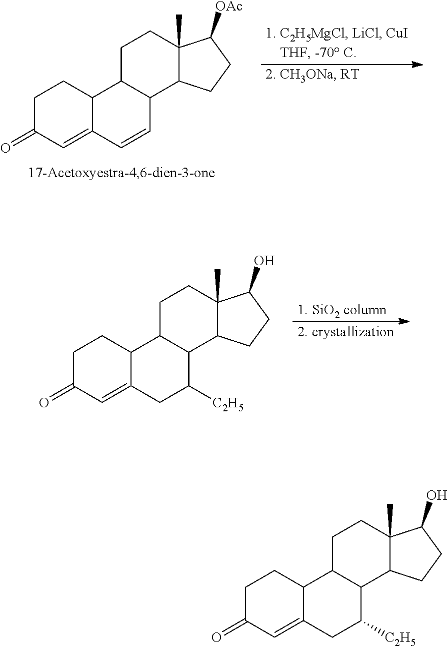

Under nitrogen, a mixture of LiCl (0.6 g, 14 mmol) and CuI (1.2 g, 6.3 mmol) in dry THF (30 mL) was stirred at RT for 1 h. The resulting clear solution was added to a stirred, cold (−10° C.) solution of 17-β-Acetoxyestra-4,6-dien-3-one compound (3.2 g, 10 mmol) in dry THF (30 mL), followed by the addition of TMSCl (8.6 mL, 68 mmol). The resulting mixture was then cooled to −75° C. (dry ice/acetone bath). The commercial 2.0 M EtMgCl in THF (16 mL, 32 mmol) was diluted with same amount of THF (16 mL) then added dropwise, over a period of 1 h, into the reaction mixture and temp was kept at ˜−75° C. during addition. After addition, stirring was continued for 3 h at −75° C., then water (10 mL) was added slowly and carefully. The resulting acidic reaction mixture was stirred at RT overnight. The mixture was diluted with EtOAc (50 mL) and the organic layer was separated then washed successively with 50% conc. NH₄OH (1×20 mL), 50% conc. NH₄OH/conc. NH₄Cl (1:1, 1×20 mL), water (1×20 mL) and brine (1×10 mL). All the aq. washings were re-extracted with EtOAc (1×30 mL). The organic extracts were combined, dried over MgSO₄ and evaporated to dryness. The residual oil (3.2 g) was dissolved in anhydrous methanol (20 mL). To the stirred solution was added 25% CH₃ONa—CH₃OH (3 mL), and the solution was stirred at room temperature overnight. Next day, the solution was neutralized with Mac-3 resin (4.0 g), stirred for 1 h, and filtered. The filtrate was evaporated to an oil (2.2 g). The oil was chromatographed over a flash silica gel column (70 g), using 5˜15% EtOAc/CH₂Cl₂. Based on TLC, good upper fractions (7-alpha ethyl) were pooled combined and stripped. The residue was crystallized from boiling MTBE to obtain soft solid (1.1 g). ¹H NMR partial (400 MHz, CDCl₃): δ 5.84 (s, 1H, Olefinic-H), 3.68 (t, 1H, C-17 CH), 1.18 (s, 3H, CH₃), 0.85 (t, 3H, CH₃), 0.80 (s, 3H, CH₃). HPLC: 97%. LCMS (M+1): 303.4.

### Example 46

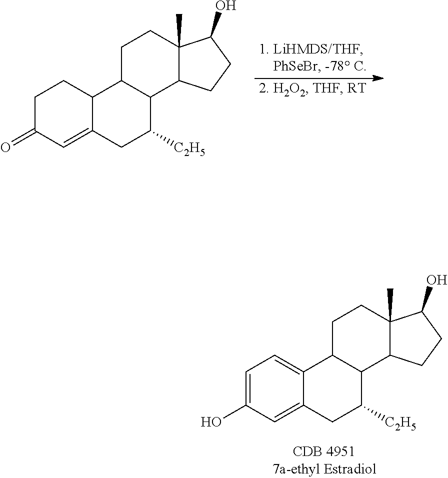

Under nitrogen, to a stirred solution of the starting material 15 (0.61 g, 2.0 mmol) in dry THF (10 mL), cooled in a dry ice/acetone bath (˜−78° C.), was added 1.0 M LiHMDS/MTBE (10.0 mL, 10.0 mmol). After stirring for 1.5 h, phenylselenyl bromide (0.95 g, 4.0 mmol) in dry THF (10 mL) was added. The resulting reaction mixture was stirred for 3 h with slow warming to 0° C. TLC indicated completion. The reaction mixture was quenched with saturated NH₄Cl(aq) (20 mL). The organic layer was separated and washed successively with water (1×20 mL) and brine (1×15 mL), then dried (MgSO₄), filtered and evaporated to dryness. The residual oil (˜1.5 g) was dissolved in THF (20 mL) at room temperature and 30% H₂O₂(aq) (0.4 mL, 3.6 mmol) was added. The resulting reddish solution was stirred at room temperature for 3 h then quenched with sat. Na₂S₂O₃(aq) (10 mL). The reaction mixture was extracted with EtOAc (2×40 mL). The combined organic layers were washed with water (1×30 mL), brine (1×30 mL), dried over MgSO₄, filtered and stripped. The crude dark red oil (˜1.6 g) was passed through a flash silica gel column (50 g) and eluted with 5˜15% EtOAc/CH₂Cl₂. The product containing fractions were pooled and stripped, vacuum pumped to dryness. This material was again re-purified over silica column to obtain pure fractions, and concentrated. The resulting beige solid (0.40 g) was recrystallized from minimum neat CH₂Cl₂ to obtain pure product as a white solid, 0.25 g (53%). ¹HNMR (Partial, 400 MHz, CDCl₃): δ=7.15 (d, J=8.0 Hz, 1H, C₁—H), 6.61 (d, J=8.0 Hz, 1H, C₂—H), 6.54 (s, 1H, C₄—H), 3.75 (t, C₁₇—H), 0.83 (d, J=7.2 Hz, 3H, C₇-Me), 0.91 (t, 3H, CH₃), 0.78 (s, 3H, C₁₃-Me). HPLC: 94.2%. LCMS (M+1): 301.1.

### Example 47

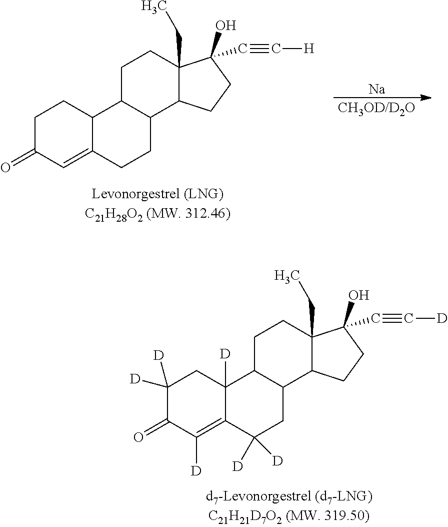

Sodium (3.0 g, 0.13 mol) was added to CH₃OD (100 mL) and gently refluxed on a steam bath, under nitrogen, for 2 h (70° C.). After cooling to 35-40° C., D₂O (20 mL) was added to above clear solution, followed by a solution of Levonorgestrel (1.5 g, 4.8 mmol) in CH₃OD (20 mL). The mixture was refluxed for additional 2 h, then allowed to stand over weekend at RT under nitrogen. The mixture was evaporated to a residue, then fresh D₂O (20 mL) and CH₃OD (120 mL) were added. The mixture was refluxed further for additional 2 h. The solvents were evaporated to give a semi solid. This was diluted with D₂O (20 mL). The slurry was cooled in an ice bath and pH was adjusted to 4 using D₂SO₄ (4 mL)/D₂O (8 mL) mixture. The resulting product was extracted with EtOAc (3×60 mL). The combined organic phase was treated with 15% sodium bicarbonate solution (100 mL) and separated. After drying over MgSO₄ (8 g), the solvent was evaporated and vacuum dried to yield crude LNG-d₇, 1.5 g. Purified over silica gel (80 g) using CH₂Cl₂/acetone gradient (0 to 5%), fractions were pooled and evaporated to afford purified LNG-d₇, 1.0 g (65%).

Half of this purified d₇-LNG (0.49 g, HPLC: 93.59% was dissolved/suspended in CH₂Cl₂ (5 mL) at RT and filtered, vacuum dried to obtain pure LNG-d₇ (0.15 g, HPLC: 99.01%) as a white solid. The clear filtrate was loaded onto a flash silica gel column (60 g), eluted with neat CH₂Cl₂ (ca. 3 L) then with 1-5% MTBE/CH₂Cl₂. Pure fractions were pooled and stripped, the residue was triturated in hexanes and filtered, air-dried then pumped under high vacuum to afford additional pure LNG-d₇ (0.26 g, HPLC: 99.49%) as a white solid. ¹H NMR partial (400 MHz, CDCl₃): δ 1.82 (s, 1H, OH), 1.01 (t, 3H, J=7.2 Hz, CH₃). HPLC: 99.5%. LC/MS: 320.3 (M+1).

### Example 48

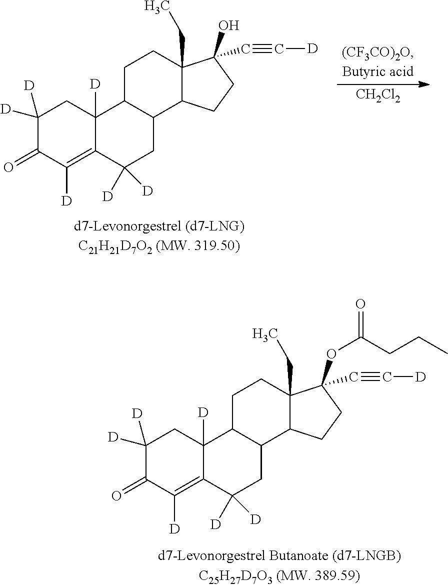 [SMILES: [2H]C#C[C@]1(O)CCC2C3CC([2H])([2H])C4=C([2H])C(=O)C([2H])([2H])CC4([2H])C3CC[C@@]21CC ; "CH2Cl2" ; "(CF3CO)2O,Butyric acid" ; [2H]C#C[C@]1(OC(=O)CCC)CCC2C3CC([2H])([2H])C4=C([2H])C(=O)C([2H])([2H])CC4([2H])C3CC[C@@]21CC]

Under N₂, in a 50 ml round-bottomed flask, 300 mg (1.49 mmol) of Trifluoro Acetic Anhydride and Butyric Acid (120 mg, 1.36 mmol) was mixed in CH₂Cl₂ (10 mL) and stirred for 45 min. then d₇-LNG (300 mg, 0.94 mmol) was added to the mixed anhydride and stirred for 2.5 h at RT. TLC indicated only trace of unreacted LNG-d, left. Water (20 mL) was added to quench the mixed anhydride and stirred for 20 min. Water was separated and CH₂Cl₂ phase was washed with aqueous 5% NaHCO₃ to remove excess acid. Additional water washes (2×10 mL), then drying over MgSO₄ (5 g) and the organic phase was evaporated to dryness. The resulting crude solid 370 mg was crystallized from a mixture of hot THF (2.5 mL) and n-Heptane (3.2 mL). Pure white solid was collected, rinsed with n-Heptane (2.5 mL) and vacuum dried to constant wt, to afford pure LNGB, 131.8 mg (38.8% yield). ¹H NMR partial (400 MHz, CDCl₃): δ 2.78 (m, 1H), 1.02 (t, 3H, J=7.2 Hz, CH₃), 0.96 (t, 3H, J=7.6 Hz, CH₃). HPLC: 98.4%. LC/MS: 390.4 (M+1).

### Example 49

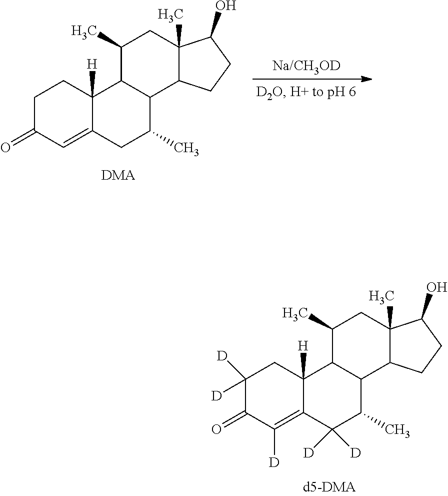

Under nitrogen, freshly cut Na (2.0 g, 87 mmol) was added to CD₃OD (100 mL, 2.4 mmol). The mixture was heated to reflux to obtain a clear solution. D₂O (14.00 mL, 774 mmol) was added to the solution followed by a solution of compound DMA (1.0 g, 3.3 mmol) in CH₃OD (10 mL). The resultant yellow solution was heated at reflux for 2 h. The reaction mixture was concentrated to a residue. To the mixture fresh D₂O (14 mL) and CD₃OD (120 mL) were added, and the resulting mixture was further heated at reflux for 2 h. The mixture was concentrated, and the resulting residue was suspended in D₂O (20 mL), cooled and carefully neutralized with 9M solution of H₂SO₄ (10 mL, 5 mL of concentrated sulfuric acid diluted with equal parts of D₂O). The resulting mixture was extracted with ethyl acetate (2×100 mL). The organics was washed with saturated NaHCO₃ solution (2×50 mL), brine (1×100 mL), dried over MgSO₄, filtered and evaporated. This material was purified over silica gel column using gradients of CH₂Cl₂/acetone to afford the deuterated compound. The residue was crystallized from MTBE to obtain d₅-DMA (CDB 4857) as white solid (0.51 g). ¹H NMR partial (400 MHz, CDCl₃): δ 3.62 (t, 1H, C-17 CH), 1.06 (d, 3H, CH₃), 0.88 (s, 3H, C-18 CH₃), 0.77 (d, 3H, C-7α CH₃). HPLC: 99.7%; LC/MS: 308.3 (M+1). Melting point: 153-155° C.

### Example 50

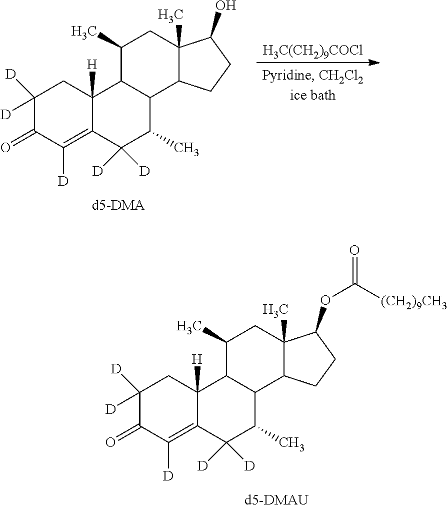

Pentadeuterated DMA (d₅-DMA, 0.31 g, 1.0 mmol) was dissolved in CH₂Cl₂ (7 mL). To the solution pyridine (0.13 g, 1.58 mmol) was added and the solution was cooled in an ice-bath (5-10° C.). To the resulting solution Undecanoyl chloride (0.30 mL, 1.35 mmol) was added and the solution was stirred at room temperature for 4 h [TLC: trace starting material]. The mixture was diluted with CH₂Cl₂ (40 mL) and the organic phase was washed successively with water (1×40 mL), 5% HCl solution (1×40 mL), brine (1×40 mL), dried over MgSO₄, filtered and evaporated. The crude residue was purified over silica gel column using gradients of CH₂Cl₂/acetone to afford the ester as oil (0.30 g). This material was crystallized from n-pentane (2 mL) to obtain d₅-DMAU (as soft solid (0.25 g). ¹H NMR partial (400 MHz, CDCl₃): δ 4.56 (t, 1H, C-17 CH), 2.33 (t, 2H, OCOCH₂₋), 1.05 (d, 3H, CH₃), 0.92 (s, 3H, C-18 CH₃), 0.77 (d, 3H, C-7α CH₃). HPLC: 99.2%; LC/MS: 476.5 (M+1). Melting point: 62-64° C.

### Example 51

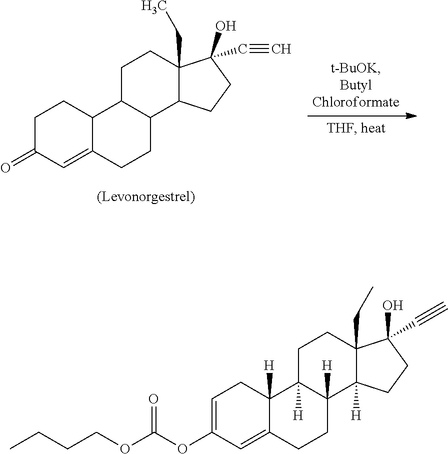

Under nitrogen, a mixture of Levonorgestrel (1.0 g, 3.2 mmol) and t-BuOK (0.36 g, 3.2 mmol) in dry THF (20 mL) was stirred at RT for 30 minutes, then a solution of Butyl Chloroformate (0.72 g, 5.2 mmol) in THF (10 mL) was added dropwise through an addition funnel over a period of 30 minutes. After addition, the reaction mixture was heated (oil bath at 50˜55° C.) overnight. TLC indicated some un-reacted Levonorgestrel, one major less polar and one minor more polar new spots. Work-up anyway. The reaction mixture was cooled to RT and stripped. The residue was dissolved in EtOAc (30 mL) then washed with water (2×30 mL). The aqueous layers were separated, combined and re-extracted with EtOAc (1×30 mL). The combined EtOAc solution was washed with brine (1×10 mL), dried (MgSO₄), filtered and evaporated. The crude residue (1.6 g) was purified via a flash silica gel column (120 g), eluted with 0˜5% EtOAc/CH₂Cl₂, to isolate the less polar major new spot as a white solid, 0.49 g (37%). ¹H NMR partial (400 MHz, CDCl₃): δ 5.87 (s, 1H, Olefinic C₄—H), 5.50 (br. s, 1H, Olefinic C₂—H), 4.17 (t, 2H, J=6.8 Hz, —OCH₂—), 2.59 (s, 1H, Acetylenic-H), 1.81 (s, 1H, —OH), 0.99 (t, 3H, J=7.2 Hz, —CH₃), 0.95 (t, 3H, J=7.6 Hz, —CH₃). HPLC: 88.3%; LC/MS: 413.4 (M+1).

### Example 52

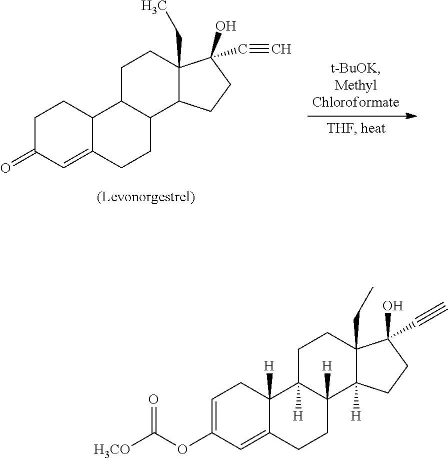

Under nitrogen, a mixture of Levonorgestrel (0.50 g, 1.6 mmol) and t-BuOK (0.18 g, 1.6 mmol) in dry THF (10 mL) was stirred at RT for 30 minutes, then a solution of Methyl Chloroformate (0.25 g, 2.6 mmol) in THF (5 mL) was added dropwise through an addition funnel over a period of 30 minutes. After addition, the reaction mixture was heated (oil bath at 50˜55° C.) overnight. TLC indicated some un-reacted Levonorgestrel, one major less polar and one minor more polar new spots. Work-up anyway. The reaction mixture was cooled to RT and stripped. The residue was dissolved in EtOAc (15 mL) then washed with water (2×15 mL). The aqueous layers were separated, combined and re-extracted with EtOAc (1×15 mL). The combined EtOAc solution was washed with brine (1×10 mL), dried (MgSO₄), filtered and evaporated. The crude residue (0.64 g) was purified via a flash silica gel column (60 g), eluted with 0˜5% EtOAc/CH₂Cl₂, to isolate the less polar major new spot as a white solid, 0.19 g (32%), as Lot No. ML-17-252-1. ¹H NMR partial (400 MHz, CDCl₃): δ 5.87 (s, 1H, Olefinic C₄—H), 5.51 (br. s, 1H, Olefinic C₂—H), 3.82 (s, 3H, —OCH₃), 2.59 (s, 1H, Acetylenic-H), 1.81 (s, 1H, —OH), 0.99 (t, 3H, J=7.2 Hz, —CH₃). HPLC: 97.9%; LC/MS: 371.4 (M+1).

### B. Oligomeric Compound Embodiments

### Example 53

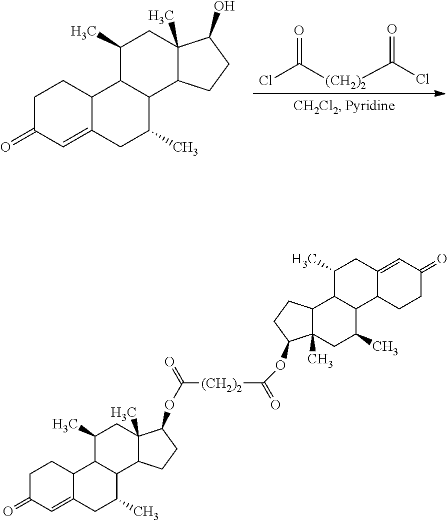

DMA (0.30 g, 1.00 mmol) was dissolved in CH₂Cl₂ (7 mL). To the solution pyridine (0.15 g, 1.50 mmol) was added and the solution was cooled in an ice-bath (5-100 C). To the resulting solution succinyl chloride (0.078 g, 0.500 mmol) was added and the solution was stirred at room temperature for 1 hour [TLC: ca. 20% new non-polar spot]. The solution was stirred at room temp for an additional 2 hours [TLC: unchanged]. To the solution, additional acid chloride 0.15 g (1.00 mmol) was added and the solution was stirred for 30 minutes [TLC: ca. 90% product +10% starting material]. The solution was diluted with CH₂Cl₂. The organic phase was washed successively with water (1×75 mL), 5% HCl solution (1×75 mL), brine (1×75 mL), dried over MgSO₄, filtered and evaporated. The crude residue was purified over silica gel column to afford the dimer as a foam (0.20 g). Similarly, another batch of DMA (0.25 g) was processed in the same manner to obtain additional 0.14 g compound. Both batches were combined and crystallized from ether/petroleum ether to obtain 0.29 g white solid (CDB 4866). LCMS (MW: 686.45 confirmed). HPLC: 97.6%. ¹H NMR partial (400 MHz, CDCl₃): δ 5.86 (s, 2H, C-4 CH), 4.60 (q, 2H, C-17 CH), 2.61 (t, 4H, OCO—CH₂), 1.07 (d, 6H, C-11β CH₃), 0.94 (s, 6H, C-13 CH₃), 0.78 (d, 6H, C-7α CH₃).

### Example 54

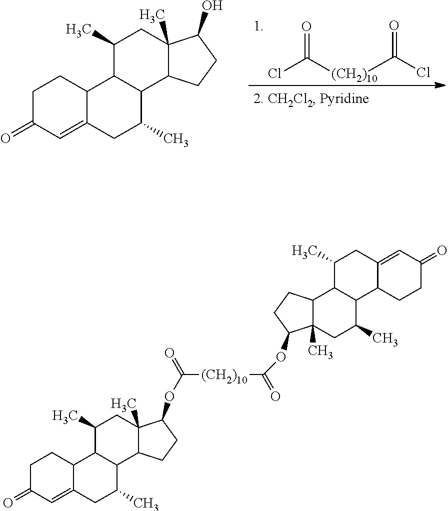

DMA (0.60 g, 2.00 mmol) was dissolved in CH₂Cl₂ (15 mL). To the solution pyridine (0.30 g, 2.25 mmol) was added and the solution was cooled in an ice-bath (5-100 C). To the resulting solution dodecanedionyl chloride (0.56 g, 2.00 mmol) was added and the solution was stirred at room temperature for 4 hours [TLC: trace starting material]. The mixture was diluted with CH₂Cl₂ (75 mL) and the organic phase was washed successively with water (1×75 mL), 5% HCl solution (1×75 mL), brine (1×75 mL), dried over MgSO₄, filtered and evaporated. The crude residue was purified over silica gel column using gradients of CH₂Cl₂/acetone to afford the dimer as colorless oil (0.36 g) (CDB 4867). LCMS (MW: 798.5 confirmed). HPLC: 95.6%. ¹H NMR partial (400 MHz, CDCl₃): δ 5.87 (s, 2H, C-4 CH), 4.60 (q, 2H, C-17 CH), 1.07 (d, 6H, C-11β CH₃), 0.93 (s, 6H, C-13 CH₃), 0.80 (d, 6H, C-7α CH₃).

### Example 55

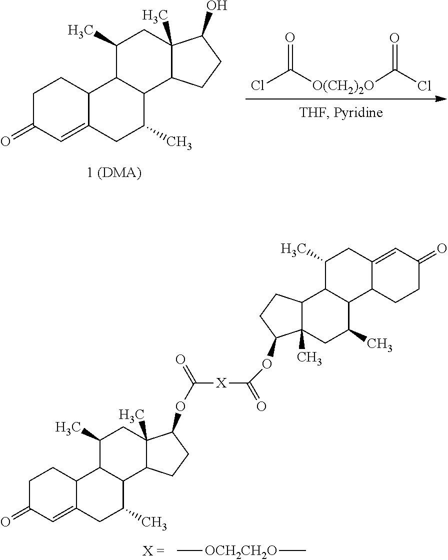

DMA (0.60 g, 2.00 mmol) was dissolved in anhydrous THF (15 mL). To the solution pyridine (0.30 g, 3.00 mmol), followed by ethylene bischloroformate (0.56 g, 3.00 mmol) were added and the resulting stirred solution was heated at reflux for 4 hours [TLC: starting material disappeared and three very close new non-polar spot]. Reaction was worked up and the major non-polar spot was isolated through silica gel column to obtain 0.31 g product as white foam (CDB 4868). LCMS (MW: 718 confirmed). HPLC: 98%. ¹H NMR partial (400 MHz, CDCl₃): δ 5.86 (s, 2H, C-4 CH), 4.52 (q, 2H, C-17 CH), 4.35 (t, 4H, OCO—OCH₂), 1.08 (d, 6H, C-11β CH₃), 0.96 (s, 6H, C-13 CH₃), 0.78 (d, 6H, C-7α CH₃).

### Example 56

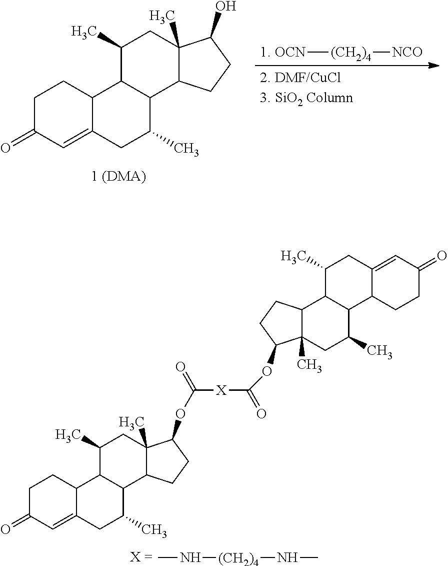

1,4-Diisocyanatobutane (0.32 g, 2.20 mmol) was added to a heterogeneous mixture of DMA (0.60 g, 2.00 mmol) in DMF (12 mL) containing CuCl (0.2 g, 2.2 mmol). The mixture was stirred at room temperature for 1 hour [TLC: formation of a new polar spot]. The reaction mixture was diluted with ether (1×140 mL) and the organic layer was washed successively with water (1×100 mL) and brine (1×100 mL). After drying over MgSO₄, the organic phase was evaporated and the crude oil was purified over silica column to obtain the bis-carbamate as an oil (0.30 g, CDB 4870). HPLC: 97.7%. Mass: 744.45 (confirmed). ¹H NMR partial (400 MHz, CDCl₃): δ 5.85 (s, 2H, C-4 CH), 4.52 (q, 2H, C-17 CH), 1.09 (d, 6H, C-11β CH₃), 0.92 (s, 6H, C-13 CH₃), 0.80 (d, 6H, C-7α CH₃).

### Example 57

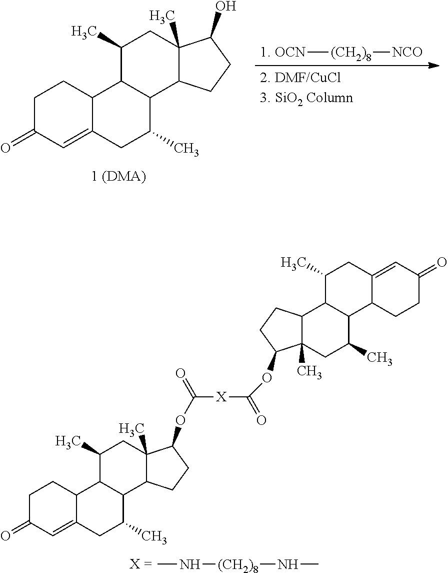

1,4-Diisocyanatooctane (0.30 g, 1.50 mmol) was added to a heterogeneous mixture of DMA (0.45 g, 1.50 mmol) in DMF (10 mL) containing CuCl (0.15 g, 1.55 mmol). The mixture was stirred at room temperature for 1 hour [TLC: formation of a new polar spot]. The reaction mixture was diluted with ether (1×100 mL) and the organic layer was washed successively with water (1×75 mL) and brine (1×75 mL). The organic layer was dried over MgSO₄ and evaporated to a crude oil, which was purified over silica column to obtain the bis-carbamate as an oil (0.28 g, CDB 4871). HPLC: 98%. Mass: 800.4 (confirmed). ¹H NMR partial (400 MHz, CDCl₃): δ 5.86 (s, 2H, C-4 CH), 4.52 (q, 2H, C-17 CH), 1.08 (d, 6H, C-11β CH₃), 0.92 (s, 6H, C-13 CH₃), 0.78 (d, 6H, C-7α CH₃).

### Example 58

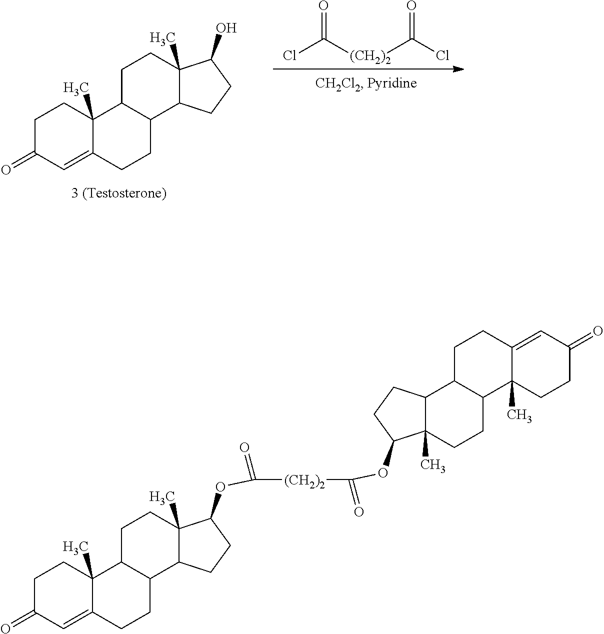

Testosterone (0.88 g, 3.00 mmol) was dissolved in CH₂Cl₂ (22 mL). To the solution pyridine (0.45 g, 4.50 mmol) was added and the solution was cooled in an ice-bath (5-100 C). To the resulting solution succinyl chloride (0.24 g, 1.50 mmol) was added and the solution was stirred at room temperature for 1 hour [TLC: ca. 50% new non-polar spot]. To the solution, additional acid chloride 0.24 g (1.50 mmol) was added and the solution was stirred for an additional 1 hour [TLC: ca. 95% product +5% starting material]. The solution was diluted with CH₂Cl₂ (50 mL). The organic phase was washed successively with water (1×50 mL), 10% HCl solution (1×50 mL), brine (1×50 mL), dried over MgSO₄, filtered and evaporated to obtain a crude residue. The crude residue was purified over silica gel column (30.0 g silica). Pure product containing fractions were pooled and evaporated to afford the dimer as a foam (0.56 g, CDB 4877). LCMS (MW: 658.91 confirmed). ¹H NMR partial (400 MHz, CDCl₃): δ 5.78 (s, 2H, C-4 CH), 4.65 (q, 2H, C-17 CH), 2.64 (s, 4H, OCO—CH₂), 1.22 (s, 6H, C-10 CH₃), 0.84 (s, 6H, C-13 CH₃).

### Example 59

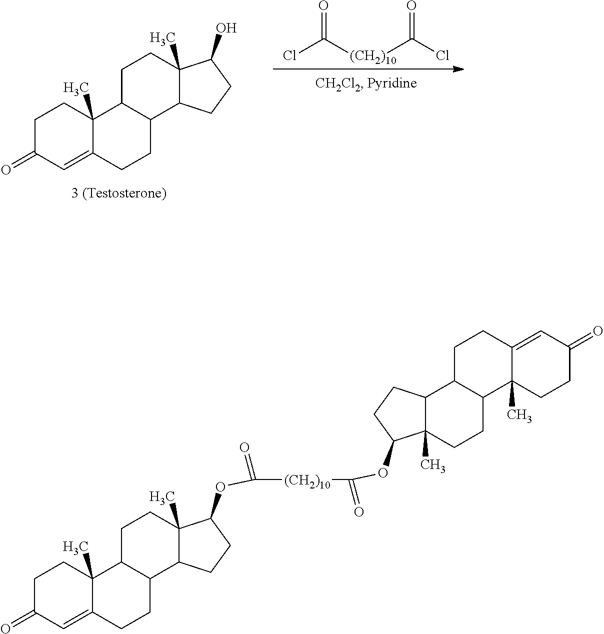

Testosterone (0.88 g, 3.00 mmol) was dissolved in CH₂Cl₂ (22 mL). To the solution pyridine (0.45 g, 4.50 mmol) was added and the solution was cooled in an ice-bath (3-50 C). To the resulting solution dodecanedionyl chloride (0.63 g, 2.25 mmol) was added and the solution was stirred at room temperature for 4 hours [TLC: trace starting material]. The mixture was diluted with CH₂Cl₂ (100 mL) and the organic phase was washed successively with water (1×75 mL), 5% HCl solution (1×75 mL), brine (1×75 mL), dried over MgSO₄, filtered and evaporated. The crude residue was purified two times over silica gel column using gradients of CH₂Cl₂/acetone to afford the dimer as colorless oil (0.56 g, CDB 4878). LCMS (MW: 771 confirmed). ¹H NMR partial (400 MHz, CDCl₃): δ 5.75 (s, 2H, C-4 CH), 4.63 (q, 2H, C-17 CH), 1.22 (s, 6H, C-10 CH₃), 0.84 (s, 6H, C-13 CH₃).

### Example 60

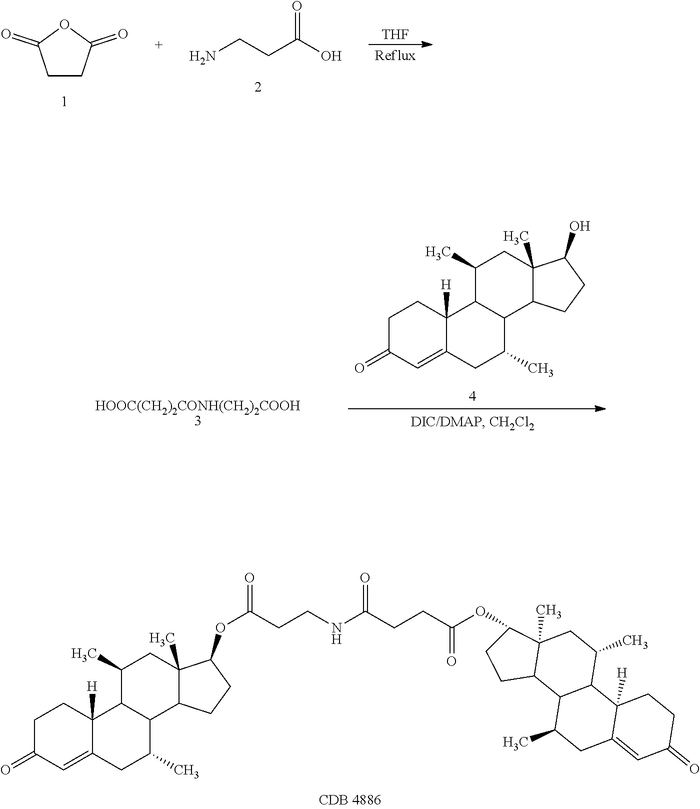

4-[(2-Carboethoxyethyl)amino]-4-oxo-butanoic acid (3): A mixture of succinic anhydride 1 (3.0 g, 30 mmol) and β-Alanine 2 (2.67 g, 30.0 mmol) in anhydrous THF (45 mL) was heated at reflux for 5 hours. The resulting clear solution was concentrated to ca. 20 mL and allowed to stand at room temperature overnight. The separated solid was collected via filtration and air-dried to constant weight to yield 3.8 g of white solid 3. Melting point: 141-143° C. Calculated for C₇H₁₁NO₅ (189.16): C, 44.44; H, 5.86; N, 7.40. Found: C, 44.38; H, 5.88 and N, 7.44. To a stirred mixture of 4-[(2-carboxyethyl)amino]-4-oxo-butanoic acid 3, 0.48 g, 2.50 mmol) in anhydrous CH₂Cl₂ (25 mL), DMA 4 (1.5 g, 5.0 mmol), diisopropylcarbodiimide (2.0 mL, 12.7 mmol) and DMAP (0.15 g, 1.25 mmol) were added under nitrogen, and the resulting reaction mixture was stirred at room temperature overnight. Next day, the resulting mixture was filtered through a celite pad and the filtrate was diluted with CH₂Cl₂ (75 mL). The organic phase was washed successively with water (1×75 mL), 5% HCl solution (1×75 mL) and brine (1×75 mL). After drying over MgSO₄, the filtrate was evaporated. The crude residue was purified over silica gel column using gradients of CH₂Cl₂/acetone to isolate the non-polar spot (0.72 g, HPLC: 98.4%, CDB 4886). LCMS (MW: 758.04 confirmed). ¹H NMR partial (400 MHz, CDCl₃): δ 6.21 (t, 1H, NH), 5.86 (s, 2H, C-4 CH), 4.60 (q, 2H, C-17 CH), 3.54 (m, 2H, OCO—CH₂), 1.16 (d, 6H, C-11β CH₃), 0.93 (s, 6H, C-18 CH₃), 0.80 (q, 6H, C-7α CH₃).

### Example 61

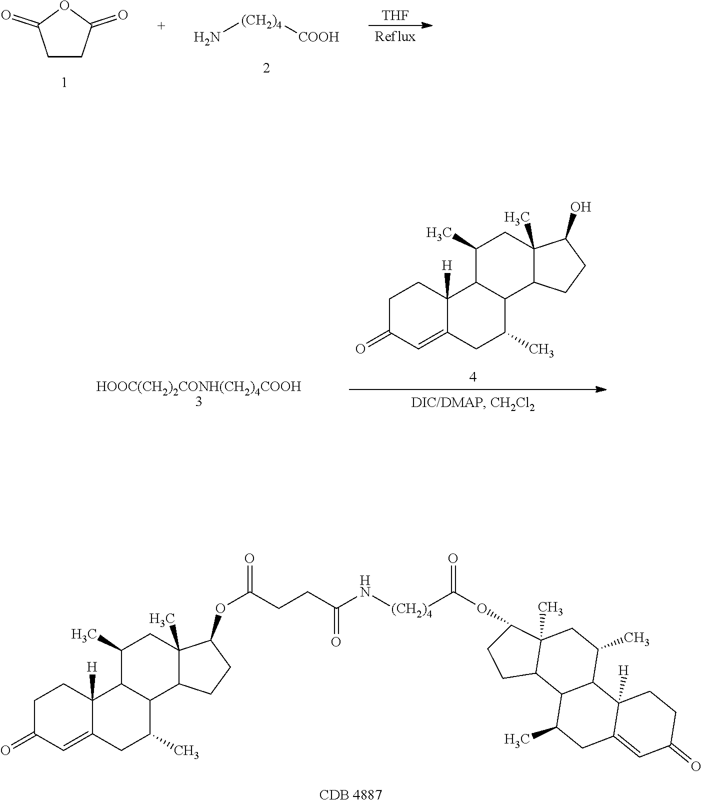

A mixture of succinic anhydride (2.0 g, 20 mmol) and 5-aminovaleric acid (2.34 g, 20.0 mmol) in anhydrous THF (30 mL) was heated at reflux for 5 hours. The resulting clear solution was concentrated to ca. 15 mL and allowed to stand at room temperature overnight. The separated solid was collected via filtration and air-dried to constant weight to yield 2.2 g of white solid 3. ¹H NMR partial (400 MHz, DMSO-d₆): δ 7.80 (s, 1H, NH), 3.02 (t, 2H, CH₂), 2.27 (m, 6H, 3×CH₂), 1.36 (q, 4H, 2×CH₂). To a stirred mixture of 5-(3-hydroperoxybut-3-enamido)pentanoic acid 3 (0.55 g, 2.50 mmol) in anhydrous CH₂Cl₂ (25 mL), DMA 4 (1.5 g, 5.0 mmol), diisopropylcarbodiimide (2.0 mL, 12.7 mmol) and DMAP (0.15 g, 1.25 mmol) were added under nitrogen, and the resulting reaction mixture was stirred at room temperature overnight. Next day, the resulting mixture was filtered through a celite pad and the filtrate was diluted with CH₂Cl₂ (75 mL). The organic phase was washed successively with water (1×75 mL), 5% HCl solution (1×75 mL) and brine (1×75 mL). After drying over MgSO₄, the filtrate was evaporated. The crude residue was purified over silica gel column using gradients of CH₂Cl₂/acetone to isolate the non-polar spot (0.44 g, HPLC: 98.3%, CDB 4887). LCMS (MW: 785.6 confirmed). ¹H NMR partial (400 MHz, CDCl₃): δ 5.94 (t, 1H, NH), 5.83 (s, 2H, C-4 CH), 4.55 (q, 2H, C-17 CH), 3.26 (m, 2H, OCO—CH₂), 1.13 (dd, 6H, C-11β CH₃), 0.91 (s, 6H, C-18 CH₃), 0.78 (d, 6H, C-7α CH₃).

### Example 62

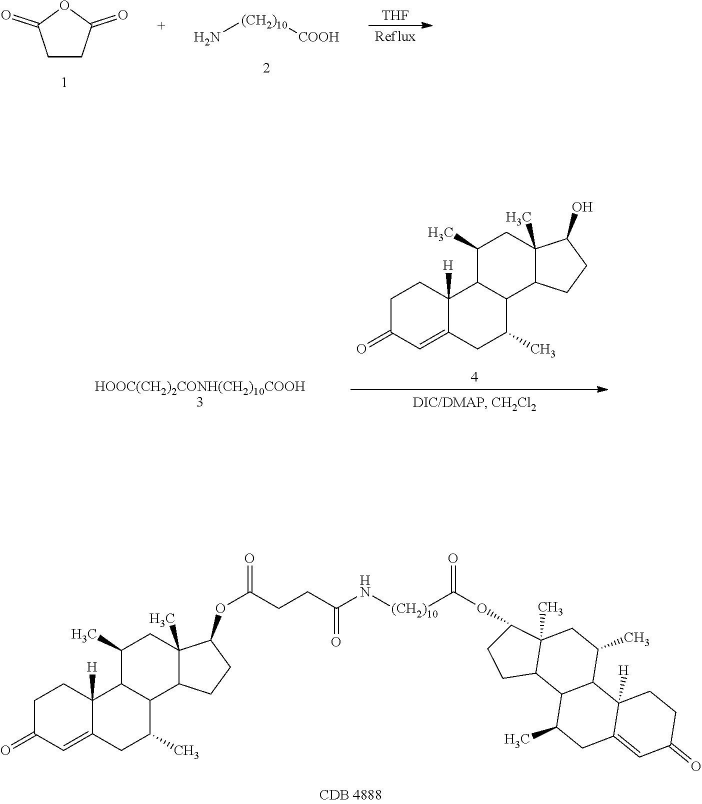

A mixture of succinic anhydride (1.0 g, 10 mmol) and 11-aminoundecanoic acid (2.01 g, 10.0 mmol) in anhydrous THF (30 mL) was heated at reflux for 7 hours. The resulting mixture was concentrated to ca. 15 mL and allowed to stand at room temperature overnight. The separated solid was collected via filtration and air-dried to constant weight to yield 1.7 g of white solid 3. ¹H NMR partial (400 MHz, DMSO-d₆₊D₂O): δ 3.02 (t, 2H, CH₂), 2.36 (t, 2H, CH₂), 2.27 (t, 2H, CH₂), 2.16 (t, 2H, CH₂), 1.45 (t, 2H, CH₂), 1.33 (t, 2H, CH₂) 1.20 (m, 12H, 3×CH₂). To a stirred mixture of 11-(3-hydroperoxybut-3-enamido)undecanoic acid 3 (0.76 g, 2.50 mmol) in anhydrous CH₂Cl₂ (25 mL), DMA 4 (1.5 g, 5.0 mmol), diisopropylcarbodiimide (2.0 mL, 12.7 mmol) and DMAP (0.15 g, 1.25 mmol) were added under nitrogen, and the resulting reaction mixture was stirred at room temperature for 72 hours. Next day, the resulting mixture was filtered through a celite pad and the filtrate was diluted with CH₂Cl₂ (75 mL). The organic phase was washed successively with water (1×75 mL), 5% HCl solution (1×75 mL) and brine (1×75 mL). After drying over MgSO₄, the filtrate was evaporated. The crude residue was purified over silica gel column using gradients of CH₂Cl₂/acetone to isolate the non-polar spot (0.61 g, HPLC: 98.6%, CDB 4888). LCMS (MW: 870.6 confirmed). ¹H NMR partial (400 MHz, CDCl₃): δ 5.74 (t, 1H, NH), 5.85 (s, 2H, C-4 CH), 4.57 (q, 2H, C-17 CH), 3.26 (q, 2H, OCO—CH₂), 2.68 (q, 2H, OCO—CH₂), 1.08 (dd, 6H, C-11β CH₃), 0.91 (s, 6H, C-18 CH₃), 0.78 (dd, 6H, C-7α CH₃).

### Example 63

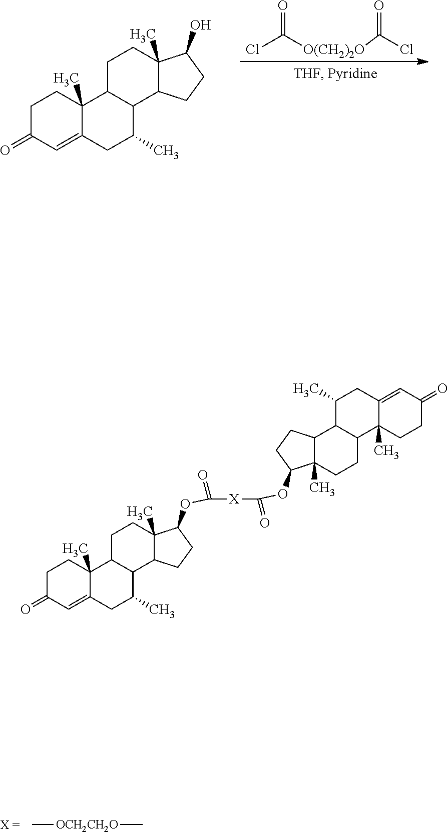

7-α-MT (0.10 g, 0.33 mmol) was dissolved in anhydrous THF (8 mL). To the solution pyridine (0.10 g, 1.00 mmol), followed by ethylene bischloroformate (0.2 g, 1.00 mmol) were added and the resulting stirred solution was heated at reflux for 4 hours [TLC: ca. 40% SM left]. To the solution additional chloroformate (0.2 g) was added and reflux continued for 2 hours [TLC: ca. 90% complete]. Reaction was worked up and the major non-polar spot was isolated through silica gel column to obtain 0.12 g product as thick oil (CDB 4928). NMR: product (contains some bisformate). The oil (0.1 g) was repurified over silica column to afford foam (66 mg). ¹H NMR partial (400 MHz, CDCl₃): δ 5.85 (s, 2H, C-4 CH), 4.49 (q, 2H, C-17 CH), 4.35 (t, 4H, OCH₂—CH₂O), 0.91 (s, 6H, C-18 CH₃), 0.82 (d, 6H, C-7α CH₃). HPLC: 97%; LCMS (M+1): 719.6.

### Example 64

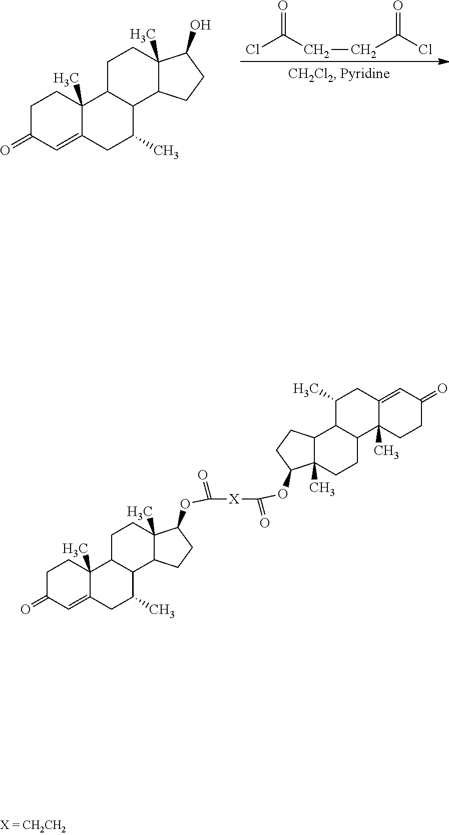

7-α-MT (0.10 g, 0.33 mmol) was dissolved in anhydrous CH₂Cl₂ (8 mL). To the solution pyridine (0.10 g, 1.00 mmol), followed by succinyl chloride (0.15 g, 1.00 mmol) were added and the resulting stirred solution was stirred at room temperature for 4 hours [TLC: ca. 20% SM left]. To the solution additional succinyl chloride (0.1 g) was added and stirring continued for 2 hours [TLC: ca. 90% complete]. The solution was diluted with CH₂Cl₂. The organic phase was washed successively with water (1×30 mL), 5% HCl solution (1×30 mL), brine (1×40 mL), dried over MgSO₄, filtered and evaporated. Material was purified over silica column to obtain 100 mg foam. This material was repurified over silica column and the residue was crystallized from ether/petroleum ether to obtain product as off-colored solid 73 mg (CDB 4927). ¹H NMR partial (400 MHz, CDCl₃): δ 5.85 (s, 2H, C-4 CH), 4.58 (q, 2H, C-17 CH), 2.63 (s, 4H, CH₂—CH₂), 1.15 (s, 6H, CH₃), 0.90 (s, 6H, C-18 CH₃), 0.83 (d, 6H, C-7α CH₃). LCMS: 687.5 (M+1); HPLC: 98%.

### Example 65

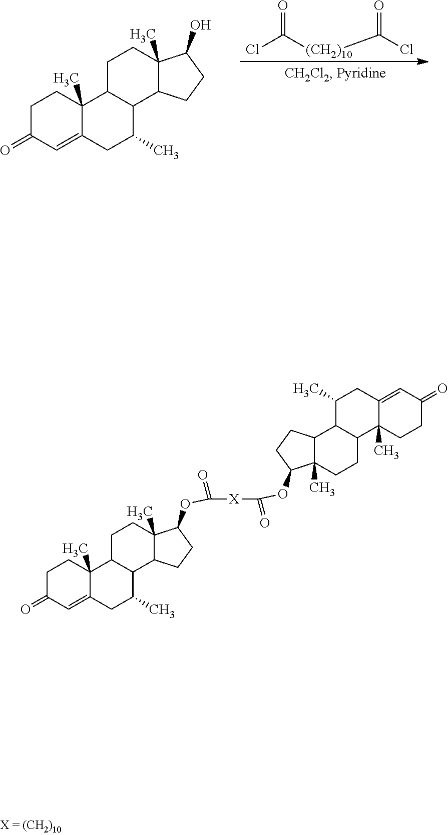

7-α-MT (0.10 g, 0.33 mmol) was dissolved in anhydrous CH₂Cl₂ (8 mL). To the solution pyridine (0.10 g, 1.00 mmol), followed by Dodecyl chloride (0.26 g, 1.00 mmol) were added and the resulting stirred solution was stirred at room temperature for 4 hours [TLC: ca. 20% SM left]. To the solution additional chloride (0.2 g) was added and stirring continued for 2 hours [TLC: ca. 90% complete]. The solution was diluted with CH₂Cl₂. The organic phase was washed successively with water (1×30 mL), 5% HCl solution (1×30 mL), brine (1×40 mL), dried over MgSO₄, filtered and evaporated. The crude residue was purified over silica gel column using gradients of CH₂Cl₂/acetone to afford the dimer as colorless oil. The oil was further purified over silica gel column using gradients of CH₂Cl₂/EtOAC to afford the dimer as low melting solid (48 mg, CDB 4929). ¹H NMR partial (400 MHz, CDCl₃): δ 5.85 (s, 2H, C-4 CH), 4.6 (q, 2H, C-17 CH), 0.90 (s, 6H, C-18 CH₃), 0.83 (d, 6H, C-7α CH₃). LCMS: 800 (M+1); HPLC: 95%.

### Example 66

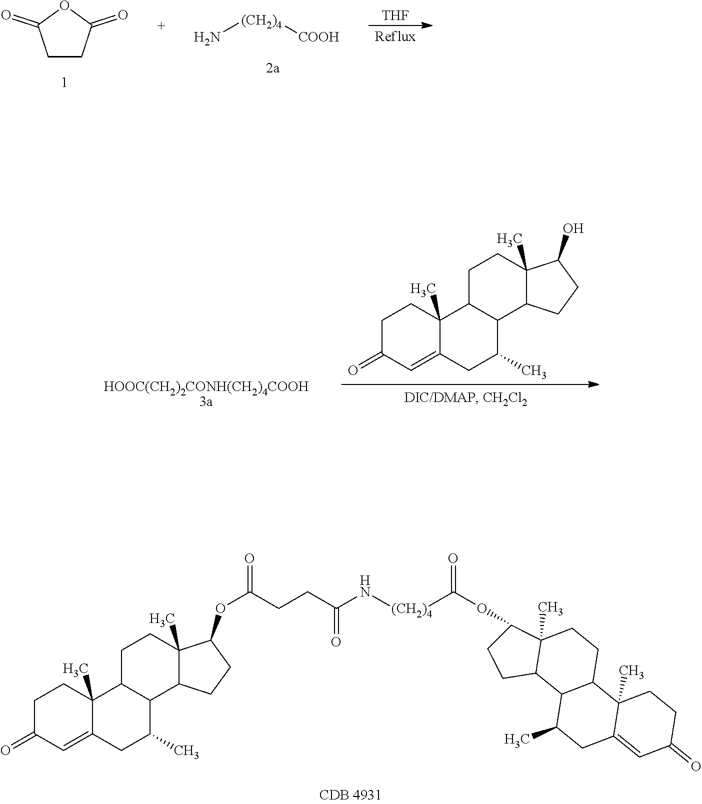

To a stirred mixture of 5-(3-hydroperoxybut-3-enamido)pentanoic acid 3a (60 mg, 0.25 mmol) in anhydrous CH₂Cl₂ (5 mL), 7-α-MT (0.1 g, 0.33 mmol), diisopropylcarbodiimide (0.3 mL, 1.9 mmol) and DMAP (20 mg, 0.12 mmol) were added under nitrogen, and the resulting reaction mixture was stirred at room temperature overnight. Next day, the resulting mixture was filtered through a celite pad and the filtrate was diluted with CH₂Cl₂ (15 mL). The organic phase was washed successively with water (1×15 mL), 5% HCl solution (1×15 mL) and brine (1×20 mL). After drying over MgSO₄, the filtrate was evaporated (0.13 g). The crude residue was purified over silica gel column using gradients of CH₂Cl₂/acetone to isolate the non-polar spot CDB 4931, (0.083 g). ¹H NMR partial (400 MHz, CDCl₃): δ 5.76 (t, 1H, NH), 5.85 (s, 2H, C-4 CH), 4.56 (q, 2H, C-17 CH), 3.26 (m, 2H, OCO—CH₂), 1.04 (s, 6H, C-18 CH₃), 0.85 (d, 6H, C-7α CH₃). HPLC: 98.3%; LCMS (M+1: 787).

### Example 67

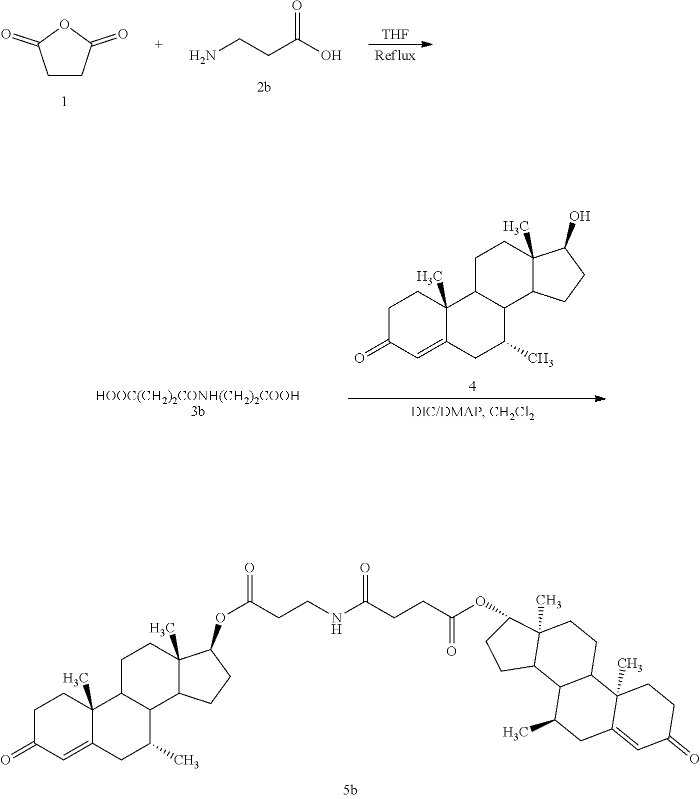

A mixture of succinic anhydride 1 (1.5 g, 15 mmol) and β-Alanine 2 (1.34 g, 15.0 mmol) in anhydrous THF (25 mL) was heated at reflux for 5 hours. The resulting clear solution was concentrated to ca. 10 mL and allowed to stand at room temperature overnight. The separated solid was collected via filtration and air-dried to constant weight to yield 1.9 g of white solid 3b. Melting point: 141-143° C. Preparation of 5b: To a stirred mixture of 4-[(2-carboxyethyl)amino]-4-oxo-butanoic acid 3b (50 mg, 0.25 mmol) in anhydrous CH₂Cl₂ (3 mL), 7-α-MT (0.15 g, 0.50 mmol), diisopropylcarbodiimide (0.3 mL, 1.27 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (10 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the non-polar spot CDB 4930 (110 mg). This material was further purified to obtain 92 mg solid. ¹H NMR partial (400 MHz, CDCl₃): δ 6.15 (t, 1H, NH), 5.84 (s, 2H, C-4 CH), 4.56 (q, 2H, C-17 CH), 3.51 (m, 2H, OCO—CH₂), 0.91 (s, 6H, C-18 CH₃), 0.78 (q, 6H, C-7α CH₃). HPLC: 96%; LCMS (M+1): 759.3.

### Example 68

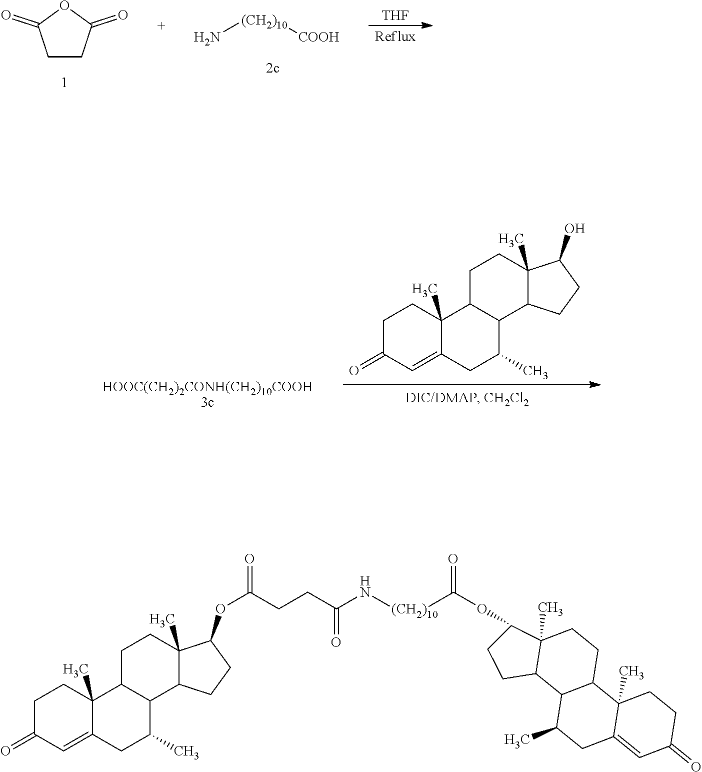

To a stirred mixture of 11-(3-hydroperoxybut-3-enamido)undecanoic acid 3c (0.076 g, 0.25 mmol) in anhydrous CH₂Cl₂ (5 mL), 7-α-MT (0.15 g, 0.50 mmol), diisopropylcarbodiimide (0.3 mL, 1.27 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (10 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the non-polar spot (93 mg, CDB 4932). ¹H NMR partial (400 MHz, CDCl₃): δ 5.64 (t, 1H, NH), 5.85 (s, 2H, C-4 CH), 4.56 (q, 2H, C-17 CH), 3.23 (q, 2H, OCO—CH₂), 2.7 (q, 2H, OCO—CH₂), 0.92 (s, 6H, C-18 CH₃), 0.77 (dd, 6H, C-7α CH₃). HPLC: 95%; LCMS (M+1): 872.3.

### Example 69

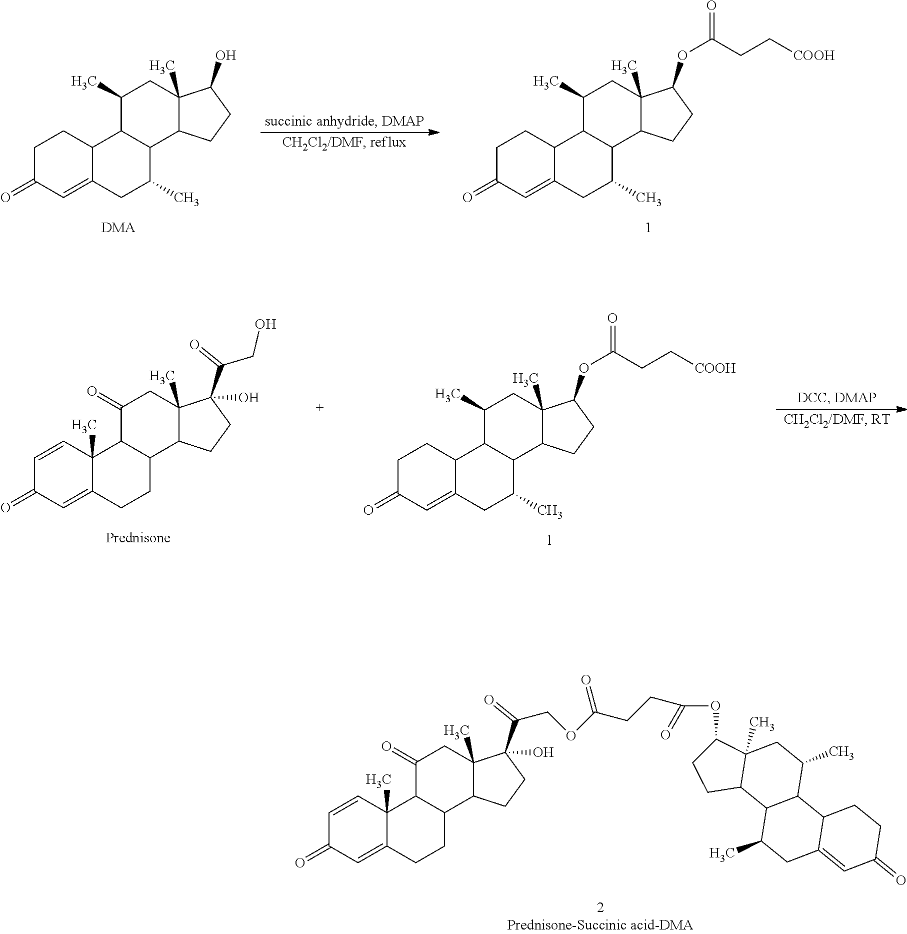

A mixture of DMA (3.0 g, 10.0 mmol), succinic anhydride (2.1 g, 20.9 mmol) and DMAP (2.4 g, 19.6 mmol) in a mixed solvent of CH₂Cl₂ (50 mL) and DMF (5 mL) was heated at reflux overnight. The reaction mixture was cooled to room temperature and washed with 10% H₃PO₄(aq)(1×20 mL), brine (1×20 mL), then dried over MgSO₄, filtered and stripped. The resulting oil (4.5 g) was dissolved in MTBE (10 mL) and stood at room temperature for 1 hour. The crystallized solid was collected by filtration, rinsed with cold MTBE (1×5 mL), air-dried then pumped under high vacuum to constant wt. to afford pure 1 as an off-white solid, 3.1 g (77%). Under nitrogen, a mixture of Prednisone (0.18 g, 0.50 mmol), DMA-succinic acid ester 1 (0.20 g, 0.50 mmol), DCC (0.35 g, 1.7 mmol) and DMAP (10 mg, 0.08 mmol) in a mixed solvent of CH₂Cl₂ (7 mL) and DMF (3 mL) was stirred at room temperature overnight. The reaction mixture was stripped and the residue was digested in CH₂Cl₂ (15 mL) and filtered to remove insoluble solid. The clear filtrate was washed successively with 5% H₃PO₄(aq) (1×10 mL), sat. aq. NaHCO₃ (1×10 mL) and with brine (1×10 mL), then dried over MgSO₄, filtered and stripped. The crude residue (0.60 g) was digested in hot IPA (5 mL) for 10 minutes, cooled to room temperature and filtered, vacuum dried to obtain pure product (CDB 4941) as a white solid, 0.35 g (94%). ¹H NMR partial (400 MHz, CDCl₃): δ 7.67 (d, 1H, J=10.4 Hz, Olefinic-H), 6.21 (d, 1H, J=10.4 Hz, Olefinic-H), 6.09 (s, 1H, Olefinic-H), 5.85 (s, 1H, Olefinic-H), 5.08 (d, 1H, J=17.6 Hz), 4.72 (d, 1H, J=17.6 Hz), 4.58 (t, 1H, J=8.4 Hz), 1.44 (s, 3H, CH₃), 1.05 (d, 3H, J=7.6 Hz, CH₃), 0.92 (s, 3H, CH₃), 0.78 (d, 3H, J=7.6 Hz, CH₃), 0.71 (s, 3H, CH₃). HPLC: 94.0%. LC/MS: 744.0 (M+1).

### Example 70

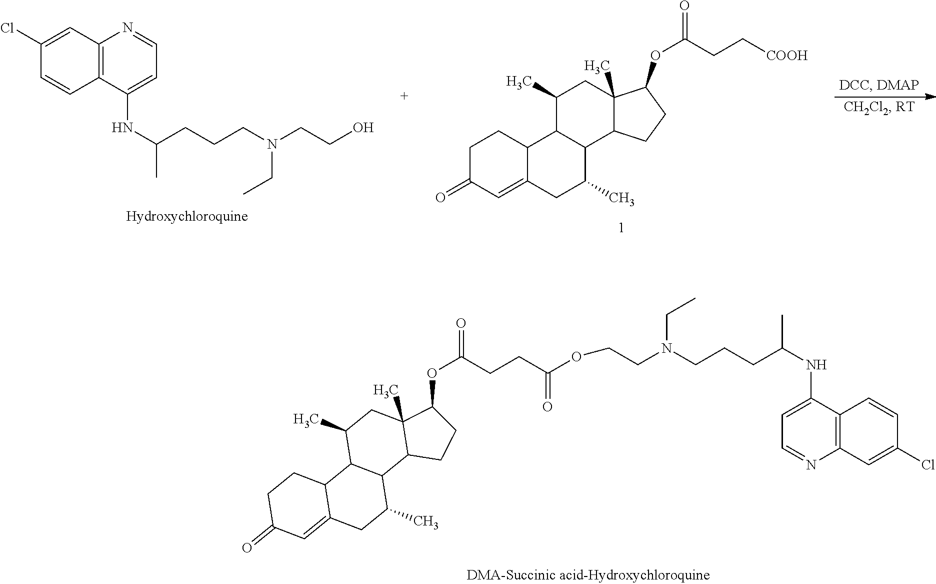

The commercial hydroxychloroquine sulfate salt (0.20 g, 0.46 mmol) was liberated to the corresponding free base by dissolving in water (10 mL), pH˜5, which was adjusted to pH˜8 by adding NaHCO₃ powder. The resulting solution was extracted with CH₂Cl₂ (3×15 mL). The organic extracts were combined and dried over MgSO₄, filtered, stripped then pumped under high vacuum to give 0.15 g (97%) as colorless gum. This was used for the coupling immediately without purification. Under nitrogen, a mixture of freshly prepared crude Hydroxychloroquine (0.15 g, 0.45 mmol), DMA-succinic acid ester 1 (0.20 g, 0.5 mmol), DCC (0.35 g, 1.7 mmol) and DMAP (10 mg, 0.08 mmol) in CH₂Cl₂ (10 mL) was stirred at room temperature overnight. The reaction mixture was filtered to remove insoluble solid. The clear filtrate was washed successively with sat. aq. NaHCO₃ (1×10 mL) and with brine (1×10 mL), then dried over MgSO₄, filtered and stripped. The resulting crude oil (0.63 g) was purified via a flash silica gel column (30 g), eluted with 0˜10% MeOH/CH₂Cl₂. The fractions containing pure product (by TLC) were pooled, stripped and vacuum dried to constant wt. to obtain 3 (CDB 4945) as an off-white foam, 0.29 g (89%). ¹H NMR partial (400 MHz, CDCl₃): δ 8.50 (d, 1H, J=5.2 Hz, Ar—H), 7.96 (s, 1H, Ar—H), 7.71 (d, 1H, J=8.8 Hz, Ar—H), 7.35 (d, 1H, J=8.8 Hz, Ar—H), 6.42 (d, 1H, J=5.2 Hz, Ar—H), 5.84 (s, 1H, Olefinic-H), 5.22 (s, 1H), 4.53 (t, 1H, J=8.0 Hz), 4.17 (t, 2H, J=6.0 Hz), 3.72 (t, 1H, J=6.0 Hz), 0.89 (s, 3H, CH₃), 0.76 (d, 3H, J=6.8 Hz, CH₃). HPLC: 96.1%. LC/MS: 721.9 (M+1).

### Example 71

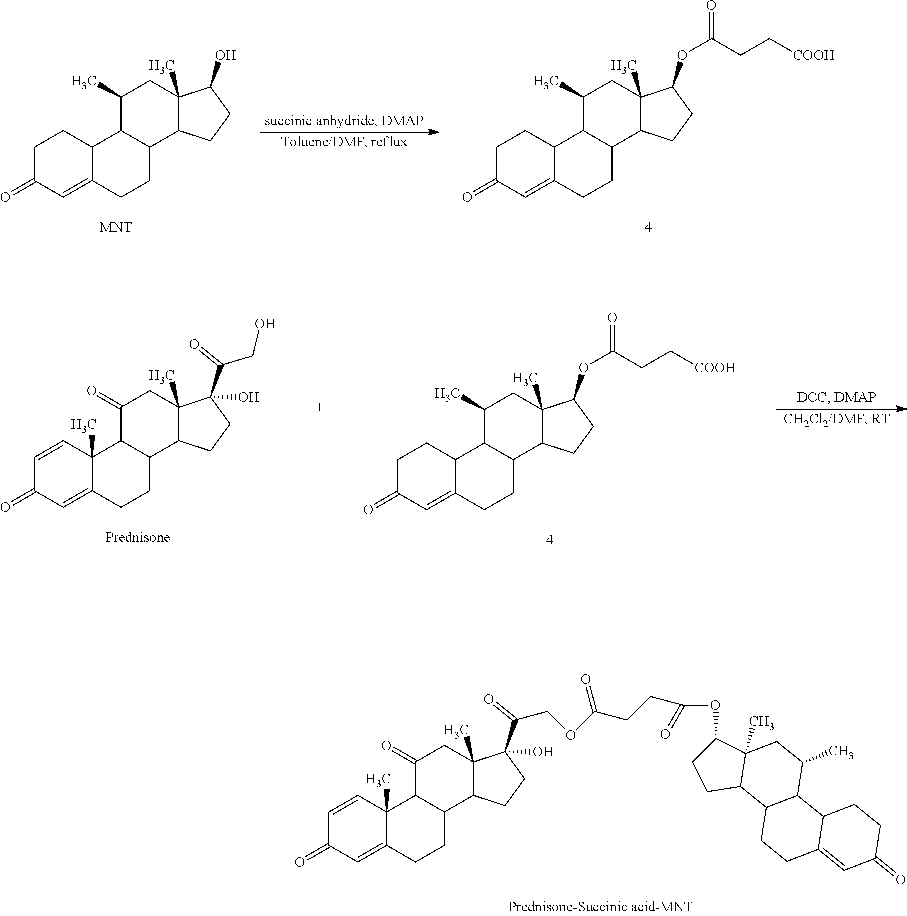

A mixture of MNT (3.0 g, 10.4 mmol), succinic anhydride (1.5 g, 15.0 mmol) and DMAP (2.4 g, 19.6 mmol) in a mixed solvent of toluene (45 mL) and DMF (5 mL) was heated at reflux overnight. The reaction mixture was cooled to room temperature and washed with 10% H₃PO₄(aq) (1×20 mL), brine (1×20 mL), then dried over MgSO₄, filtered and stripped. The resulting dark brown oil (4.65 g) was dissolved in CH₂Cl₂ (5 mL) and loaded onto a flash silica gel column (90 g), then eluted with 0˜7% MeOH/CH₂Cl₂. Fractions containing product (by TLC) were pooled and stripped. The resulting residue was triturated in MTBE (10 mL) at room temperature and filtered, air-dried then pumped under high vacuum to constant wt. to afford pure 4 as an off-white solid, 3.25 g (80%). Under nitrogen, a mixture of Prednisone (0.18 g, 0.50 mmol), MNT-succinic acid ester 4 (0.20 g, 0.51 mmol), DCC (0.35 g, 1.7 mmol) and DMAP (10 mg, 0.08 mmol) in a mixed solvent of CH₂Cl₂ (7 mL) and DMF (3 mL) was stirred at room temperature overnight. The reaction mixture was stripped and the residue was digested in CH₂Cl₂ (15 mL) and filtered to remove insoluble solid. The clear filtrate was washed successively with 5% H₃PO₄(aq) (1×10 mL), sat. aq. NaHCO₃ (1×10 mL) and with brine (1×10 mL), then dried over MgSO₄, filtered and stripped. The resulting crude oil (0.76 g) was dissolved in warm IPA (3 mL) and cooled to room temperature, then in an ice/water bath for 10 minutes. The crystallized solid was collected and vacuum dried to afford the product (CDB 4942), 0.27 g (75%), as an off-white solid. ¹H NMR partial (400 MHz, CDCl₃): δ 7.67 (d, 1H, J=10.4 Hz, Olefinic-H), 6.21 (d, 1H, J=10.4 Hz, Olefinic-H), 6.09 (s, 1H, Olefinic-H), 5.84 (s, 1H, Olefinic-H), 5.09 (d, 1H, J=17.6 Hz), 4.71 (d, 1H, J=17.6 Hz), 4.57 (t, 1H, J=8.8 Hz), 1.44 (s, 3H, CH₃), 1.06 (d, 3H, J=7.6 Hz, CH₃), 0.92 (s, 3H, CH₃), 0.71 (s, 3H, CH₃). HPLC: 88.9%. LC/MS: 730.5 (M+1).

### Example 72

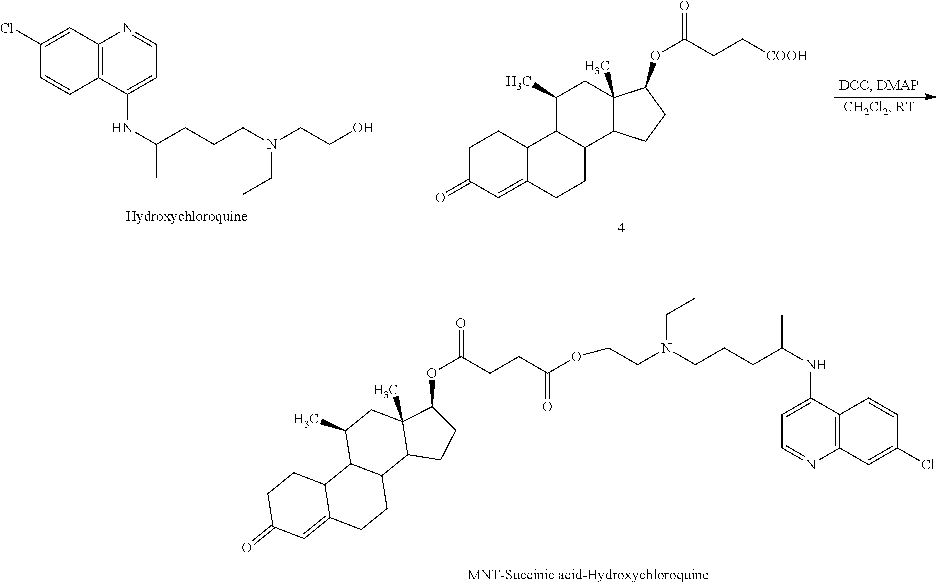

The commercial hydroxychloroquine sulfate salt (0.20 g, 0.46 mmol) was liberated to the corresponding free base by dissolving in water (10 mL), pH˜5, which was adjusted to pH˜8 by adding NaHCO₃ powder. The resulting solution was extracted with CH₂Cl₂ (3×15 mL). The organic extracts were combined and dried over MgSO₄, filtered, stripped then pumped under high vacuum to give 0.15 g (97%) as colorless gum. This was used for the coupling immediately without purification. Under nitrogen, a mixture of freshly prepared crude Hydroxychloroquine (0.15 g, 0.45 mmol), MNT-succinic acid ester 4 (0.20 g, 0.5 mmol), DCC (0.35 g, 1.7 mmol) and DMAP (10 mg, 0.08 mmol) in CH₂Cl₂ (10 mL) was stirred at room temperature overnight. The reaction mixture was filtered to remove insoluble solid. The clear filtrate was washed successively with sat. aq. NaHCO₃ (1×10 mL) and with brine (1×10 mL), then dried over MgSO₄, filtered and stripped. The resulting crude oil (0.61 g) was purified via a flash silica gel column (30 g), eluted with 0˜10% MeOH/CH₂Cl₂. The fractions containing pure product (by TLC) were pooled, stripped and vacuum dried to constant wt. to obtain the product (CDB 4946) as an off-white foam, 0.17 g (53%). ¹H NMR partial (400 MHz, CDCl₃): δ 8.50 (d, 1H, J=5.2 Hz, Ar—H), 7.96 (s, 1H, Ar—H), 7.71 (d, 1H, J=8.8 Hz, Ar—H), 7.35 (d, 1H, J=8.8 Hz, Ar—H), 6.42 (d, 1H, J=5.2 Hz, Ar—H), 5.84 (s, 1H, Olefinic-H), 5.21 (s, 1H), 4.52 (t, 1H, J=8.0 Hz), 4.17 (t, 2H, J=5.6 Hz), 3.72 (t, 1H, J=6.0 Hz), 1.32 (d, 3H, J=6.4 Hz, CH₃), 0.89 (s, 3H, CH₃>. HPLC: 94.8%. LC/MS: 707.5 (M+1).

### Example 73

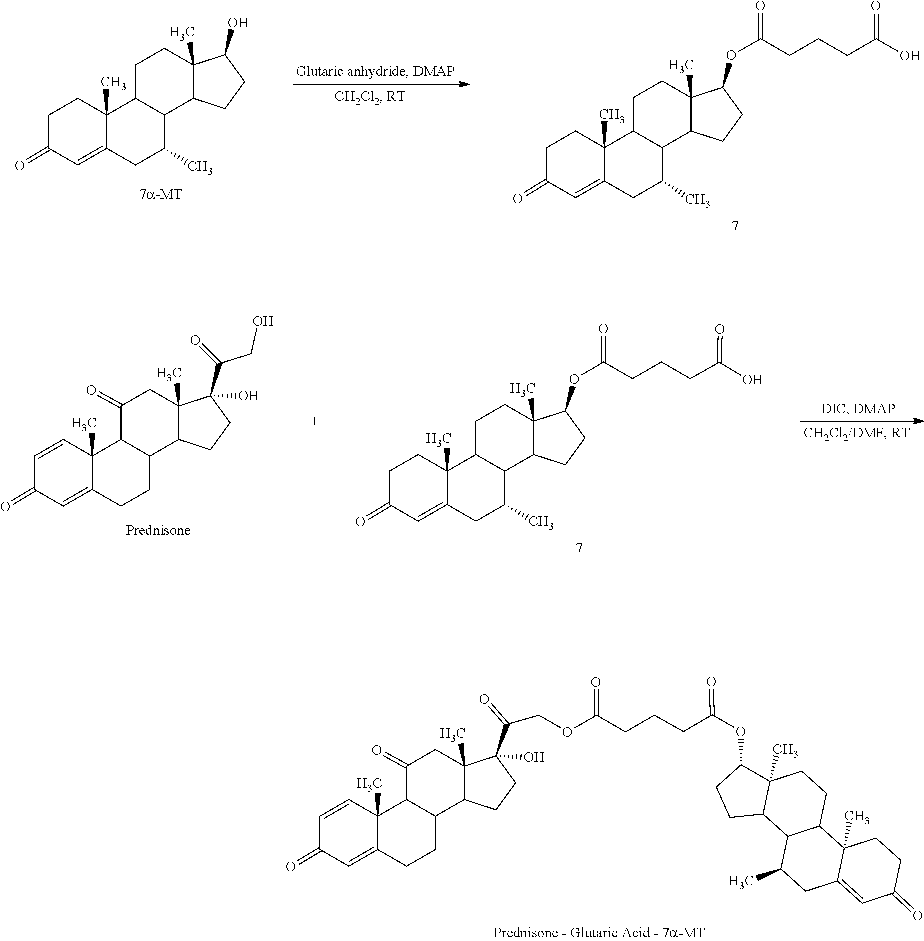 [SMILES: C[C@@H]1CC2=CC(=O)CC[C@]2(C)C2CC[C@@]3(C)C(CC[C@@H]3O)C21 ; C[C@@H]1CC2=CC(=O)CC[C@]2(C)C2CC[C@@]3(C)C(CC[C@@H]3OC(=O)CCCC(=O)O)C21 ; C[C@]12C=CC(=O)C=C1CCC1C2C(=O)C[C@@]2(C)C1CC[C@]2(O)C(=O)CO ; C[C@@H]1CC2=CC(=O)CC[C@]2(C)C2CC[C@@]3(C)C(CC[C@@H]3OC(=O)CCCC(=O)O)C21 ; C[C@@H]1CC2=CC(=O)CC[C@]2(C)C2CC[C@@]3(C)C(CC[C@@H]3OC(=O)CCCC(=O)OCC(=O)[C@@]3(O)CCC4C5CCC6=CC(=O)C=C[C@]6(C)C5C(=O)C[C@@]43C)C21]

A mixture of 7α-MT (1.0 g, 3.3 mmol), glutaric anhydride (0.76 g, 6.6 mmol) and DMAP (0.81 g, 6.6 mmol) in CH₂Cl₂ (20 mL) was stirred at room temperature overnight. The reaction mixture was washed with 10% H₃PO₄(aq) (1×10 mL), brine (1×10 mL), then dried over MgSO₄, filtered and stripped. The resulting residue (1.60 g) was dissolved in CH₂Cl₂ (5 mL) and loaded onto a flash silica gel column (50 g), first eluted with 10% EtOAc/CH₂Cl₂ to remove less polar impurity spot, then eluted with 5˜10% MeOH/CH₂Cl₂ to collect product spot. Fractions containing product (by TLC) were pooled and stripped, pumped under high vacuum to constant wt. to afford pure 7 as an off-white solid, 1.06 g (77%), mp. 174-180° C. Under nitrogen, a mixture of Prednisone (0.18 g, 0.50 mmol), 7α-MT-Glutaric acid ester 7 (0.20 g, 0.48 mmol), DIC (0.19 g, 1.5 mmol) and DMAP (10 mg, 0.08 mmol) in a mixed solvent of CH₂Cl₂ (7 mL) and DMF (3 mL) was stirred at room temperature overnight. The reaction mixture was stripped and the residue was re-dissolved in CH₂Cl₂ (30 mL) and washed successively with 5% H₃PO₄(aq) (1×10 mL), and with brine (1×10 mL), then dried over MgSO₄, filtered and stripped. The resulting crude oil (0.56 g) was dissolved in CH₂Cl₂ (5 mL) and loaded onto a flash silica gel column (40 g), then eluted with 1˜3% MeOH/CH₂Cl₂. Fractions containing product (by TLC) were pooled and stripped, then pumped under high vacuum to constant wt. to afford pure product (CDB 4943), 0.18 g (50%), as an off-white foam. ¹H NMR partial (400 MHz, CDCl₃): δ 7.68 (d, 1H, J=10.4 Hz, Olefinic-H), 6.22 (d, 1H, J=10.4 Hz, Olefinic-H), 6.09 (s, 1H, Olefinic-H), 5.73 (s, 1H, Olefinic-H), 5.07 (d, 1H, J=17.6 Hz), 4.70 (d, 1H, J=17.6 Hz), 4.63 (t, 1H, J=8.4 Hz), 3.83 (m, 2H), 1.44 (s, 3H, CH₃), 1.20 (s, 3H, CH₃), 0.84 (s, 3H, CH₃), 0.77 (d, 3H, J=6.8 Hz, CH₃), 0.71 (s, 3H, CH₃). HPLC: 98.0%. LC/MS: 757.6 (M+1).

### Example 74

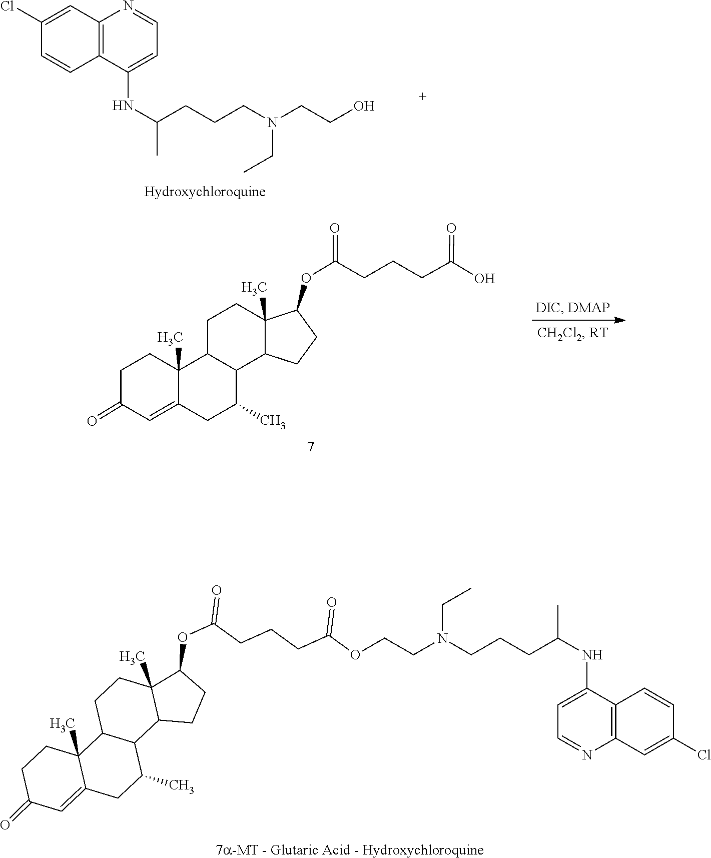 [SMILES: CCN(CCO)CCCC(C)Nc1ccnc2cc(Cl)ccc12 ; C[C@@H]1CC2=CC(=O)CC[C@]2(C)C2CC[C@@]3(C)C(CC[C@@H]3OC(=O)CCCC(=O)O)C21 ; CCN(CCCC(C)Nc1ccnc2cc(Cl)ccc12)CCOC(=O)CCCC(=O)O[C@H]1CCC2C3C(CC[C@@]21C)[C@@]1(C)CCC(=O)C=C1C[C@H]3C]

The commercial Hydroxychloroquine Sulfate salt (0.20 g, 0.46 mmol) was liberated to the corresponding free base by dissolving in water (10 mL), pH˜5, which was adjusted to pH˜8 by adding NaHCO₃ powder. The resulting solution was extracted with CH₂Cl₂ (3×15 mL). The organic extracts were combined and dried over MgSO₄, filtered, stripped then pumped under high vacuum to give 0.15 g (97%) as colorless gum. This was used for the coupling immediately without purification. Under nitrogen, a mixture of freshly prepared crude Hydroxychloroquine (0.15 g, 0.45 mmol), 7α-MT-Glutaric acid ester 7 (0.20 g, 0.48 mmol), DIC (0.19 g, 1.5 mmol) and DMAP (10 mg, 0.08 mmol) in CH₂Cl₂ (10 mL) was stirred at room temperature overnight. The reaction mixture was washed successively with sat. aq. NaHCO₃ (1×10 mL) and with brine (1×10 mL), then dried over MgSO₄, filtered and stripped. The resulting crude oil (0.49 g) was purified via a flash silica gel column (40 g), eluted with 0˜10% MeOH/CH₂Cl₂. The fractions containing pure product (by TLC) were pooled, stripped and vacuum dried to constant wt. to afford pure product (CDB 4947), 0.15 g (43%), as an off-white foam. ¹H NMR partial (400 MHz, CDCl₃): δ 8.51 (d, 1H, J=5.6 Hz, Ar—H), 7.95 (s, 1H, Ar—H), 7.71 (d, 1H, J=8.8 Hz, Ar—H), 7.35 (d, 1H, J=8.8 Hz, Ar—H), 6.42 (d, 1H, J=5.6 Hz, Ar—H), 5.72 (s, 1H, Olefinic-H), 5.17 (d, 1H, J=5.6 Hz), 4.58 (t, 1H, J=8.4 Hz), 4.15 (t, 2H, J=6.0 Hz), 3.72 (m, 1H), 2.69 (t, 2H, J=6.0 Hz), 1.32 (d, 3H, J=6.0 Hz, CH₃), 1.18 (s, 3H, CH₃), 1.00 (t, 3H, J=6.8 Hz, CH₃), 0.81 (s, 3H, CH₃), 0.76 (d, 3H, J=7.2 Hz, CH₃). HPLC: 97.1%. LC/MS: 735.5 (M+1).

### Example 75

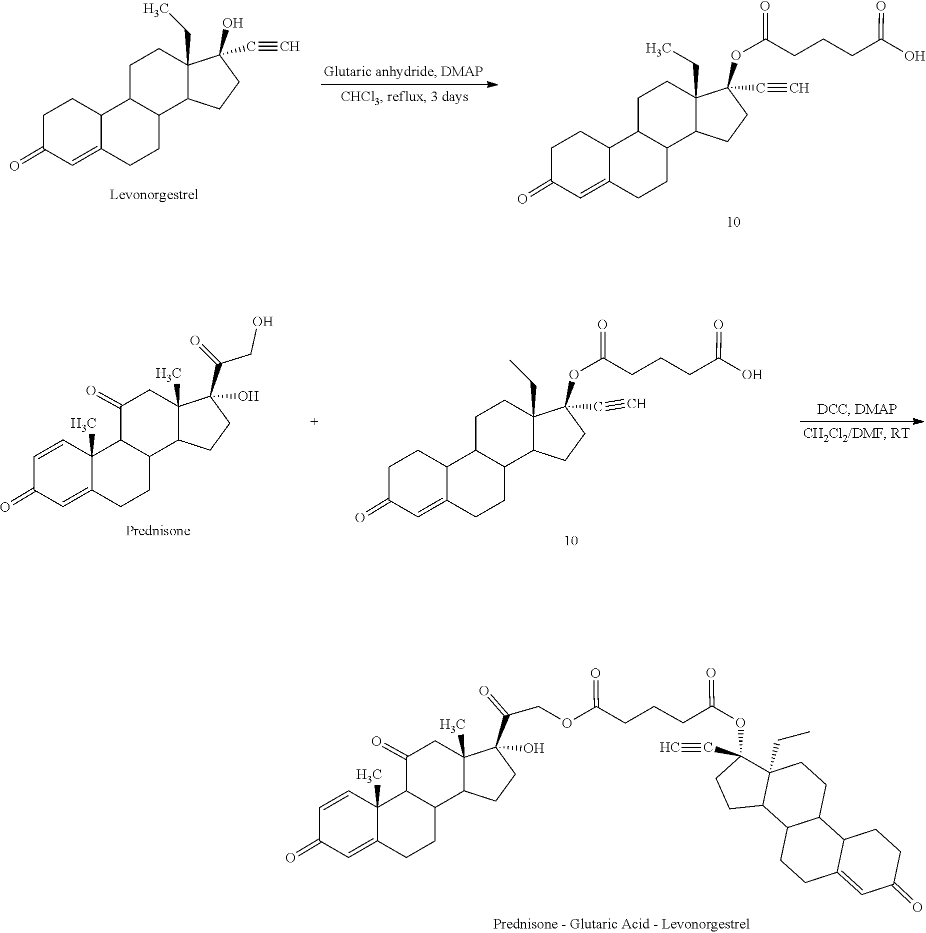 [SMILES: C#C[C@]1(O)CCC2C3CCC4=CC(=O)CCC4C3CC[C@@]21CC ; C#C[C@]1(OC(=O)CCCC(=O)O)CCC2C3CCC4=CC(=O)CCC4C3CC[C@@]21CC ; C[C@]12C=CC(=O)C=C1CCC1C2C(=O)C[C@@]2(C)C1CC[C@]2(O)C(=O)CO ; C#C[C@]1(OC(=O)CCCC(=O)O)CCC2C3CCC4=CC(=O)CCC4C3CC[C@@]21CC ; C#C[C@]1(OC(=O)CCCC(=O)OCC(=O)[C@@]2(O)CCC3C4CCC5=CC(=O)C=C[C@]5(C)C4C(=O)C[C@@]32C)CCC2C3CCC4=CC(=O)CCC4C3CC[C@@]21CC]

A mixture of Levonorgestrel (1.0 g, 3.2 mmol), glutaric anhydride (0.73 g, 6.4 mmol) and DMAP (0.78 g, 6.4 mmol) in CHCl₃ (15 mL) was heated at reflux for 3 days. The reaction mixture was washed with 10% H₃PO₄(aq) (1×10 mL), brine (1×10 mL), then dried over MgSO₄, filtered and stripped. The resulting residue (1.62 g) was dissolved in CH₂Cl₂ (5 mL) and loaded onto a flash silica gel column (50 g), first eluted with 10% EtOAc/CH₂Cl₂ to remove less polar impurity spot, then eluted with 5˜10% MeOH/CH₂Cl₂ to collect product spot. Fractions containing product (by TLC) were pooled and stripped, pumped under high vacuum to constant wt. to obtain pure 10 as an off-white solid, 1.08 g (79%), mp. 162-171° C. Under nitrogen, a mixture of Prednisone (0.18 g, 0.50 mmol), Levonorgestrel-Glutaric acid ester 10 (0.20 g, 0.47 mmol), DCC (0.30 g, 1.5 mmol) and DMAP (10 mg, 0.08 mmol) in a mixed solvent of CH₂Cl₂ (7 mL) and DMF (3 mL) was stirred at room temperature overnight. The reaction mixture was stripped and the residue was digested in CH₂Cl₂ (15 mL) and filtered. The clear filtrate was washed successively with 5% H₃PO₄(aq) (1×10 mL), and with brine (1×10 mL), then dried over MgSO₄, filtered and stripped. The resulting crude oil (0.74 g) was dissolved in CH₂Cl₂ (5 mL) and loaded onto a flash silica gel column (40 g), then eluted with 1˜3% MeOH/CH₂Cl₂. Fractions containing product (by TLC) were pooled and stripped, then pumped under high vacuum to constant wt. to afford pure product (CDB 4944) (, 0.26 g (72%), as an off-white foam. ¹H NMR partial (400 MHz, CDCl₃): δ 7.68 (d, 1H, J=10.0 Hz, Olefinic-H), 6.21 (d, 1H, J=10.0 Hz, Olefinic-H), 6.09 (s, 1H, Olefinic-H), 5.83 (s, 1H, Olefinic-H), 5.07 (d, 1H, J=17.6 Hz), 4.68 (d, 1H, J=17.6 Hz), 2.61 (s, 1H), 1.44 (s, 3H, CH₃), 1.01 (t, 3H, J=7.2 Hz, CH₃), 0.71 (s, 3H, CH₃). HPLC: 96.8%. LC/MS: 767.5 (M+1).

### Example 76

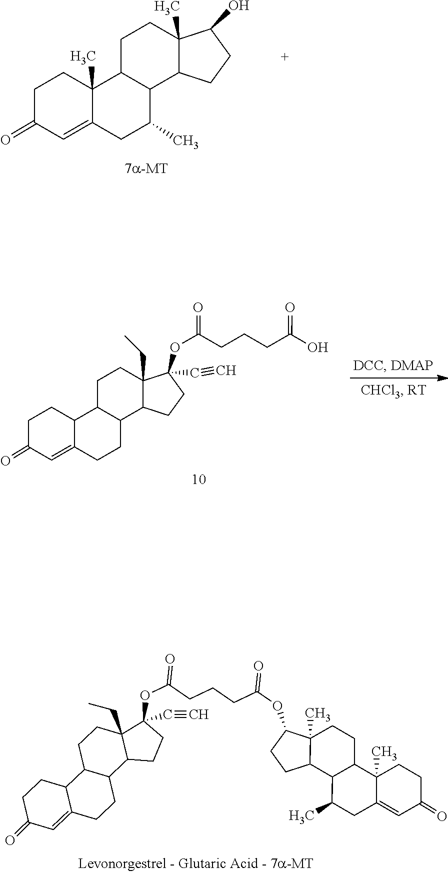 [SMILES: C[C@@H]1CC2=CC(=O)CC[C@]2(C)C2CC[C@@]3(C)C(CC[C@@H]3O)C21 ; C#C[C@]1(OC(=O)CCCC(=O)O)CCC2C3CCC4=CC(=O)CCC4C3CC[C@@]21CC ; C#C[C@]1(OC(=O)CCCC(=O)O[C@H]2CCC3C4C(CC[C@@]32C)[C@@]2(C)CCC(=O)C=C2C[C@H]4C)CCC2C3CCC4=CC(=O)CCC4C3CC[C@@]21CC]

Under nitrogen, a mixture of 7α-MT (0.21 g, 0.50 mmol), Levonorgestrel-Glutaric acid ester 10 (0.20 g, 0.47 mmol), DIC (0.19 g, 1.5 mmol) and DMAP (10 mg, 0.08 mmol) in CHCl₃ (10 mL) was stirred at room temperature overnight. The reaction mixture was washed successively with 10% H₃PO₄(aq) (1×10 mL) and with brine (1×10 mL), then dried over MgSO₄, filtered and stripped. The crude residue (0.62 g) was purified via a flash silica gel column (40 g), eluted with 5% Acetone/CH₂Cl₂. The fractions containing pure product (by TLC) were pooled, stripped and vacuum dried to constant wt. to afford 12 (CDB 4940), 0.12 g (36%), as an off-white foam. ¹H NMR partial (400 MHz, CDCl₃): δ 5.84 (s, 1H, Olefinic-H), 5.73 (s, 1H, Olefinic-H), 4.65 (t, 1H, J=8.4 Hz), 2.61 (s, 1H), 1.20 (s, 3H, CH₃), 1.01 (t, 3H, J=7.2 Hz, CH₃), 0.84 (s, 3H, CH₃), 0.77 (d, 3H, J=6.8 Hz, CH₃). HPLC: 87.7%. LC/MS: 712.5 (M+1).

### Example 77

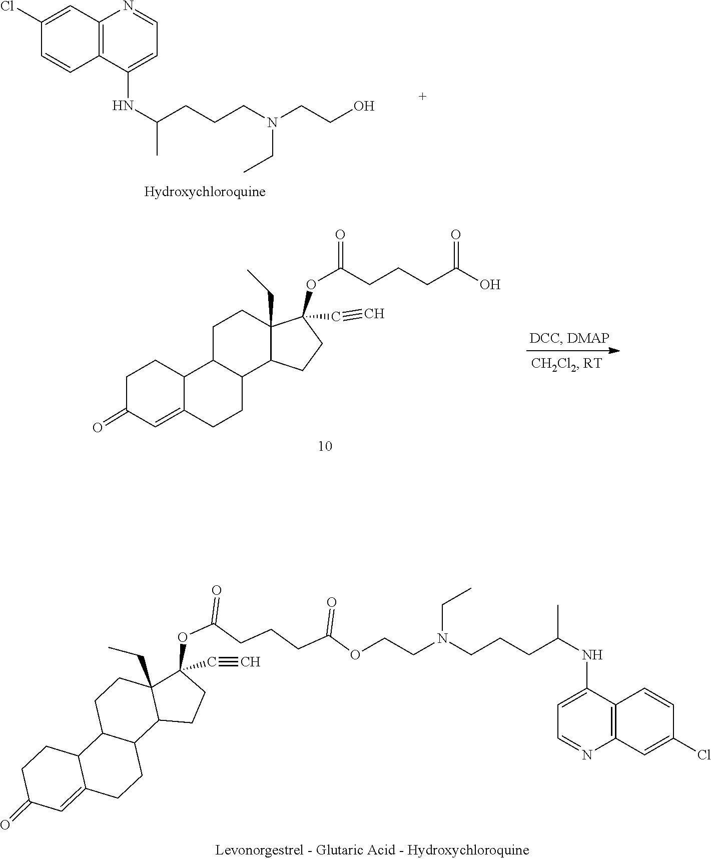 [SMILES: CCN(CCO)CCCC(C)Nc1ccnc2cc(Cl)ccc12 ; C#C[C@]1(OC(=O)CCCC(=O)O)CCC2C3CCC4=CC(=O)CCC4C3CC[C@@]21CC ; C#C[C@]1(OC(=O)CCCC(=O)OCCN(CC)CCCC(C)Nc2ccnc3cc(Cl)ccc23)CCC2C3CCC4=CC(=O)CCC4C3CC[C@@]21CC]

The commercial hydroxychloroquine sulfate salt (0.20 g, 0.46 mmol) was liberated to the corresponding free base by dissolving in water (10 mL), pH˜5, which was adjusted to pH˜8 by adding NaHCO₃ powder. The resulting solution was extracted with CH₂Cl₂ (3×15 mL). The organic extracts were combined and dried over MgSO₄, filtered, stripped then pumped under high vacuum to give 0.15 g (97%) as colorless gum. This was used for the coupling immediately without purification. Under nitrogen, a mixture of freshly prepared crude Hydroxychloroquine (0.15 g, 0.45 mmol), Levonorgestrel-Glutaric acid ester 10 (0.20 g, 0.47 mmol), DCC (0.30 g, 1.5 mmol) and DMAP (10 mg, 0.08 mmol) in CH₂Cl₂ (10 mL) was stirred at room temperature overnight. The reaction mixture was filtered to remove insoluble solid. The clear filtrate was washed successively with sat. aq. NaHCO₃ (1×10 mL) and with brine (1×10 mL), then dried over MgSO₄, filtered and stripped. The resulting crude oil (0.63 g) was dissolved in CH₂Cl₂ (5 mL) and loaded onto a flash silica gel column (40 g), then eluted with 0˜10% MeOH/CH₂Cl₂. Fractions containing product (by TLC) were pooled and stripped, then pumped under high vacuum to constant wt. to afford pure product (CDB 4948), 0.21 g (63%), as an off-white foam. ¹H NMR partial (400 MHz, CDCl₃): δ 8.51 (d, 1H, J=5.2 Hz, Ar—H), 7.97 (s, 1H, Ar—H), 7.72 (d, 1H, J=9.2 Hz, Ar—H), 7.35 (d, 1H, J=9.2 Hz, Ar—H), 6.43 (d, 1H, J=5.2 Hz, Ar—H), 5.83 (s, 1H, Olefinic-H), 5.18 (br.s, 1H), 4.15 (t, 1H, J=6.4 Hz), 3.72 (m, 1H), 2.69 (t, 2H, J=6.0 Hz), 1.32 (d, 3H, J=6.4 Hz, CH₃), 1.00 (t, 3H, J=7.2 Hz, CH₃), 0.98 (t, 3H, J=8.0 Hz, CH₃). HPLC: 95.8%. LC/MS: 745.5 (M+1).

### Example 78

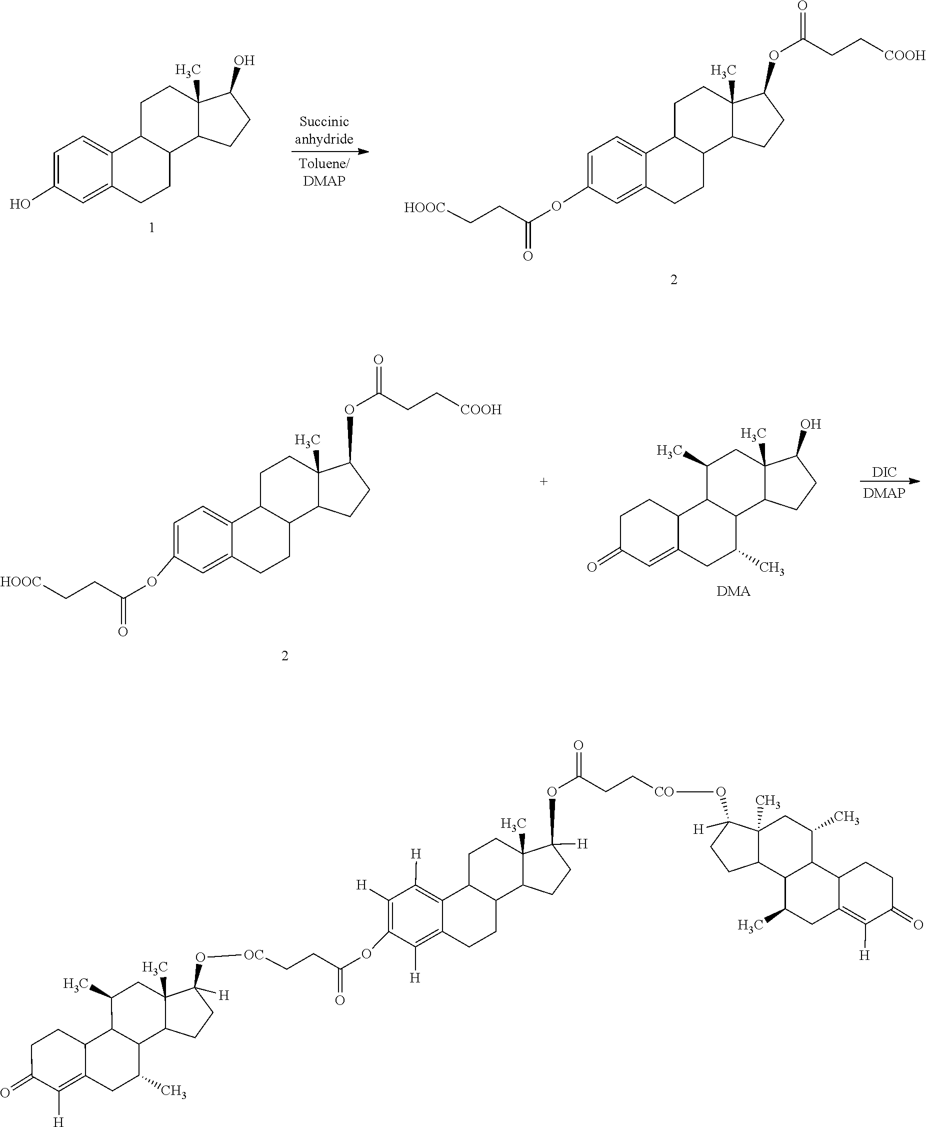

A mixture of succinic anhydride 1 (3.0 g, 30 mmol), pyridine (1 mL) and Estradiol (1.36 g, 5.0 mmol) in anhydrous Toluene (30 mL) was heated at reflux overnight. After cooling to room temperature, the separated solid (excess succinic anhydride) was removed via filtration. The filtrate was concentrated to a residue. The material was dried in high vacuum to obtain a semi-solid mass (1.9 g). To a stirred mixture of DMA-Di-Succinic acid ester (0.47 g, 1 mmol) in anhydrous CH₂Cl₂ (10 mL), DMA (0.61 g, 2 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue (mixture of unreacted DMA+2 spots) was purified over silica gel column using gradients of CH₂Cl₂/acetone to isolate the most non-polar spot as white foam (240 mg). ¹H NMR partial (400 MHz, CDCl₃): δ 7.25 (d, 1H, Ar—H), 6.84 (dd, 1H, Ar—H), 6.79 (s, 1H, Ar—H), 5.85 (s, 2H, Olefinic-H), 4.60 (t, 1H, C-17 CH), 4.58 (t, 2H, C-17 CH), 2.63 (t, 4H, OCOCH₂—CH₂COO), 1.18 (s, 3H, CH₃), 0.92 (s, 6H, CH₃), 0.82 (d, 6H, CH₃), 0.80 (d, 6H, CH₃). HPLC: 70%; LCMS (M+1): 1043.4.

### Example 79

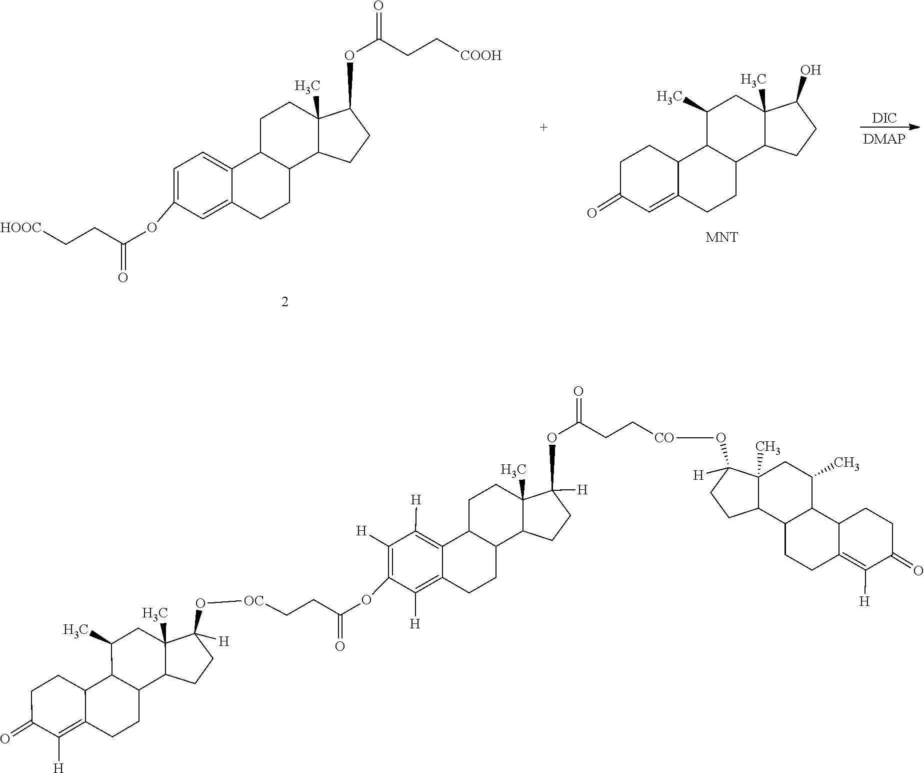

To a stirred mixture of DMA-Di-Succinic acid ester (0.47 g, 1 mmol) in anhydrous CH₂Cl₂ (10 mL), MNT (0.57 g, 2 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue (mixture of trace unreacted MNT+2 spots) was purified over silica gel column using gradients of CH₂Cl₂/acetone to isolate the most non-polar spot as white foam (210 mg). ¹H NMR partial (400 MHz, CDCl₃): δ 7.26 (d, 1H, Ar—H), 6.83 (dd, 1H, Ar—H), 6.81 (s, 1H, Ar—H), 5.85 (s, 2H, Olefinic-H), 4.71 (t, 1H, C-17 CH), 4.59 (t, 2H, C-17 CH), 2.63 (t, 4H, OCOCH₂—CH₂COO), 1.068 (s, 3H, CH₃), 0.93 (d, 6H, CH₃>, 0.81 (s, 6H, CH₃). HPLC: 78%. LCMS (M+1): 1014.4.

### Example 80

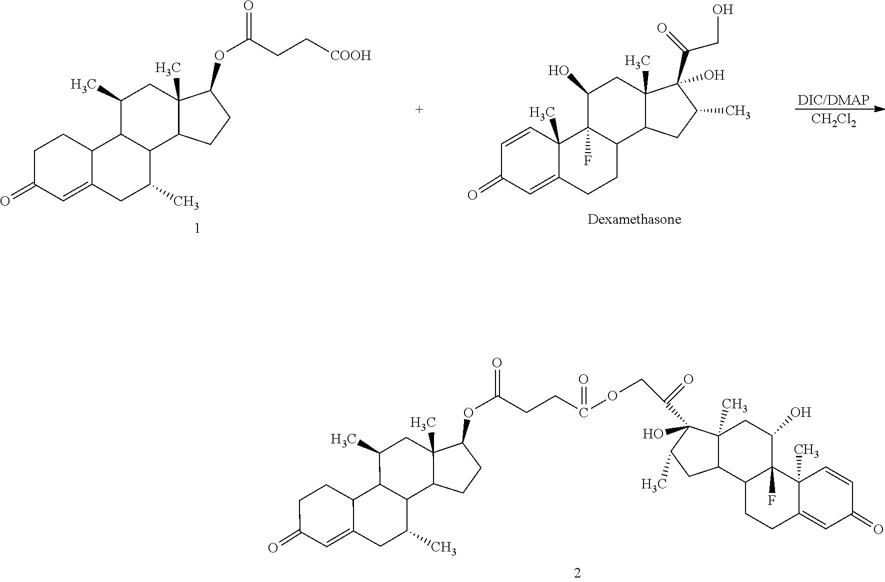

To a stirred mixture of DMA-17-Succinic acid ester (PKG-15-259, 0.37 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot as white solid (0.32 g). White solid; Melting Point: 182-183° C. ¹H NMR partial (400 MHz, CDCl₃): δ 7.18 (d, 1H, Ar—H), 6.35 (d, 1H, Ar—H), 6.13 (s, 1H, Ar—H), 5.84 (s, 1H, C-4 CH), 4.55 (t, 1H, C-17 CH), 2.72 t, 2H, OCOCH₂—CH₂COO), 1.15 (2s, 6H, CH₃), 0.91 (s, 3H, C-18 CH₃), 0.78 (d, 3H, C-7α CH₃>. HPLC: 99.3%. LC/MS: 777.8 (M+1).

### Example 81

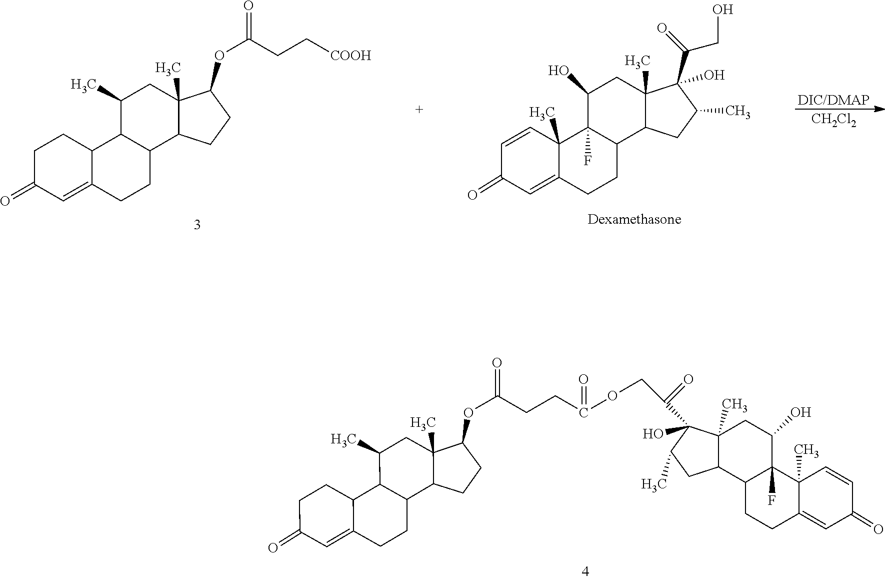

To a stirred mixture of MNT-17-Succinic acid ester (PKG-15-251, 0.39 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot. Pure fractions were combined and stripped to obtain white solid. The resulting residue was triturated with MTBE (5 mL) at RT and filtered. Resulting solid was air-dried and then dried under high vacuum to afford pure coupled product 4 as white solid (0.31 g). White solid; Melting Point: 189-191° C. ¹H NMR partial (400 MHz, CDCl₃): δ 7.17 (d, 1H, Ar—H), 6.33 (d, 1H, Ar—H), 6.11 (s, 1H, Ar—H), 5.84 (s, 1H, C-4 CH), 4.57 (t, 1H, C-17 CH), 2.72 t, 2H, OCOCH₂—CH₂COO), 1.15-1.16 (2s, 6H, CH₃), 1.04 (d, 3H, CH₃), 0.91 (s, 3H, C-18 CH₃). HPLC: 96.4%. LC/MS: 763.8 (M+1).

### Example 82

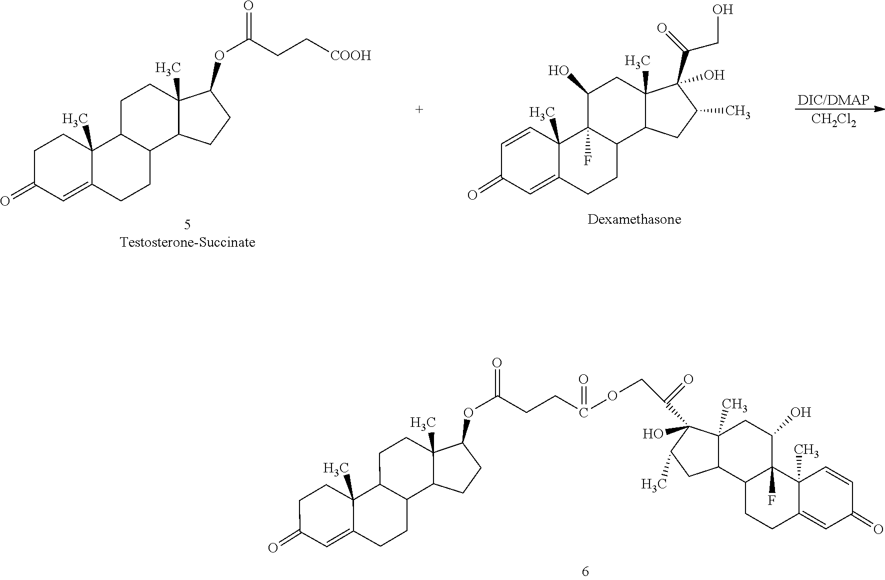

To a stirred mixture of testosterone-17-Succinic acid ester (PKG-15-262, 0.39 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot as white solid (0.37 g). White solid; Melting Point: 258-261° C. ¹H NMR partial (400 MHz, CDCl₃): δ 7.18 (d, 1H, Ar—H), 6.33 (d, 1H, Ar—H), 6.11 (s, 1H, Ar—H), 5.73 (s, 1H, C-4 CH), 4.63 (t, 1H, C-17 CH), 2.72 t, 2H, OCOCH₂—CH₂COO), 1.15-1.16 (2s, 6H, CH₃), 1.03 (s, 3H, CH₃), 0.91 (d, 3H, CH₃), 0.84 (s, 3H, C-18 CH₃). HPLC: 95%; LC/MS: 763.9 (M+1).

### Example 83

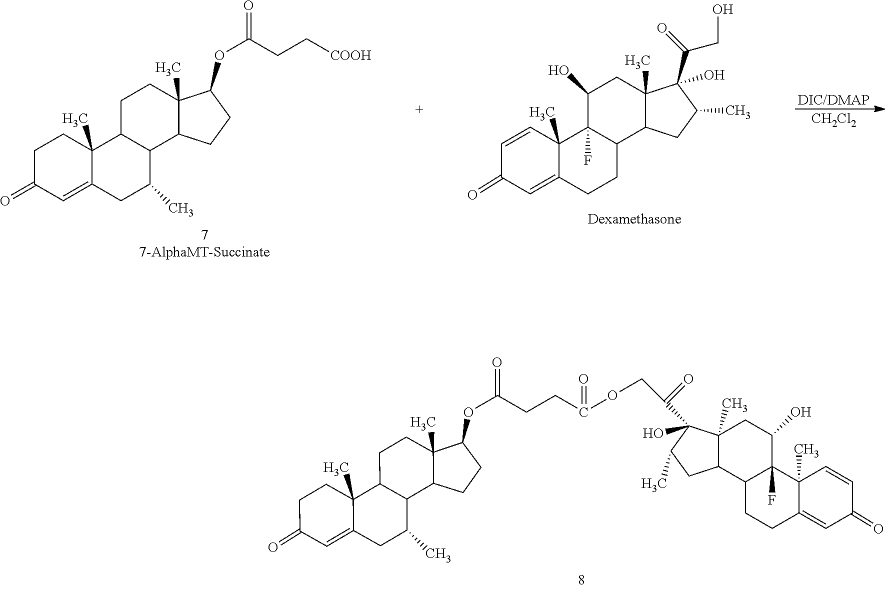

To a stirred mixture of 7-AlphaMT-17-Succinic acid ester (PKG-15-265, 0.37 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot as white solid (0.31 g). White solid; Melting Point: 183-185° C. ¹H NMR partial (400 MHz, CDCl₃): δ 7.19 (d, 1H, Ar—H), 6.33 (d, 1H, Ar—H), 6.11 (s, 1H, Ar—H), 5.73 (s, 1H, C-4 CH), 4.62 (t, 1H, C-17 CH), 2.63 t, 2H, OCOCH₂—CH₂COO), 1.05 (s, 3H, CH₃), 0.91 (d, 3H, CH₃), 0.84 (s, 3H, C-18 CH₃), 0.77 (d, 3H, C-7α CH₃>. HPLC: 99.5%; LC/MS: 777.7 (M+1).

### Example 84

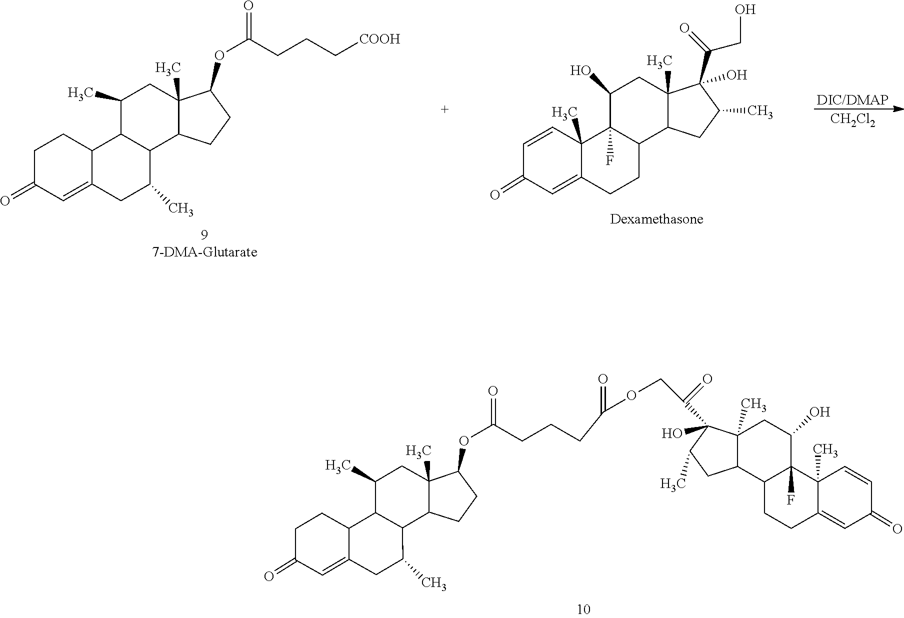

To a stirred mixture of 7-DMA-17-glutaric acid ester (PKG-15-272, 0.21 g, 0.5 mmol) in anhydrous CH₂Cl₂ (7 mL), Dexamethasone (0.2 g, 0.5 mmol), diisopropylcarbodiimide (0.2 mL, 0.86 mmol) and DMAP (0.01 g, 0.0.08 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot as white solid (0.14 g). White solid; Melting Point: 186-188° C. ¹H NMR partial (400 MHz, CDCl₃): δ 7.18 (d, 1H, Ar—H), 6.35 (d, 1H, Ar—H), 6.13 (s, 1H, Ar—H), 5.84 (s, 1H, C-4 CH), 4.55 (t, 1H, C-17 CH), 2.72 t, 2H, OCOCH₂—CH₂COO), 1.15 (2s, 6H, CH₃), 0.91 (s, 3H, C-18 CH₃), 0.78 (d, 3H, C-7α CH₃). HPLC: 95.2%; LC/MS: 791.6 (M+1).

### Example 85

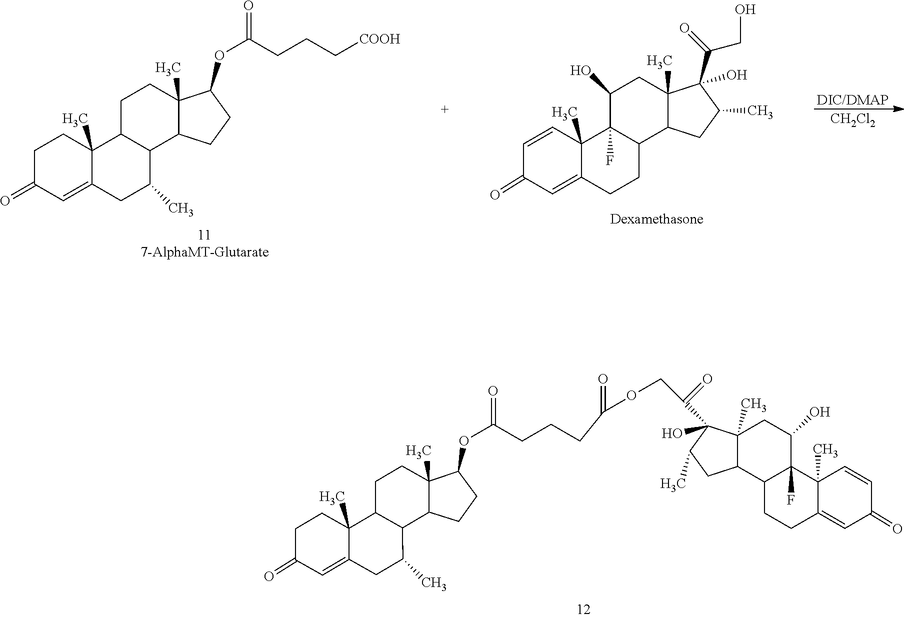

To a stirred mixture of 7α-MT-17-glutaric acid ester (PKG-15-270, 0.41 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot as white solid (0.21 g). Melting point: 185-187° C. ¹H NMR partial (400 MHz, CDCl₃): δ 7.20 (d, 1H, Ar—H), 6.33 (dd, 1H, Ar—H), 6.11 (s, 1H, Ar—H), 5.73 (s, 1H, C-4 CH), 4.89 (s, 2H), 4.63 (t, 1H, C-17 CH), 1.14 (s, 3H, CH₃), 0.91 (d, 3H, CH₃), 0.84 (s, 3H, C-18 CH₃), 0.76 (d, 3H, C-7α CH₃>. HPLC: 98.4%; LC/MS: 791.8 (M+1).

### Example 86

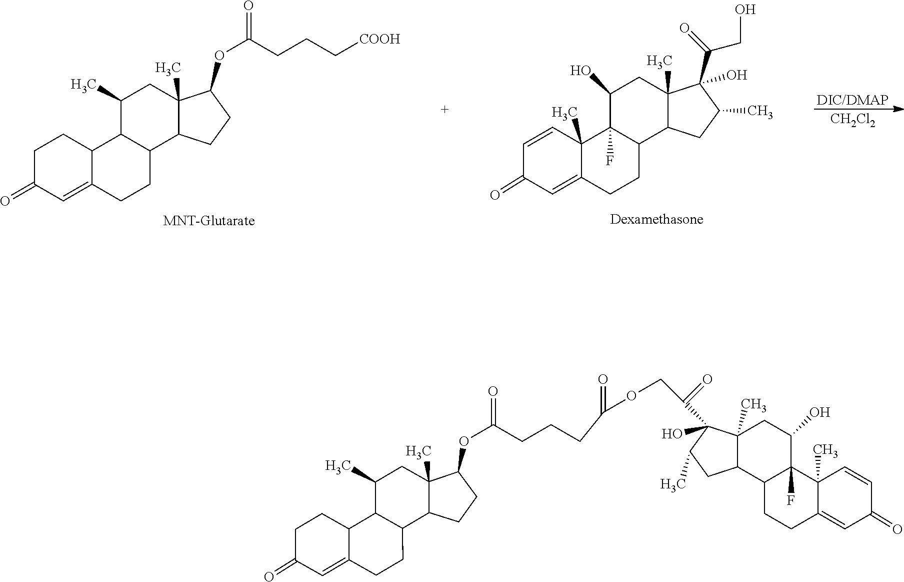

To a stirred mixture of MNT-17-glutarate (PKG-15-280, 0.40 g, 1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot. Pure fractions were combined and stripped to obtain white solid. The resulting residue was triturated with MTBE (5 mL) at RT and filtered. Resulting solid was air-dried and then dried under high vaccum to afford pure coupled product as white solid (0.28 g). Melting point: 183-185° C. ¹H NMR partial (400 MHz, CDCl₃): δ 7.18 (d, 1H, Ar—H), 6.35 (d, 1H, Ar—H), 6.11 (s, 1H, Ar—H), 5.85 (s, 1H, C-4 CH), 4.58 (t, 1H, C-17 CH), 2.72 t, 2H, OCOCH₂—CH₂COO), 1.15-1.16 (2s, 6H, CH₃), 1.04 (d, 3H, CH₃), 0.92 (s, 3H, C-18 CH₃). HPLC: 99.6%; LCMS: 777.3 (M+1).

### Example 87

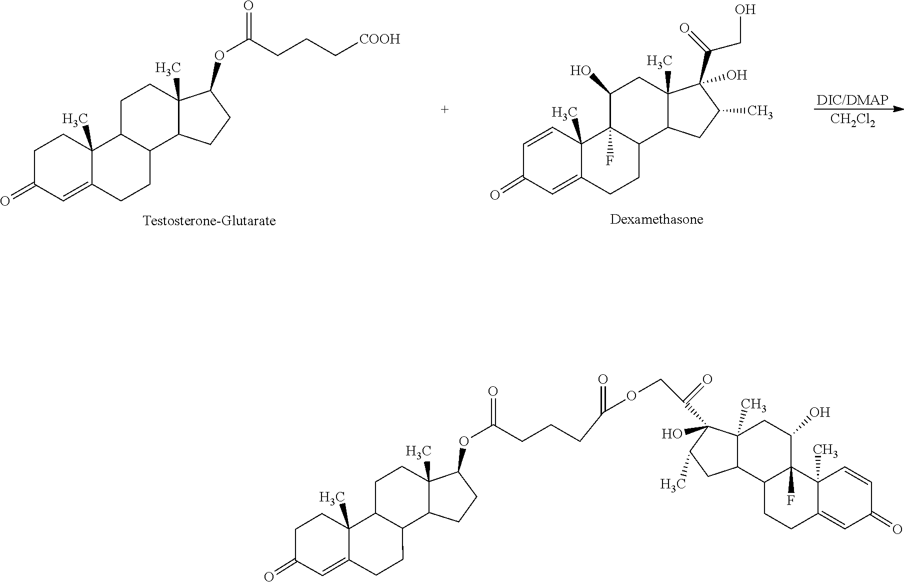

To a stirred mixture of Testosterone 17-glutarate (1 mmol) in anhydrous CH₂Cl₂ (10 mL), Dexamethasone (0.392 g, 1 mmol), diisopropylcarbodiimide (0.4 mL, 1.72 mmol) and DMAP (0.015 g, 0.13 mmol) were added under nitrogen, and the resulting reaction mixture was stirred at room temperature overnight. Next day, the resulting mixture was filtered through a celite pad and the filtrate was diluted with CH₂Cl₂ (20 mL). The organic phase was washed successively with water (1×10 mL), 5% HCl solution (1×10 mL) and brine (1×10 mL). After drying over MgSO₄, the filtrate was evaporated. The crude residue was purified over silica gel column using gradients of CH₂Cl₂/acetone to isolate the polar spot. Pure fractions were combined and stripped to obtain white solid. The resulting residue was triturated with MTBE (5 mL) at RT and filtered. Resulting solid was air-dried and then dried under high vacuum to afford pure coupled product as off-white solid (0.19 g). Melting Point: 253-257° C. ¹H NMR partial (400 MHz, CDCl₃): δ 7.20 (d, 1H, Ar—H), 6.33 (d, 1H, Ar—H), 6.10 (s, 1H, Ar—H), 5.84 (s, 1H, C-4 CH), 4.56 (t, 1H, C-17 CH), 3.80 (m, 2H, OCOCH₂—CH₂COO), 1.15-1.17 (2s, 6H, CH₃), 1.04 (d, 3H, CH₃), 0.91 (s, 3H, C-18 CH₃). HPLC: 88.14%; LCMS: 777.8 (M+1).

### C. Stability Examples

### Example I

In this example, an HPLC method was used to evaluate stability of monomeric compound embodiments. Isocratic elution on a reversed-phase (C18) column with a mobile phase that was 90% acetonitrile 10% water and containing 0.1% formic acid was used. On each day of assay, two monomeric compound embodiments were evaluated using the following methodology:
1. Determination of optimal mass spectral detection with the triple quadrupole detector, which involved infusing the monomeric compounds into the mass spectrometer, and determining best voltage, gas, and temperature setting to get the most sensitive response for the precursor to product ions formed during measurement; and
2. Determination of the retention time of the monomeric compound on the HPLC method, so that an appropriate internal standard, i.e., one with a similar retention time can be selected. In general, the internal standard was another monomeric compound, not being assayed on that day.

Stock solutions of the monomeric compound embodiments being assayed were prepared at a concentration of 10 mM in acetonitrile, and these were used for preparation of the spiking solutions for the plasma stability study, and for the preparation of the calibration standards used in the assay.

Plasma Stability Assay—Ninety nine volumes of human male plasma were spiked with 1 volume of a spiking solution that was prepared in 50% acetonitrile 50% water at concentrations of 1.0 or 0.1 mM, to yield final spiked plasma concentrations of 10 μM and 1 [symbol] M, respectively. The stability studies were conducted at 37° C., and therefore the plasma was equilibrated to this temperature prior to receiving the spiking solution. Every stability sample only contained one monomeric compound embodiment being evaluated.

The internal standard solution used in these studies was prepared fresh on the day of assay, and it was typically composed of selected internal standards at a concentration of 2 μg/ml in a solution that was 50% methanol, 50% water. Fifty microliters of the internal standard solution were pre-aliquoted into the assay tubes (1.5 ml plastic microcentrifuge tubes), and the tubes were kept closed until the time of sample assay. The stability timepoints were (in hours): 0, 0.25, 0.5, 1, 2, 4, and 24 hours.

The assay of monomeric compounds then proceeded as follows:
1. Pre-warm plasma and spiking solutions to 37° C.
2. At T=0 add spiking solution 1:99 to plasma (15 μl plus 1.485 ml plasma)
3. and immediately transfer duplicate 50 μl aliquots to tubes with IS & add solvent and extract.
4. At times indicated above remove duplicate 50 μl samples from incubations samples to tubes with IS and extract by adding 1 ml ethyl acetate:hexane (50:50, v:v) and vortex 60 sec.
5. Store samples at room temperature until all timepoints are collected, then vortex all samples 10 min on plate vortexer.
6. Centrifuge extracts 10 min, 18000 g.
7. Transfer 0.85 ml upper layers to individual wells of 96 well plate.
8. Dry down under stream of nitrogen.
9. Reconstitute with 100 μl 90% ACN, centrifuge plate 2301 g 15 min, and analyze by LCMS.

Quantification of the amount of monomeric compound in each plasma sampling time was made by carrying a set of calibration standards prepared in human plasma through the above extraction steps. In particular embodiments, the evaluated monomeric compounds comprised an R² functional group at the C17 hydroxyl group and the calibrations standards were also prepared containing a form of the monomeric compound without the C17 hydroxyl R² functional group to quantify the amount that had been generated by hydrolysis of the R² functional group.

Results for some additional samples are provided below in Table 1.

TABLE 1
Human male plasma stability

| | Parent molecule | |
|---|---|---|
| | % remaining after | |
| | 24 hours at 37° C. | |
| Compound | 1 μM | 10 μM |
| Levonorgestrel | 72 | 84 |
| CDB-902 | 100≤ | 100≤ |
| CDB-4746 | 99\hat{ } | 97\hat{ }\hat{ } |
| CDB-1321 | 100≤* | 92** |
| CDB-4866 | 91 | 90 |
| CDB-4867 | 100≤ | 100≤ |
| CDB-4868 | 80 | 58 |
| CDB-4870 | 89 | 83 |
| CDB-4871 | 91 | 100≤ |
| CDB-4879 | 88 | 100≤ |
| CDB-4880 | 95 | 91 |
| CDB-4883 | 85 | 81 |
| CDB-4884 | 83 | 97 |
| CDB-4886 | 78 | 69 |
| CDB-4887 | 89 | 74 |
| CDB-4888 | 89 | 76 |
| CDB-4890 | 83 | 66 |
| CDB-4891 | 82 | 86 |
| CDB-4892 | 81 | 99 |
| CDB-4910 | 100≤ | 95 |
| CDB-4912 | 66 | 76 |
| CDB-4913 | 100≤ | 93 |
| CDB-4914 | 100≤ | 88 |
| CDB-4923 | 84 | 75 |

\hat{ }Concentration at 1.73 μM;
\hat{ }\hat{ }Concentration at 17.3 μM;
*Concentration at 1.65 μM;
**Concentration at 16.5 μM

With reference to Table 1, compound CDB-902 is 7-α-methylnortestosterone; CDB-4746 is 11β-methyl-19-nortestosterone; CDB-1321 is dimethandrolone; CDB-4883 is testosterone dodecylcarbonate; and CDB-4884 is 7-α-methylnortestosterone dodecylcarbonate.

Liver S9 Fraction Stability Assay—Stability in liver S9 samples also was evaluated. In particular examples, the monomeric compound embodiments were incubated at two concentrations (1 and 10 μM) with 0.5 mg/ml male human S9 fraction, 2.5 mM NADPH, and 3.3 mM MgCl₂ (Sigma) in 0.1M phosphate buffer, pH 7.4, at 37° C. Aliquots (100 μl) were removed at 0, 15, 30, 60, 90 and 120 minutes and mixed with 200 μl of acetonitrile containing either 100 ng/ml (for 1 μM) or 1000 ng/ml (for 10 μM) d3-testosterone as internal standard. Incubations with certain monomeric compound embodiments and heat-killed S9 were removed at 0 and 120 minutes. Fifty microliters (for 1 μM) or 100 μl (for 10 μM) of each supernatant was diluted with 100 or 200 μl of 0.1% formic acid in water, vortexed, then analyzed by LC-MS/MS in multiple reaction monitoring mode using positive-ion electrospray ionization. Human liver S9 were purchased from XenoTech, LLC. A decrease of the monomeric compound embodiment remaining with time when incubated with active microsomes indicates that the parent monomeric compound was metabolized in the conditions used. The liver S9-fraction stability test was conduct with and without co-factor NADPH to determine the stability of these compounds in the presence of CYP activation. Results for some compounds are provided below in Table 2.

TABLE 2
% parent molecule remaining after 2 hours at 37° C.

| | With NADPH | | W/O NADPH | |
|---|---|---|---|---|
| Compound | 1 μM | 10 μM | 1 μM | 10 μM |
| CDB-4883 | 64 | 85 | 74 | 84 |
| CDB-4884 | 75 | 82 | 89 | 75 |
| CDB-4892 | 75 | 82 | 88 | 77 |
| CDB-4893 | 54 | 51 | 60 | 55 |
| CDB-4895 | 55 | 60 | 51 | 62 |
| CDB-4880 | 54 | 76 | 78 | 86 |
| CDB-4889 | 12 | 56 | 100≤ | 100≤ |
| CDB-4912 | 0 | 0 | 5 | 0 |
| CDB-4913 | 11 | 13 | 5 | 17 |
| CDB-4914 | 52 | 76 | 62 | 86 |

With reference to Table 2, compound CDB-4893 is DMA oleate; and 4895CDB-4746 is DMA linolate

### D. Performance Assays

Receptor Binding Assay Information—In certain of the following examples, hormone receptor binding assays were used to determine the receptor binding activities of monomeric compound embodiments to androgen (AR), progesterone (PR), estrogen alpha (ERα) or glucocorticoid (GR) receptors. For AR, PR or ERα, the respective receptors are added to novel, tight-binding, selective ligands tagged with a fluorescent molecule, to form a receptor-ligand complex with a high polarization value. This complex is then added to individual monomeric compound embodiments that are contained in well plates. If the monomeric compound binds to the respective receptors, it displaces the fluorescent ligand from the complex, resulting in a low polarization value. In such embodiments, the monomeric compound is characterized as a competitor. If the monomeric compound does not bind to the respective receptors, it will not displace the fluorescent ligand from the complex, resulting in the polarization value remaining high. In such embodiments, the monomeric compound is characterized as a noncompetitor. The shift in polarization value in the presence of the monomeric compound is used to determine the relative affinity of monomeric compound for the receptor.

For the androgen, progesterone, and estrogen receptor binding assays described herein, the preparation of reagents and test procedures is performed according to the manufacturer's instructions (Life Technologies, Carlsbad, Calif.). The plates are covered to protect the reagents from light and incubated at 20-25° C. for 4-8 hrs. The fluorescence polarization of the samples is measured with 485 nm excitation and 530 nm emission interference filters.

### Example II

In this example, AR binding activity and ER binding activity of certain reference compounds and monomeric compound embodiments wherein the R² group is not present was assessed (such as to test compound activity after R² cleavage). The AR fluorescence polarization (FP) assay was used and provides a sensitive and efficient method for high-throughput screening of potential AR ligands. The kit uses rat AR ligand-binding domain tagged with His and GST [AR-LBD (His-GST)], and a tight-binding, selective fluorescent androgen ligand (Fluormone™ AL Green) in a homogenous mix-and-read assay format. The androgen receptor [AR-LBD (His-GST)] was added to a fluorescent androgen ligand (Fluormone™ AL Green) to form an AR-LBD (His-GST)/Fluormone™ AL Green complex resulting in a high polarization value. This complex was then added to individual compound embodiments in 96-well plates. Competitors displaced the fluorescent Fluormone™ AL Green ligand from the AR-LBD (His-GST)/Fluormone™ AL Green complex, causing the fluorescent ligand to tumble rapidly during its fluorescence lifetime, resulting in a low polarization value. Noncompetitors did not displace the fluorescent ligand from the complex, so the polarization value remained high. The shift in polarization value in the presence of compound embodiments used to determine relative affinity of compound embodiments for AR-LBD (His-GST). The fluorescence polarization of the samples was measured with 485 nm excitation and 530 nm emission interference filters.

For ERα data, the ERα fluorescence polarization (FP) assay was used, which provides a sensitive and efficient method for high-throughput screening of potential ER ligands. The kit uses insect cell-expressed, full-length, untagged, human estrogen receptors and a tight-binding, fluorescent estrogen ligand (Fluormone™ ES2) in a homogenous mix-and-read assay format. ERα was added to a fluorescent estrogen ligand to form an ERα/Fluormone™ ES2 complex. This complex was then added to individual compound embodiments contained in 96-well plates. If the compound embodiment did not compete with Fluormone™ ES2 for binding to the ERα, then the ERα/Fluormone™ ES2 complex remained intact. Thus, the Fluormone™ ES2 tumbled slowly during its fluorescence lifetime, resulting in a high polarization value. Competing compound embodiments displaced the Fluormone™ ES2 ligand from ERα, permitting it to tumble rapidly and resulted in a low polarization value. The change in polarization value in the presence of a compound embodiment was used to determine relative affinity of a compound embodiment for ERα. The fluorescence polarization of the samples was measured with 485 nm excitation and 530 nm emission interference filters.

Results for both the AR and ERα assays are provided in Table 3.

TABLE 3

| Sample | CDB | | IC₅₀ (nM) | |
|---|---|---|---|---|
| Number | Number | Name | AR | ERα |
| 1 | 4910 | 7α-methyl testosterone | 9.7 | 7833 |
| 2 | 4911 | 7β-methyl testosterone | 13.1 | ND |
| 3 | 4915 | 7α-methyl estradiol | 20.1 | 10.26 |
| 4 | 4916 | 7α, 11β-dimethyl estradiol | 17.8 | 13.82 |
| 5 | 4917 | 11β-methyl estradiol | 12.8 | 12.02 |
| 6 | 4918 | 7α-ethyl testosterone | 16.3 | 10450 |
| 7 | 4919 | 7α-t-butyl testosterone | 587.6 | 10420 |
| 8 | 4920 | 7α-phenyl testosterone | 43.1 | 13460 |
| 9 | 4921 | 7β-phenyl testosterone | 891.7 | ND |
| 10 | 4922 | 7α-methyldihydro testosterone | 57.1 | 26340 |
| 11 | 4890 | 11β, 4,9 diene-MNT | 15.5 | 6675 |
| 12 | 107 | Levonorgestrel | 6.66 | ND |
| 18 | 111 | Testosterone | 8.6 | ND |
| 19 | n/a | Estradiol | 21.7 | 13.6 |

### Example III

In this example, PR binding activity was assessed using the PR fluorescence polarization (FP) assay, which provides a sensitive and efficient method for high-throughput screening of potential PR ligands. The kit uses human PR ligand-binding domain tagged with GST [PR-LBD (GST)], and a tight-binding, selective fluorescent progesterone ligand (Fluormone™ PL Green) in a homogenous mix-and-read assay format. Progesterone receptor [PR-LBD (GST)] was added to a fluorescent progesterone ligand (Fluormone™ PL Green) to form a PR-LBD (GST)/Fluormone™ PL Green complex resulting in a high polarization value. This complex was then added to individual compound embodiments in 96-well plates. Competitors displaced the fluorescent Fluormone™ PL Green ligand from the PR-LBD (GST)/Fluormone™ PL Green complex, causing the fluorescent ligand to tumble rapidly during its fluorescence lifetime, resulting in a low polarization value. Noncompetitors did not displace the fluorescent ligand from the complex, thus the polarization value remained high. The shift in polarization value in the presence of compound embodiments was used to determine relative affinity of compound embodiments for PR-LBD (GST). The fluorescence polarization of the samples was measured with 485 nm excitation and 530 nm emission interference filters. Results for monomeric compound embodiments wherein the R² groups is not present (such as to test compound activity after R² cleavage) are provided in Table 4.

TABLE 4

| CDB | | IC₅₀ |
|---|---|---|
| Number | Compound | (nM) |
| 4910 | 7α-methyl testosterone | 394.8 |
| 4911 | 7β-methyl testosterone | 415.2 |
| 4918 | 7α-ethyl testosterone | 671.6 |
| 4924 | 7β-ethyl testosterone | 841.6 |
| 4920 | 7α-phenyl testosterone | 463.3 |

TABLE 4

| CDB | | IC₅₀ |
|---|---|---|
| Number | Compound | (nM) |
| 4921 | 7β-phenyl testosterone | 786.8 |
| 4919 | 7β-t-butyl testosterone | 1399 |
| 4922 | 7α-methyldihydro testosterone | 1892 |
| 4890 | 11β, 4,9 diene-MNT | 43.45 |
| 4915 | 7α-methyl estradiol | 502.2 |
| 4916 | 7α, 11β-dimethyl estradiol | 164.5 |
| 4917 | 11β-methyl estradiol | 120.3 |

### Example IV

In this example, AR, PR, and GR transactivation assays were conducted and are described below.

In this example, HEC-1-B (human endometrial adenocarcinoma), HEK-293 (human embryonic kidney) and T47D-KBluc (human breast carcinoma with luciferase reporter) were purchased from ATCC (Manassas, Va.) and maintained according to ATCC suggested culture method. HEC-1-B and HEK-293 cells were transfected with the reporter vector using Fugene6 (Roche, Indianapolis, Ind.) transfection method and cultured in growth medium containing Hygromycin B (Invitrogen, Carlsbad, Calif.) at the final concentration of 200 μg/ml for selection of stable transfectants. Cells were grown in the Hygromycin B-containing medium for three weeks then Hydromycin B-resistant colonies were isolated using cloning cylinders (EMD Millipore Corp., Billerica, Mass.) and expanded. HEC-1-B cell line stably transfected with pGL4.36[luc2P/MMTV/Hygro] was transfected with expression vectors for AR (pEZ-AR) and GR (pEZ-GR) to generate stable cell lines, HEC1B-MMTV-AR and HEC1B-MMTV-GR, respectively. HEK293 cell line stable transfected with pGL4.36[luc2P/MMTV/Hygro] was transfected with expression vector for PR (pEZ-PR) to generate stable cell line HEK293-MMTV-PR. Selection of stable transfectants were initiated by adding Geneticin and Hygromycin B (Invitrogen, Carlsbad, Calif.) to the culture medium at final concentration of 200 μg/ml each. Antibiotics-resistant stable reporter cells were propagated and used for compound profiling.

Also, in this example, the luciferase assays were performed in the high throughput screening (HTS) 96-well format using HTS instruments. As described below, HEC1B-MMTV-AR, HEC1B-MMTV-GR, T47D-KBluc and HEK293-MMTV-PR cells were passaged as a 1:5 dilution in culture medium containing 5% CD-FBS and incubated at 37° C. with 5% CO₂. The media was changed every 2 or 3 days until cells were ˜90% confluent then the cells were plated in 96-well plates on Day 1. Reference compounds and compound embodiments were serially diluted in 100% ethanol in the range of 10 mM to 1 μM. On Day 2, cells were treated with the reference and test articles and incubated at 37° C. in 5% CO₂ for 22 to 26 hours. At the end of this period, the luciferase activity of each well was measured using the Luciferase assay system according to the manufacturer's instructions (Promega, Madison, Wis.). Data were analyzed by GraphPad Prism 6.0 statistical software and EC50/IC50 values were generated by fitting data from the luciferase reporter assay by nonlinear regression function. Values are reported as mean±standard deviation (SD).

AR Transactivation Assay—Human Endometrial Carcinoma (HEC-1-B) cells were used for androgen receptor transcriptional activity tests to screen compound embodiments (e.g., compounds disclosed herein wherein the R² group of a monomeric compound embodiment has been cleaved, or wherein a linker group of an oligomeric compound embodiment has been cleaved) for potential androgenic and anti-androgenic activity. MMTV-Luc, the reporter gene construct consisted of MMTV LTR (Murine Mammary Tumor Virus Long Terminal Repeat) which contains hormone response element (HRE) that regulates the expression of a luciferase reporter gene in response to activation of several nuclear receptors such as AR, PR and GR. Transient transfection of this promoter-reporter construct along with pCMV-hAR construct expressing human full-length androgen receptor and Rluc, Renilla luciferase construct as a transfection control, to HEC-1-B cells resulted in a sensitive and responsive system for detecting AR activation upon ligand binding. Stable transfection of MMTV reporter construct and pEZ-AR construct expressing human full-length androgen receptor resulted in HEC1B-MMTV-AR stable cell line.

Upon compound entry into the cell, androgen receptor ligands bind to the AR and the two ligand-bound receptors dimerize and bind to coactivators. The activated dimer binds to the HRE on the reporter gene construct and this, in turn, activates transcription and then translation of the luciferase reporter gene. The presence of the luciferase enzyme is then assayed by measuring the light produced when the enzyme substrate, luciferin, and appropriate cofactors are added to the cell lysate. The amount of light produced by the reporter enzyme is directly related to the degree of androgenic activity of the compound embodiment. In this example, an androgenic compound is one that induces dose-dependent luciferase activity that is specifically inhibited by an anti-androgenic compound such as hydroxyflutamide (OHF). In agonist format, cells were incubated with compound embodiments or reference agonist in various concentrations. In antagonist format, cells were incubated with compound embodiments or reference antagonist in various concentrations and co-treated with a fixed concentration of reference agonist.

AR was introduced into the respective cell lines via transient or stable transfection of expressions vectors (pEZ-AR) along with the luciferase reporter vector (MMTV-Luc). MMTV-Luc, the reporter gene construct consisted of MMTV LTR (Murine Mammary Tumor Virus Long Terminal Repeat) contains hormone response element (HRE) that regulates the expression of a luciferase reporter gene in response to activation of androgen or progesterone receptors. Reference compounds and monomeric compound embodiments were serially diluted in 100% DMSO in the range of 10 mM to 1 μM. Cells expressing each receptor and MMTV-Luc were treated with the reference compound and the monomeric compound embodiments and incubated at 37° C. in 5% CO₂ for 22 to 26 hrs. At the end of this period, the luciferase activity of each well was measured using the Luciferase assay system according to the manufacturer's instructions (Promega, Madison, Wis.). EC50 values were generated by nonlinear regression analysis using the relative luminescence unit (RLU) data. A steroid receptor-fluorescent ligand complex, AR-LBD (HisGST)/Fluormone™—AL Green (Invitrogen, Carlsbad, Calif.), was added to individual monomeric compound samples in each 96-well plate according to the manufacturer's instructions. The plates were covered to protect the reagents from light and incubated at 20-25° C. for 4-8 hrs. The fluorescence polarization of the samples was measured with 485 nm excitation and 530 nm emission interference filters. Results are provided in Table 5 below.

TABLE 5

| | EC₅₀ | |
|---|---|---|
| | (nM) | R² |
| Testosterone | 0.2382 | 0.9025 |
| Dihydrotestosterone | 0.7889 | 0.8464 |
| 7α-ethyl testosterone | 11.5 | 0.9677 |
| 7α-methyl estradiol | 38.99 | 0.6005 |
| 7α, 11β-dimethyl estradiol | 3.677 | 0.9741 |
| 7α-methyl testosterone | 0.1491 | 0.6544 |
| 7β-methyl testosterone | 0.3199 | 0.8016 |

### Example V

In this example, the ability of monomeric compound embodiments to undergo aromatization to corresponding estradiol-based compounds is evaluated. Monomeric compound embodiments and/or or compounds obtained therefrom, such as by cleavage of the R² group, are incubated at one concentration (10 μM) with 0.5 mg/ml human CYP19+P450 reductase Supersomes™, 2.5 mM NADPH, and 3.3 mM MgCl₂ in 0.1M phosphate buffer, pH 7.4, at 37° C. Aliquots (100 μl) are removed at 0, 15, 30 and 60 minutes and mixed with 200 μl of acetonitrile containing 1000 ng/ml d3-testosterone as internal standard. Incubations with the compounds, but no NADPH, are removed at 0 and 60 minutes. Samples are further diluted with the addition of 100 μl of 0.1% formic acid in water, then briefly vortexed and centrifuged for 5 minutes. Fifty microliters of each supernatant are diluted with 200 μl of 0.1% formic acid in water, vortexed, then analyzed by LC-MS/MS in multiple reaction monitoring mode using positive-ion electrospray ionization. CYP19+P450 reductase Supersomes™ are purchased from Corning Discovery Labware (Woburn, Mass.).

Testosterone, a known substrate of aromatase that is metabolized to estradiol, was included as a positive control. As expected, when incubated at 10 μM with recombinant aromatase, it was rapidly metabolized, with 31.4% remaining at 15 min and only 0.1% remaining at both 30 and 60 minutes. Monomeric compound embodiments were metabolized by to varying degrees when incubated with recombinant human aromatase, with 7α-ethyltestosterone (CDB-4918) being metabolized most rapidly. The rank order of most to least metabolized, based on % remaining at 15 minutes for rapidly metabolized compound embodiments or % remaining at 60 minutes for compound embodiments that were metabolized more slowly, was: testosterone (31.4, 0.1 and 0.1% remaining at 15, 30 and 60 min, respectively)˜7α-ethyltestosterone (CDB-4918) (33.0, 0.2 and 0.1% remaining at 15, 30 and 60 min, respectively) >7α-methyltestosterone (CDB-4910) (57.6, 20.2 and 0.1% remaining at 15, 30 and 60 min)>7β-ethyltestosterone (CDB-4924) (58.2, 25.4 and 0.3% remaining at 15, 30 and 60 min)>7β-methyltestosterone (CDB-4911) (62.3, 33.5 and 3.4% remaining at 15, 30 and 60 min)>7α-methyldihydrotestosterone (CDB-4922) (66.8, 38.0 and 10.1% remaining at 15, 30 and 60 min)>7β-phenyltestosterone (CDB-4921) (62.5% remaining at 60 min)>7α-phenyltestosterone (CDB-4920) (63.6% at 60 min)>dimethandrolone (85.3% remaining at 60 min)>7α-tbutyltestosterone (CDB-4919) (92.0% remaining at 60 min). All monomeric compound embodiments, with the exception of CDB-4919, underwent aromatization from full to varying degrees when incubated with recombinant human aromatase.

### Example VI

In this example, a Hershberger Assay (standard and extended) was used to evaluate in vivo activity of certain monomeric and oligomeric compound embodiments. The Hershberger assay is a traditional in vivo bioassay to evaluate androgenic/anabolic properties of compounds. This standard assay is used to evaluate the hormonal activities affecting the target organ (i.e. prostate, seminal vehicles and levator ani muscles) after multiple daily doses for 1 week. The extended version of this assay evaluates the efficacy and duration of the tested compounds over a period of time (8-14 weeks) after administration of a single dose. The study design involves assessment of the target organs every one or two weeks.

Dose formulations were prepared by adding the appropriate amount of reference or test article in the vehicle (ASV) to achieve the desired concentrations. The formulations were mixed using a vortex mixer and sonicator for 15 to 45 min. All formulations in ASV were observed to be white suspensions. Oil dose formulations were prepared by dissolving the appropriate amount of the test article in 1 part of EtOH then adding 9 parts of sesame oil to achieve target concentration in 10% EtOH/90% sesame oil. The formulations were mixed by vortexing, stirring and sonication for 2 to 8 min after each addition of vehicle component. The formulations were observed to be clear, faint yellow to yellow solutions. Castor Oil/BBZ dose formulations were prepared by dissolving the appropriate amount of the test article in the prepared vehicle (70% castor oil/30% BBZ) to achieve the target concentration. The formulations were mixed by vortexing and sonication for 5 to 10 min. The formulations were observed to be clear, slightly yellow solutions.

Nursing Sprague-Dawley intact male rat pups (40-70 g) were weaned and castrated at age 21 days (PND 21) and randomized by body weight into groups.

For standard Hershberger assays, starting on the day of castration (Day 1) and continuing for six consecutive days (Days 2-7), rats were subcutaneously injected with the vehicle control (ASV, castor oil/BBZ or sesame oil/ethyl alcohol), reference control (methyl testosterone or testosterone) or oligomeric compound embodiments. All rats received a set dose volume of 0.2 mL per injection. On Day 8, approximately 24 hours post final dose, the final body weights were recorded, and rats were euthanized.

For extended Hershberger assays, the rats received a single dose of vehicle control, reference control (methyl testosterone or testosterone) or a compound embodiment on Day 1 (via subcutaneous administration) following castration, then euthanized at weekly or biweekly intervals for 14 weeks. Seminal vesicles, ventral prostate glands, and levator ani muscle were excised and weights recorded. Results for certain embodiments are discussed below and summarized in Table 6 and Table 7.

TABLE 6
Area Under the Curve (AUC)

| | | | | | AUC |
|---|---|---|---|---|---|
| | | Dose | | AUC | (Levator |
| Compound | | Level | Dose | (Ventral | ani |
| Embodiment | Vehicle | (μmol) | Conc. | Prostate) | muscle) |
| Vehicle | Sesame | 0 | 0 | 136.5 | 1708 |
| | Oil/EtOH | | | | |
| CDB-4866 | Sesame | 1.25 | | 1464 | 10355 |
| | Oil/EtOH | | | | |
| CDB-4867 | Sesame | 1.25 | | 529.5 | 5059 |
| | Oil/EtOH | | | | |
| CDB-4868 | Sesame | 1.25 | | 2233 | 10730 |
| | Oil/EtOH | | | | |
| CDB-4877 | Sesame | 1.25 | | 524.4 | 2173 |
| | Oil/EtOH | | | | |

TABLE 7

| | Conc. in | Ventral Prostate |
|---|---|---|
| Compound | mmole | (weight in gram) |
| Testosterone | 6.9 | 0.12 |
| 7α-methyl testosterone | 0.7 | 0.076 |
| 7α-methyl testosterone | 2.8 | 0.13 |
| 7α-methyl testosterone | 11.2 | 0.14 |

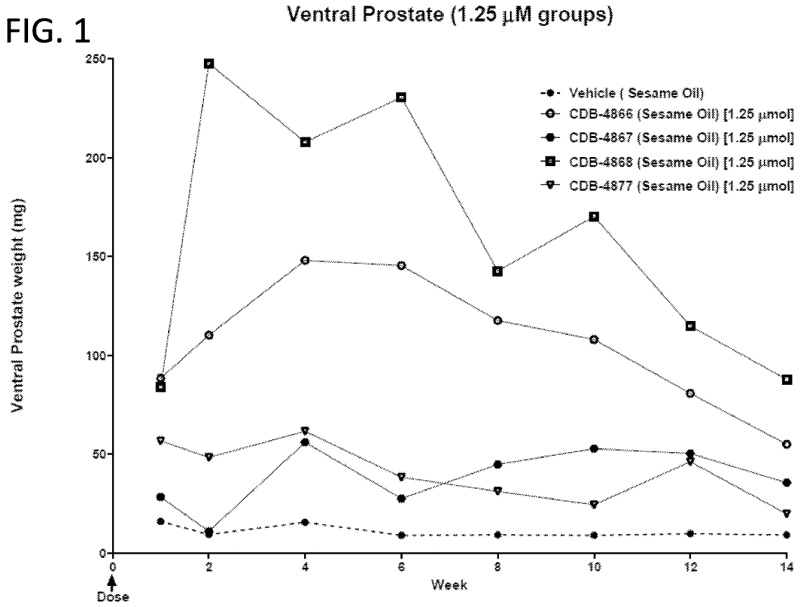

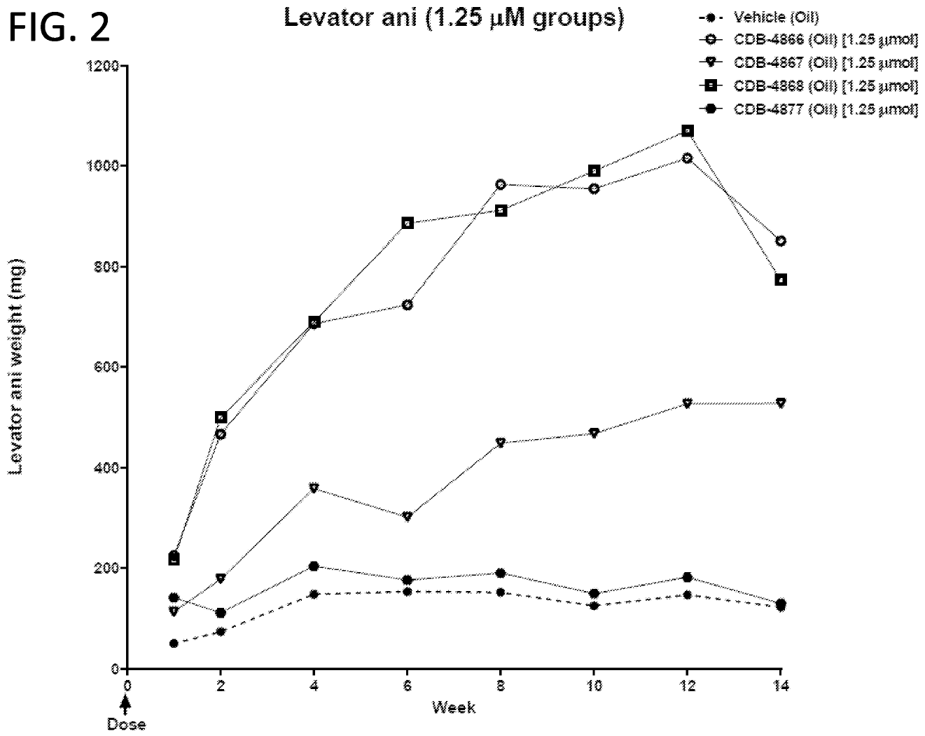

In Sesame oil/ethyl alcohol (Sesame Oil) formulation of oligomers for CDB-4866, CDB-4867, CDB-4868, and CDB-4877, ventral prostate gland weights increased in all treated animals relative to vehicle. Prominent androgenic effects with ventral prostate weight increase was sustained until Day 99 (Week 14) for all treated animals. See FIG. 1 for a graph of results. Ventral prostate as observed from AUC values for most androgenic to least androgenic were CDB-4868>CDB-4866>CDB-4867>CDB-4877. Anabolic effects with levator ani muscle weight increase sustained until Day 99 (Week 14) were observed for CDB-4866, CDB-4867, CDB-4868, CDB-4886. Levator ani as observed from AUC values for most anabolic to least anabolic were CDB-4868˜CDB-4866>CDB-4867. See FIG. 2 for a graph of results.

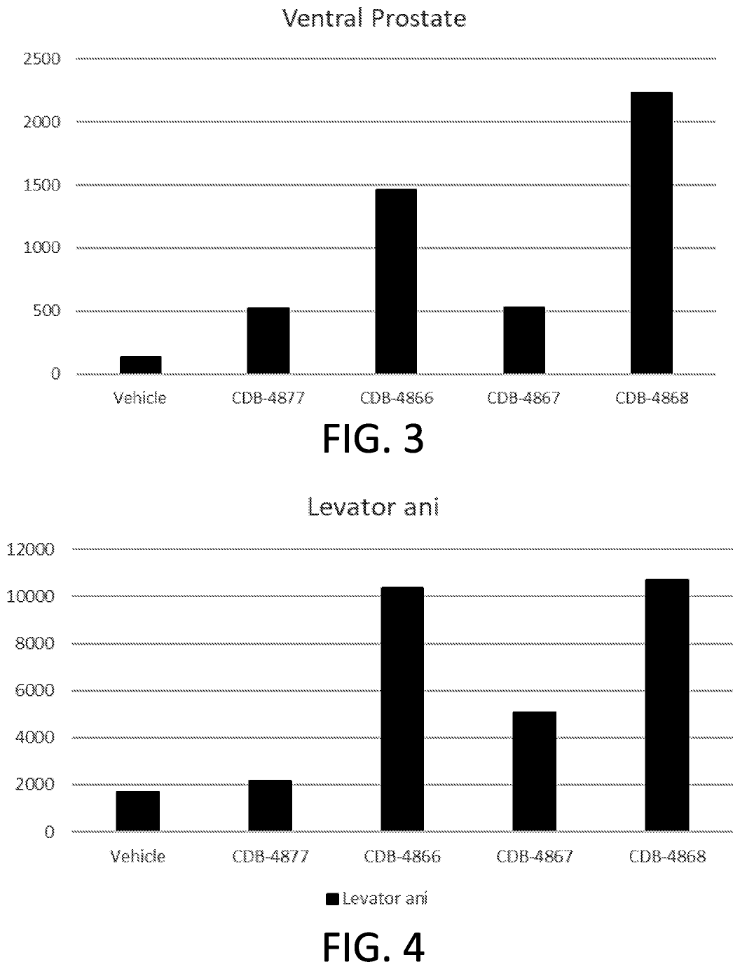

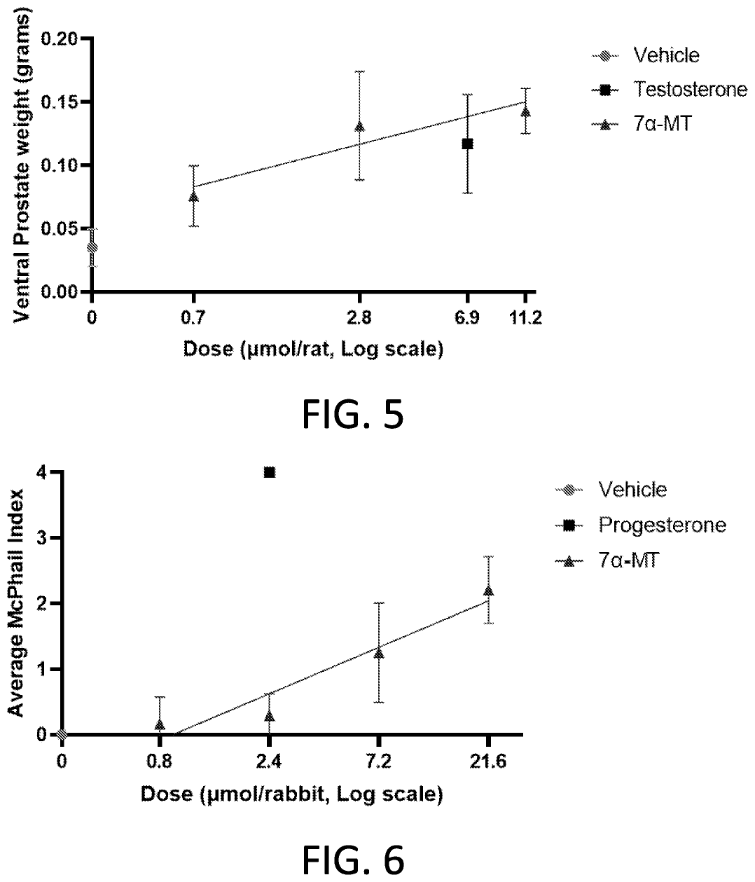

CDB-4868 showed the greatest androgenic effects with AUC values over 2000 mg*week on ventral prostate glands weights. CDB-4866 and CDB-4868 in Sesame Oil showed the greatest anabolic effects with AUC values over 10,000 mg*week on levator ani muscle weights (Table 5). These results clearly demonstrate the androgenicity and anabolic properties of these oligomers. Further, it clearly demonstrates the different linker length and linker type effect differences in the androgenic and anabolic activities. Results for particular oligomeric compound embodiments also are summarized in FIGS. 3 and 4. Results for 7α-MT are shown in FIG. 5.

### Example VII

In this example, a Clauberg Assay (standard and extended) is used to evaluate in vivo activity of certain compound embodiments. The Clauberg assay is a traditional in vivo bioassay used to evaluate progestogenic properties of compounds. This standard assay is used to evaluate the hormonal activities affecting the target organ (i.e. uterus arborization scoring for Clauberg assay) after multiple daily doses for 1 week. The extended version of this assay is used to evaluate the efficacy and duration of the tested compounds over a period of time (8-14 weeks) after administration of a single dose. The study design involves assessment of the target organs every one or two weeks.

New Zealand White 4-6 week old (800-1100 g) immature female rabbits are randomized by body weight into groups (N=3-5 rabbits per group). Rabbits are injected with 17p-estradiol (5 μg/day) subcutaneously for six days (priming). For standard Clauberg assays, rabbits are treated with the vehicle control (ethyl alcohol in castor oil), reference control (progesterone) or a compound embodiment once daily on Days 7-11, then on Day 12, approximately 24 hr post final dose, the final body weights are recorded, and rabbits are euthanized. For extended Clauberg assays, rabbits receive a single dose of vehicle control, reference control (progesterone) or a compound embodiment on Day 7, and are then euthanized on Days 10, 14, 28, 42 and 56. Uteri are excised intact and weights are recorded. Sections (5 μm) of fixed uteri re-evaluated for endometrial glandular arborization based on the scoring system of McPhail.

Results of the dose response of endometrial transformation scored as represented by the McPhail index for 7α-methyl testosterone are provided below in Table 8 and are shown graphically in FIG. 6.

TABLE 8

| | | Average |
|---|---|---|
| | Conc. in | McPhail |
| Compound | mmole | Index |
| 7α-methyl testosterone | 0.8 | 0.17 |
| 7α-methyl testosterone | 2.4 | 0.29 |
| 7α-methyl testosterone | 7.2 | 1.25 |
| 7α-methyl testosterone | 21.6 | 2.21 |

In view of the many possible embodiments to which the principles of the present disclosure may be applied, it should be recognized that the illustrated embodiments are only preferred examples and should not be taken as limiting scope. Rather, the scope is defined by the following claims. We therefore claim as our invention all that comes within the scope and spirit of these claims.

1. A compound of Formula I, or a pharmaceutically acceptable salt, a prodrug, a solvate, or a tautomer thereof 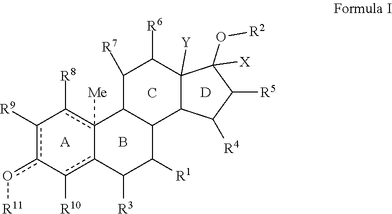
wherein
R¹ is selected from aliphatic, H, D, halogen, aromatic, heteroaliphatic, haloaliphatic, haloheteroaliphatic, heteroaliphatic, or an organic functional group;
R² is selected from —C(O)R^{a}, —C(O)OR^{a}, —C(O)NR^{b}R^{c}, wherein each R^{a} independently is selected from aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; —P(O)(OR^{a})₂, wherein each R^{a} independently is H, aliphatic, aromatic, heteroaliphatic, haloaliphatic, haloheteroaliphatic, heteroaliphatic, or an organic functional group; —S(O)₂R^{a} wherein R^{a} is H, aliphatic, aromatic, heteroaliphatic, haloaliphatic, haloheteroaliphatic, heteroaliphatic, or an organic functional group; or R² can be H or D when R¹¹ is present;
each of R³, R⁴, R⁵, R⁶, R⁷, R⁸, R⁹, and R¹⁰ independently is selected from H, aliphatic, D, aromatic, heteroaliphatic, haloaliphatic, haloheteroaliphatic, heteroaliphatic, or an organic functional group;
R¹¹, if present, is selected from hydrogen, aliphatic, heteroaliphatic, aromatic, heteroaliphatic, haloaliphatic, haloheteroaliphatic, or an organic functional group;
X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group; and
Y is aliphatic; and
provided that R² is not, or is other than, any of the following:
—C(O)(CH₂)₅CH₃; —C(O)CH₂SO₂OR, wherein R is methyl, ethyl, cyclopentyl, cyclohexyl, cycloheptyl, phenyl, phenoxy-lower-alkyl, and lower-alkoxy-phenyl; —C(O)CH₂SO₂OEt; —C(O)Ph; —C(O)Me; —C(O)Et; —C(O)OCH₂adamantyl; —C(O)Oadamantyl; 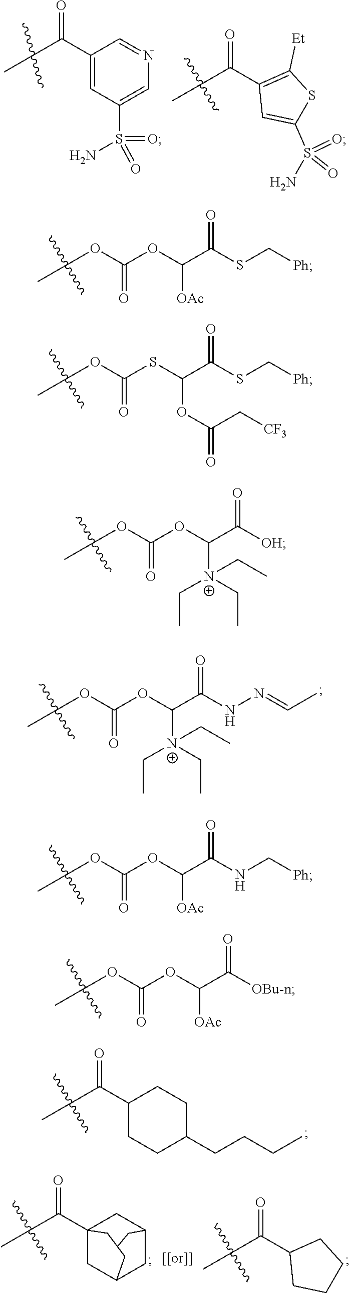
or a heteroaliphatic group comprising a structure selected from

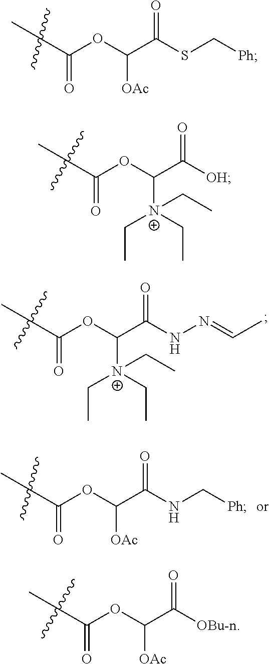

2. The compound of claim 1, wherein:
R¹ is selected from alkyl, H, D, Cl, F, I, Br, alkenyl, alkynyl, aryl, heteroaryl, heteroalkyl, heteroalkenyl, heteroalkynyl, or any combination of these groups;
R² is selected from —C(O)R^{a}, —C(O)OR^{a}, —C(O)NR^{b}R^{c}, wherein each R^{a} independently is selected from alkyl, alkenyl, alkynyl, aryl, heteroaryl, heteroalkyl, heteroalkenyl, heteroalkynyl, halogen, or another organic functional group, and each of R^{b} and R^{c} independently is H, alkyl, alkenyl, alkynyl, aryl, heteroaryl, heteroalkyl, heteroalkenyl, heteroalkynyl, halogen, or another organic functional group; —P(O)(OR^{a})₂, wherein each R^{a} independently is H, alkyl, alkenyl, alkynyl, aryl, heteroaryl, heteroalkyl, heteroalkenyl, heteroalkynyl, or an organic functional group; or —S(O)₂R^{a} wherein R^{a} is H, alkyl, alkenyl, alkynyl, aryl, heteroaryl, heteroalkyl, heteroalkenyl, heteroalkynyl, or an organic functional group;
each of R³, R⁴, R⁵, R⁶, R⁷, R⁸, R⁹, and R¹⁰ independently is selected from H, D, —OH, halogen, alkyl, alkenyl, alkynyl, aryl, heteroaryl, heteroalkyl, heteroalkenyl, heteroalkynyl, or any combination of these groups;
R¹¹, if present, is H, D, or a group selected from an R² group;
X is selected from H, D, aliphatic, heteroaliphatic, —OH, —C(O)R^{a}, —C(O)OR^{a}, or —C(O)NR^{b}R^{c}, wherein each R^{a} independently is alkyl, alkenyl, alkynyl, aryl, heteroaryl, heteroalkyl, heteroalkenyl, heteroalkynyl, or an organic functional group; and each of R^{b} and R^{c} independently is H, alkyl, alkenyl, alkynyl, aryl, heteroaryl, heteroalkyl, heteroalkenyl, heteroalkynyl, or an organic functional group; and
Y is alkyl, alkenyl, or alkynyl.

3. The compound of claim 1, wherein:
R¹ is selected from lower alkyl, Cl, F, I, Br, or phenyl;
R² is selected from —C(O)R^{a}, —C(O)OR^{a}, or —C(O)NR^{b}R^{c}, wherein each R^{a} independently is selected from C₁₋₂₀alkyl, Cl, Br, F, I, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl, and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; —P(O)(OR^{a})₂, wherein each R^{a} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; or —S(O)₂R^{a} wherein R^{a} is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group;
each of R³, R⁴, R⁵, R⁶, R⁷, R⁸, R⁹, and R¹⁰ independently is selected from H, D, —OH, Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, or any combination of these groups;
X is selected from H, D, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, —OH, —C(O)R^{a}, —C(O)OR^{a}, or —C(O)NR^{b}R^{c}, wherein each R^{a} independently is Cl, Br, F, I, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, or C₁₋₁₅heteroaryl; and each of R^{b} and R^{c} independently is H, C₁₋₂₀alkyl, C₂₋₂₀alkenyl, C₂₋₂₀alkynyl, C₁₋₂₀heteroalkyl, C₂₋₂₀heteroalkenyl, C₂₋₂₀heteroalkynyl, C₅₋₁₅aryl, C₁₋₁₅heteroaryl, Cl, Br, F, I, or another organic functional group; and
Y is lower alkyl.

4. The compound of claim 1, wherein
R¹ is selected from methyl, ethyl, Cl, F, I, Br, t-butyl, or phenyl;
R² is selected from —C(O)nPr, —C(O)(CH₂)₉CH₃, —C(O)(CH₂)₁₀CH₃, —C(O)(CH₂)₇C(H)═C(H)(CH₂)₇CH₃, —C(O)(CH₂)₇C(H)═C(H)CH₂C(H)═C(H)(CH₂)₄CH₃, —C(O)O(CH₂)₉CH₃, —C(O)Cl, —C(O)Ome, —C(O)OC(CH₃)₃, —C(O)O(CH₂)₃CH₃, —C(O)O(CH₂)₄CH₃, —C(O)NH(CH₂)₉CH₃, —C(O)O(CH₂)₁₁CH₃, —C(O)NH(CH₂)₁₁CH₃, —C(O)N(CH₃)CH₃, —C(O)N(H)CH₃, —C(O)N(CH₂)₄CH₃, —C(O)N(H)(CH₂)₄CH₃, —C(O)N(CH₃)C(O)N(H)CH₃, or —S(O)₂Ph-p-Me;
each of R³, R⁹, and R¹⁰ independently is H or D;
R⁴, R⁵, and R⁶ are H;
R⁷ is H or Me;
R⁸ is H;
X is H or —CCH; and
Y is methyl or ethyl.

5. The compound of claim 1, wherein the compound is represented by Formula II, III, or IV

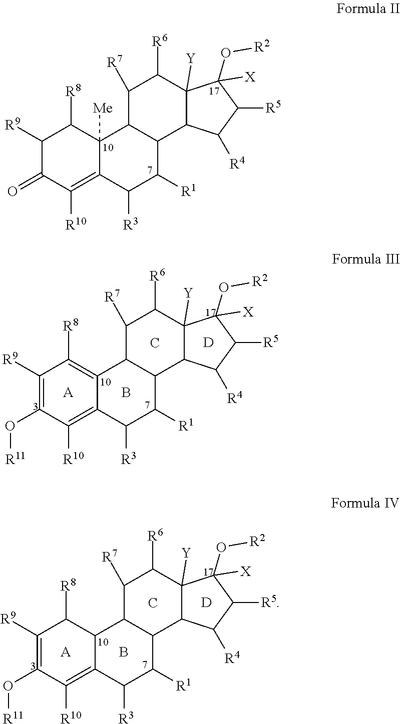

6. The compound of claim 5, wherein;
(i) the compound has a Formula II, and R¹ is phenyl; R³ is H or D; R⁴, R⁵, and R⁶ are H; R⁷ is H or Me; R⁸ is H; R⁹ is H or D; R¹⁰ is H or D; X is H or —CCH; Y is methyl or ethyl; or
(ii) the compound has a Formula III and R¹ is aliphatic or aromatic: R² is hydrogen; R³ is H or D; R⁴, R⁵, and R⁶ are H; R⁷ is H or Me; R⁸ is H; R⁹ is H or D; and R¹⁰ is H or D; R¹¹ is H or D; X is H or —CCH; and Y is methyl or ethyl.

7. (canceled)

8. The compound of claim 1, wherein the compound is represented by Formula IA, IB, IIA, IIB, IIIA, IIIB, IVA, or IVB, or a pharmaceutically acceptable salt, a prodrug, a solvate, or a tautomer thereof

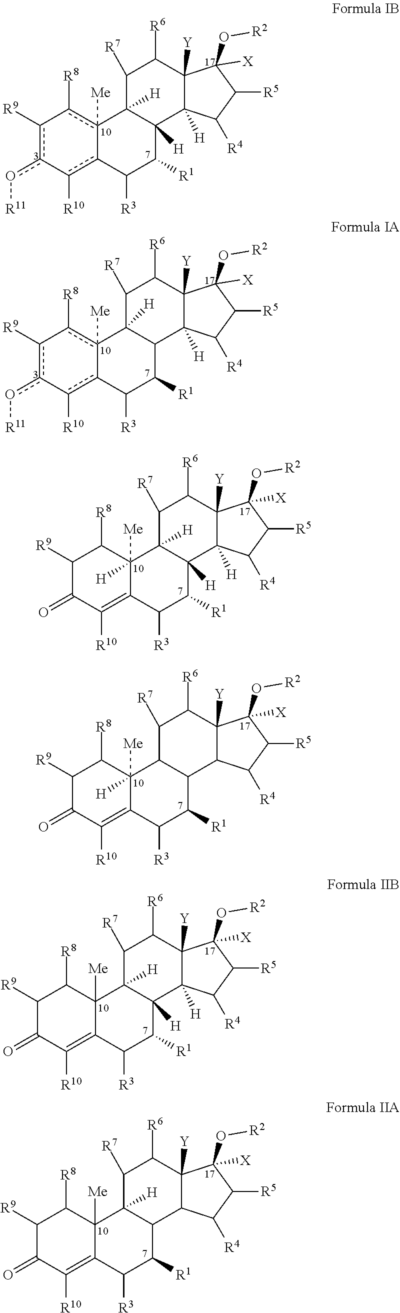

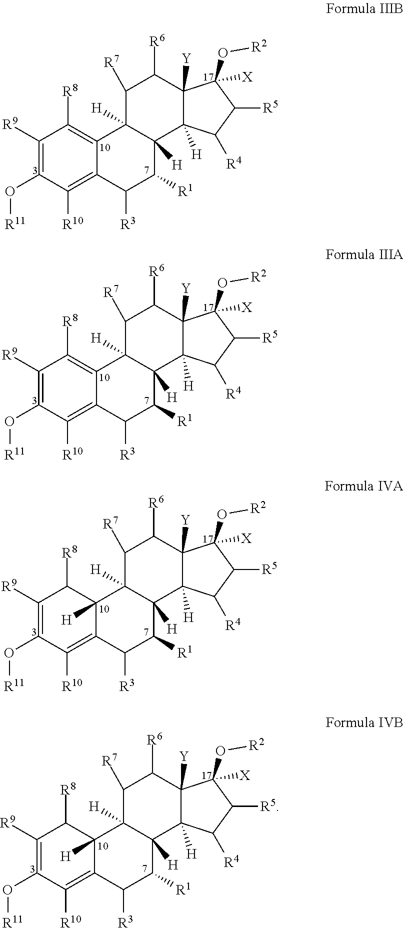

9. The compound of claim 1, wherein the compound is represented by Formula V, VI, VII, or VIII or a pharmaceutically acceptable salt, a prodrug, a solvate, or a tautomer thereof

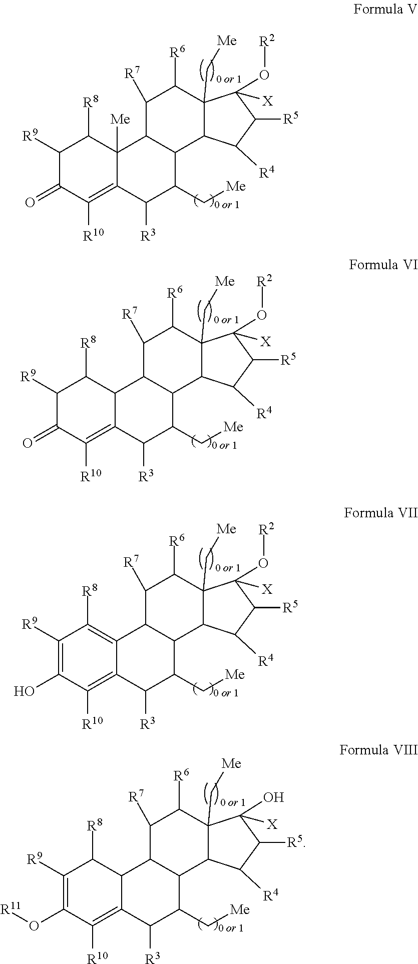

10. The compound of claim 9, wherein
R² is —C(O)(CH₂)ₙCH₃ or —C(O)O(CH₂)ₙCH₃, wherein n is an integer selected from 0 to 15; —C(O)NR^{b}R^{c}, wherein R^{b} is H or lower alkyl and R^{c} is lower alkyl, or heteroaliphatic; or —S(O)₂R^{a} wherein R^{a} is aromatic;
each of R³, R⁴, R⁵, R⁶, R⁷, R⁸, R⁹, and R¹⁰ is H or D;
R¹¹ is —C(O)O(CH₂)ₙCH₃, wherein n is an integer selected from 0 to 15; and
X is H or —CCH.

11. (canceled)

12. The compound of claim 1, wherein the compound is selected from

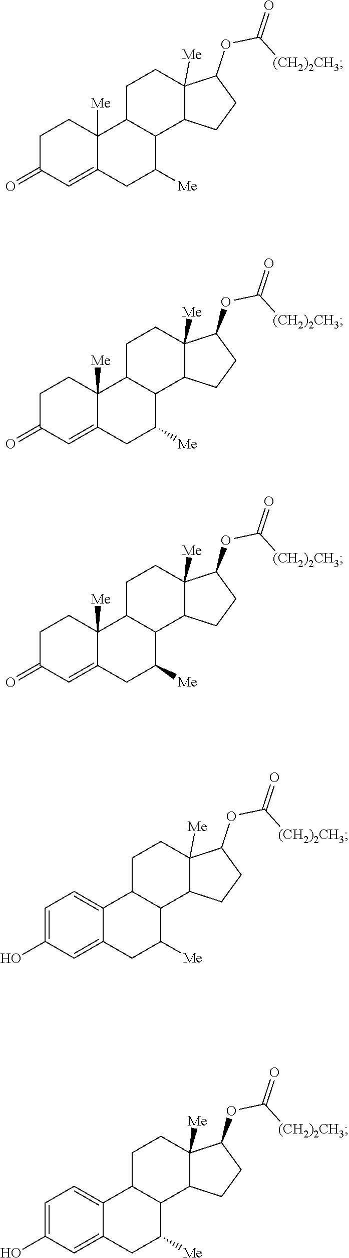

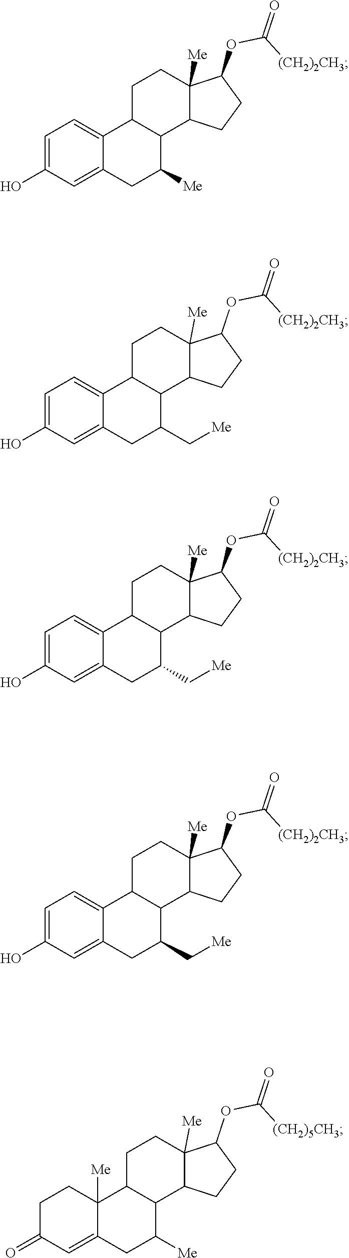

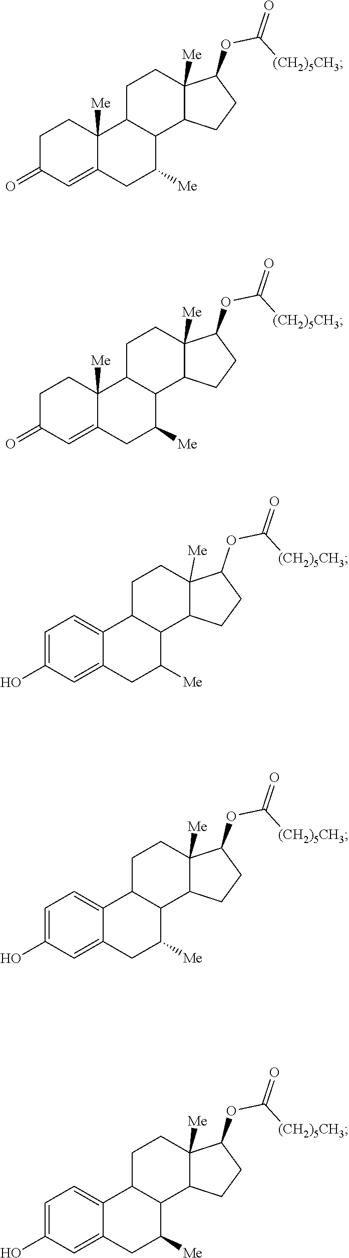

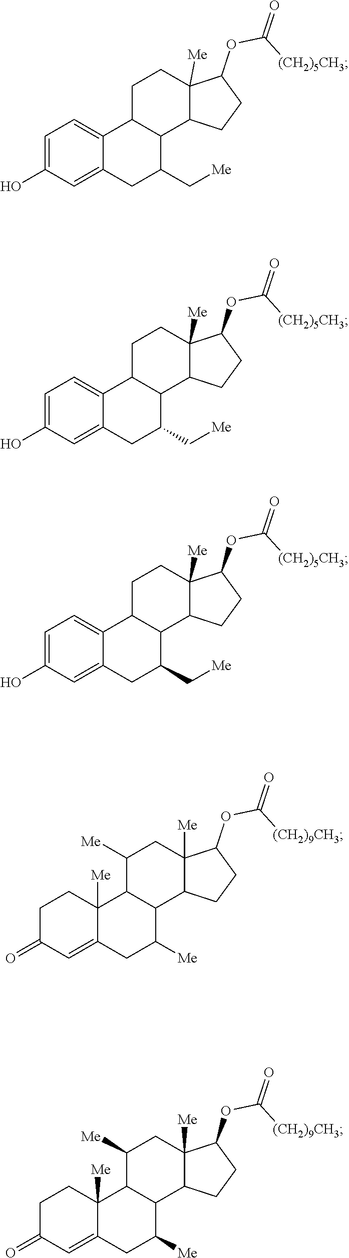

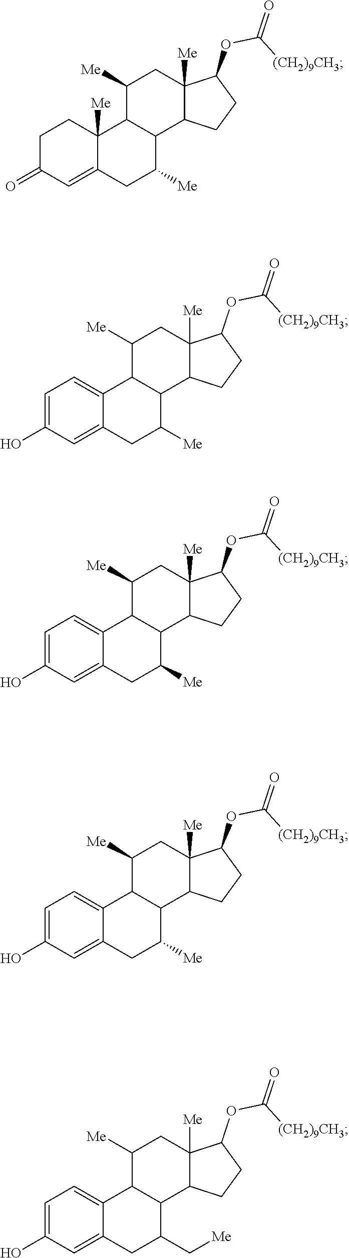

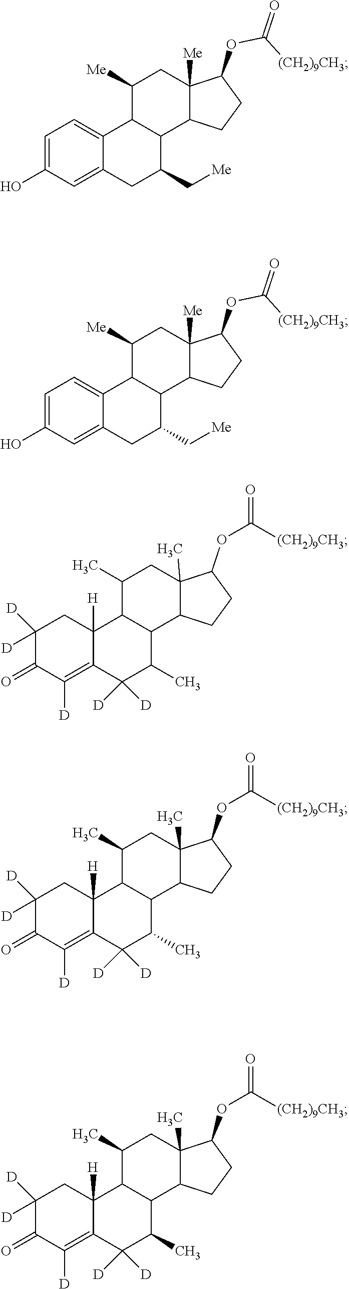

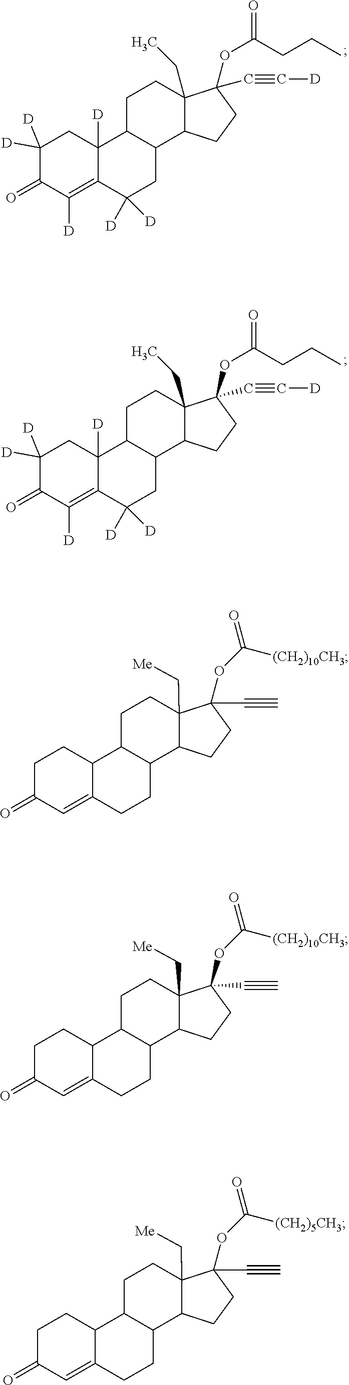

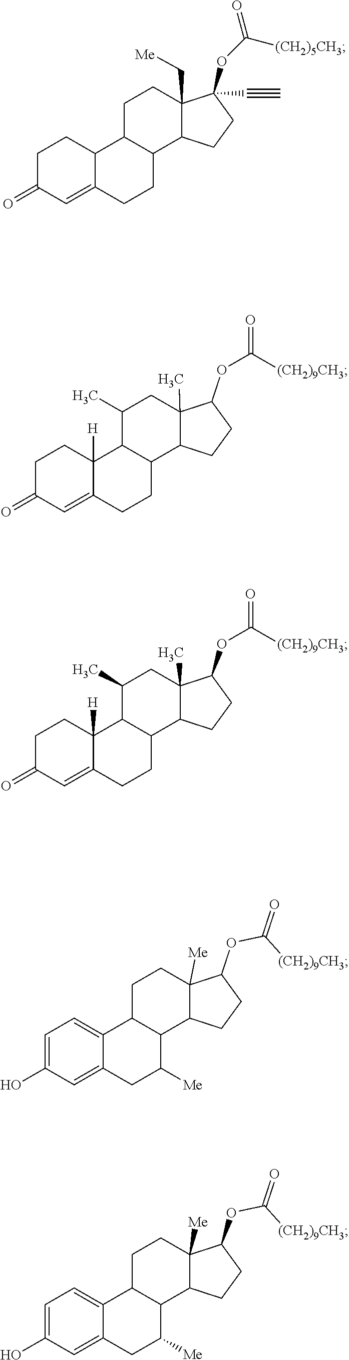

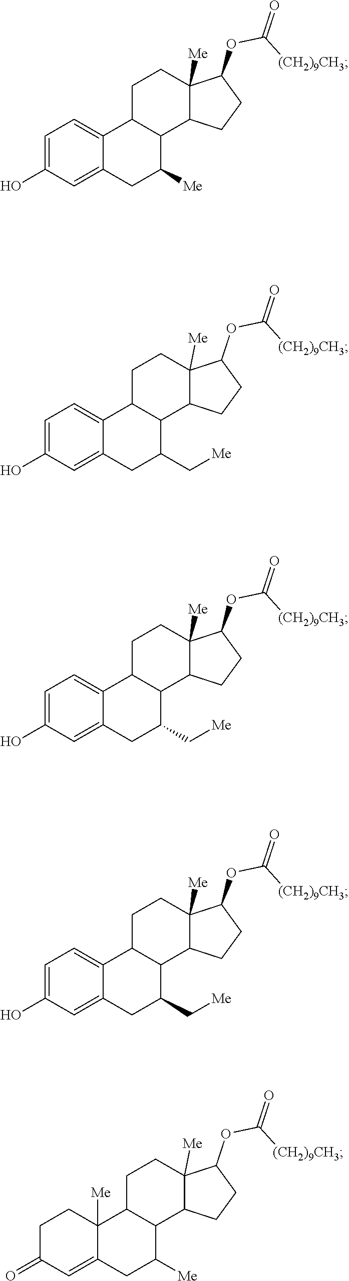

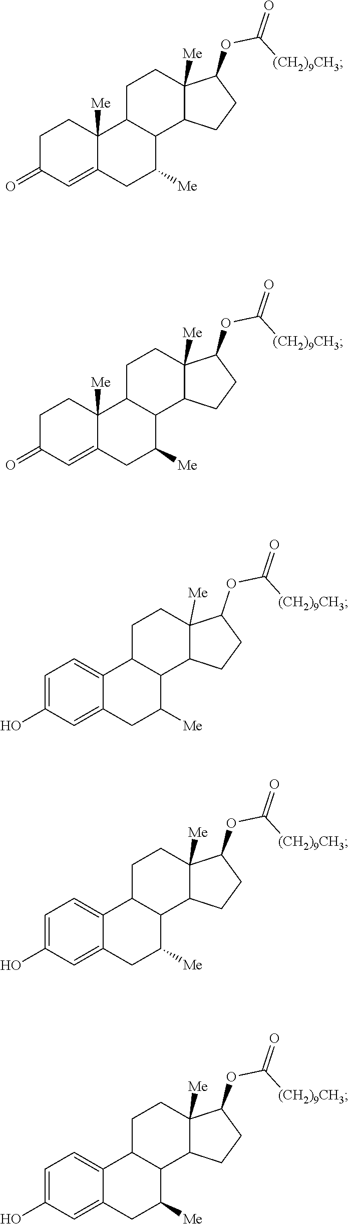

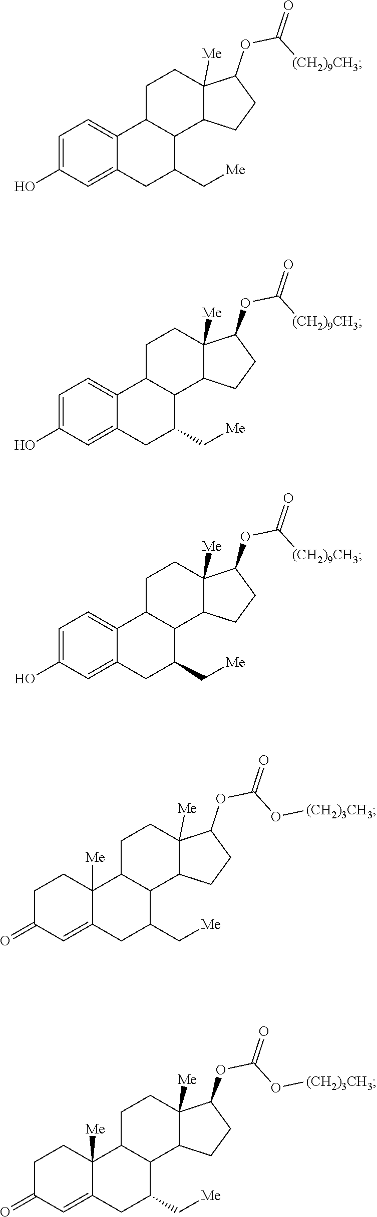

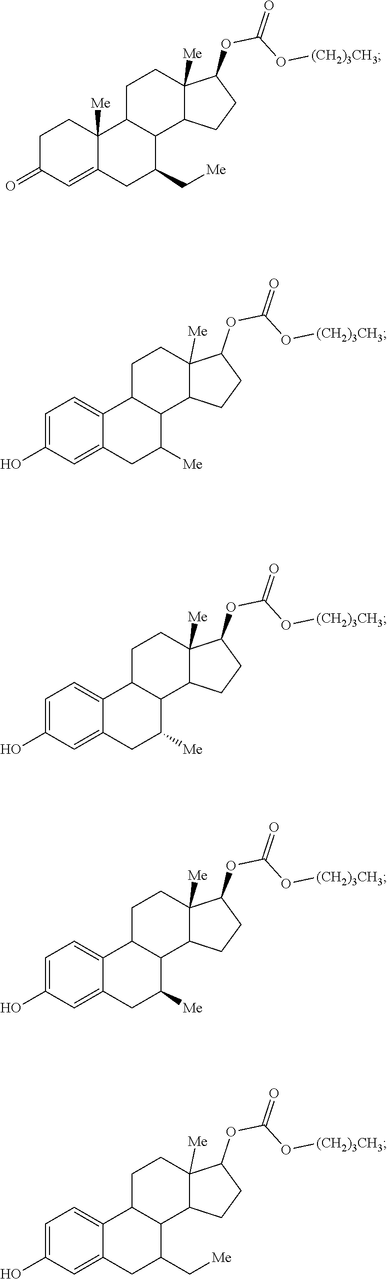

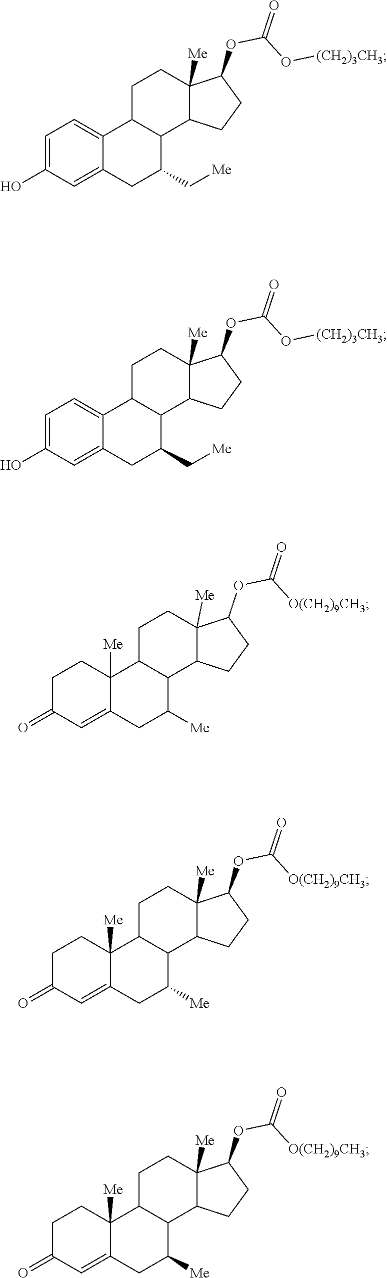

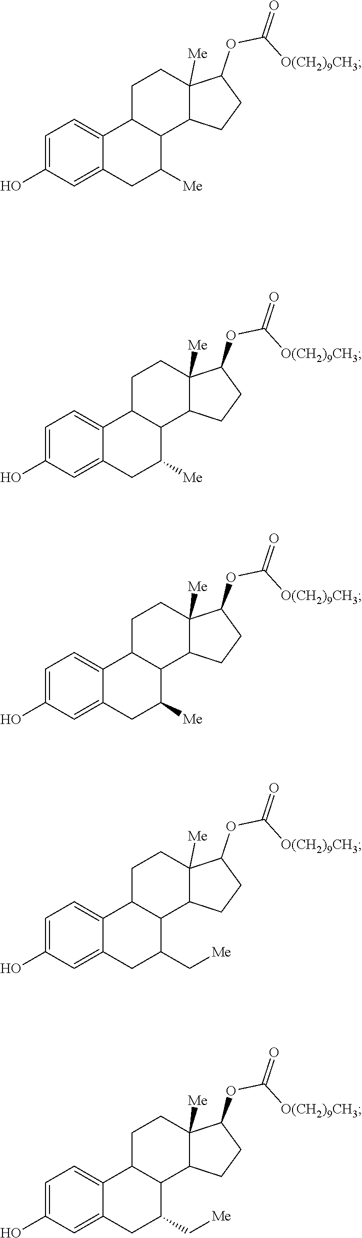

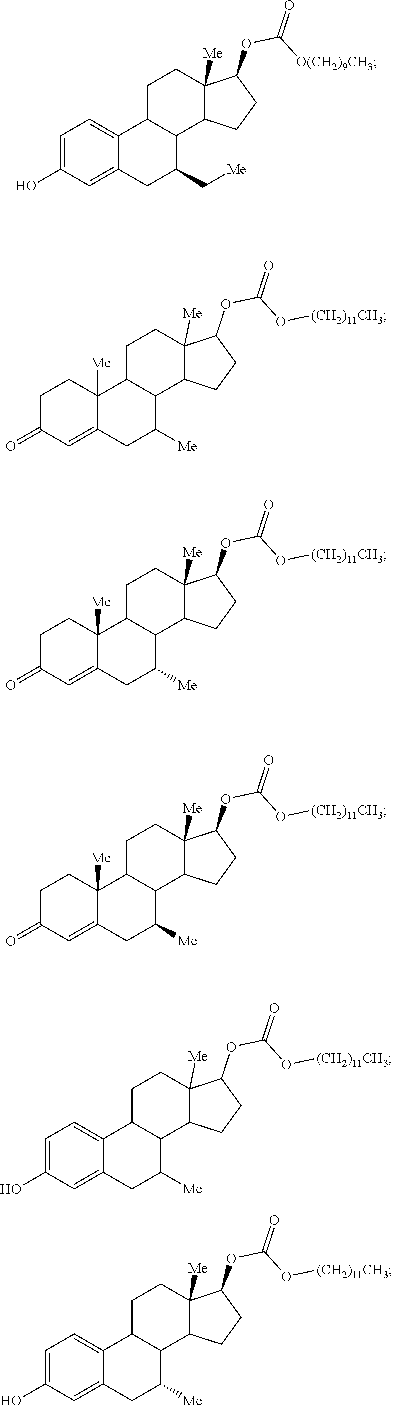

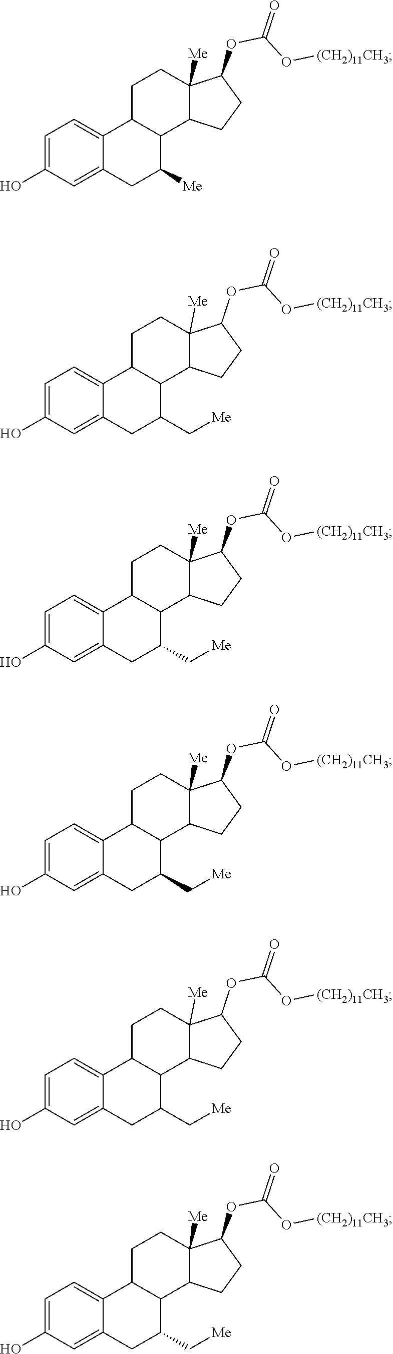

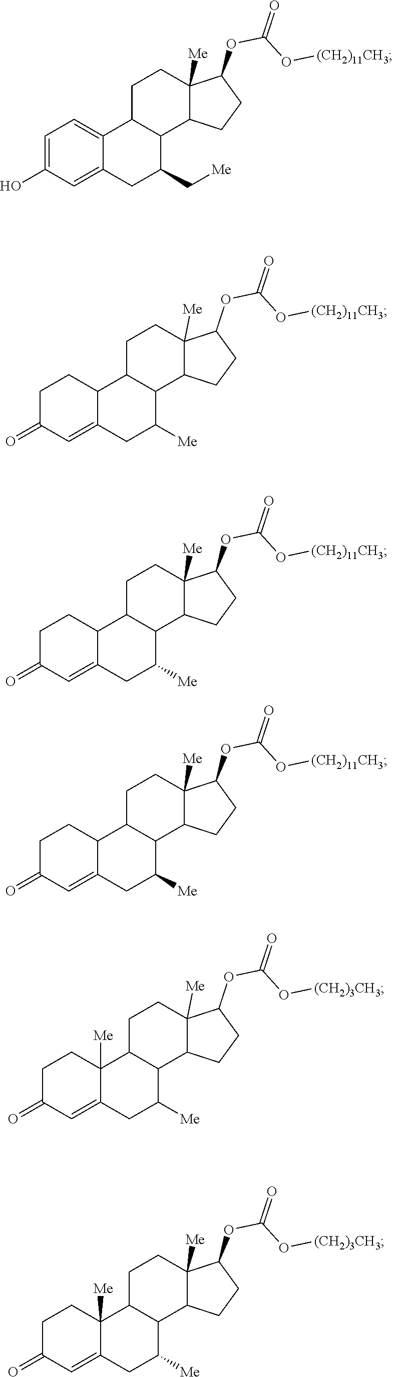

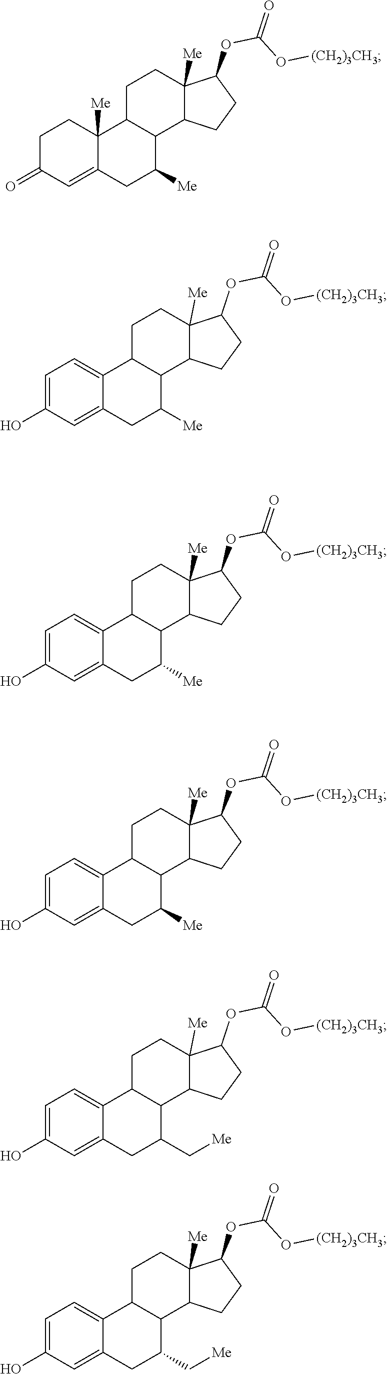

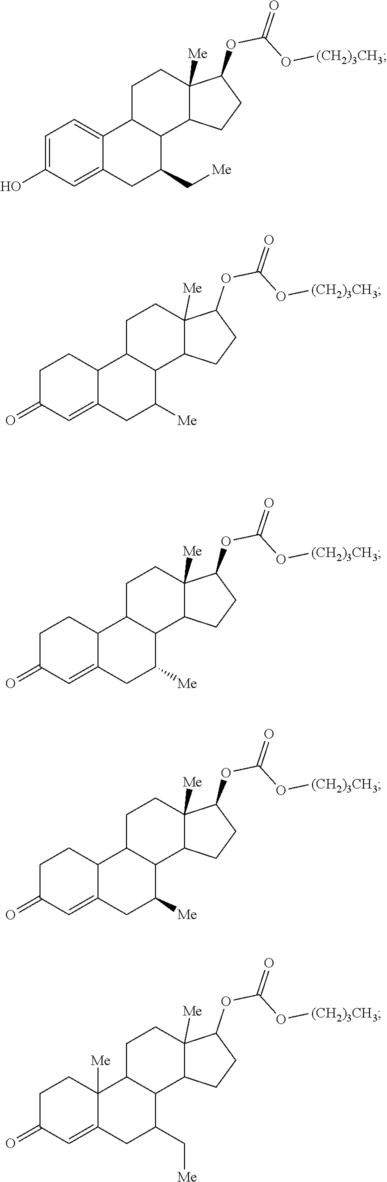

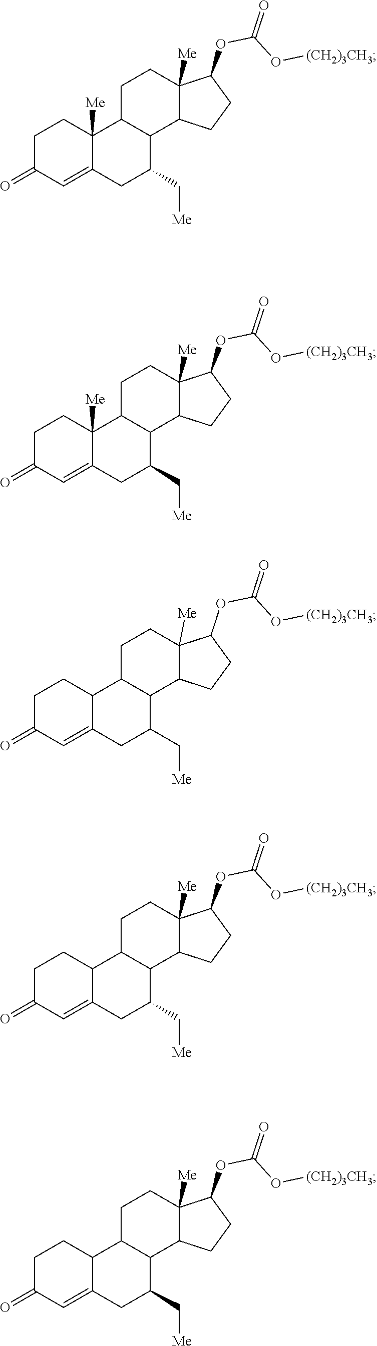

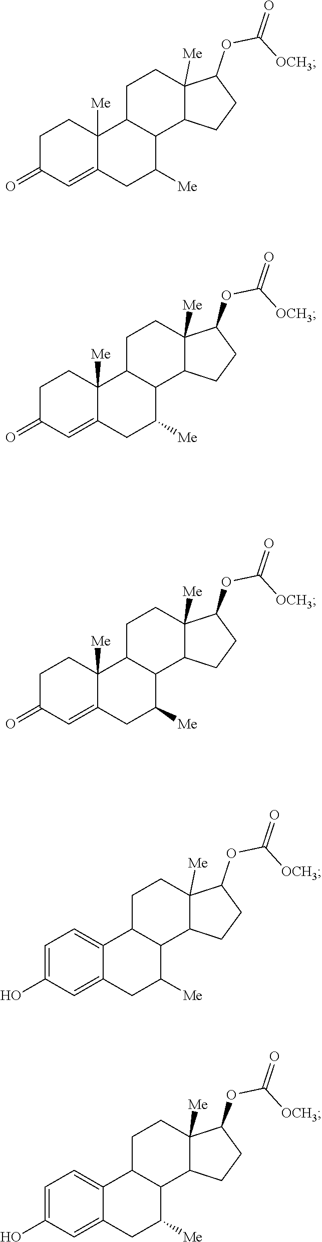 [SMILES: COC(=O)OC1CCC2C3C(C)CC4=CC(=O)CCC4(C)C3CCC12C ; COC(=O)O[C@H]1CCC2C3C(CC[C@@]21C)[C@@]1(C)CCC(=O)C=C1C[C@H]3C ; COC(=O)O[C@H]1CCC2C3C(CC[C@@]21C)[C@@]1(C)CCC(=O)C=C1C[C@@H]3C ; COC(=O)OC1CCC2C3C(C)Cc4cc(O)ccc4C3CCC12C ; COC(=O)O[C@H]1CCC2C3C(CC[C@@]21C)c1ccc(O)cc1C[C@H]3C]

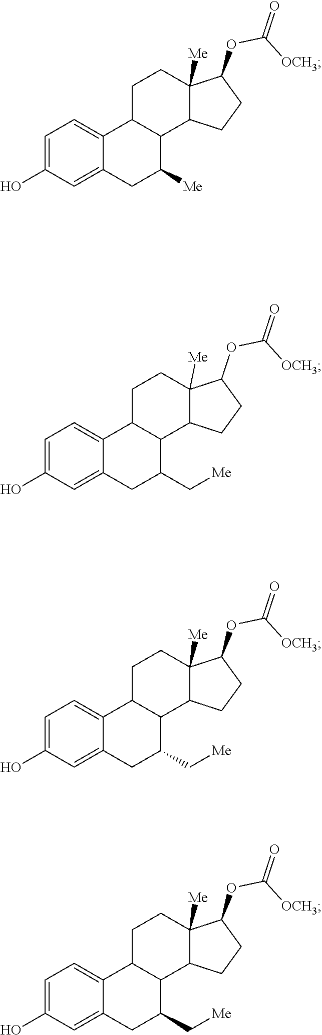 [SMILES: COC(=O)O[C@H]1CCC2C3C(CC[C@@]21C)c1ccc(O)cc1C[C@@H]3C ; CCC1Cc2cc(O)ccc2C2CCC3(C)C(OC(=O)OC)CCC3C12 ; CC[C@@H]1Cc2cc(O)ccc2C2CC[C@@]3(C)C(CC[C@@H]3OC(=O)OC)C21 ; CC[C@H]1Cc2cc(O)ccc2C2CC[C@@]3(C)C(CC[C@@H]3OC(=O)OC)C21]

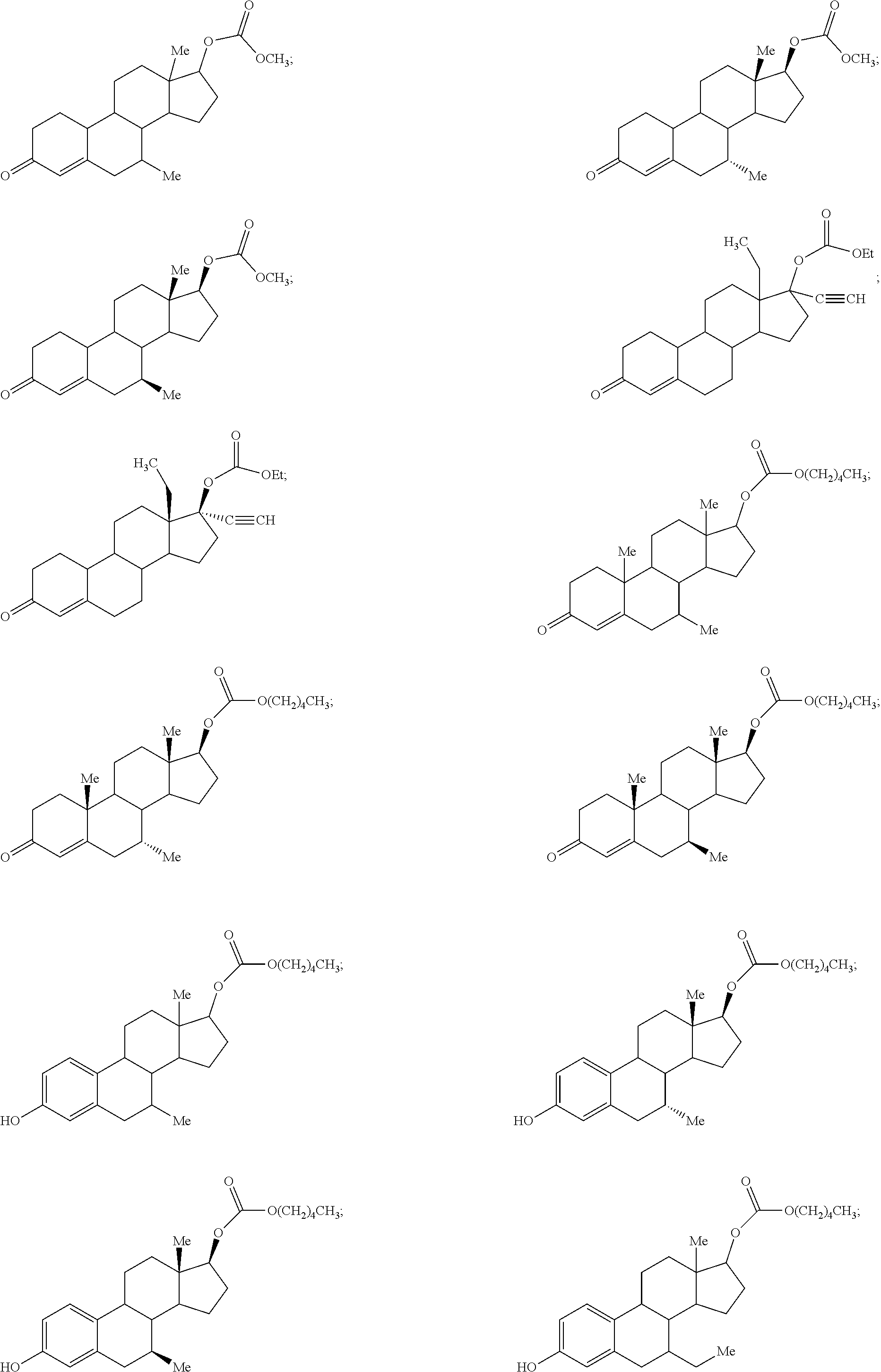

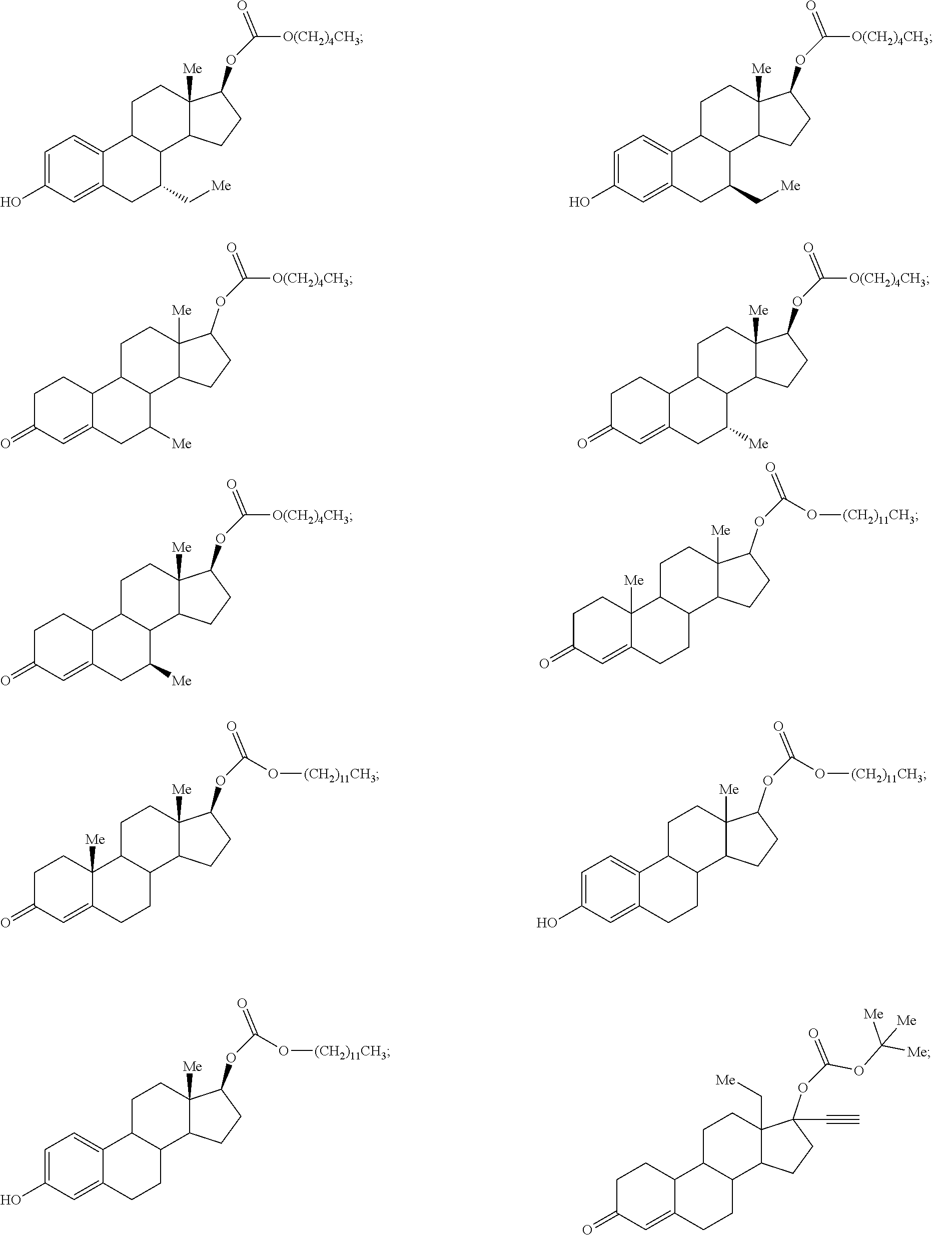

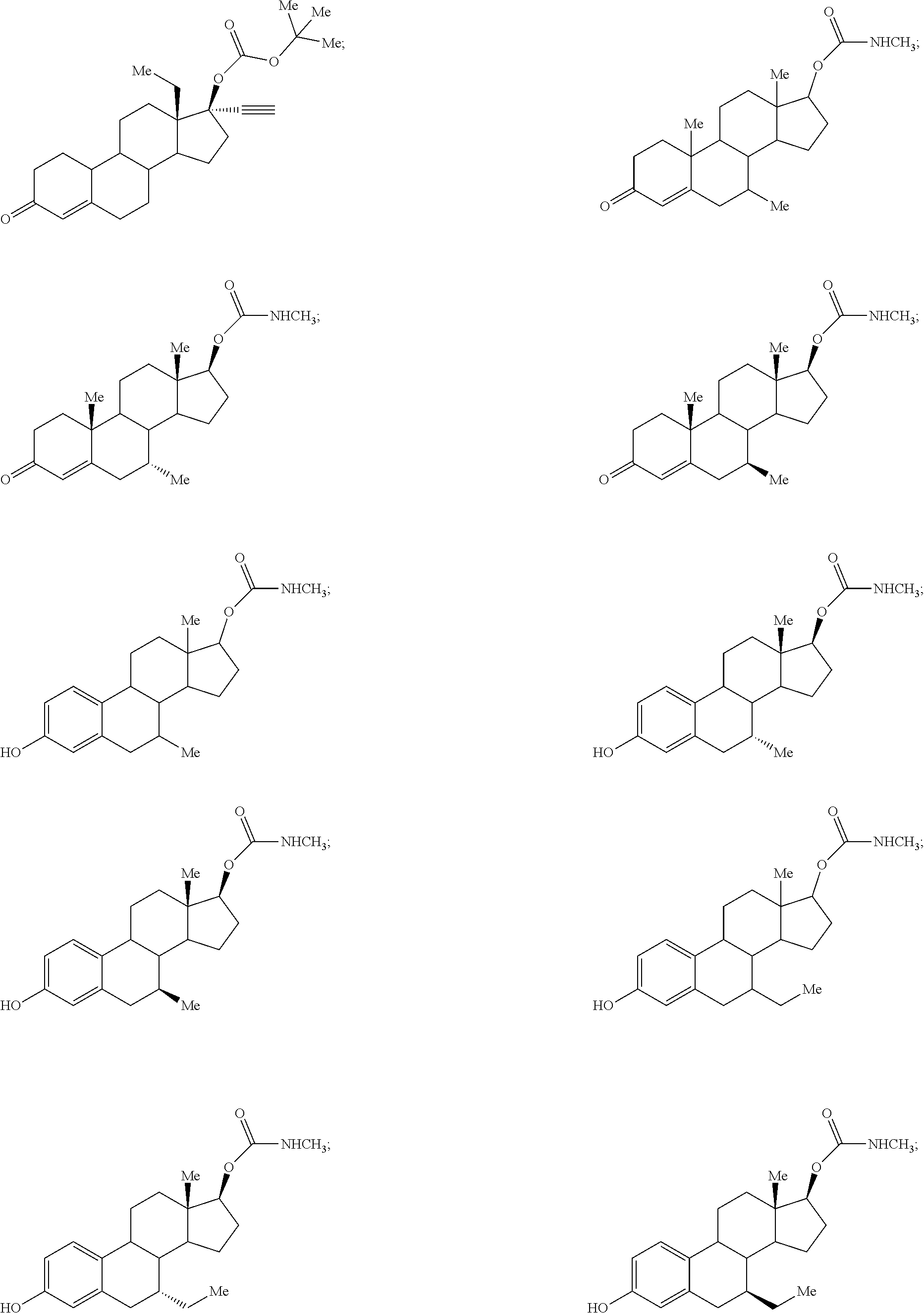

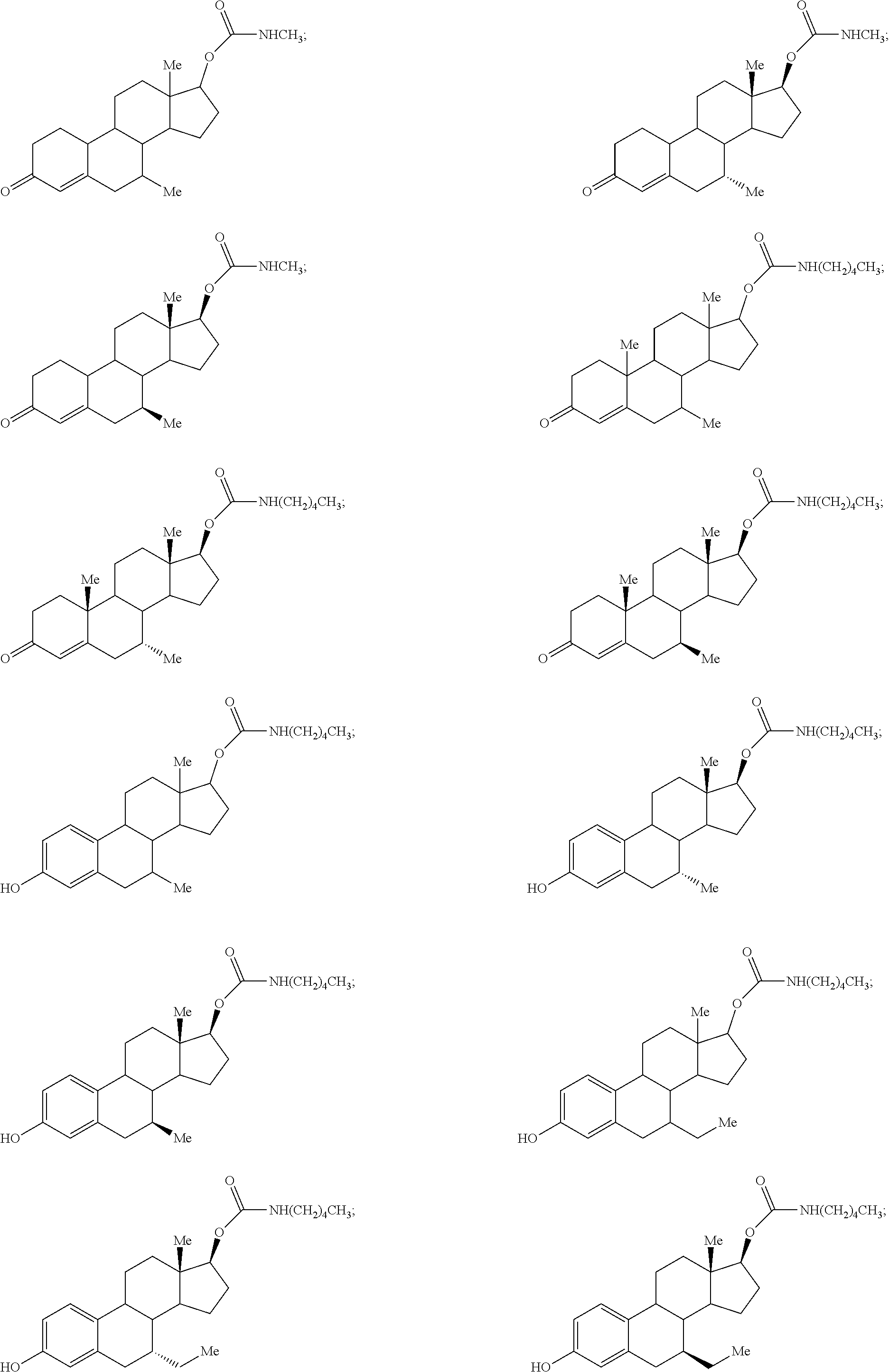

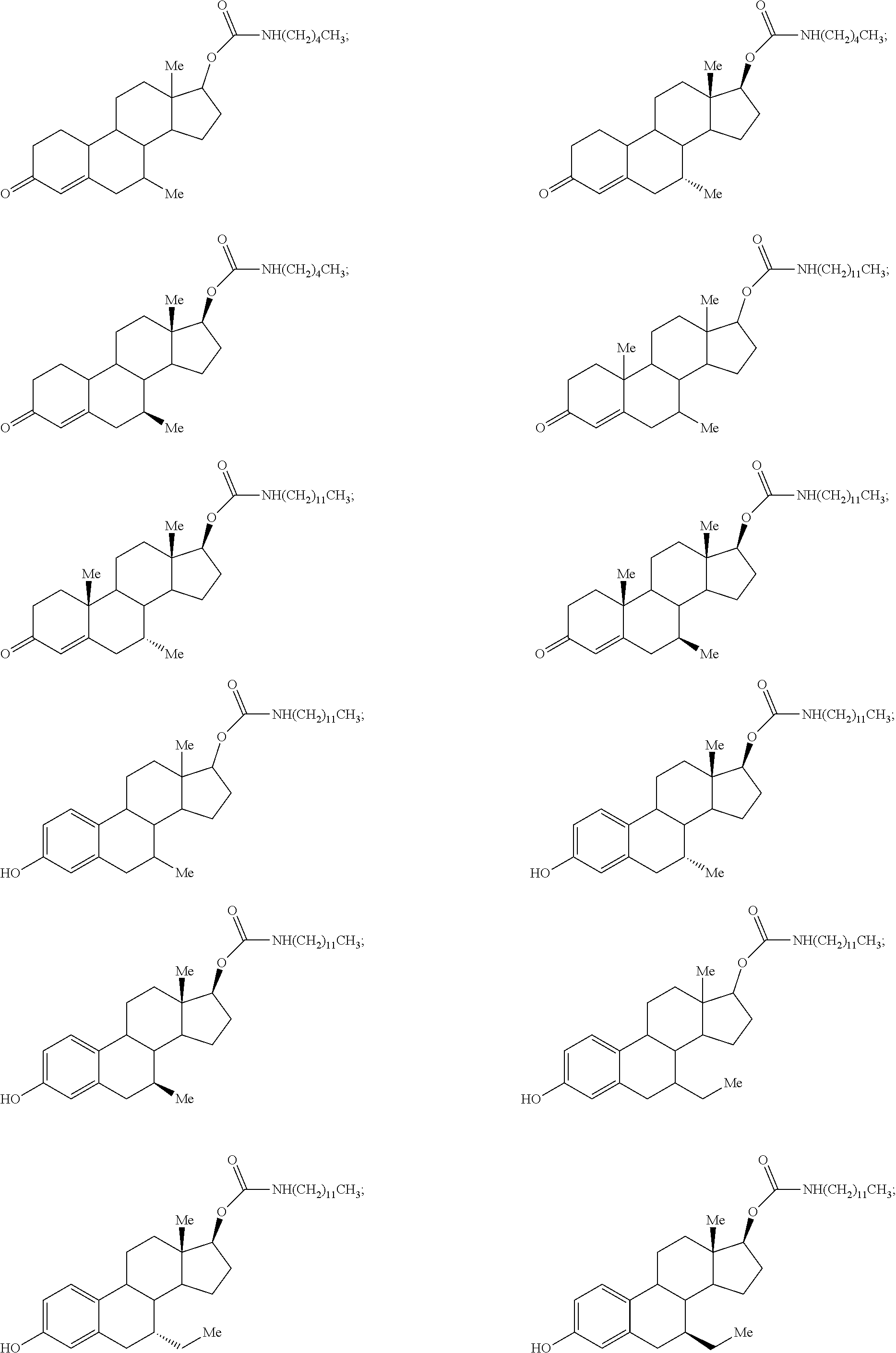

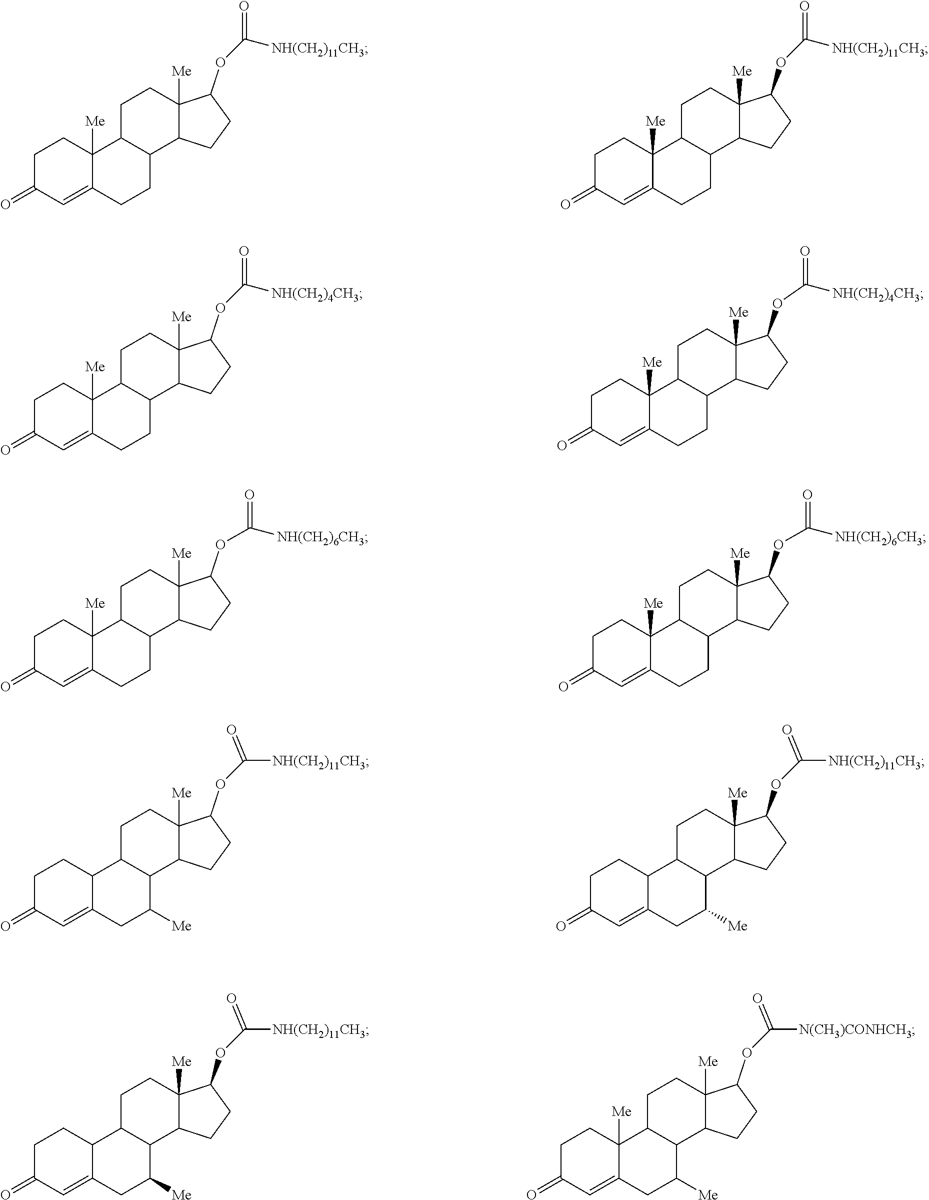

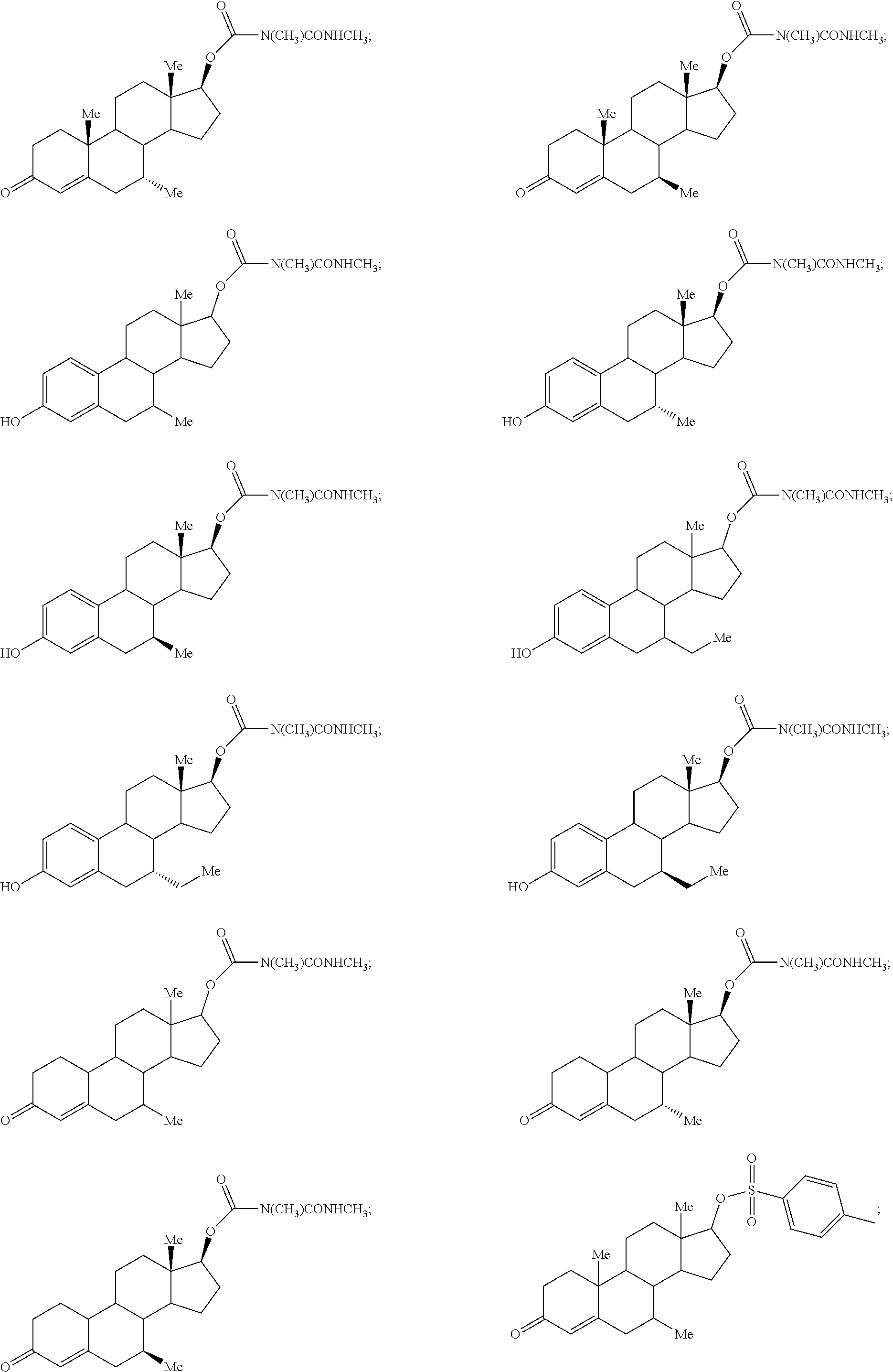

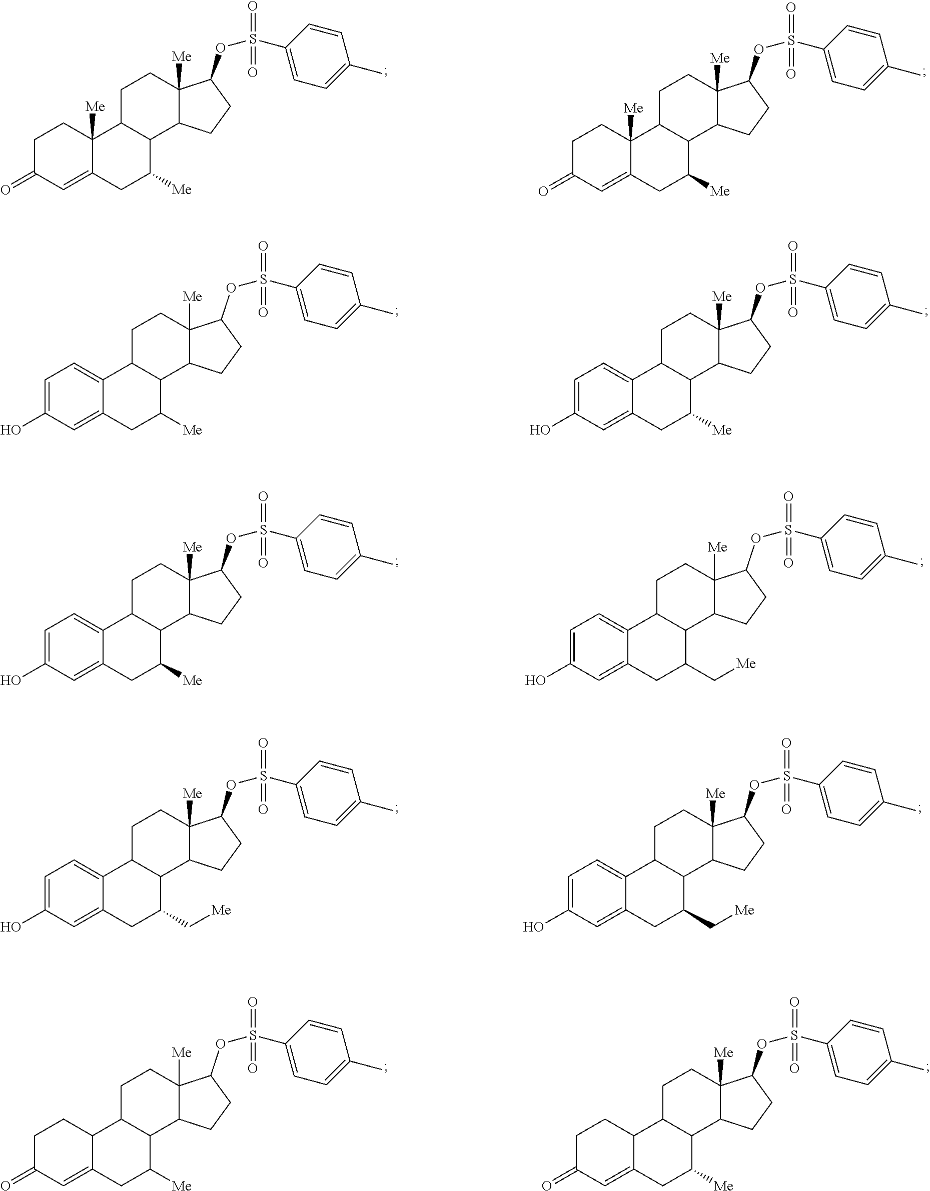

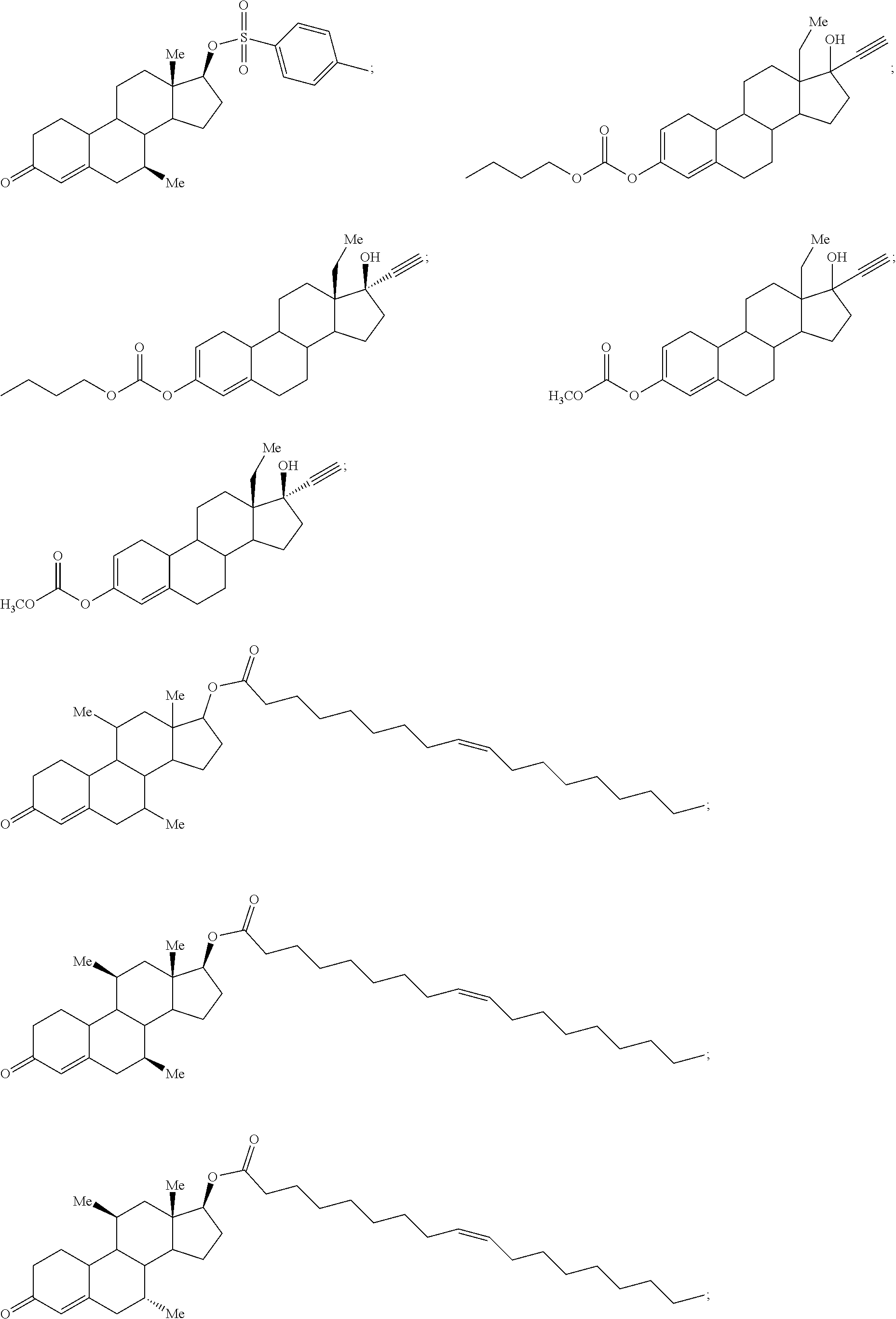

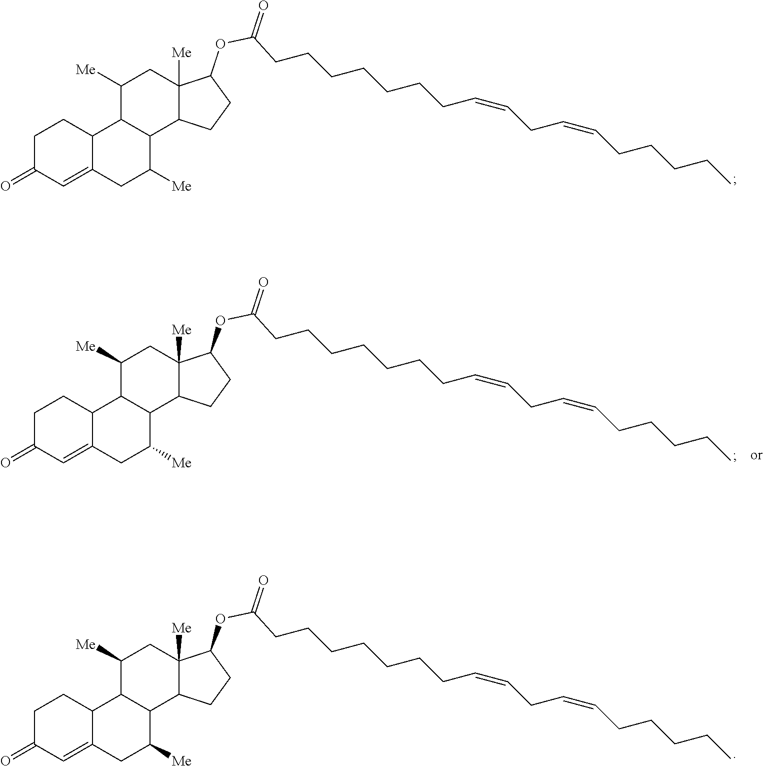 [SMILES: CCCCC/C=C\C/C=C\CCCCCCCC(=O)OC1CCC2C3C(C)CC4=CC(=O)CCC4C3C(C)CC12C ; CCCCC/C=C\C/C=C\CCCCCCCC(=O)O[C@H]1CCC2C3C(C4CCC(=O)C=C4C[C@H]3C)[C@@H](C)C[C@@]21C ; CCCCC/C=C\C/C=C\CCCCCCCC(=O)O[C@H]1CCC2C3C(C4CCC(=O)C=C4C[C@@H]3C)[C@@H](C)C[C@@]21C]

13. (canceled)

14. A dosage form, comprising the compound of claim 1, or a pharmaceutically acceptable salt, a prodrug, a solvate, or a tautomer thereof; or a pharmaceutically acceptable composition thereof, wherein the dosage form is a tablet, a capsule, an implant, a patch, a microneedle array, an aerosol, or gel.

15. An oligomer compound, comprising a first steroidal-based compound covalently coupled to a first linker group via an oxygen atom attached to a functional group positioned at C17 of the steroidal-based compound; wherein the first linker group is further covalently coupled to a second steroidal-based compound or a therapeutic agent and wherein the first steroidal-based compound has a Formula IX, or a pharmaceutically acceptable salt, a prodrug, a solvate, or a tautomer thereof 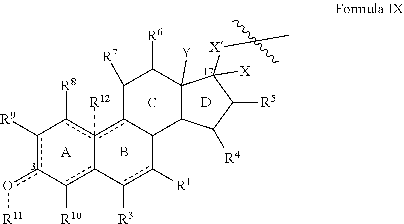
wherein X′ is bound to the first linker group; and wherein
R¹ is selected from aliphatic, H, D, halogen, heteroaliphatic, aromatic, heteroaliphatic, haloaliphatic, haloheteroaliphatic, or an organic functional group;
each of R³, R⁴, R⁵, R⁶, R⁸, R⁹, and R¹⁰ independently is selected from H, D, aliphatic, aromatic, heteroaliphatic, haloaliphatic, haloheteroaliphatic, heteroaliphatic, or an organic functional group;
R⁷ is selected from H, ═O, D, aliphatic, aromatic, heteroaliphatic, haloaliphatic, haloheteroaliphatic, heteroaliphatic, or an organic functional group;
R¹¹, when present, is selected from H, aliphatic, heteroaliphatic, aromatic, heteroaliphatic, haloaliphatic, haloheteroaliphatic, an organic functional group, or a second linker group;
R¹², when present, is H, D, or aliphatic;
X is selected from H, D, aliphatic, heteroaliphatic, —OH, —C(O)R^{a}, —C(O)OR^{a}, or —C(O)NR^{b}R^{c}, wherein each R^{a} independently is H, aliphatic, aromatic, heteroaliphatic, haloaliphatic, haloheteroaliphatic, heteroaliphatic, or an organic functional group, and each R^{b} and R^{c} independently is H, aliphatic, aromatic, heteroaliphatic, haloaliphatic, haloheteroaliphatic, heteroaliphatic, or an organic functional group;
X′ is oxygen or —C(O)(CH₂)ₚ—, wherein p is an integer selected from 1 to 10; and
Y is aliphatic.

16. (canceled)

17. The oligomer compound of claim 15, wherein
each of R³, R⁴, R⁵, R⁶, R⁸, R⁹, and R¹⁰ are H or D;
each of R⁷, R¹², and Y independently is lower alkyl;
R¹ is alkyl, Cl, F, I, Br, alkenyl, alkynyl, aryl, heteroaryl, heteroalkyl, heteroalkenyl, heteroalkynyl, or any combination thereof;
X is hydrogen, —OH, or —CCH; and
R¹¹, when present, is H, lower alkyl, or a second linker group, —C(O)Ph, or —C(Z)(CH₂)_{q}CH₃, wherein Z is S, O, or NH and q is an integer selected from 0 to 10.

18. The oligomer compound of claim 15, wherein the first steroidal-based compound is selected from 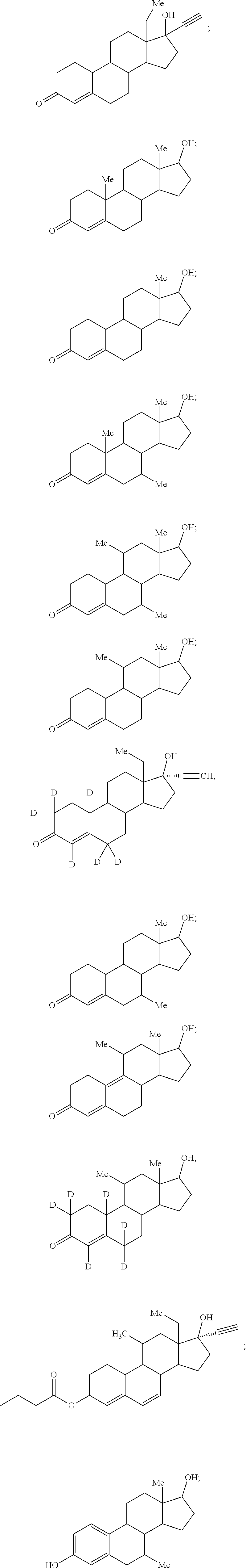 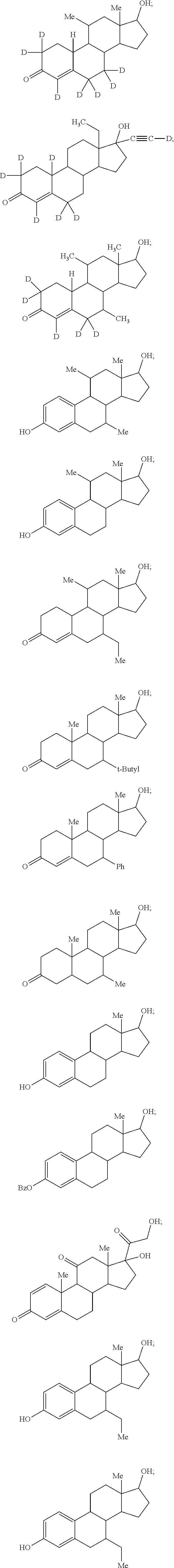 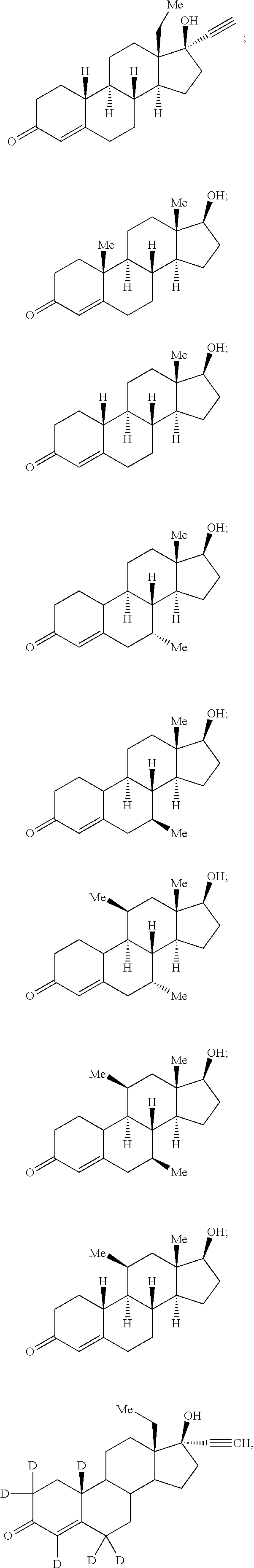 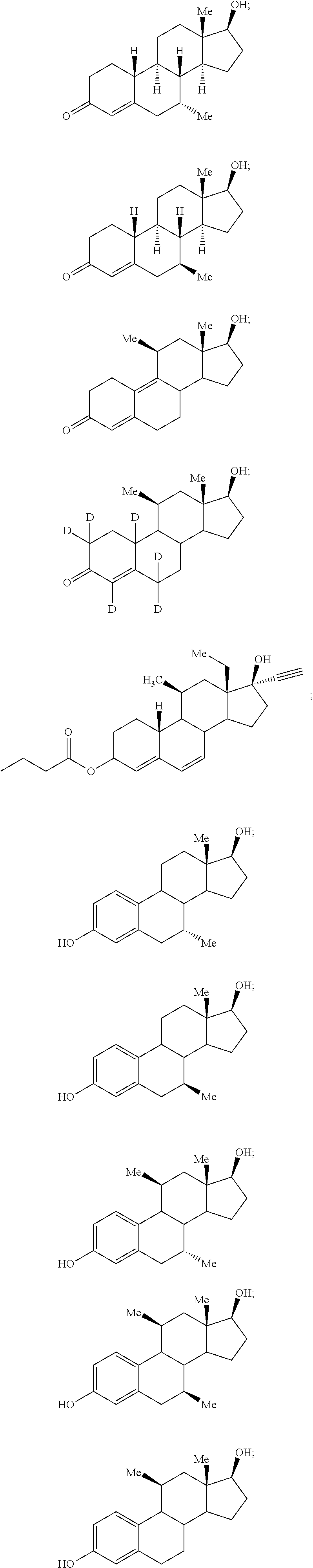 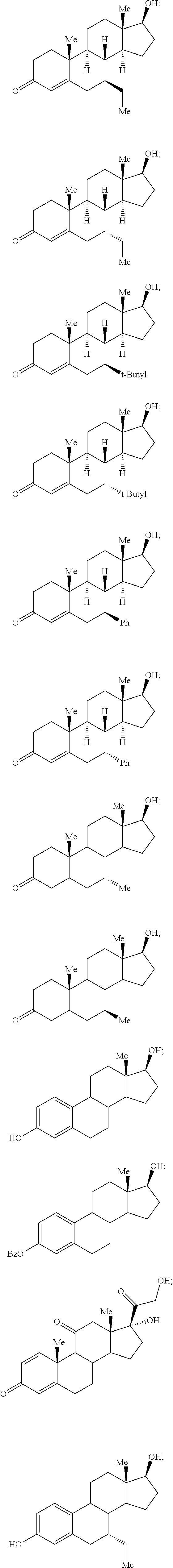 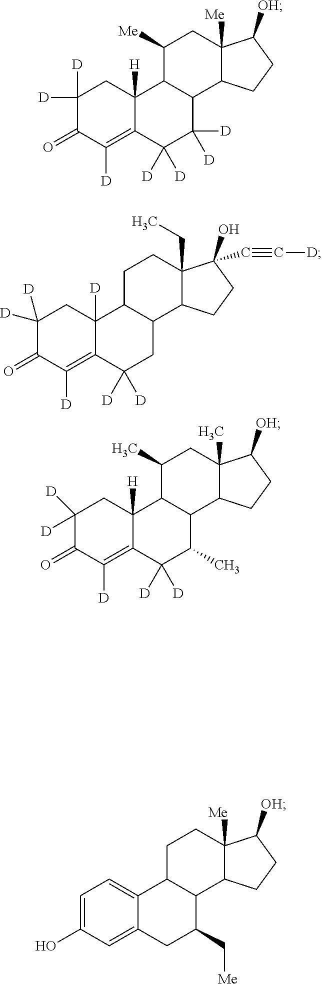
or a pharmaceutically acceptable salt, a prodrug, a solvate, or a tautomer thereof; and wherein the first linker is attached to the oxygen atom of the C17 hydroxyl group, or a hydroxyl group of a —C(O)CH₂OH group attached at C17, such that the hydrogen atom of any such C17 hydroxyl group is replaced with the bond to the first linker; and the second linker group, if present, is attached to the oxygen atom of the C3 hydroxyl group.

19. The oligomer compound of claim 15, wherein the first linker group and/or the second linker group has a Formula X 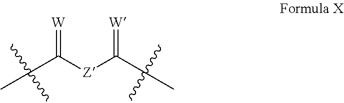
wherein
each of W and W′ independently are oxygen or sulfur; and
Z′ is selected from —(CH₂)ₘ—; —O(CH₂)ₘO—; —NR^{e}(CH₂)ₘNR^{e}—; or —(CH₂)ₘNR^{e}C(O)(CH₂)ₘ wherein R^{e} is H, aliphatic, heteroaliphatic, aromatic, heteroaliphatic, haloaliphatic, haloheteroaliphatic, or a combination thereof and each of m and m′ independently is an integer ranging from 1 to 20.

20-21. (canceled)

22. The oligomer compound of claim 15, wherein the oligomer compound comprises the therapeutic agent, which is a gonadotropin-releasing hormone (“GnRH”) antagonist and/or agonist, a E3 ubiquitin ligase recruiting ligand, an anticancer agent, a kinase antagonist and/or agonist, a GPCR antagonist and/or agonist, an antimalarial agent, an antifungal agent, an antiviral agent, an antibacterial agent, an immunosuppressant, an anti-inflammatory agent, or a pulmonary agent.

23. The oligomer compound of claim 15, wherein the oligomer compound comprises the second steroidal-based compound, which is the same or different from the first steroidal-based compound, and wherein the first linker group is covalently coupled to the first steroidal-based compound via the functional group at the C17 position and via a functional group at the C17 position of the second steroidal-based compound.

24. (canceled)

25. The oligomer compound of claim 15, wherein the oligomer compound comprises a third steroidal-based compound.

26. The oligomer compound of claim 15, wherein the oligomer compound comprises the therapeutic agent and wherein the first linker group is covalently coupled to the first steroidal-based compound via the functional group at the C17 position and via a functional group of the therapeutic agent, and wherein the oligomer compound further comprises an additional steroidal-based compound.

27. The oligomer compound of claim 15, wherein the oligomer compound is selected from 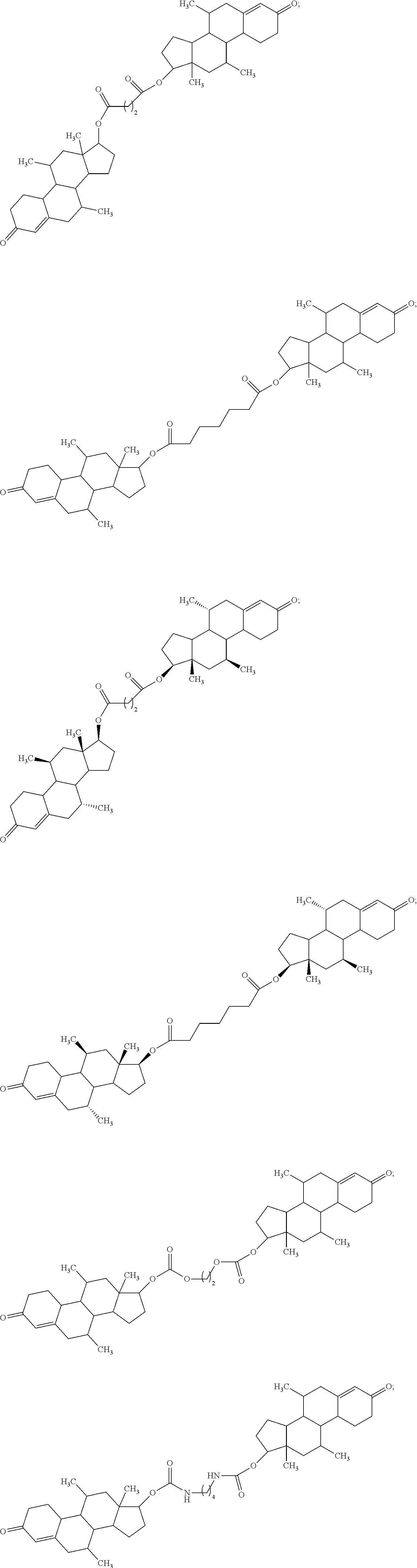 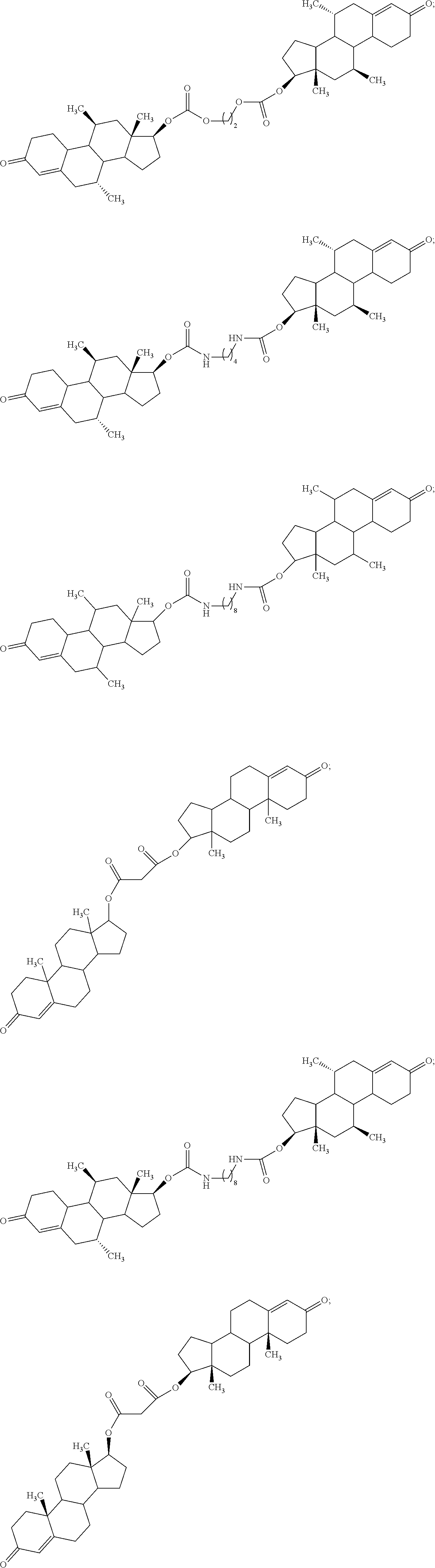 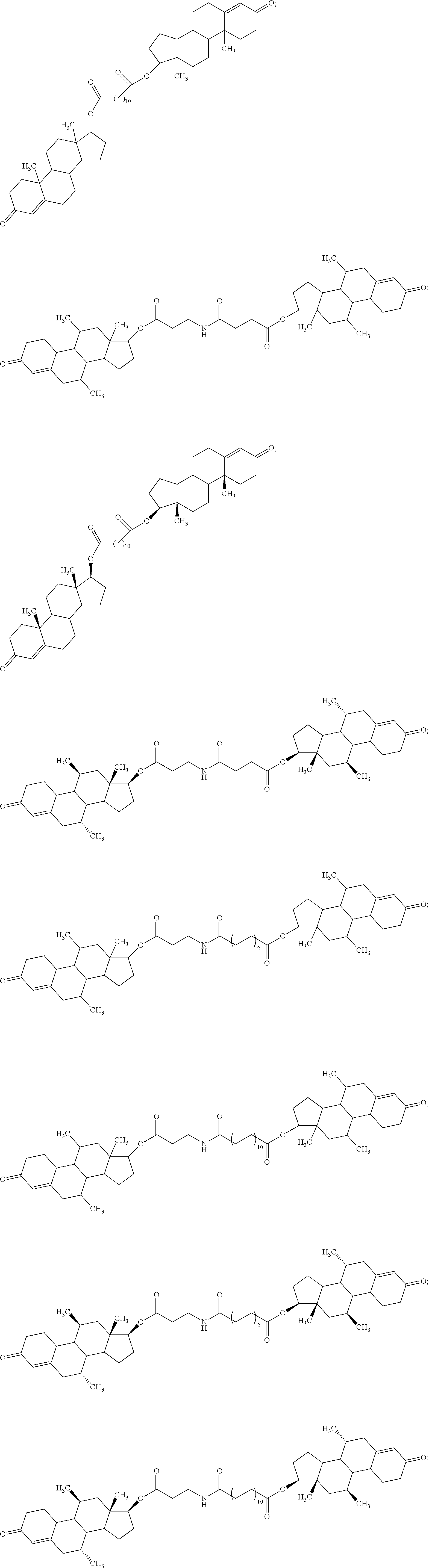 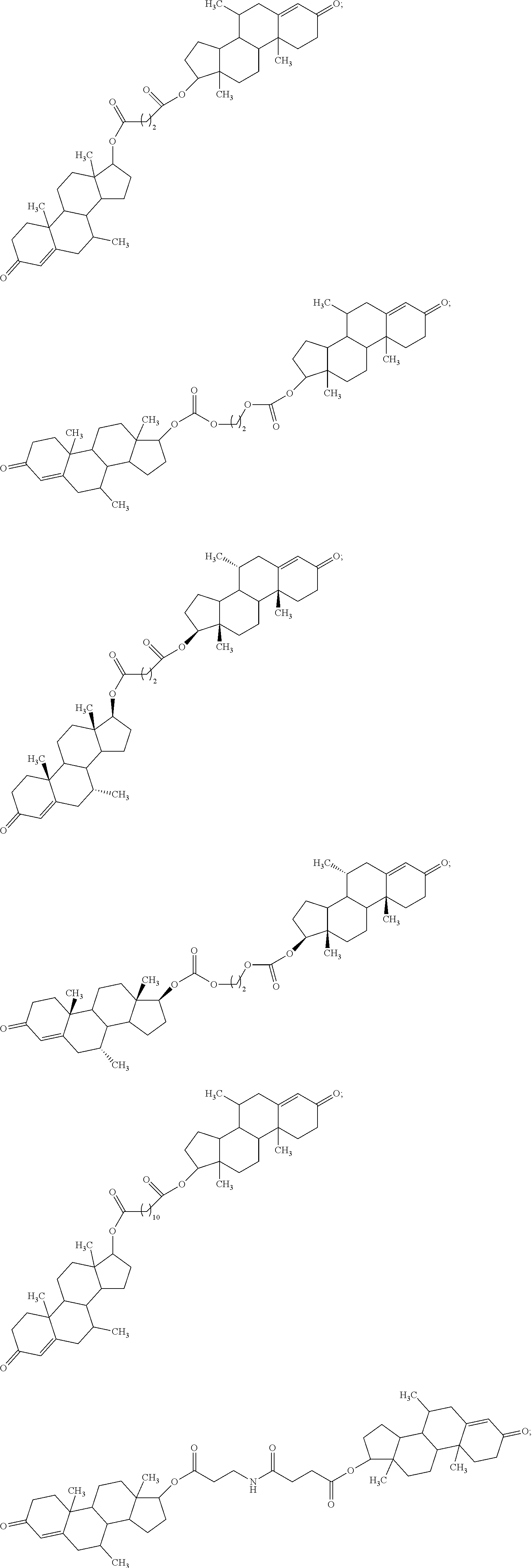 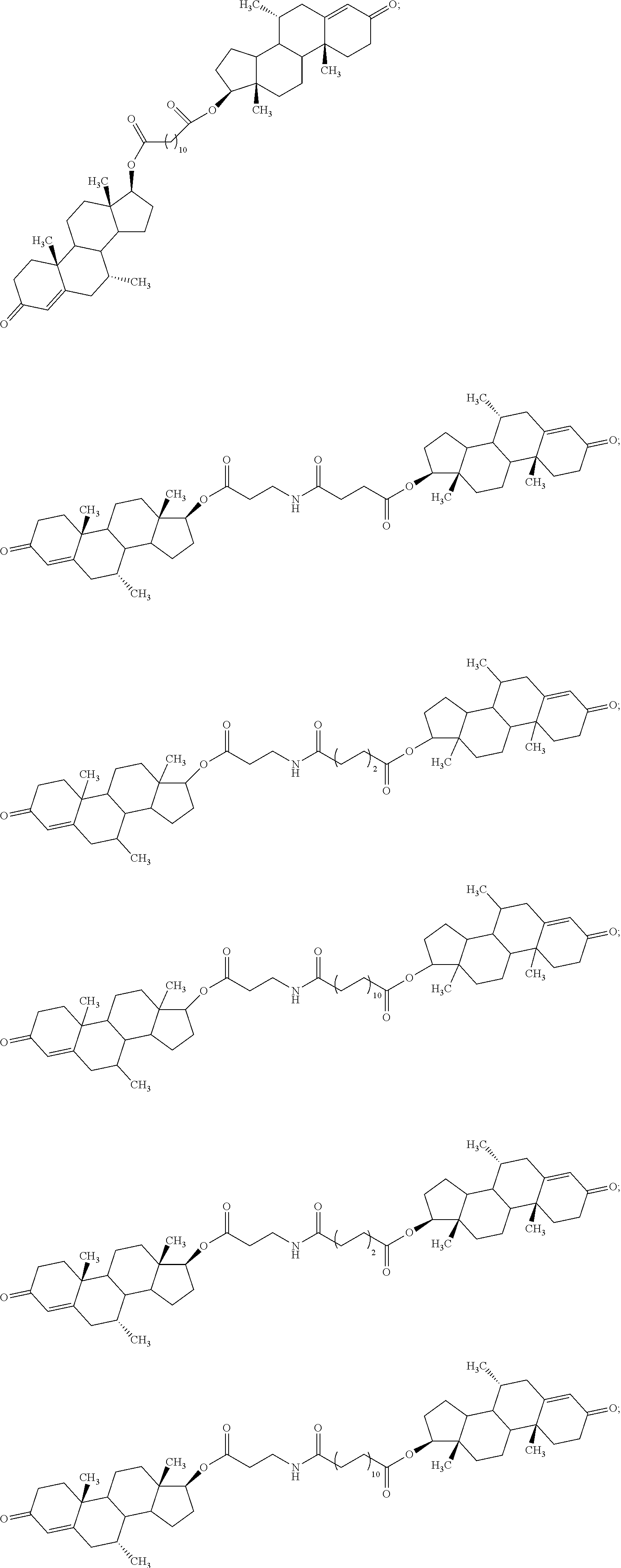 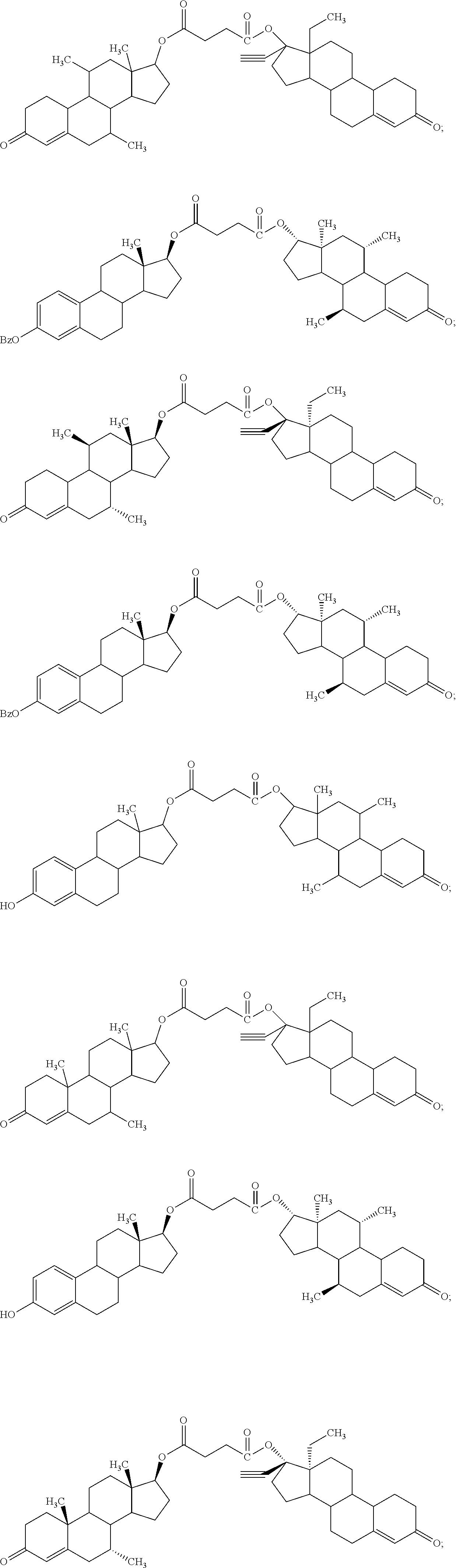 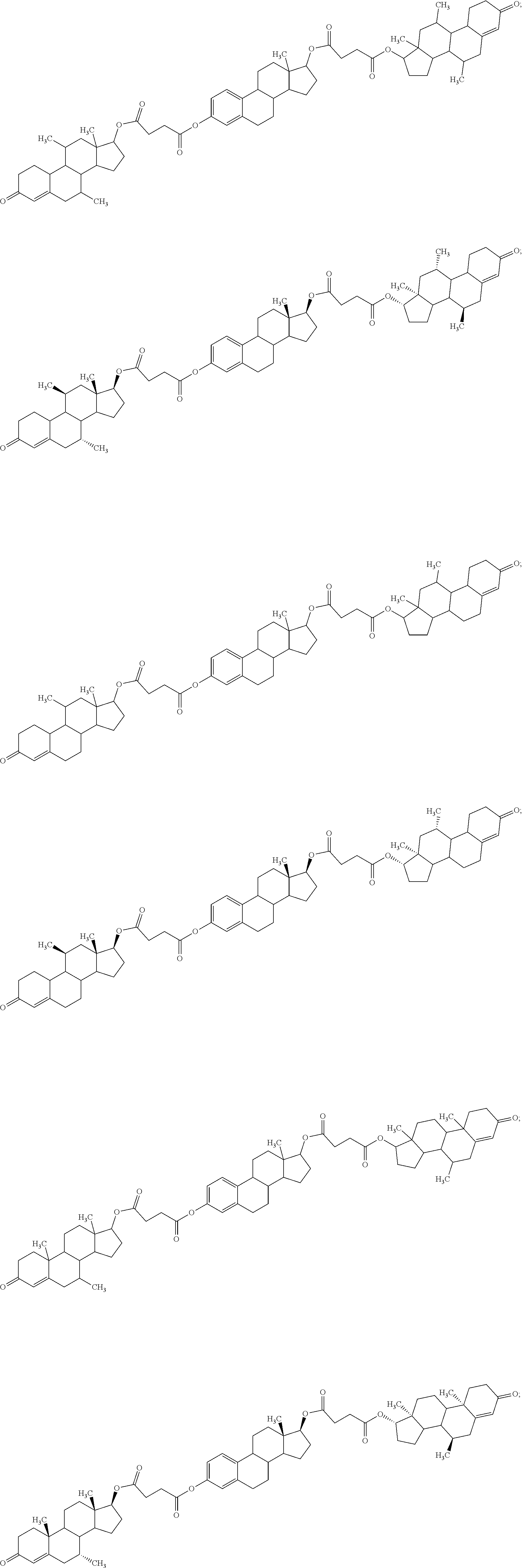 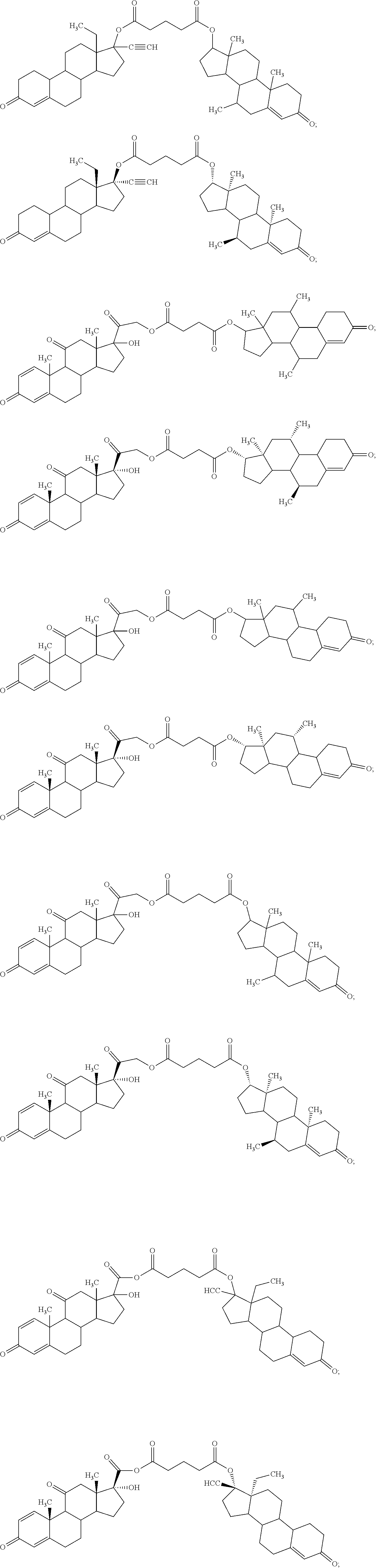 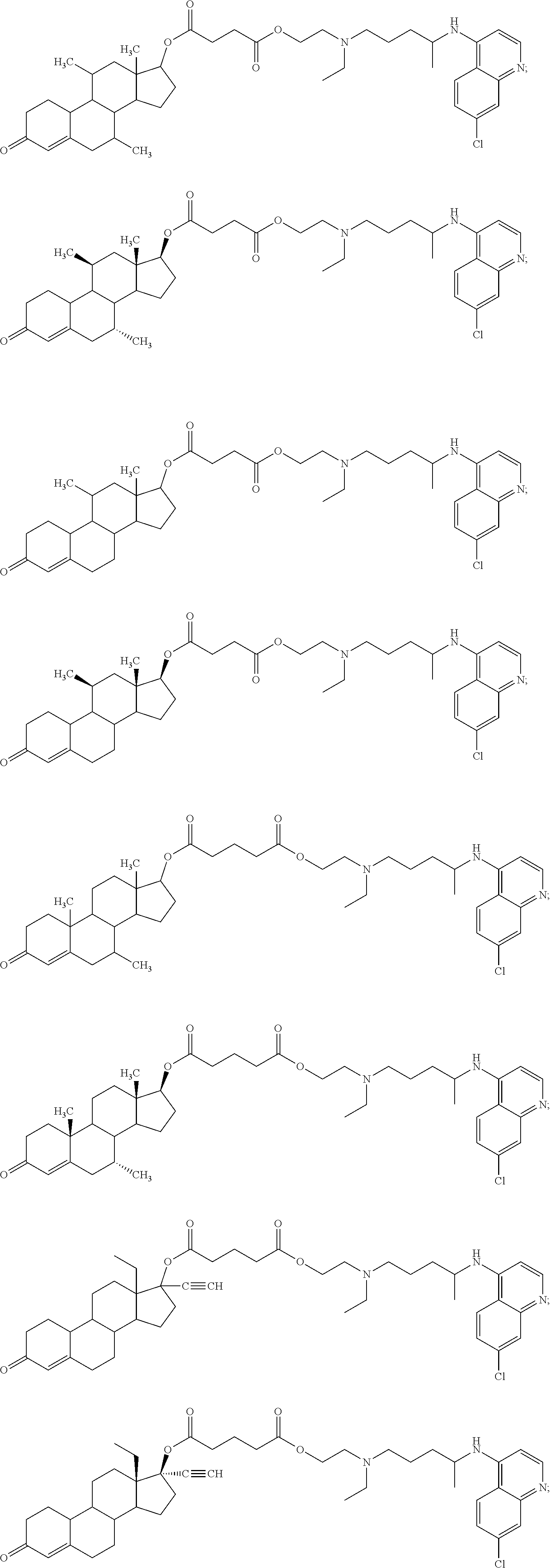 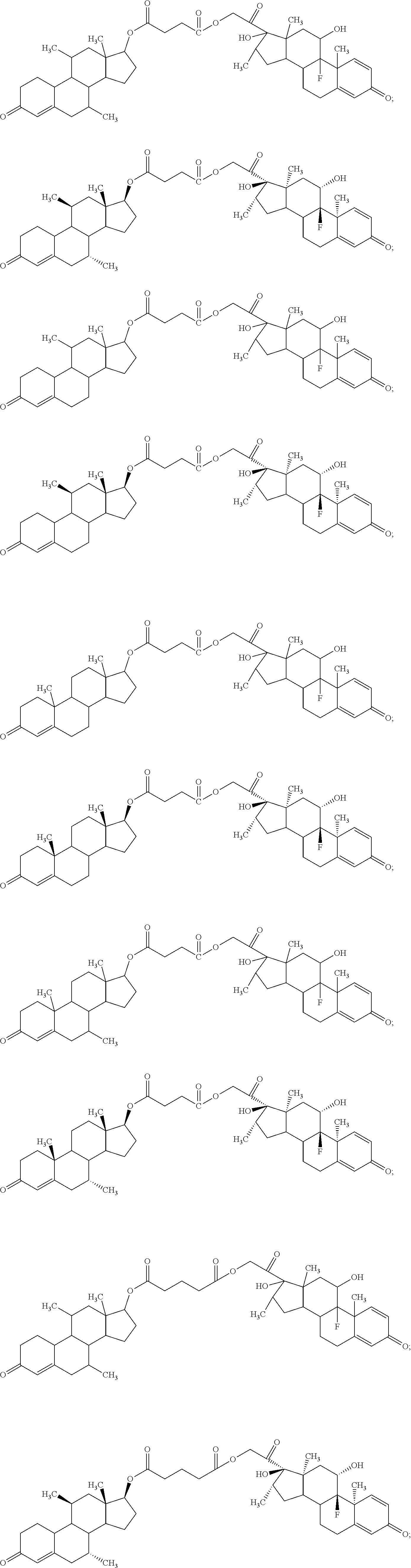 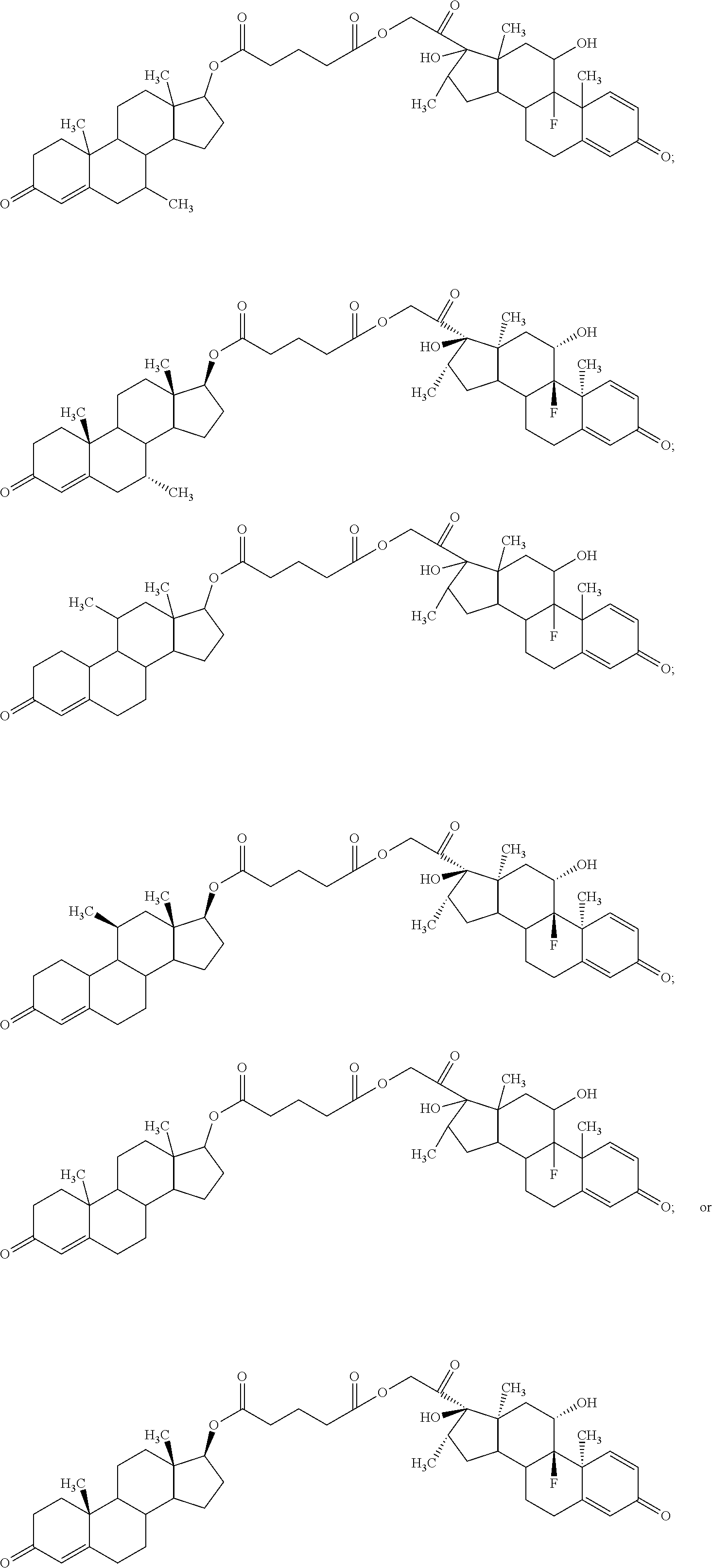
or a pharmaceutically acceptable salt, a prodrug, a solvate, or a tautomer thereof.

28-29. (canceled)

30. A dosage form, comprising the oligomer compound of claim 15, or a pharmaceutically acceptable salt, a prodrug, a solvate, or a tautomer thereof; or a pharmaceutical composition thereof; wherein the dosage form is a tablet, a capsule, an implant, a patch, a microneedle array, an aerosol, or gel.

31. A method, comprising administering to a subject a compound according to claim 1, or a dosage form comprising the compound, for hormonal therapy as male contraception or to treat a disease or disorder selected from cancer, sickle cell anemia, leukemia, an autoimmune disorder, a cardiovascular disease, a fungal disease, a bacterial disease, a viral disease, endometriosis, a metabolic disease, a pulmonary disease, a gastrointestinal disease, a hypogonadism disorder, sarcopenia, muscle atrophy, or any combination thereof.

32. (canceled)

33. A method for making the compound of claim 1, comprising:
performing a conjugate addition and deprotection reaction on a protecting-group containing precursor compound using a lithium compound, a catalyst, a Grignard reagent, and a silyl reagent to provide a substituted, deprotected product; and
functionalizing the substituted, deprotected product to provide the compound; wherein the protecting-group containing precursor compound has a formula 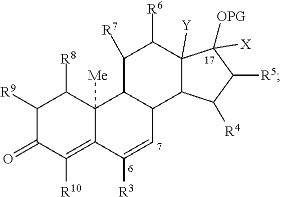
the substituted, deprotected product has a formula

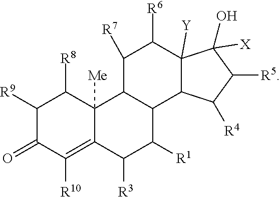

34. (canceled)

35. A method for making the oligomer compound of claim 15, comprising:
covalently coupling a linker group precursor and (i) one of the first steroidal-based compound or the second steroidal-based compound, or (ii) the therapeutic agent using an esterifying reagent to form either a linker-functionalized steroidal-based compound or a linker-functionalized therapeutic agent; and
covalently coupling the linker-functionalized steroidal-based compound to the other of the first or second steroidal-based compound; or
covalently coupling the linker-functionalized therapeutic agent to the first steroidal-based compound.

36. (canceled)

37. A method, comprising administering to a subject an oligomer compound according to claim 15, or a dosage form thereof, for hormonal therapy as male contraception or to treat a disease or disorder selected from cancer, sickle cell anemia, leukemia, an autoimmune disorder, a cardiovascular disease, a fungal disease, a bacterial disease, a viral disease, endometriosis, a metabolic disease, a pulmonary disease, a gastrointestinal disease, a hypogonadism disorder, sarcopenia, muscle atrophy, or any combination thereof.
